(19) United States
(12) Patent Application Publication
(10) Pub. No.: US 2026/0266848 A1
(43) Pub. Date: Sep. 10, 2026

(54) COMPOSITIONS AND METHODS FOR DIAGNOSING AND TREATING DYSTONIA

(71) Applicant: Duke University, Durham, NC (US)

(72) Inventors: Nicole Calakos, Durham, NC (US); Zachary Caffall, Durham, NC (US); Connor King, Durham, NC (US)

(21) Appl. No.: 19/489,163

(22) PCT Filed: May 31, 2024
(86) PCT No.: PCT/US2024/032076

§ 371 Date: Dec. 1, 2025

Related U.S. Application Data
(60) Provisional application No. 63/470,238, filed on Jun. 1, 2023.

(51) Int. Cl. G01N 33/68; A61K 31/202; A61K 31/4453; A61K 31/46; A61K 31/4745; A61K 31/4985; A61K 31/542; A61K 31/5513; A61P 25/08

(52) U.S. Cl. CPC G01N 33/6896; A61K 31/202; A61K 31/4453; A61K 31/46; A61K 31/4745; A61K 31/4985; A61K 31/542; A61K 31/5513; A61P 25/08; G01N 2800/2835; G01N 2800/52; G01N 2800/60

(57) ABSTRACT

Disclosed herein are compositions, kits, and methods for identifying protein, miRNA, or metabolite biomarkers for dystonia, for treating a subject having a dystonia, for selecting a subject for a clinical trial, for selecting a therapeutic agent, for treatment of dystonia in a subject, for classifying a subject having dystonia, and for predicting responsiveness to a dystonia treatment.

### CROSS-REFERENCE TO RELATED APPLICATIONS

This application claims the benefit of priority to U.S. Provisional Patent Application No. 63/470,238 filed Jun. 1, 2023, which is incorporated by reference herein in its entirety.

### GOVERNMENT SUPPORT

This invention was made with government support under W81XWH-19-1-0018 awarded by the Department of Defense. The government has certain rights in the invention.

### SEQUENCE LISTING

This patent or application file contains a sequence listing in Computer Readable Format (CRF), which is incorporated by reference thereto in its entirety. The CRF is entitled “21_2024_WO2_SL.xml”, which was created on May 31, 2024 and is 13,140 bytes in size.

### BACKGROUND

Dystonias are centrally driven movement disorders characterized by sustained involuntary postures and/or slow twisting movements that lead to motor disability and pain. Presentations range from focal dystonias, affecting single limbs or other body parts, to generalized dystonias where most of the body is involved in abnormal posturing and/or slow uncontrolled twisting movements. Once symptoms manifest, they typically endure throughout an individual's lifetime leading to a notable burden of disability and pain. In all its forms, dystonia is the third most common movement disorder after Parkinson's disease and essential tremor and can arise in many clinical settings—from sporadic and inherited forms to those that occur in association with traumatic brain injury, stroke, neurodegenerative diseases, metabolic disorders, or antipsychotic medication use.

The mainstays for oral medication treatment are anticholinergic drugs, benzodiazepines, and muscle relaxants. These medications typically reduce the intensity of, but do not eliminate, dystonia symptoms. The narrow therapeutic window that further limits the utility of these medications. While pallidal deep brain stimulation surgery has been shown to be beneficial for some subsets of patients with dystonia, including those with DYT1 dystonia, this is a highly invasive treatment available only at tertiary care centers. None of these treatments are disease modifying. Thus, there is a major unmet need for the efficient diagnosis of a dystonia and for effective, affordable, and easily accessible dystonia treatments.

### BRIEF SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

One aspect of the present disclosure provides a method of treating dystonia in a subject, comprising administering to the subject one or more therapeutic agents when one or more dystonia biomarkers in the subject is aberrant compared to a reference level.

Another aspect of the present disclosure provides a method of treating dystonia in a subject, comprising administering to the subject an amount of one or more therapeutic agents, wherein the amount of the one or more therapeutic agents is increased or reduced from a preliminary dosage amount when one or more dystonia biomarkers in the subject is aberrant compared to a reference level. The preliminary dosage amount may be from an approved pharmaceutical product label for the therapeutic agent, or the starting dosage for the therapeutic agent in a clinical trial.

Another aspect of the present disclosure provides a method of selecting a subject for a clinical trial, the method comprising measuring one or more dystonia biomarkers in the subject from a biological sample obtained from the subject, and enrolling the subject in the clinical trial when the one or more dystonia biomarkers in the subject is aberrant compared to a reference level, or not enrolling the subject in the clinical trial when the one or more dystonia biomarkers in the subject is not aberrant compared to a reference level.

Another aspect of the present disclosure provides a method of selecting a therapeutic agent for treatment of dystonia in a subject, the method comprising measuring one or more dystonia biomarkers in the subject from a biological sample obtained from the subject, and selecting the therapeutic agent when the one or more dystonia biomarkers in the subject is aberrant compared to a reference level; or not aberrant compared to a reference level.

Another aspect of the present disclosure provides a method of classifying a subject having dystonia, the method comprising measuring one or more dystonia biomarkers in the subject from a biological sample obtained from the subject, and classifying the subject as having the one or more dystonia biomarkers when the one or more dystonia biomarkers in the subject is aberrant compared to a reference level, or not having the one or more dystonia biomarker when the one or more dystonia biomarkers in the subject is not aberrant compared to a reference level.

Another aspect of the present disclosure provides a method of identifying a dystonia biomarker in a subject, the method comprising obtaining a biosample from a subject having a dystonia, obtaining a biosample from a subject not having a dystonia, determining the reference level of one or more proteins, miRNAs, and/or metabolites in the biosample from a subject having a dystonia, and identifying those proteins, miRNAs, and/or metabolites that are aberrant in the biosample obtained from the subject having a dystonia when compared to the biosample from the subject not having a dystonia, wherein those aberrant proteins, miRNAs, and/or metabolites are biomarkers of dystonia.

In another aspect, a method as described herein may further comprising treating a subject having a dystonia, wherein treating the subject comprises administering one or more therapeutic agents, optionally wherein the one or more therapeutic agents modulates the expression level of one or more aberrant dystonia biomarker.

A dystonia biomarker may be selected from a protein, metabolite, or an miRNA. An aberrant dystonia biomarker may be below its reference level, or above its reference level. A dystonia biomarker profile may comprise at least one or more aberrant dystonia biomarkers below its reference level and/or at least one or more aberrant dystonia biomarkers above its reference level. An aberrant biomarker may be at least ±0.5 fold, at least ±1.0 fold, at least ±1.5 fold, at least ±2.0 fold, at least ±3.0 fold, or at least ±4.0 fold different relative to a reference level. The reference level of the one or more dystonia biomarkers may be determined from a subject or group of subjects, each of which does not have dystonia. The reference level of the one or more dystonia biomarkers may be a normal or desired value known in the art.

Determining the level of one or more proteins or metabolites in a biosample as a dystonia biomarker may comprise using liquid chromatography with tandem mass spectrometry (LC-MS-MS), parallel reaction monitoring (PRM), or multiple reaction monitoring (MRM). Determining the level of one or more miRNAs in a biosample as a dystonia biomarker may comprise using RNASeq or RT-qPCR. The biosample may comprise extracellular vesicle (EVs) or whole plasma.

A dystonia biomarkers comprise may be selected from a biomarker as provided in any of TABLES 2-7 and S1-S8.

A dystonia biomarker may be a protein selected from List-A1, List-A2, List-A3, List-A4, List-B1, List-B2, List-B3, List-C1, List-C2, List-C3, List-D1, List-D2, List-D3, List-E1, List-E2, List-E3, List-F1, List-F2, List-F3, List-G1, List-G2, List-G3, TABLE 4, TABLE 5, TABLE 6, TABLE 7, TABLE 8, TABLE 9, TABLE S1, TABLE S3, TABLE S4, TABLE S5, TABLE S6, TABLE S7, or a combination thereof.

In one aspect, a dystonia biomarker is selected from an miRNA molecule selected from List-MR1, TABLE 2, TABLE 3, or a combination thereof.

In one aspect, a dystonia biomarker is selected from an metabolite molecule selected from List-ML1, or a combination thereof.

The dystonia may be selected from focal dystonia, blepharospasm, cervical dystonia, oromandibular dystonia, task-specific or occupational dystonia, spasmodic dysphonia, generalized dystonia, segmental dystonia, DYT1-related dystonia, DYT6-related dystonia, DYT28-related dystonia, dopa-responsive dystonia, myoclonic dystonia, X-linked dystonia-Parkinsonism, rapid-onset dystonia-Parkinsonism, paroxysmal dystonia choreoathetosis, paroxysmal kinesigenic dystonia, paroxysmal nonkinesigenic dyskinesia, paroxysmal exertion-induced dyskinesia, primary dystonia, acquired dystonia, tardive dyskinesia, or tardive dystonia.

Another aspect of the present disclosure provides a pharmaceutical composition for treatment of a dystonia in a subject in need thereof, comprising one or more therapeutic agent and a pharmaceutically acceptable carrier, wherein the treatment comprises administering the pharmaceutical composition to the subject when one or more dystonia biomarkers in the subject is aberrant compared to a reference level.

The one or more therapeutic agent may treat, reduce, ameliorate, prevent, or inhibit a symptom of a neurological disorder. The therapeutic agent may be an anticholinergic, benzodiazepine, dopaminergic agent, or a dopamine-depleting agent. The therapeutic agent may be any agent as described herein. The therapeutic agent may be ritonavir or salurbinal

### BRIEF DESCRIPTION OF THE DRAWINGS

The patent or application file contains at least one drawing executed in color. Copies of this patent or patent application with color drawing(s) will be provided by the Office by request and payment of the necessary fee.

The accompanying Figures and Examples are provided by way of illustration and not by way of limitation. The foregoing aspects and other features of the disclosure are explained in the following description, taken in connection with the accompanying example figures (also “FIG.”) relating to one or more embodiments, in which:

FIGS. 1A-1F depict quantitative proteomics showing DYT-TOR1A-dependent changes in protein composition of EVs isolated from murine embryonic fibroblasts. FIG. 1A depicts an experimental workflow schematic. FIG. 1B shows total protein in EV samples quantified by BCA, where 1, 2, and 3 indicate specific biological replicates. FIG. 1C shows quantification of TSG101 from western blot in FIG. 1D, normalized to the mean WT abundance, where 1, 2, and 3 indicate specific biological replicates. FIG. 1D shown western blot of whole cell lysates (4 g protein) and EV samples (5% of total EV sample by volume) for TSG101 (EV marker), calnexin (ER marker), and actin. See FIG. 4 for complete blot images. FIG. 1E shows mass spectrometry protein abundances (Prot. Abund.) of EV markers normalized to WT mean abundance, where 1, 2, and 3 indicate specific biological replicates. Significance testing in FIGS. 1B, 1C, and 1E performed by unpaired Student's t tests. FIG. 1F depicts a volcano plot showing genotype effects on EV proteome, comparing protein abundances of 1974 detected EV proteins between DYT-TOR1A and WT. Symbols of data points indicate proteins significantly (p<0.05) more (top right dots) or less (top left dots) abundant in DYT-TOR1A relative to WT. Lower horizontal dashed line indicates p-value of 0.05 threshold. Upper gray horizontal dashed line indicates Bonferroni-adjusted p-value threshold of 2.5e-5. Uncorrelated P of 0.05 threshold used to call genotype effects. All experiments reported were performed and initially analyzed blind to experimental variables. Differences in abundance are represented as fold change (using log 2 transformation) and p-value is calculated by unpaired t-test for each protein (n=3 biological replicates).

FIGS. 2A-2C show effects of ritonavir treatment on protein composition of EVs isolated from DYT-TOR1A MEF cultures. FIG. 2A depicts a volcano plot comparing protein abundances (Prot. Abund.) between EVs isolated from DYT-TOR1A MEF cultures treated with ritonavir (DYT+RTV) vs. vehicle (DYT) for 24 h. For FIGS. 2A and 2B, differences in protein abundance are represented as fold change (using log 2 transform) and p-value is calculated by unpaired t-test for each protein (n=3 biological replicates). Horizontal dashed line indicates uncorrected p-value of 0.05. Color-coded data points indicate the original genotype disrupted proteins and genotype-directionality from FIG. 1F with coloring showing the protein's genotype effect (red being increased and blue being decreased in DYT/WT). Corrective directionality changes where observed for the majority of proteins. Inset shows genotype results from FIG. 1F. FIG. 2B shows comparison of genotype (DYT/WT) and ritonavir (DYT+RTV/DYT) effects on protein abundances (log 2 transformed). Ritonavir shows proteome-wide corrective effects. The highlighted data points identify original genotype effect and directionality (of FIG. 1F). FIG. 2C depicts an unsupervised hierarchical clustering heatmap of WT, DYT, and DYT+RTV protein abundances for proteins significantly different in DYT relative to WT (n=363). The clustering segregates DYT1 from WT and ritonavir-treated DYT1 samples. Z scores for 363 genotype-disrupted proteins shown for all samples.

FIGS. 3A-3C show effects of ISR tool compounds on MEF EV protein composition. FIG. 3A depicts a volcano plot showing ISRIB effects on protein abundances in WT MEFs. For FIGS. 3A-3D, highlighted data points indicate proteins significantly increased or decreased in DYT-TOR1A MEF EVs compared to WT experiment shown in FIG. 1F. Differences in protein abundance are represented as fold change (using log 2 transform) and p-value is calculated by unpaired t-test for each protein (n=3 biological replicates). Horizontal dashed line indicates p-value of 0.05. FIG. 3B shows correlation of protein abundance fold changes between DYT genotype effects (DYT/WT) and ISRIB effects (WT+ISRIB/WT). FIG. 3C depicts a volcano plot showing the effect of salubrinal treatment of DYT MEFs (DYT+SAL) on EV protein abundances compared to vehicle control (DYT). FIG. 3D shows correlation between ritonavir and salubrinal treatment effects on protein abundances in DYT MEF EVs.

FIG. 4 shows non-cropped western blots for samples in FIG. 1D. Blinded samples were loaded onto gels by EV preparation batches. Unconditioned media (UCM) is the ‘pellet’ material recovered from ultracentrifugation of 1% dEV FBS unconditioned media as a negative control processed in parallel with conditioned media EV samples. Blots were probed for TSG101 (EV marker), calnexin (ER marker), and actin. Rows 2-4 show the same blots probed for both mouse anti-actin and rabbit anti-TSG101. Row 4 is shown with both red (700 nm) and green (800 nm) channels to demonstrate specificity of multiplexed primary and secondary antibodies. “n.s.” indicates non-specific bands.

FIGS. 5A-5B show presence of imputed values in LC-MS/MS raw abundance data from FIG. 1F. EV proteins from vehicle-treated WT or DYT EV samples are grouped by number of missing (and imputed) values prior to normalization for all data (n=1974) (FIG. 5A), or the subset of DYT genotype-dependent hits (n=363) (FIG. 5B). (see Methods for imputation paradigm). “0 imputed values” indicates proteins were detected in all WT or DYT samples. 1, 2, or 3 imputed values show proteins not detected in the respective number of samples in each condition.

FIGS. 6A-6C depict small RNAseq analysis of the RNA isolated from the DYT1 vs WT MEF secreted EVs also show genotype and ritonavir pharmacodynamic effects. FIG. 6A shows genotype effects on EV miRNAs. N=3 independent culture preparations per genotype. Same cultures used in proteomic experiments. EV fraction was divided and separately analyzed for proteins and RNAs. Uncorrelated P of 0.05 threshold used to call genotype effects. FIG. 6B shows Ritronavir effects on DYT1 EV miRNA disruptions. 24 hour culture exposure. Genotype-disrupted miRNAs and genotype-directionality as identified in panel E are indicated by blue and red dots. FIG. 6C shows Ritonavir demonstrates stronger correlation of corrective effects on genotype-disrupted subset of miRNAs. Highlighted data points identify original genotype effect and directionality (in FIG. 6A). 14 miRNAs surpass FDR-corrected p-value thresholds. Genotype Z scores range from −4.99 to 4.11

FIG. 7 depicts a heat map showing Z scores for the genotype disrupted mRNAs.

FIG. 8 depicts a volcano plot showing differentially expressed miRNAs in DYT1 patient whole plasma as compared to Healthy Control plasma. Plasma from 13 DYT1 patients and healthy controls identified candidate human miRNA markers.

FIG. 9A depicts cross-species proteomic overlap between the human plasma proteome and mouse EV proteome. FIG. 9B shows the 147 proteins in the MEF data hits that have homolog detected in human total plasma proteomics.

FIG. 10 depicts a human plasma pilot study, where plasma from 13 DYT1 patients and healthy controls identified candidate human miRNA markers.

FIGS. 11A and 11B show subject level data examples for each of an up-regulated and down-regulated miRNA (miRNAs hsa-let-7g-5p and hsa-mIR-23-5p, respectively).

FIG. 12 shows subject level data for each patient/control for the 14 miRNA significantly differentially abundant in whole plasma following FDR correction.

FIG. 13 depicts an unsupervised hierarchical heat map clustering (utilizing the FDR hits), which show separation of DYT1 from controls.

FIG. 14 shows cross-species comparison of DYT-1 genotype effects on miRNAs with sequence homology (241 miRNAs).

FIG. 15 depicts a volcano plot showing differentially abundant metabolites in DYT1 patient whole plasma as compared to Healthy Control plasma.

FIG. 16 depicts shows electrophoresis of extracellular vesicles isolated from human plasma using ExoQuick ULTRA EV isolation system, which were enriched in exosome marker CD63 and depleted of plasma protein albumin; six separate plasma sample enrichments are depicted.

FIG. 17A depicts a volcano plot showing differentially expressed proteins (DEPs) in DYT1 patient plasma derived EVs as compared to Healthy Controls. Four proteins were significantly differential after FDR correction, which are marked (e.g., ADAP2; PTMA; PRSS1; SLC4A1). FIG. 17B shows subject level data for each patient/control for the 4 DEP significant following FDR correction

FIG. 18A depicts cross-species proteomic overlap between the human plasma proteome and mouse EV proteome. 863 proteins in MEF data hits were found to have a homolog detected in human plasma derived EV proteomics. FIGS. 18B-18C show directionality of the human-fold change DYT1/HC between and DYT1/WT. Seven proteins identified were significantly different in both mouse and human EVs compared to WT (HC) and in the same direction (lower)

FIG. 19A depicts a volcano plot of human plasma EV protein. Eight proteins were significant and common in both murine and human. FIG. 19B shows subject level data for each patient/control for the eight common proteins.

FIG. 20 depicts a volcano plot of human plasma EV proteins that were significantly different by DYT1 genotype (p<0.05) and whose levels are regulated in a similar direction in mouse DYT1 EV experiment (MEF) are labeled.

FIG. 21 depicts a volcano plot of human plasma EV proteins that were significantly different by DYT1 genotype, and whose levels are modulated by ritonavir in a corrective direction in mouse DYT1 EV experiment (MEF) (labeled).

FIG. 22 depicts a volcano plot of human plasma EV proteins that were significantly different by DYT1 genotype, and whose levels are modulated by salubrinal in a corrective direction in mouse DYT1 EV experiment (MEF) (labeled).

FIG. 23 depicts a volcano plot of human data highlighting homologs of EV proteins made disease-like in WT by ISRIB treatment in DYT1 MEF EVs.

FIG. 24 shows a graphical representation of the human study design for identification and use of protein, miRNA, and metabolite dystonia biomarkers.

### DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

For the purposes of promoting an understanding of the principles of the present disclosure, reference are made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

### A. Definitions

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 10% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The term “biological sample” or “biosample” are used interchangeably herein and include, but are not limited to, a sample containing tissues, cells, and/or biological fluids isolated from a subject. Examples of biological samples include, but are not limited to, tissues, cells, biopsies, blood, lymph, serum, plasma, urine, saliva, mucus and tears. A biological sample may be obtained directly from a subject (e.g., by blood or tissue sampling) or from a third party (e.g., received from an intermediary, such as a healthcare provider or lab technician).

As used herein, the term “diagnosed” means having been subjected to an examination by a person of skill, for example, a physician, and found to have a condition that can be diagnosed or treated by one or more of the disclosed agents, disclosed therapeutic agents, disclosed pharmaceutical formulations, or a combination thereof, or by one or more of the disclosed methods. For example, “diagnosed with a dystonia” means having been subjected to an examination by a person of skill, for example, a physician, and found to have a condition that can be treated by one or more of the disclosed agents, disclosed therapeutic agents, disclosed pharmaceutical formulations or a combination thereof, or by one or more of the disclosed methods. For example, “suspected of having a dystonia” can mean having been subjected to an examination by a person of skill, for example, a physician, and found to have a condition that can likely be treated by one or more of the disclosed agents, disclosed therapeutic agents, disclosed pharmaceutical formulations or a combination thereof, or by one or more of the disclosed methods. In an aspect, an examination can be physical, can involve various tests (e.g., blood tests, genotyping, biopsies, etc.) and assays (e.g., enzymatic assay), or a combination thereof.

A “patient” can refer to a subject that has been diagnosed with or is suspected of having a dystonia. In an aspect, a patient can refer to a subject that has been diagnosed with or is suspected of having a dystonia such as for example, DYT1, and is seeking treatment or receiving treatment for a dystonia (such as DYT1).

As used herein, the phrase “identified to be in need of treatment for a disorder,” or the like, refers to selection of a subject based upon need for treatment of the disorder. For example, a subject can be identified as having a need for treatment of a disorder (e.g., such as a dystonia) based upon an earlier diagnosis by a person of skill and thereafter subjected to treatment for the disorder (e.g., a dystonia). In an aspect, the identification can be performed by a person different from the person making the diagnosis. In an aspect, the administration can be performed by one who performed the diagnosis.

The term “disease” as used herein includes, but is not limited to, any abnormal condition and/or disorder of a structure or a function that affects a part of an organism. It may be caused by an external factor, such as an infectious disease, or by internal dysfunctions, such as cancer, cancer metastasis, and the like.

As used herein, the terms “neurological diseases” or “neurological disorders” are used interchangeably and refer to a host of undesirable conditions affecting neurons in the brain of a subject. These diseases include but are not limited to the following: Alzheimer's disease, Parkinson's disease, Huntington's disease, Pick's disease, Kufs disease, Lev-.y body disease, neurofibrillary tangles, Rosenthal fibers, Mallory's hyaline, senile dementia, myasthenia gravis, Gilles de la Tourette's syndrome, multiple sclerosis (MS), amyotrophic lateral sclerosis (ALS), progressive supranuclear palsy (PSP), epilepsy, Creutzfeldt-Jakob disease, deafness-dystonia syndrome, Leigh syndrome, Leber hereditary optic neuropathy (LHON), parkinsonism, dystonia, motor neuron disease, neuropathy-ataxia and retinitis pimentosa (NARP), maternal inherited Leigh syndrome (MILS), Friedreich ataxia, hereditary spastic paraplegia, Mohr-Tranebjaerg syndrome, Wilson disease, sporadic Alzheimer's disease, sporadic amyotrophic lateral sclerosis, sporadic Parkinson's disease, autonomic function disorders, hypertension, sleep disorders, neuropsychiatric disorders, depression, schizophrenia, schizoaffective disorder, Korsakoff s psychosis, mania, anxiety disorders, phobic disorder, learning or memory disorders, amnesia or age-related memory loss, attention deficit disorder, dysthymic disorder, major depressive disorder, obsessive-compulsive disorder, psychoactive substance use disorders, panic disorder, bipolar affective disorder, severe bipolar affective {mood) disorder (BP-1), migraines, hyperactivity and movement disorders.

As used herein, the term “movement disorder” includes neurological diseases or disorders that involve the motor and movement systems, resulting in a range of abnormalities that affect the speed, quality, and ease of movement disorders are often caused by or related to abnormalities in brain structure and/or function. Movement disorders include, but are not limited to (i) tremors: including, but not limited to, the tremor associated with Parkinson's Disease, physiologic tremor, benign familial tremor, cerebellar tremor, rubral tremor, toxic tremor, metabolic tremor, and senile tremor; (ii) chorea, including, but not limited to, chorea associated with Huntington's Disease, Wilson's Disease, ataxia telangiectasia, infection, drug ingestion, or metabolic, vascular or endocrine etiology (e.g., chorea gravidarum or thyrotoxicosis); (iii) ballism (defined herein as abruptly beginning, repetitive, wide, flinging movements affecting predominantly the proximal limb and girdle muscles); (iv) athetosis (defined herein as relatively slow, twisting, writhing, snake-like movements and postures involving the trunk, neck, face and extremities); (v) dystonia (defined herein as a movement disorder consisting of twisting, turning tonic skeletal muscle contractions, most, but not all of which are initiated distally); (vi) paroxysmal choreoathetosis and tonic spasm; (vii) tics (defined herein as sudden, behaviorally related, irregular, stereotyped, repetitive movements of variable complexity); (viii) tardive dyskinesia; (ix) akathisia, (x) muscle rigidity, defined herein as resistance of a muscle to stretch; (xi) postural instability; (xii) bradykinesia; (xiii) difficulty in initiating movements; (xiv) muscle cramps; (xv) dyskinesias and (xvi) myoclonus. In an aspect, a movement disorder comprises a dystonia. As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e. living organism, such as a patient).

Unless otherwise defined, all technical terms used herein have the same meaning as commonly understood by one of ordinary skill in the art to which this disclosure belongs.

In an aspect, any disclosed method can be applied in the context of one or more neurological diseases or disorders. For example, a disclosed method of identifying a dystonia biomarker can be applied instead with a subject having Alzheimer's disease, depression, or anxiety, or any other disclosed neurological disease or disorder. The same applies to the other methods disclosed herein, including but not limited to, methods of treating a subject having a neurological disease or disorder, methods of predicting penetrance and/or severity of a neurological disease or disorder, and/or methods of predicting responsiveness to a treatment.

As used herein, the term “agent which prevents or reduces symptoms of the neurological disorder” or “agent used for the treatment of a neurological disorder” refers to those drugs that are used for the treatment of one or more of the disclosed neurological diseases and disorders. Examples of such agents include, but are not limited to, the following: anticholinergics, such as trihexyphenidyl (Artane®), benztropine (Cogentin®), ethopropazine (Parsitan®); benzodiazepines, such as diazepam (Valium®), clonazepam (Klonopin®), lorazepam (Ativan®); baclofen (Lioresal®), dopaminergic agents such as levodopa (Sincmet®) and bromocriptine (Parlodel®); tetrabenazine (Xenazine®), dopamine-depleting agents, ritonavir, lopinavir, and the like. In an aspect, the agent comprises ritonavir.

Other agents that treat, prevent, inhibit, and/or ameliorate symptoms and/or complications of a neurological disorder and/ora neurodegenerative disease include the following: Acamprosate tablets (Campral EC), Adrenaline (epinephrine) (Emerade, EpiPen, Jext), Agomelatine tablets (Valdoxan), Almotriptan (Almogran), Amantadine, Amisulpride (Solian), Amitriptyline (Elavil), Apomorphine (APO-go, Dacepton), Aripiprazole (Abilify), Aripiprazole long-acting injection (Ability Maintena), Asenapine tablets (Sycrest), Atomoxetine (Strattera), Baclofen (Lyflex, Lioresal), Botulinum toxin type A (Botox), Bromocriptine (Parlodel), Buccal midazolam (Buccolam, Epistatus), Buprenorphine (BuTrans, Hapoctasin, Temgesic, Tephine, Transtec), Buspirone, Cabergoline tablets (Dostinex, Cabaser), Carbamazepine (Curatil, Tegretol), Chlordiazepoxide (Librium), Chlorpromazine, Citalopram (Cipramil, Celexa), Clobazam (Frisium, Perizam, Tapclob, Zacco), Clomethiazole, Clomipramine, Clonazepam, Clozapine (Clozaril, Denzapine, Zaponex), Co-beneldopa (Madopar), Co-careldopa (Sinemet), Dantrolene (Dantrium), Dexamfetamine (Amfexa), Diazepam (Diazemuls, Stesolid), Divalproex sodium (Depakote), Donepezil (Aricept), Doxepin capsules, Duloxetine (Cymbalta, Depalta, Duciltia), Eletriptan (Relpax), Entacapone (Comtess), Escitalopram (Cipralex), Eslicarbazepine (Zebinix), Ethosuximide, Fingolimod capsules (Gilenya), Fluoxetine (Olena, Prozac, Prozep), Flupentixol long-acting injection (Depixol, Psytixol), Flupentixol tablets (Depixol, Fluanxol), Fluphenazine long-acting injection (Modecate), Fluvoxamine tablets (Faverin), Frovatriptan for migraine (Migard), Gabapentin (Neurontin), Galantamine (Acumor, Consion, Elmino, Gaalin, Galsya, Galzemic, Gatalin, Gazylan, Lotprosin, Luventa, Reminyl), Haloperidol (Haldol, Serenace), Haloperidol long-acting injection (Haldol Decanoate), Hydromorphone (Palladone), Imipramine tablets and liquid medicine, Lacosamide (Vimpat), Lamotrigine (Lamictal), Levetiracetam for epilepsy (Keppra, Desitrend), Levomepromazine tablets (Nozinan), Lisdexamfetamine (Elvanse), Lithium tablets and liquid medicine (Camcolit, Liskonum, Priadel, Li-Liquid), Lofepramine, Loprazolam, Lorazepam, Lormetazepam tablets, Lurasidone (Latuda), Melatonin tablets (Circadin, Slenyto), Memantine (Ebixa, Nemtadine), Methylphenidate (Concerta, Equasym, Medikinet, Ritalin, Tranquilyn), Mianserin, Midodrine (Bramox), Mirtazapine (Zispin SolTab), Moclobemide (Manerix), Modafinil tablets (Provigil), Morphine (Morphgesic, Oramorph, Zomorph), Naratriptan (Naramig), Neostigmine, Nitrazepam (Mogadon), Nortriptyline tables, Olanzapine (Zalasta, Zyprexa), Olanzapine long-acting injection (Zypadhera), Orlistat capsules (Alii, Beacita, Orlos, Xenical), Orphenadrine, Oxazepam, Oxcarbazepine (Trileptal), Oxycodone (Abtard, Longtec, OxyContin, OxyNorm, Shortec), Paliperidone (Invega), Paliperidone long-acting injection (Xeplion, Trevicta), Paroxetine (Seroxat), Perampanel (Fycompa), Pergolide, Pericyazine, Phenobarbital, Phenytoin (Epanutin), Piracetam (Nootropil), Pizotifen tablets, Pramipexole tablets (Mirapexin, Oprymea, Pipexus, Glepark), Pregabalin (Alzain, Axalid, Lecaent, Lyrica), Primidone, Prochlorperazine (Buccastem, Stemetil), Procyclidine (Kemadrin), Pyridostigmine (Mestinon), Quetiapine (Seroquel), Rasagiline (Azilect), Reboxetine tablets (Edronax), Risperidone (Risperdal), Risperidone long-acting injection (Risperdal Consta), Rivastigmine (Alzest, Exelon, Nimvastid), Rizatriptan for migraine (Maxalt), Ropinirole tablets (Requip, Adartrel), Rotigotine patches (Neupro), Rufmamide for epilepsy (Inovelon), Selegiline (Eldepryl), Sertraline (Lustral, Zoloft), Sodium oxybate (Xyrem), Sodium valproate (Epilim, Episenta, Epival, Convulex), Sulpiride, Sumatriptan (Imigran), Temazepam, Tetrabenazine tablets (Tardiben, Xenazine), Tiagabine (Gabitril), Tizanidine, Tolcapone (Tasmar), Topiramate (Topamax), Topiramate (Topamax), Trazodone (Molipaxin), Trihexyphenidyl, Trimipramine, Valproate semisodium (Belvo, Depakote, Syonell), Venlafaxine (Efexor XL, Effexor XR), Vigabatrin (Sabril, Kigabeq), Vortioxetine (Brintellix), Zolmitriptan (Zomig), Zolpidem tablets (Stilnoct), Zonisamide (Zonegran, Desizon), Zopiclone tablets (Zimovane), and Zuclopenthixol (Clopixol). In an aspect, any one or combination of these agents can be a therapeutic agent used in a disclosed method.

As used herein, “inhibit,” “inhibiting”, and “inhibition” mean to diminish or decrease an activity, level, response, condition, severity, disease, or other biological parameter. This can include, but is not limited to, the complete ablation of the activity, level, response, condition, severity, disease, or other biological parameter. This can also include, for example, a 10% inhibition or reduction in the activity, level, response, condition, severity, disease, or other biological parameter as compared to the native or control level (e.g., a subject not having a dystonia). Thus, in an aspect, the inhibition or reduction can be a 10%, 20%, 30%, 40%, 50%, 60%, 70%, 80%, 90%, 100%, or any amount of reduction in between as compared to native or control levels. In an aspect, the inhibition or reduction can be 10-20%, 20-30%, 30-40%, 40-50%, 50-60%, 60-70%, 70-80%, 80-90%, or 90-100% as compared to native or control levels. In an aspect, the inhibition or reduction can be 0-25%, 25-50%, 50-75%, or 75-100% as compared to native or control levels. In an aspect, a native or control level can be a pre-disease or pre-disorder level.

The words “treat” or “treating” or “treatment” include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder (such as a dystonia). In an aspect, the terms cover any treatment of a subject, including a mammal (e.g., a human), and includes: (i) preventing the undesired physiological change, disease, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating a dystonia (such as DYT1) can reduce the severity of an established dystonia in a subject by 1%-100% as compared to a control (such as, for example, an individual not having a dystonia). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of a dystonia. For example, treating a dystonia can reduce one or more symptoms of a dystonia in a subject by 1%-100% as compared to a control (such as, for example, an individual not having a dystonia). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established dystonia (such as DYT1). It is understood that treatment does not necessarily refer to a cure or complete ablation or eradication of a dystonia. However, in an aspect, treatment can refer to a cure or complete ablation or eradication of a dystonia.

As used herein, a “biomarker” refers to a defined characteristic that is measured as an indicator of normal biological processes, pathogenic processes, or response to an exposure of intervention. In an aspect, a biomarker can be diagnostic (i.e., detects or classifies a pathological condition), prognostic (i.e., predicts the probability of disease occurrence or progression), pharmacodynamic/responsive (i.e., identifies a change in response to a therapeutic intervention), predictive (i.e., predicts how an individual or subject might respond to a particular intervention or event). In an aspect, a biomarker can be diagnostic, prognostic, pharmacodynamic/responsive, and/or predictive at the same time. In an aspect, a biomarker can be diagnostic, prognostic, pharmacodynamic/responsive, and/or predictive at different times (e.g., first a biomarker can be diagnostic and then later, the same biomarker can be prognostic, pharmacodynamic/responsive, and/or predictive). A biomarker can be an objective measure that can be linked to a clinical outcome assessment. A biomarker can be used by the skilled person to make a clinical decision based on its context of use.

A “diagnostic biomarker” refers to a biomarker that distinguishes between subjects with a particular disease/ailment/condition and those who do not have the disease/ailment/condition.

A “prognostic biomarker” provides information on the likely course of disease/ailment/condition in an individual. A prognostic biomarker can inform the skilled person about the aggressiveness of the disease/ailment/condition and/or the expectation of how a particular subject would fare in the absence of therapeutic intervention. Typically, a prognostic biomarker can identify a patient who is probabilistically at either higher risk for adverse disease-related events or a faster rate of decline in his health status.

A “predictive biomarker” is linked to treatment it provides a forecast of the potential for a subject to respond in some identified manner (which may be favorable or unfavorable) to one or more specific treatments.

A “response biomarker” is a dynamic assessment that shows a biological response has occurred in a subject after having received a therapeutic intervention.

Parallel reaction monitoring (PRM) is an ion monitoring technique based on high-resolution and high-precision mass spectrometry. The principle of this technique is comparable to selected reaction monitoring (SRM/MRM), but it is more convenient in assay development for absolute quantification of proteins and peptides. It is most suitable for quantification of multiple proteins in complex sample with an attomole-level detection. Parallel reaction monitoring (PRM) is an increasingly popular alternative to SRM for targeted proteomics. PRM's strengths over SRM are that it monitors all product ions in a single spectrum, thus eliminating the need to select interference-free product ions prior to data acquisition, and that it is most frequently performed on high-resolution instruments, such as quadrupole-orbitrap and quadrupole-time of flight instruments.

As used herein, the term means “increased risk” is used to mean that a subject has an increased chance of developing or acquiring a dystonia when compared a subject known not to have a dystonia (e.g., a control subject). The increased risk may be relative or absolute and may be expressed qualitatively or quantitatively. For example, an increased risk can be expressed as simply determining a subject's proteomic and/or miRNA profile and placing the subject in an “increased risk” category, based upon previous population studies. Alternatively, a numerical expression of the subject's increased risk can be determined based upon the proteomic and/or miRNA profile. As used herein, examples of expressions of an increased risk of developing or acquiring a dystonia can include but are not limited to, odds, probability, odds ratio, p-values, attributable risk, relative frequency, positive predictive value, negative predictive value, and relative risk.

For example, the correlation between a subject's proteomic and/or miRNA profile and the likelihood of developing or acquiring a dystonia can be measured by an odds ratio (OR) and by the relative risk (RR). If P(R+) is the probability of developing or acquiring a dystonia for subjects with the risk profile (R) and P(R−) is the probability of developing memory impairment for individuals without the risk profile, then the relative risk is the ratio of the two probabilities: RR=P(R+)/P(R″).

In case-control studies, however, direct measures of the relative risk often cannot be obtained because of sampling design. The odds ratio allows for an approximation of the relative risk for low-incidence diseases and can be calculated: OR=(F+/(1−F+))/(F/(1−F)), where F+ is the frequency of a risk profile in cases studies and F″ is the frequency of risk profile in controls. F+ and F″ can be calculated using the proteomic and/or miRNA profile frequencies of the study.

The attributable risk (AR) can also be used to express an increased risk. The AR describes the proportion of individuals in a population exhibiting memory impairment due to a specific member of the proteomic and/or miRNA risk profile. AR may also be important in quantifying the role of individual components (specific member) in disease etiology and in terms of the public health impact of the individual marker. The public health relevance of the AR measurement lies in estimating the proportion of cases of memory impairment in the population that could be prevented if the profile or individual component were absent. AR may be determined as follows: AR=PE(RR−1)/(P£(RR−1)+1), where AR is the risk attributable to a profile or individual component of the profile, and PE is the frequency of exposure to a profile or individual component of the profile within the population at large. RR is the relative risk, which can be approximated with the odds ratio when the profile or individual component of the profile under study has a relatively low incidence in the general population.

In an aspect, the increased risk of a subject can be determined from p-values that are derived from association studies. Specifically, associations with specific profiles can be performed using regression analysis by regressing the proteomic and/or miRNA profile with developing or acquiring a dystonia. In addition, the regression may or may not be corrected or adjusted for one or more factors. The factors for which the analyses may be adjusted include, but are not limited to age, sex, weight, ethnicity, geographic location, fasting state, state of pregnancy or post-pregnancy, menstrual cycle, general health of the subject, alcohol or drug consumption, caffeine or nicotine intake and circadian rhythms, and the subject's p-EIF2α dysfunction and/or ISR dysfunction to name a few.

Increased risk can also be determined from p-values that are derived using logistic regression. Binomial (or binary) logistic regression is a form of regression which is used when the dependent is a dichotomy and the independents are of any type. Logistic regression can be used to predict a dependent variable on the basis of continuous and/or categorical independents and to determine the percent of variance in the dependent variable explained by the independents; to rank the relative importance of independents; to assess interaction effects; and to understand the impact of covariate control variables. Logistic regression applies maximum likelihood estimation after transforming the dependent into a “logit” variable (the natural log of the odds of the dependent occurring or not). In this way, logistic regression estimates the probability of a certain event occurring. These analyses are conducted with the program SAS. SAS (“statistical analysis software”) is a general purpose package (similar to Stata and SPSS). Ready-to-use procedures handle a wide range of statistical analyses, including but not limited to, analysis of variance, regression, categorical data analysis, multivariate analysis, survival analysis, psychometric analysis, cluster analysis, and nonparametric analysis.

As used herein, a “Z-score” refers to a standard score that is a very useful statistic because it (a) allows one to calculate the probability of a score occurring within the normal distribution and (b) enables one to compare two scores that are from different normal distributions. The standard score does this by converting (in other words, standardizing) scores in a normal distribution to Z-scores in what becomes a standard normal distribution. A Z-score is a measure of how many standard deviations below or above the population mean a raw score is. A Z-score can be placed on a normal distribution curve. Z-scores range from −3 standard deviations (which would fall to the far left of the normal distribution curve) up to +3 standard deviations (which would fall to the far right of the normal distribution curve).

As used herein, “Cohen's D” or “standardized mean difference” refers to one of the most common ways to measure effect size. An effect size is how large an effect is. For example, medication A has a larger effect than medication B. While a p-value can tell you if there is an effect, it won't tell you how large that effect is. Cohen's D specifically measures the effect size of the difference between two means. The formula for Cohen's D (for equally sized groups) is: d=(M1−M2)/Spooled, where M1=mean of group 1, M2=mean of group 2, spooled=pooled standard deviations for the two groups. The formula is: V [(si 2+s22)/2].

As used herein, the phrase “proteomic profile” means the combination of proteins found in a subject's biosample, which includes but is not limited to isolated EVs. The proteomic profile is a collection of measurements, such as but not limited to a quantity or concentration, for individual proteins taken from a subject's biosample. Techniques to determine the levels of individual components of the proteomic profile from biosamples are well known to the skilled technician and include, but are not limited to, mass spectrometry, ultra-performance liquid chromatography (UPLC), high-performance liquid chromatography (HPLC), mass spectrometry in conjunction with UPLC, LC/MS/MS, ELISA, and western blots.

As used herein, the phrase “miRNA profile” means the combination of miRNAs found in a subject's biosample, which includes but is not limited to isolated EVs. The miRNA profile is a collection of measurements, such as but not limited to a quantity or concentration, for individual miRNAs taken from a subject's biosample. Techniques to determine the levels of individual components of the miRNA profile from biosamples are well known to the skilled technician and include, but are not limited to, RNAseq and RT-qPCR.

The assessment of the levels of the individual components of the miRNA profile can be expressed as absolute or relative values and may or may not be expressed in relation to another component, a standard, an internal standard, or another molecule of compound known to be in the sample. If the levels are assessed as relative to a standard or internal standard, then the standard can be added to the test sample prior to, during, or after sample processing.

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing a dystonia is intended. The words “prevent” and “preventing” and “prevention” also refer to prophylactic or preventative measures for protecting or precluding a subject (e.g., an individual) not having a given dystonia or dystonia-related complication from progressing to that complication.

As used herein, the terms “administering” and “administration” refer to any method of providing one or more of the disclosed therapeutic agents, disclosed pharmaceutical formulations, or a combination thereof to a subject. Such methods are well known to those skilled in the art and include, but are not limited to, the following routes: oral administration, transdermal administration, administration by inhalation, nasal administration, topical administration, in utero administration, intrahepatic administration, intravaginal administration, ophthalmic administration, intraaural administration, otic administration, intracerebral administration, rectal administration, sublingual administration, buccal administration, and parenteral administration, including injectable such as intravenous administration, intra-CSF administration, intra-arterial administration, intramuscular administration, and subcutaneous administration. Administration can also include hepatic intra-arterial administration or administration through the hepatic portal vein (HPV). Administration of a disclosed therapeutic agent, a disclosed pharmaceutical composition, or a combination thereof can comprise administration directly into the CNS (e.g., intraparenchymal, intracerebroventricular, intrathecal cisternal, intrathecal (lumbar), deep gray matter delivery, convection-enhanced delivery to deep gray matter) or the PNS. Administration can be continuous or intermittent.

In an aspect, a “therapeutic agent” can be a “biologically active agent” or “biologic active agent” or “bioactive agent”, which refers to an agent that is capable of providing a local or systemic biological, physiological, or therapeutic effect in the biological system to which it is applied. For example, the bioactive agent can act to control infection or inflammation, enhance cell growth and tissue regeneration, control tumor growth, act as an analgesic, promote anti-cell attachment, and enhance bone growth, among other functions. Other suitable bioactive agents can include anti-viral agents, vaccines, hormones, antibodies (including active antibody fragments sFv, Fv, and Fab fragments), aptamers, peptide mimetics, functional nucleic acids, therapeutic proteins, peptides, or nucleic acids. Other bioactive agents include prodrugs, which are agents that are not biologically active when administered but, upon administration to a subject are converted to bioactive agents through metabolism or some other mechanism. Additionally, any of the compositions of the invention can contain combinations of two or more bioactive agents. It is understood that a biologically active agent can be used in connection with administration to various subjects, for example, to humans (i.e., medical administration) or to animals (i.e., veterinary administration). As used herein, the recitation of a biologically active agent inherently encompasses the pharmaceutically acceptable salts thereof.

As used herein, the term “pharmaceutically active agent” includes a “drug” or a “vaccine” and means a molecule, group of molecules, complex or substance administered to an organism for diagnostic, therapeutic, preventative medical, or veterinary purposes. This term includes externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term may also be used in reference to agricultural, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a bioactive effect, for example deoxyribonucleic acid (DNA) or ribonucleic acid (RNA). Pharmaceutically active agents include the herein disclosed categories and specific examples. It is not intended that the category be limited by the specific examples. Those of ordinary skill in the art will recognize also numerous other compounds that fall within the categories and that are useful according to the invention. Examples include a radiosensitizer, the combination of a radiosensitizer and a chemotherapeutic, a steroid, a xanthine, a beta-2-agonist broncho dilator, an anti-inflammatory agent, an analgesic agent, a calcium antagonist, an angiotensin-converting enzyme inhibitors, a beta-blocker, a centrally active alpha-agonist, an alpha-1-antagonist, carbonic anhydrase inhibitors, prostaglandin analogs, a combination of an alpha agonist and a beta blocker, a combination of a carbonic anhydrase inhibitor and a beta blocker, an anticholinergic/antispasmodic agent, a vasopressin analogue, an antiarrhythmic agent, an antiparkinsonian agent, an antianginal/antihypertensive agent, an anticoagulant agent, an antiplatelet agent, a sedative, an anxiolytic agent, a peptidic agent, a biopolymeric agent, an antineoplastic agent, a laxative, an antidiarrheal agent, an antimicrobial agent, an antifungal agent, or a vaccine. In a further aspect, the pharmaceutically active agent can be coumarin, albumin, brimonidine, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenoterol, clenbuterol, bambuterol, salmeterol, fenoterol; anti-inflammatory agents, including anti-asthmatic anti-inflammatory agents, antarthritic anti-inflammatory agents, and non-steroidal anti-inflammatory agents, examples of which include but are not limited to sulfides, mesalamine, budesonide, salazopyrin, diclofenac, pharmaceutically acceptable diclofenac salts, nimesulide, naproxen, acetaminophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, timol hemihydrate, levobunolol hydrochloride, esmolol hydrochloride, carteolol, propanoic 1 hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists (i.e., alpha adrenergic receptor agonist) such as clonidine, brimonidine tartrate, and apraclonidine hydrochloride; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; prostaglandin analogs such as latanoprost, travoprost, and bimatoprost; cholinergics (i.e., acetylcholine receptor agonists) such as pilocarpine hydrochloride and carbachol; glutamate receptor agonists such as the N-methyl D-aspartate receptor agonist memantine; anti-Vascular endothelial growth factor (VEGF) aptamers such as pegaptanib; anti-VEGF antibodies (including but not limited to anti-VEGF-A antibodies) such as ranibizumab and bevacizumab; carbonic anhydrase inhibitors such as methazolamide, brinzolamide, dorzolamide hydrochloride, and acetazolamide; antiarrhythmic agents such as quinidine, lidocaine, tocainide hydrochloride, mexiletine hydrochloride, digoxin, verapamil hydrochloride, propafenone hydrochloride, flecainide acetate, procainamide hydrochloride, moricizine hydrochloride, and disopyramide phosphate; antiparkinsonian agents, such as dopamine, L-Dopa/Carbidopa, selegiline, dihydroergocryptine, pergolide, lisuride, apomorphine, and bromocriptine; antiangina agents and antihypertensive agents such as isosorbide mononitrate, isosorbide dinitrate, propranolol, atenolol and verapamil; anticoagulant and antiplatelet agents such as coumadin, warfarin, acetylsalicylic acid, and ticlopidine; sedatives such as benzodiazepines and barbiturates; anxiolytic agents such as lorazepam, bromazepam, and diazepam; peptidic and biopolymeric agents such as calcitonin, leuprolide and other LHRI-I agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxin hydrochloride, loperamide hydrochloride, furazolidone, diphenoxylate hydrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides. It is understood that a pharmaceutically active agent can be used in connection with administration to various subjects, for example, to humans (i.e., medical administration) or to animals (i.e., veterinary administration). As used herein, the recitation of a pharmaceutically active agent inherently encompasses the pharmaceutically acceptable salts thereof.

In an aspect, a “therapeutic agent” can be any agent that effects a desired clinical outcome in a subject having a dystonia, suspected of having a dystonia, and/or likely to develop or acquire a dystonia. In an aspect, a disclosed therapeutic agent can be an oligonucleotide therapeutic agent. A disclosed oligonucleotide therapeutic agent can comprise a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable.

In an aspect, the skilled person can determine an efficacious dose, an efficacious schedule, and an efficacious route of administration for one or more of the disclosed agents, disclosed therapeutic agents, disclosed pharmaceutical formulations or a combination thereof (such as an agent that modulates ISR dysfunction and/or eIFα2 dysfunction) so as to treat or prevent a dystonia (such as DYT1). In an aspect, the skilled person can also alter, change, or modify an aspect of an administering step to improve efficacy of one or more of the disclosed agents, disclosed therapeutic agents, disclosed pharmaceutical formulations, or a combination thereof. In an aspect, the skilled person can determine an efficacious dose, an efficacious schedule, and an efficacious route of administration for any disclosed agent. These disclosed agents include but are not limited to anticholinergic drugs, benzodiazepines, muscle relaxants, agents that modulate the expression level of one or more disclosed differentially expressed proteins, agents that target eIF2α signaling, ritonavir, nelfinavir, lopinavir, saquinavir, deshydroxy-lopinavir, cobicistat, deshydroxy-ritonavir, or any combination thereof.

As used herein, “modifying the method” can comprise modifying or changing one or more features or aspects of one or more steps of a disclosed method. For example, in an aspect, a method can be altered by changing the amount of one or more of the disclosed agents, disclosed therapeutic agents, disclosed pharmaceutical formulations or a combination thereof administered to a subject, or by changing the frequency of administration of one or more of the disclosed agents, disclosed therapeutic agents, disclosed pharmaceutical formulations or a combination thereof, or by changing the duration of time one or more of the disclosed agents, disclosed therapeutic agents, disclosed pharmaceutical formulations or a combination thereof are administered to a subject.

As used herein, “concurrently” means (1) simultaneously in time, or (2) at different times during the course of a common treatment schedule.

The term “contacting” as used herein refers to bringing one or more of the disclosed therapeutic agents, disclosed pharmaceutical formulations, or a combination thereof together with a target area or intended target area in such a manner that the one or more of the disclosed agents, disclosed therapeutic agents, disclosed pharmaceutical formulations or a combination thereof exert an effect on the intended target or targeted area either directly or indirectly. A target area or intended target area can be one or more of a subject's organs (e.g., lungs, heart, liver, kidney, brain, etc.). In an aspect, a target area or intended target area can be any cell or any organ infected by a dystonia (such as DYT1). In an aspect, a target area or intended target area can be the brain or various neuron populations.

As used herein, “determining” can refer to measuring or ascertaining the presence and severity of a dystonia, such as, for example, DYT1. Methods and techniques used to determine the presence and/or severity of a dystonia are typically known to the medical arts. For example, the art is familiar with the ways to identify and/or diagnose the presence, severity, or both of a dystonia (such as, for example, a DYT1).

In an aspect, “determining” can also refer to measuring or ascertaining the level of one or more proteins or peptides in a biosample, or measuring or ascertaining the level or one or more RNAs or miRNAs in a biosample. Methods and techniques for determining the level of proteins/peptides and RNAs/miRNAs are known to the art and are disclosed herein.

The level of differential expression of miRNAs in a biosample when compared to a reference biosample (or any other biosample) can vary. For example, the level of any one or more differentially expressed miRNAs in a biosample can be at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 times or fold lower than that of a reference biosample. Or, the levels of any one or more differentially expressed miRNAs can be at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 times or fold higher than that of a reference biosample. In an aspect, the number of “times” the level of one or more differentially expressed proteins and/or miRNAs is lower or higher than that of a reference level can be a relative or an absolute number of times. Or, in an aspect, the level of the proteins and/or miRNAs can be normalized to a standard and these normalized levels can then be compared to one another to determine whether the differentially expressed proteins and/or miRNAs is lower or higher.

As used herein, “effective amount” and “amount effective” can refer to an amount that is sufficient to achieve the desired result such as, for example, the treatment and/or prevention of a dystonia (e.g., DYT1) or a suspected dystonia. As used herein, the terms “effective amount” and “amount effective” can refer to an amount that is sufficient to achieve the desired an effect on an undesired condition (e.g., a dystonia). For example, a “therapeutically effective amount” refers to an amount that is sufficient to achieve the desired therapeutic result or to have an effect on undesired symptoms, but is generally insufficient to cause adverse side effects. In an aspect, “therapeutically effective amount” means an amount of a disclosed pharmaceutical formulation, a disclosed agent, and/or disclosed therapeutic agent that (i) treats the particular disease, condition, or disorder (e.g., a dystonia such as DYT1), (ii) attenuates, ameliorates, or eliminates one or more symptoms of the particular disease, condition, or disorder (e.g., a dystonia), or (iii) delays the onset of one or more symptoms of the particular disease, condition, or disorder described herein (e.g., a dystonia). The specific therapeutically effective dose level for any particular patient will depend upon a variety of factors including the disorder being treated and the severity of the disorder; the disclosed therapeutic agents or disclosed pharmaceutical formulations employed; the disclosed methods employed; the age, body weight, general health, sex and diet of the patient; the time of administration; the route of administration; the rate of excretion of the disclosed isolated therapeutic agents or disclosed pharmaceutical formulations employed; the duration of the treatment; drugs used in combination or coincidental with the disclosed therapeutic agents or disclosed pharmaceutical formulations employed, and other like factors well known in the medical arts. For example, it is well within the skill of the art to start doses of the disclosed therapeutic agents or disclosed pharmaceutical formulations at levels lower than those required to achieve the desired therapeutic effect and to gradually increase the dosage until the desired effect is achieved. If desired, then the effective daily dose can be divided into multiple doses for purposes of administration. Consequently, a single dose of the disclosed therapeutic agents or disclosed pharmaceutical formulations can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition, such as, for example, a dystonia.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microcapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, Pa., which is hereby incorporated by reference in its entirety.

As used herein, “small molecule” can refer to any organic or inorganic material that is not a polymer. Small molecules exclude large macromolecules, such as large proteins (e.g., proteins with molecular weights over 2,000, 3,000, 4,000, 5,000, 6,000, 7,000, 8,000, 9,000, or 10,000), large nucleic acids (e.g., nucleic acids with molecular weights of over 2,000, 3,000, 4,000, 5,000, 6,000, 7,000, 8,000, 9,000, or 10,000), or large polysaccharides (e.g., polysaccharides with a molecular weight of over 2,000, 3,000, 4,000, 5,000, 6,000, 7,000, 8,000, 9,000, or 10,000). In an aspect, a “small molecule”, for example, can be a drug that can enter cells easily because it has a low molecular weight. In an aspect, a disclosed small molecule can penetrate the blood-brain-barrier (BBB).

As known to the art, “microRNAs” or “miRNAs” are small non-coding RNAs that regulate the expression of protein coding RNAs. The binding of an antisense compound to a microRNA prevents that microRNA from binding to its messenger RNA targets, and thus interferes with the function of the microRNA. MicroRNA mimics can enhance native microRNA function. miRNAs are generally about 17 to about 25 nucleotide bases (nt) in length in their biologically active form. In an aspect, a disclosed miRNA can regulate gene expression post transcriptionally by decreasing target mRNA translation. In an aspect, a disclosed miRNA can function as a negative regulator. In an aspect, a disclosed miRNA is about 17 to about 25, about 17 to about 24, about 17 to about 23, about 17 to about 22, about 17 to about 21, about 17 to about 20, about 17 to about 19, about 18 to about 25, about 18 to about 24, about 18 to about 23, about 18 to about 22, about 18 to about 21, about 18 to about 20, about 19 to about 25, about 19 to about 24, about 19 to about 23, about 19 to about 22, about 19 to about 21, about 20 to about 25, about 20 to about 24, about 20 to about 23, about 20 to about 22, about 21 to about 25, about 21 to about 24, about 21 to about 23, about 22 to about 25, about 22 to about 24, or about 22 nucleotides in length. Generally, there are three forms of miRNAs: primary miRNAs (pri-miRNAs), premature miRNAs (pre-miRNAs), and mature miRNAs, all of which are within the scope of the present disclosure.

As used herein, “exosomes” refer to small membrane vesicles found in cell culture supernatants and in different biological fluids. Exosomes form in a particular population of endosomes, called multivesicular bodies (MVBs), by inward budding into the lumen of the compartment. Upon fusion of MVBs with the plasma membrane, these internal vesicles are secreted. Exosomes possess a defined set of membrane and cytosolic proteins. Exosomes are implicated in multiple biological processes.

As used herein, “extracellular vesicles” or “EVs” are known to facilitate intercellular communication in diverse cellular processes such as immune responses and coagulation. EVs can be broadly classified into exosomes, microvesicles (MVs) and apoptotic bodies according to their cellular origin as shown in TABLE 1 below.

### Table 1

TABLE 1

| | Exosomes | Microvesicles | Apoptotic Bodies |
|---|---|---|---|
| Origin | Endocytic | Plasma | Plasma |
| | pathway | membrane | membrane |
| Size | 40-120 nm | 50-1,000 nm | 500-2,000 nm |
| Function | Intercellular communication | Intercellular communication | Facilitate phagocytosis |
| Markers | Alix, Tsg101, tetraspanins (CD81, | Integrins, selectins, CD40 | Annexin V, |
| | CD63, CD9), flotillin | | phosphatidylserine |
| Contents | Proteins and nucleic acids (mRNA, | Proteins and nucleic acids (mRNA, | Nuclear fractions, cell |
| | miRNA and other non-coding RNAs) | miRNA and other non-coding RNAs) | organelles |

As used herein, the term “package insert” is used to refer to instructions customarily included in commercial packages of therapeutic products, that contain information about the indications, usage, dosage, administration, contraindications and/or warnings concerning the use of such therapeutic products.

As used herein, the term “in combination” in the context of the administration of one or more of the disclosed agents, disclosed therapeutic agents, disclosed pharmaceutical formulations or a combination thereof includes the use of more than one therapy (e.g., additional therapeutic agents). Administration “in combination with” one or more additional therapeutic agents includes simultaneous (e.g., concurrent) and consecutive administration in any order. The use of the term “in combination” does not restrict the order in which therapies are administered to a subject. By way of non-limiting example, a first therapy (e.g., one or more of the disclosed agents, disclosed therapeutic agents, disclosed pharmaceutical formulations, or a combination thereof) may be administered prior to (e.g., 1 minute, 15 minutes, 30 minutes, 45 minutes, 1 hour, 2 horns, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., one or more of the disclosed agents, disclosed therapeutic agents, disclosed pharmaceutical formulations, or a combination thereof or one or more additional therapeutic agents) to a subject having or diagnosed with a dystonia (such as DYT1).

Disclosed are the components to be used to prepare the disclosed agents, disclosed therapeutic agents, and/or the disclosed pharmaceutical formulations as well the disclosed agents, disclosed therapeutic agents, and/or the disclosed pharmaceutical formulations used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific aspects or combination of aspects of the disclosed methods.

### B. Dystonias

A plethora of heterogeneous movement disorders is grouped under the umbrella term “dystonia”. The clinical presentation ranges from isolated dystonia to multi-systemic disorders where dystonia is only a co-occurring sign. In the past, definitions, nomenclature, and classifications have been repeatedly refined, adapted, and extended to reflect novel findings and increasing knowledge about the clinical, etiologic, and scientific background of dystonia. Currently, dystonia is suggested to be classified according to 2 axes. The first axis offers precise categories for the clinical presentation grouped into age at onset, body distribution, temporal pattern, and associated features. The second, etiologic, axis discriminates pathological findings, as well as inheritance patterns, mode of acquisition, or unknown causality. Furthermore, the recent recommendations regarding terminology and nomenclature of inherited forms of dystonia and related syndromes are illustrated in this article. Thus, dystonias are a group of chronic movement-disabling disorders for which highly effective oral medications or disease-modifying therapies are lacking. The most effective treatments require invasive procedures such as deep brain stimulation.

Presently, at least 24 genetic loci have been associated with isolated or combined heritable dystonias (Balint B., et al. (2015) Eur. J. Neurol. 22:610-617). While some cellular processes are implicated by those dystonia-associated genes for which there are known functions, the cellular mechanisms for dystonia remain largely unknown (Bragg D C, et al. (2011) Neurobiol. Dis. 42:136-147). The first identified gene was named TOR1A by Ozelius and colleagues and is associated with early-onset torsion dystonia (Ozelius L J, et al. (1997). Nat. Genet. 17:40-48). TOR1A encodes the protein torsinA. All torsinA-related dystonia cases found so far are due to the deletion of one of a pair of glutamate residues (E302/303) toward the C terminus of the encoded protein. TorsinA is part of the large AAA+ family of ATPases and the glutamate deletion is near to the ATP binding region. An interesting aspect of the genetics of torsinA dystonia is that there is greatly reduced penetrance. About one third of patients who carry the causal DE302/303 mutation develop dystonia, while the rest remaining asymptomatic. There also appears to be a time-dependent window for susceptibility. Generally, mutation carriers who are asymptomatic in their early 20s remain so throughout life, although there may be exceptions (Bressman S B, et al. (2000) Curr Treat Options Neurol. 2(3):275-285; Bressman S B. (2000) Clin Neuropharmacol. 23(5):239-251; Bressman S B, et al. (2000) Neurology. 54(9):1746-52). This implies that there is a critical timing for the expression of symptoms and indicates that dystonia is a developmental disease.

Inherited forms of dystonia require a confirmed genetic origin and can again be subdivided into multiple groups according to the pattern of inheritance. There are several forms of autosomal dominant dystonia such as DYT-TOR1A (Ozelius L J, et al. (1997). Nat. Genet. 17:40-48), DYT/PARK-GCH1 (Ichinose H, et al. (1994) Nat Genet. 8(3):236-242; Segawa M, et al. (2003) Ann Neurol. 54 Suppl 6:S32-S45), DYT-THAP1 (Fuchs T, et al. (2009) Nat Genet. 41(3):286-288), DYT-SGCE (Zimprich A, et al. (2001) Nat Genet. 29(1):66-69), and DYT/PARK-ATP1A3 (de Carvalho A P, et al. (2004). Neuron. 43(2):169-175). Autosomal recessive forms of dystonia include as DYT-ATP7B, also known as Wilson disease (Bull P C, et al. (1993) Genomics. 16(3):593-598), NBIA/DYT-PANK2 or pantothenate kinase-associated neurodegeneration (PKAN) (Zhou Y, et al. (2001) Neuropharmacology. 41(5):601-608), and NBIA/DYT/PARKa-PLA2G6 or PLA2G6-associated neurodegeneration (PLAN) (Morgan N V, et al. (2006) Nat Genet. 38(7):752-754). Also, multiple metabolic disorders can be found in this category. X-linked recessive dystonias include DYT/PARK-TAF1 (Makino S, et al. (2007) Am J Hum Genet. 80(3):393-406), DYT/CHOR-HPRT or Lesch-Nyhan syndrome (Gibbs R A, et al. (1987) Science. 236(4799):303-305), and DYT-TIMM8A, also known as Mohr-Tranebjaerg syndrome (Tranebjaerg L, et al. (2000) Adv Otorhinolaryngol. 56:176-180). Inherited forms with mutations in the mitochondrial genome are, for example, Leigh syndrome or DYT-mt-ND6 (Leber optic atrophy and dystonia) (Kim C E, et al. (2010). Proc. Natl. Acad. Sci. USA. 107:9861-9866). Notably, a large proportion of the recessive forms (autosomal and X-linked) as well as the mitochondrial forms are classified as complex dystonia forms, whereas all isolated dystonias with a known genetic causality are inherited in an autosomal dominant fashion (Klein C, et al. (2017) GeneReviews).

Several causal factors for the acquisition of dystonia have been documented so far. These factors include perinatal brain injury (e.g., dystonic cerebral palsy, delayed onset dystonia), infection/inflammation (e.g., viral encephalitis, encephalitis lethargica, subacute sclerosing panencephalitis, human immunodeficiency virus (HIV) infection, autoimmune causes, tuberculosis, syphilis), drugs (levodopa and dopamine agonists, neuroleptics like dopamine receptor blocking drugs, anticonvulsants, and calcium channel blockers), toxic (e.g., manganese, cobalt, carbon disulfide, cyanide, methanol, disulfiram, and 3-nitropropionic acid), vascular (ischemia, hemorrhage, and arteriovenous malformation including aneurysm), neoplastic (e.g., brain tumor, and paraneoplastic encephalitis), brain injury (e.g., head trauma, brain surgery including stereotactic ablations, and electrical injury).

For example, DYT1 dystonia is a rare, early-onset, generalized form of dystonia. DYT1 is caused by an in-frame trinucleotide deletion in the TOR1A gene, leading to loss of a glutamic acid residue (DE) from the AAA+ ATPase Torsin1a (Ozelius L J, et al. (1997). Nat. Genet. 17:40-48). Both the normal function of Torsin1a and significance of the mutant protein for disease pathogenesis have been intensively studied and at least five cellular processes have been suggested, including roles in nuclear transport, synaptic vesicle cycling, lipid metabolism, and endoplasmic reticulum (ER) stress (Burdette et al. (2010) Cell Stress Chaper. 15:605-617; Chen P, et al. (2010). Hum. Mol. Genet. 19:3502-3515; Goodchild R E, et al. (2005) Neuron. 48:923-932; Granata A, et al. (2011). EMBO J. 30, 181-193; Granata A, et al. (2008). J. Biol. Chem. 283:7568-7579; Grillet M, et al. (2016). Dev. Cell. 38:235-247; Jokhi V, et al. (2013). Cell Rep. 3:988-995; Nery F C, et al. (2011). Nat. Commun. 2:393).

Normally, wild-type (WT) Torsin1a cycles between the outer nuclear envelope (NE) and ER lumen in an ATP-dependent fashion, with the bulk of the protein detected in the ER (Goodchild R E, et al. (2004) Proc. Natl. Acad. Sci. U.S.A. 101:847-852; Naismith T V, et al. (2004). Proc. Natl. Acad. Sci. USA. 101:7612-7617). In contrast, when ΔE Torsin1a is the major species, as in overexpression experiments or homozygous knockin mouse models, it predominantly co-localizes with nuclear envelope markers and disrupts the normal subcellular NE membrane structure in a manner that is suggestive of a membrane-trafficking defect (Goodchild R E, et al. (2004) Proc. Natl. Acad. Sci. U.S.A. 101:847-852; Jokhi V, et al. (2013). Cell Rep. 3:988-995; Naismith T V, et al. (2004). Proc. Natl. Acad. Sci. USA. 101:7612-7617). At the light microscopic level, ΔE Torsin1a distribution appears as an abnormal punctate pattern. The ΔE TorsinA mislocalizing propensity is also observed independent of whether the protein is expressed as a fusion protein or not. Lastly, ΔE Torsin1a has been associated with activation of the unfolded protein response (UPR) (Bragg D C, et al. (2011) Neurobiol. Dis. 42:136-147; Chen P, et al. (2010). Hum. Mol. Genet. 19:3502-3515; Hewett J W, et al. (2007). Proc. Natl. Acad. Sci. USA. 104:7271-7276; Nery F C, et al. (2011). Nat. Commun. 2:393), and in DYT1 patient-derived fibroblasts, the chemical chaperone, phenylbutyric acid (PBA) reduces indicators of UPR activation (Cao S, et al. (2010). Dis. Model. Mech. 3:386-396). PBA significantly reduced punctate pathology. Thus, Torsin1a localization phenotypes predict known Torsin1a biology.

### C. Integrated Stress Response

The integrated stress response (ISR) is an elaborate signaling pathway present in eukaryotic cells, which is activated in response to a range of physiological changes and different pathological conditions. Such stresses commonly include cell extrinsic factors such as hypoxia, amino acid deprivation, glucose deprivation, and viral infection. However, cell intrinsic stresses such as endoplasmic reticulum (ER) stress, caused by the accumulation of unfolded proteins in the ER, can also activate the ISR. Furthermore, in the context of cancer biology, the ISR can be triggered by oncogene activation. The common point of convergence for all the stress stimuli that activate ISR is phosphorylation of the alpha subunit of eukaryotic translation initiation factor 2 (eIF2a) on serine 51. In mammalian cells, this is catalyzed by a family of four serine/threonine (S/T) eIF2a kinases that are activated by distinct stress stimuli. eIF2a phosphorylation causes a reduction in global protein synthesis while allowing the translation of selected genes including activating transcription factor 4 (ATF4), aiding cell survival and recovery. However, if the cellular stress is severe, either in intensity or in duration, it will overwhelm the capacity of the adaptive response to resolve it and additional components become activated to execute cell death.

Dephosphorylation of eIF2a signals termination of the ISR and return to normal protein synthesis. It is likely that the duration and level of eIF2a phosphorylation, as well as ATF4 regulation and its interactions with other proteins, determine the ultimate ISR outcome resulting from different environmental and physiological stresses. The initiation of the ISR relies on four evolutionarily related eIF2α kinases that each senses specific insults and signals by phosphorylating eIF2α. On the other hand, dephosphorylation of eIF2α and subsequent restoration of the translational capacity is emerging as a key event that controls the complete recovery from stress and ISR termination. Two cellular cofactors of the protein phosphatase-1 (PP1c) can specifically reverse the phosphorylation of eIF2α. The first one is GADD34, which is induced by the ISR to specifically direct PP1c to dephosphorylate eIF2α, allowing termination of the response and restoration of the homeostatic pace of translation. The second one is the protein CReP (Constitutive Repressor of eIF2α Phosphorylation), also known as PPP1R15B, that is expressed ubiquitously in unstressed cells and was identified as a key factor maintaining low levels of eIF2α phosphorylation.

The “eukaryotic translation initiation factor 2” or “eIF2” refers to a heterotrimeric GTPase composed of a, b, and c subunits, which can bind GTP and methionine initiator tRNA to form a ternary complex. In conjunction with translation machinery, ternary complex scans along the 5′ untranslated region of mRNAs to detect the translation start site. Once the AUG start codon is decoded, GTP is hydrolyzed and eIF2-GDP is released as a binary complex from the ribosome. Exchange of GDP for GTP enables a new round of translation initiation. This occurs with the aid of a dedicated nucleotide exchange factor, translation initiation factor 2B (eIF2B), which is a decameric nucleotide exchange factor composed of two copies of subunits α, β, γ, δ, and ε.

The eIF2a kinases act as early responders to disturbances in cellular homeostasis. There are four members of the family: PKR-like ER kinase (PERK), double-stranded RNA-dependent protein kinase (PKR), heme-regulated eIF2a kinase (HRI), and general control nonderepressible 2 (GCN2). All four eIF2a kinases share extensive homology in their kinase catalytic domains, but possess distinct regulatory domain. Each eIF2a kinase dimerizes and autophosphorylates for full activation. However, each kinase responds to distinct environmental and physiological stresses, which reflects their unique regulatory mechanisms. But, when ER stress, viral infection, and other cellular stress signals activate PERK, PKR, HRI, and GCN2 kinases, they converge on phosphorylation of eIF2a, the core of ISR. Upon ISR induced phosphorylation, eIF2 is converted from substrate to competitive inhibitor of eIF2B, arresting general protein synthesis and upregulating translation of a select few mRNAs containing upstream open reading frames. These mRNAs encode stress-responsive factors such as the transcription factor ATF4. This leads to global attenuation of Cap-dependent translation while concomitantly initiates the preferential translation of ISR-specific mRNAs, such as ATF4.

ATF4 is a basic leucine zipper (bZIP) transcription factor that belongs to the activating transcription factor/cyclic AMP response element binding protein (ATF/CREB family) [100-102]. ATF4 has several dimerization partners that influence its regulation of gene transcription and can guide cellular outcome. In fact, ATF4 is a key deciding factor in cellular fate in response to ISR activation. It is regulated at the transcriptional, translational, and post-translational level, and moreover, its ability to interact with other transcription factors provides a further level of regulation. For example, ATF4 forms homo- and heterodimers that bind to DNA targets to control the expression of genes involved in cellular adaptation. Termination of the ISR is regulated by the constitutively expressed CReP and stress-inducible phosphatase GADD34 that dephosphorylate eIF2a.

Although multiple stresses converge on eIF2a phosphorylation to activate the ISR, the cellular outcome is not always the same. The effect of ISR activation depends not only on the nature of the stress, its duration and severity, but also on the extent of eIF2a phosphorylation and translation of ATF4 mRNA and other bZIP transcription factors. It is commonly accepted that a short-lived ISR is an adaptive, pro-survival response aiming at resolving stress and restoring homeostasis, while a prolonged ISR can signal toward cell death induction. Therefore, this dual effect of eIF2a phosphorylation raises an important question concerning how the switch between pro-survival and pro-death signaling by ISR is regulated, as well as whether a threshold of cell stress signals exists that favors the activation of cell death proteins.

The ISR, together with other cellular adaptation pathways, functions as an important part of the cellular defense strategy in response to stress. It does this mainly through altering global protein synthesis and through the regulation of genes that promote pro-survival signaling such as through the activation of autophagy, or that counteract pathways that lead to cell death such as apoptosis or proteotoxicity (impairment of cell function due to the effects of misfolded proteins). Notably, there is also a cross-talk between the ISR and other pro-survival pathways such as the UPR, phosphatidylinositol-3 kinase (PI3K) signaling, autophagy, and the ubiquitin-proteasome system.

One of the major effects of the ISR is on protein synthesis. The initial repression of global mRNA translation plays a very important role in promoting cell survival in the face of different stresses that activate the ISR. The accumulation of unfolded proteins in the ER induces a condition of ER stress, which is relieved by the reduced level of incoming proteins when global protein synthesis is inhibited. PKR activation of the ISR during viral infection helps to reduce the translation of viral mRNAs, thus protecting the cells. Under conditions of amino acid depletion, activation of the ISR by GCN2 reduces the need for amino acids for protein synthesis, thus alleviating this stress. Activation of the ISR by HRI under conditions of low heme lessens the need for heme by attenuating the translation of globin mRNAs, thus reducing the stress and promoting survival. It is important, however, to note that timely termination of the ISR also plays a key role in promoting long-term cell survival, by re-starting synthesis of essential proteins. This is achieved through dephosphorylation of eIF2a by the phosphatase GADD34, which is induced by ATF4 and its downstream targets CHOP and ATF3. Through the activation of macroautophagy, commonly known as autophagy, the ISR can regulate cell survival and cell death path.

In vitro human genome-wide screening and human genetics revealed weakened phosphor-eIF2α signaling in a variety of dystonias. For example, Prkra is gene discovered in familial DYT16 (Camargos S, et al. (2008). Lancet Neurol. 7:207-215), which is also associated with sporadic dystonia (Dos Santos C O, et al. (2018) Parkinsonism Relat. Disord. 48:93-96). THAP1 is a gene discovered in familial DYT6 (Fuchs T, et al. (2009) Nat Genet. 41(3):286-288), which also shows dysregulated eIF2a in mouse model (Zakirova Z, et al. (2018) PLoS Genet. 14(1):e1007169). Eif2ak1 and Eif2ak2 are two new genetic syndromes reported with features that include prominent dystonia (Mao D, et al. (2020) Am. J. Hum. Genet. 106:570-583 (2020)). EIF2B shows vanishing white matter disease symptoms include dystonia, torticollis (Klingelhoefer L, et al. (2014) Clin Med (Lond). 14(5):520-524).

Although the specific disruptions in the eIF2a pathway are distinct among these three dystonias, they converge upon a common consequence of reduced eIF2a pathway signaling. In DYT1 patient-derived fibroblasts, there was an attenuated ISR alongside high basal levels of a negative feedback regulator of eIF2a phosphorylation, the CReP/PPP1R15B phosphatase. In sporadic cervical dystonia patients, there was significant enrichment of rare ATF4 missense variants that reduce transcriptional activity. Transcriptional regulation by ATF4 is a major downstream effector of the ISR (Pakos-Zebrucka K, et al. (2016). EMBO Rep. 17:1374-1395). In DYT16, it is noteworthy that the most common PRKRA mutation is associated with both initially decreased and delayed excessive stress-induced phosphorylation of eIF2a among other effects such as promoting apoptosis.

Integrated stress response inhibitor (ISRIB) is a small drug-like that targets eIF2B. In vitro studies determined that ISRIB activates and stabilizes a decameric eIF2B complex. Specifically, ISRIB renders cells insensitive to eIF2a phosphorylation and thus inhibits the ISR downstream of eIF2a phosphorylation resulting in the attenuation of ATF4 synthesis. ISRIB restores the translational capacity and thus impairs the adaptation of cells to chronic ER stress. Additionally, ISRIB has been shown to prevent the formation of stress granules caused by eIF2a phosphorylation. In rodents, ISRIB is effective in a number of disease models in that treatment with the molecule can reverse cognitive deficits following traumatic brain injury, protect against prion-induced neurodegeneration, and prevent metastasis of a subset of cancers. ISRIB was shown to alter the normal subcellular localization of WT TorsinA in a way that was more similar to the distribution of dE TorsinA in DYT1.

Salubrinal is an agent that prolongs ISR activation by inhibiting or prevent eIF2α dephosphorylation by inhibiting the protein complex GADD34/protein phosphatase 1 (PP1), which consists of the general cellular serine/threonine phosphatase PP1 and the non-enzymatic cofactor GADD34. Salubrinal also inhibits CReP-PP1 complexes that dephosphorylate eIF2α. Salubrinal has a MW of 479.8 and a molecular formula of C21H17C13N4OS. Salubrinal is also known as PubChem ID 5717801. Salubrinal acts by slowing down protein synthesis, allowing increased time for protein folding within the ER, and thus protecting the cells from the deleterious effects of proteotoxicity.

Sal 003 is a cell-permeable inhibitor of cellular phosphatase complexes that dephosphorylate eukaryotic translation initiation factor 2 subunit α (eIF2α). Sal-003 is an analog of salubrinal with improved aqueous solubility. Sal-003 has a molecular weight of 463.21 and a molecular formula of C18H15C14N3OS. Sal 003 has the PubChem ID No. 5717737.

Guanabenz inhibits the stress-induced phosphatase GADD34 that causes the dephosphorylation of eIF2α. Guanabenz is not a selective GADD34 inhibitor though. Guanabenz has a MW of 231.08 and a molecular formula of C8H8C12N4. Guanabenz is also known as PubChem ID 5353646. Salubrinal acts by slowing down protein synthesis, allowing increased time for protein folding within the ER, and thus protecting the cells from the deleterious effects of proteotoxicity. Sephin 1, which is a guanabenz derivative, is a safe and selective GADD34-specific inhibitor.

Pharmacological approaches to targeting ISR signaling include (i) stimulating eIF2a phosphorylation through chemical activators of eIF2a kinases such as histidinol, asparaginase, halofuginone, arginine deiminase, BTdCPU, BEPP monohydrochloride, and CCT020312, or preventing eIF2a phosphorylation using indirubin-30-monoxime, SP600125, and SyK to inhibit GSK2, (iii) blocking PERK activation using GSK2606414 and GSK2656157, (iv) modulating PKR using C16 and 2-aminopurine, (v) inhibiting HRI using aminopyrazolindane, (vi) prolonging ISR using salubrinal, (vii) blocking GADD34 using guanabenz and Sephin1, (viii) decreasing CReP expression thereby affecting CReP-PP1 complex binding to eIF2a using nelfinavir [231] decreases CReP expression and affects CReP-PP1 complex, and (ix) reversing the consequences of eIF2a phosphorylation using ISRIB. Another approach to regulate ISR involves the modulation of ATF4 post-translational modifications. For example, phosphorylation of ATF4 at S251 can be blocked by SL0101, an RSK2 kinase inhibitor while the inhibition of phosphorylation of ATF4 at S254 can be achieved by inhibiting PKA with H-89. Further, nuclear to cytoplasmic shuttling of ATF4 followed by its phosphorylation-dependent proteasomal degradation can be induced by the synthesized RPL41 peptide.

### D. Dystonias

A plethora of heterogeneous movement disorders is grouped under the umbrella term “dystonia”. The clinical presentation ranges from isolated dystonia to multi-systemic disorders where dystonia is only a co-occurring sign. In the past, definitions, nomenclature, and classifications have been repeatedly refined, adapted, and extended to reflect novel findings and increasing knowledge about the clinical, etiologic, and scientific background of dystonia. Currently, dystonia is suggested to be classified according to 2 axes. The first axis offers precise categories for the clinical presentation grouped into age at onset, body distribution, temporal pattern, and associated features. The second, etiologic, axis discriminates pathological findings, as well as inheritance patterns, mode of acquisition, or unknown causality. Furthermore, the recent recommendations regarding terminology and nomenclature of inherited forms of dystonia and related syndromes are illustrated in this article. Thus, dystonias are a group of chronic movement-disabling disorders for which highly effective oral medications or disease-modifying therapies are lacking. The most effective treatments require invasive procedures such as deep brain stimulation.

Presently, at least 24 genetic loci have been associated with isolated or combined heritable dystonias (Balint B., et al. (2015) Eur. J. Neurol. 22:610-617). While some cellular processes are implicated by those dystonia-associated genes for which there are known functions, the cellular mechanisms for dystonia remain largely unknown (Bragg D C, et al. (2011) Neurobiol. Dis. 42:136-147). The first identified gene was named TOR1A by Ozelius and colleagues and is associated with early-onset torsion dystonia (Ozelius L J, et al. (1997). Nat. Genet. 17:40-48). TOR1A encodes the protein torsinA. All torsinA-related dystonia cases found so far are due to the deletion of one of a pair of glutamate residues (E302/303) toward the C terminus of the encoded protein. TorsinA is part of the large AAA+ family of ATPases and the glutamate deletion is near to the ATP binding region. An interesting aspect of the genetics of torsinA dystonia is that there is greatly reduced penetrance. About one third of patients who carry the causal DE302/303 mutation develop dystonia, while the rest remaining asymptomatic. There also appears to be a time-dependent window for susceptibility. Generally, mutation carriers who are asymptomatic in their early 20s remain so throughout life, although there may be exceptions (Bressman S B, et al. (2000) Curr Treat Options Neurol. 2(3):275-285; Bressman S B. (2000) Clin Neuropharmacol. 23(5):239-251; Bressman S B, et al. (2000) Neurology. 54(9):1746-52). This implies that there is a critical timing for the expression of symptoms and indicates that dystonia is a developmental disease.

Inherited forms of dystonia require a confirmed genetic origin and can again be subdivided into multiple groups according to the pattern of inheritance. There are several forms of autosomal dominant dystonia such as DYT-TOR1A (Ozelius L J, et al. (1997). Nat. Genet. 17:40-48), DYT/PARK-GCH1 (Ichinose H, et al. (1994) Nat Genet. 8(3):236-242; Segawa M, et al. (2003) Ann Neurol. 54 Suppl 6:S32-S45), DYT-THAP1 (Fuchs T, et al. (2009) Nat Genet. 41(3):286-288), DYT-SGCE (Zimprich A, et al. (2001) Nat Genet. 29(1):66-69), and DYT/PARK-ATP1A3 (de Carvalho A P, et al. (2004). Neuron. 43(2):169-175). Autosomal recessive forms of dystonia include as DYT-ATP7B, also known as Wilson disease (Bull P C, et al. (1993) Genomics. 16(3):593-598), NBIA/DYT-PANK2 or pantothenate kinase-associated neurodegeneration (PKAN) (Zhou Y, et al. (2001) Neuropharmacology. 41(5):601-608), and NBIA/DYT/PARKa-PLA2G6 or PLA2G6-associated neurodegeneration (PLAN) (Morgan N V, et al. (2006) Nat Genet. 38(7):752-754). Also, multiple metabolic disorders can be found in this category. X-linked recessive dystonias include DYT/PARK-TAF1 (Makino S, et al. (2007) Am J Hum Genet. 80(3):393-406), DYT/CHOR-HPRT or Lesch-Nyhan syndrome (Gibbs R A, et al. (1987) Science. 236(4799):303-305), and DYT-TIMM8A, also known as Mohr-Tranebjaerg syndrome (Tranebjaerg L, et al. (2000) Adv Otorhinolaryngol. 56:176-180). Inherited forms with mutations in the mitochondrial genome are, for example, Leigh syndrome or DYT-mt-ND6 (Leber optic atrophy and dystonia) (Kim C E, et al. (2010). Proc. Natl. Acad. Sci. USA. 107:9861-9866). Notably, a large proportion of the recessive forms (autosomal and X-linked) as well as the mitochondrial forms are classified as complex dystonia forms, whereas all isolated dystonias with a known genetic causality are inherited in an autosomal dominant fashion (Klein C, et al. (2017) GeneReviews).

Several causal factors for the acquisition of dystonia have been documented so far. These factors include perinatal brain injury (e.g., dystonic cerebral palsy, delayed onset dystonia), infection/inflammation (e.g., viral encephalitis, encephalitis lethargica, subacute sclerosing panencephalitis, human immunodeficiency virus (HIV) infection, autoimmune causes, tuberculosis, syphilis), drugs (levodopa and dopamine agonists, neuroleptics like dopamine receptor blocking drugs, anticonvulsants, and calcium channel blockers), toxic (e.g., manganese, cobalt, carbon disulfide, cyanide, methanol, disulfiram, and 3-nitropropionic acid), vascular (ischemia, hemorrhage, and arteriovenous malformation including aneurysm), neoplastic (e.g., brain tumor, and paraneoplastic encephalitis), brain injury (e.g., head trauma, brain surgery including stereotactic ablations, and electrical injury).

For example, DYT1 dystonia is a rare, early-onset, generalized form of dystonia. DYT1 is caused by an in-frame trinucleotide deletion in the TOR1A gene, leading to loss of a glutamic acid residue (DE) from the AAA+ ATPase Torsin1a (Ozelius L J, et al. (1997). Nat. Genet. 17:40-48). Both the normal function of Torsin1a and significance of the mutant protein for disease pathogenesis have been intensively studied and at least five cellular processes have been suggested, including roles in nuclear transport, synaptic vesicle cycling, lipid metabolism, and endoplasmic reticulum (ER) stress (Burdette et al. (2010) Cell Stress Chaper. 15:605-617; Chen P, et al. (2010). Hum. Mol. Genet. 19:3502-3515; Goodchild R E, et al. (2005) Neuron. 48:923-932; Granata A, et al. (2011). EMBO J. 30, 181-193; Granata A, et al. (2008). J. Biol. Chem. 283:7568-7579; Grillet M, et al. (2016). Dev. Cell. 38:235-247; Jokhi V, et al. (2013). Cell Rep. 3:988-995; Nery F C, et al. (2011). Nat. Commun. 2:393).

Normally, wild-type (WT) Torsin1a cycles between the outer nuclear envelope (NE) and ER lumen in an ATP-dependent fashion, with the bulk of the protein detected in the ER (Goodchild R E, et al. (2004) Proc. Natl. Acad. Sci. U.S.A. 101:847-852; Naismith T V, et al. (2004). Proc. Natl. Acad. Sci. USA. 101:7612-7617). In contrast, when ΔE Torsin1a is the major species, as in overexpression experiments or homozygous knockin mouse models, it predominantly co-localizes with nuclear envelope markers and disrupts the normal subcellular NE membrane structure in a manner that is suggestive of a membrane-trafficking defect (Goodchild R E, et al. (2004) Proc. Natl. Acad. Sci. U.S.A. 101:847-852; Jokhi V, et al. (2013). Cell Rep. 3:988-995; Naismith T V, et al. (2004). Proc. Natl. Acad. Sci. USA. 101:7612-7617). At the light microscopic level, ΔE Torsin1a distribution appears as an abnormal punctate pattern. The ΔE TorsinA mislocalizing propensity is also observed independent of whether the protein is expressed as a fusion protein or not. Lastly, ΔE Torsin1a has been associated with activation of the unfolded protein response (UPR) (Bragg D C, et al. (2011) Neurobiol. Dis. 42:136-147; Chen P, et al. (2010). Hum. Mol. Genet. 19:3502-3515; Hewett J W, et al. (2007). Proc. Natl. Acad. Sci. USA. 104:7271-7276; Nery F C, et al. (2011). Nat. Commun. 2:393), and in DYT1 patient-derived fibroblasts, the chemical chaperone, phenylbutyric acid (PBA) reduces indicators of UPR activation (Cao S, et al. (2010). Dis. Model. Mech. 3:386-396). PBA significantly reduced punctate pathology. Thus, Torsin1a localization phenotypes predict known Torsin1a biology.

### E. Compositions for Treating and/or Preventing a Dystonia

Disclosed herein is a pharmaceutical formulation comprising one or more disclosed agents in a pharmaceutically acceptable carrier. In an aspect, a disclosed pharmaceutical formulation can comprise one or more agents that modulate the expression level of one or more disclosed differentially expressed proteins such as, for example, increasing or decreasing the expression level. In an aspect, a disclosed pharmaceutical formulation can comprise one or more agents that target eIF2α signaling, such as, for example, contributing to the phosphorylation or de-phosphorylation of eIF2α. In an aspect, a disclosed pharmaceutical formulation can comprise ritonavir, nelfinavir, lopinavir, saquinavir, deshydroxy-lopinavir, cobicistat, deshydroxy-ritonavir, or any combination thereof. In an aspect, a disclosed pharmaceutical formulation can comprise ritonavir. In an aspect, a disclosed pharmaceutical formulation can comprise guanabenz, salubrinal, ISRIB, Sephin 1, or any combination thereof. In an aspect, a disclosed pharmaceutical formulation can comprise an agent that targets ISR signaling such as (i) stimulating eIF2a phosphorylation through chemical activators of eIF2a kinases such as histidinol, asparaginase, halofuginone, arginine deiminase, BTdCPU, BEPP monohydrochloride, and CCT020312, or preventing eIF2a phosphorylation using indirubin-30-monoxime, SP600125, and SyK to inhibit GSK2, (iii) blocking PERK activation using GSK2606414 and GSK2656157, (iv) modulating PKR using C16 and 2-aminopurine, (v) inhibiting HRI using aminopyrazolindane, (vi) prolonging ISR using salubrinal, (vii) blocking GADD34 using guanabenz and Sephin1, (viii) decreasing CReP expression thereby affecting CReP-PP1 complex binding to eIF2a using nelfinavir [231] decreases CReP expression and affects CReP-PP1 complex, and (ix) reversing the consequences of eIF2a phosphorylation using ISRIB. In an aspect, a disclosed pharmaceutical formulation can comprise an agent that targets ISR signaling such as modulation of ATF4 post-translational modifications including (i) phosphorylation of ATF4 at S251 can be blocked by SL0101, an RSK2 kinase inhibitor, (ii) inhibition of phosphorylation of ATF4 at S254 can be achieved by inhibiting PKA with H-89, and (iii) nuclear to cytoplasmic shuttling of ATF4 followed by its phosphorylation-dependent proteasomal degradation can be induced by the synthesized RPL41 peptide.

### F. Dystonia Biomarkers

A dystonia biomarker as described herein may be selected from a protein, miRNA, or a metabolite. In some embodiments, a dystonia biomarker can be a diagnostic biomarker. The diagnostic biomarker may detect or classify a pathological condition (e.g., a dystonia or related disorder as described herein). In some embodiments, a dystonia biomarker can be a prognostic biomarker. The prognostic biomarker may predict the probability of disease occurrence or progression. In some embodiments, a dystonia biomarker can be a pharmacodynamic or responsive biomarker. A pharmacodynamic or responsive biomarker may identify a change in response to a therapeutic intervention. In some embodiments, a dystonia biomarker can be a predictive biomarker. A predictive biomarker may predict how an individual or subject might respond to a particular intervention or event.

### Dystonia Biomarker Proteins

In an aspect, a biomarker protein as described herein may be a human and mouse common protein. As a skilled artisan would recognize, the complete names of the dystonia biomarker proteins corresponding to the protein abbreviations recited herein are readily available one or more of the public protein database (e.g., The UniProt Consortium, or other similar database). The corresponding protein names for certain abbreviations utilized herein may also be found in TABLES S1 and S8.

A biomarker protein may be selected from List-A1, wherein List-A1 is a group consisting of USP7, SEC31A, TARDBP, AP1B1, ZYX, RHOA, PI4KA, PSMC5, PCMT1, HNRNPC, GART, MAPK3, IP09, JAM3, CRYAB, STOM, RPS13, DLST, PRPSAP2, WDR26, PSME4, LIPG, NDNF, RAP2B, TLN1, FLNA, MRC2, SLC2A1, RPL10, PTMA, BZW2, LRRC59, RPL13A, ACAA1, H1-5, FSCN1, PSMD12, MARS1, TLN2, ARL8B, EHD4, TUBB1, MYH9, TGFB2, PITPNB, RBM3, ANO6, VCL, FBL, MYL6, CARM1, CST3, RALY, EIF2S3, TIMP3, ITGB3, ITGA6, SNAP23, DBNL, SPTBN1, CPNE3, PFN1, EIF2A, EMILIN1, PRMT5, HINT1, ATP6V1H, RPS17, HECTD1, DNAJA2, EML2, EIF5, ICAM1, OSBP, CAND1, PSMD2, MACROH2A1, TMED10, GNB1, PSAP, DHX9, PALLD, SUMO2, DPP7, CMPK1, PDGFRB, RPL23, MIF, WIPI2, SYNCRIP, PCBP2, LGALS1, PFAS, EXOC1, UPF1, EFEMP2, VAPA, RAP1B, RPL24, CACNA2D1, LLGL1, TJP2, MED23, AGRN, OTUB1, NRAS, CD9, CORO1B, RPS21, PRDX2, CD81, ARPC5, GNAI2, CTNNB1, TUBA4A, SPP1, EIF3F, PGD, NAPG, AIMP1, TTLL12, FCSK, PSME3, NAP1L1, IARS1, SUB1, SRSF3, CAPN2, SERPINH1, TPM4, MYO5A, DHX15, PPP1CB, PSMD4, RPLP2, ATP6AP2, TRIM28, RPS26, RPS27A, DYNC112, PLCB3, RALB, RALA, SEPTIN7, DDB1, ALCAM, RAB10, SPARC, FTL, DLAT, CRABP2, ARF5, DNAJB11, APP, USP14, IP07, PLXNA1, CDH2, KHDRBS1, RPL31, NAMPT, RPL23A, GPX1, HSPE1, LTBP1, PSMA4, TUBB, CTSB, SRI, ACTR10, NPM1, RTRAF, RPL18A, VIM, EIF3D, NSF, LIMS1, ACTB, SERBP1, USP15, CFL1, PPIA, RHOG, ITM2B, AIMP2, RPS20, ETF1, CD44, ADAM10, NUDT21, ATRN, AKR1A1, RPL15, FLNC, SOD1, RPS6, CALR, ITGB1, VDAC1, YWHAE, DCTN2, PPP2R1B, PSAT1, RTCB, EFR3A, EIF4G2, MAPRE2, METAP2, IDH2, HNRNPA2B1, HUWE1, NUCB1, TXNDC5, SRSF7, HSPA4L, TOLLIP, SLC25A3, CAPN1, AARS1, VASP, CTSA, STRAP, PSMA3, EIF3I, DYNLRB1, SH3GL1, YWHAZ, ESYT1, ARHGDIA, MAP4K4, PITRM1, CD151, DCTN1, CDC42, AHSA1, RPS3A, GLIPR2, PFKL, ARPCIA, GSS, PDLIM5, RPS16, SFPQ, MYO9B, HNRNPUL2, SLC9A1, PCYOX1, PPP1CA, RPL4, TUBB6, CAVIN2, TXNDC17, SRPK1, PSMD13, GNB2, HNRNPF, PSMD1, DAAM1, NAPA, MYO1C, EIF4G1, CYCS, FBLN5, DNAJB1, RABIA, LCAT, PURB, PRDX5, RPS23, EHD2, EHD1, EHD3, CD34, THBS1, RAB1B, HSPA9, AHCY, ACOT7, RPL9, C4B, UGGT1, PEF1, SDCBP, RPSA, LIMA1, APOB, SCFD1, G3BP1, GFUS, H1-4, PSMA1, VPS4B, FDPS, COL4A2, RAB5C, GLOD4, FGA, PSMB6, RNH1, TRIO, ATL3, FGG, PEBP1, AK2, ARPC2, PAICS, SNX6, RELN, TFRC, ARHGDIB, RPS18, LPL, LAP3, EIF4A1, NME1, BIRC6, LSM4, B4GALT1, PSMB4, MYO1B, PAFAH1B1, ALDOA, RAB11B, SND1, LAMB1, LOXL3, HSD17B4, COPE, HLA-B, ATIC, GJA1, DDAH2, GBA1, ADRM1, LTA4H, HS6ST1, ANXA11, RAB5A, RPL8, PITPNA, NUTF2, ILF2, H3C1, XRN2, ACTC1, EXT2, MDH2, TF, PFKP, PDIA6, SARS1, COPG1, RPL35A, PKM, RPL18, LAMP2, ELAVL1, ACTR3, CTSD, KARS1, RUVBL1, PLTP, PARP1, ILF3, LRP1, GSPT1, FARSA, TALDO1, KHSRP, GOT1, SRSF2, UBE2D3, ATP5F1A, JAK1, RBBP7, PRDX6, THBS3, WARS1, EEF1A1, BMP1, UNC5B, QSOX1, FBN1, LIPA, EIF6, CARS1, ALDH2, AKR1B1, SET, CCT6A, SNRPD1, NACA, GSTP1, HNRNPUL1, CDK4, PREP, SLC3A2, MSN, FLII, BANF1, OLA1, CLIC1, PHGDH, DDX39B, NPEPPS, MBP, BTF3, HNRNPH1, DSP, AXL, RAB5B, ADSL, PPP2CB, CTNNA1, RPL19, RPS19, MYO18A, HNRNPU, ANXA8L1, CAPRIN1, PARK7, DARS1, GMPPB, MEMO1, PDLIM1, ABCF2, HNRNPA3, APEH, DPYSL2, ACAT2, HSPA4, PSMA6, RPL13, ATP6V1E1, CSNK2A1, COPA, COL6A1, RPS9, GRN, SNRPB, IGF2R, EIF3L, TSG101, CLTC, IQGAP1, EIF2S1, PRPS1, EIF3A, MTHFD1, APRT, RPS7, GMPS, STX12, WDR1, LAMP1, PTBP1, PSMB3, DPP3, CKB, ITIH2, SNX2, HSP90AB1, LAMB2, SLIT3, HDLBP, GGH, BLVRA, ALDH16A1, HNRNPL, POSTN, YARS1, C5, COL4A1, IDE, COL18A1, CAT, GNPDA1, SERPINA10, TWF1, HYOU1, VPS35, SNRPD3, EIF3B, VPS29, DNM2, RPL17, P4HB, MAP2K4, PSMD6, ARSA, RPL27A, ITGAV, PSMA2, HSPA8, S100A6, CDC37, NRP1, MACF1, PRCP, CLUH, COL12A1, HTRA1, SRM, SRC, CALU, ALDH7A1, CORO1C, CAD, UGP2, MAPK1, LGALS3BP, GSN, TXNL1, ALB, IGFALS, MTAP, PSMB2, TXN, RPS25, COLEC10, SLK, PLXDC2, CANX, PPP2R1A, RNASE4, FABP5, ATP6V1B2, RPL29, PSME2, RPL7A, AKR1B10, ELOB, STIP1, IMPA1, TLL1, IMPDH2, IGF2, CAPNS1, TPP1, USP24, ENO1, ANXA4, ASS1, NONO, CYB5R3, ASAH1, RPL14, EZR, TPI1, IGFBP6, DAZAP1, MYL12B, S100A10, RPLP0, CALM2, AP2A1, BRD4, AP2A2, ANXA5, ARF3, KPNB1, RPS14, PCDH19, PRKAR2A, CSTB, TGFBI, ACTR1A, HEXB, CS, LDLR, SERPINF1, F13A1, HSP90B1, CCT3, GANAB, BCLAF1, NSFL1C, PRDX1, DYNLL1, PEPD, TXNRD1, SSC5D, B2M, NOP58, HARS1, FBLN1, ANXA7, DOCK7, VPS36, RAN, LAMC1, JUP, DLG1, PPP2R2A, CNN2, UBA1, SVEP1, HNRNPA1, RAI14, RPL11, DDX6, NOTCH3, CTNND1, VAT1, ATP6V1A, FTH1, PTX3, USP5, ANXA6, COPB1, COL1A2, UGDH, ATP5F1B, AK1, PA2G4, SRSF1, PSMD14, LMNA, EIF4E, CP, GARS1, PABPC1, MASP1, HSPA1A, C8B, DDX3X, PSMB8, PXDN, SRSF6, COL6A2, CAP1, DDX1, PDIA4, LSM2, GALK1, BGN, PSMB7, ACACA, RACK1, SEC23B, CIS, H4C1, HSPA5, LGMN, TGFB1, MMP2, PROS1, UMPS, PLCD1, WASHC2C, EIF3E, HGFAC, RAB6A, NARS1, SEC61B, S100A4, EIF5A, VCP, SDCBP2, RPS2, YWHAB, RPL30, NUP93, C1QTNF3, PLP2, CAPZA1, CCT2, PNP, HSPG2, PYGB, VARS1, RPL7, RAC1, MAP4, ARPC3, WASF2, RAD21, USO1, RPL26, PSMD3, MAN2A1, ACO1, ANXA3, GDI2, NME2, ACO2, DNAJA1, ATRX, NCAM1, YES1, DYNC1LI1, DYNC1H1, IDH1, CPNE1, HNRNPD, MVP, RPL3, AC1N1, S100A11, CCT8, RARS1, CBR1, RAB7A, PGAM1, FASN, RUVBL2, PWP1, CCT4, MGP, HNRNPK, FN1, RPL6, CAPZA2, ACTR2, PRKAR1A, NOTCH2, NUDC, PCBP1, PSMC4, EFEMP1, ABCE1, SLC25A5, SPTAN1, UBE2N, RAB2A, SNRPD2, NEO1, RTN4, COPB2, NAGA, XPNPEP1, TKT, ARCN1, LGALS3, NID1, PDCD6, PSMD11, FARSB, DOCK1, EIF3K, HSPD1, PPIB, HSPA2, MYG1, EPRS1, APOM, TNPO1, GIPC1, NIBAN2, GP1, PSMC3, HSD17B12, PSMD7, ABI1, MPP7, LDHA, SEPTIN2, PPA1, PRDX4, FUBP1, EIF3C, HSPB1, QARS1, RPS3, PGK1, EIF5B, PSMB5, CCT5, EEF1D, PTPRF, RAB14, DDX5, RPS4X, MAN1A1, RPS8, DSTN, RPS15A, PCSK9, ARPC4, ANXA1, EEF1G, G6PD, H3-3A, NDRG1, YWHAQ, TSKU, MVD, AMY1A, ACLY, CSE1L, TPT1, NCL, SEPHS1, ADAMTS5, HMCN1, SNRPG, YWHAH, PSMC1, SERPIND1, CLU, TSPAN14, ANXA2, SEPTIN9, ECM1, GNS, FLNB, SEC23IP, GSTO1, TUFM, GCN1, EEF1E1, TAGLN2, BLMH, GDI1, TARS1, ESD, KIF5B, RPL22, GAPDH, COL2A1, IL6ST, PSMC6, TCP1, PGM2, HSP90AA1, ME1, MDH1, MFGE8, TNC, GET3, ENO3, RPLP1, PCOLCE, RCN1, HNRNPM, HGS, PSMB1, VCAN, EEF2, LAMA2, PLEC, EEF1B2, RPS11, PDCD6IP, GLUD1, UCHL3, CAPG, COL1A1, CHMP4B, DDX17, MAPRE1, BZW1, CYFIP1, UBE2V1, CCT7, PDIA3, CTTN, ACTN4, ACTN1, HSPH1, YWHAG, RPL5, or PSMA5, In some embodiments, the biomarker protein is not CPNE3, GNB1, CD9, GNAI2, SERBP1, SLC25A3, LAMB1, RAB5A, PRDX6, MSN, CLIC1, ARF3, PROS1, MVP, or a combination thereof. Also provided is a biomarker panel, comprising two or more biomarker proteins selected from List-A1. In some embodiments, the biomarker panel comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or 25 or more biomarker proteins selected from List-A1.

A biomarker protein may be selected from List-A2, wherein List-A2 is a group consisting of the proteins USP7, SEC31A, TARDBP, AP1B1, ZYX, RHOA, PI4KA, PSMC5, PCMT1, HNRNPC, GART, MAPK3, IP09, JAM3, CRYAB, STOM, RPS13, DLST, PRPSAP2, WDR26, PSME4, LIPG, NDNF, RAP2B, TLN1, FLNA, MRC2, SLC2A1, RPL10, PTMA, BZW2, LRRC59, RPL13A, ACAA1, H1-5, FSCN1, PSMD12, MARS1, TLN2, ARL8B, EHD4, TUBB1, MYH9, TGFB2, PITPNB, RBM3, ANO6, VCL, FBL, MYL6, CARM1, CST3, RALY, EIF2S3, TIMP3, ITGB3, ITGA6, SNAP23, DBNL, SPTBN1, PFN1, EIF2A, EMILIN1, PRMT5, HINT1, ATP6V1H, RPS17, HECTD1, DNAJA2, EML2, EIF5, ICAM1, OSBP, CAND1, PSMD2, MACROH2A1, TMED10, PSAP, DHX9, PALLD, SUMO2, DPP7, CMPK1, PDGFRB, RPL23, MIF, WIPI2, SYNCRIP, PCBP2, LGALS1, PFAS, EXOC1, UPF1, EFEMP2, VAPA, RAPIB, RPL24, CACNA2D1, LLGL1, TJP2, MED23, AGRN, OTUB1, NRAS, CORO1B, RPS21, PRDX2, CD81, ARPC5, CTNNB1, TUBA4A, SPP1, EIF3F, PGD, NAPG, AIMP1, TTLL12, FCSK, PSME3, NAPIL1, IARS1, SUB1, SRSF3, CAPN2, SERPINH1, TPM4, MYOSA, DHX15, PPP1CB, PSMD4, RPLP2, ATP6AP2, TRIM28, RPS26, RPS27A, DYNC112, PLCB3, RALB, RALA, SEPTIN7, DDB1, ALCAM, RAB10, SPARC, FTL, DLAT, CRABP2, ARF5, DNAJB1l, APP, USP14, IP07, PLXNA1, CDH2, KHDRBS1, RPL31, NAMPT, RPL23A, GPX1, HSPE1, LTBP1, PSMA4, TUBB, CTSB, SRI, ACTR10, NPM1, RTRAF, RPL18A, VIM, EIF3D, NSF, LIMS1, ACTB, USP15, CFL1, PPIA, RHOG, ITM2B, AIMP2, RPS20, ETF1, CD44, ADAM10, NUDT21, ATRN, AKR1A1, RPL15, FLNC, SOD1, RPS6, CALR, ITGB1, VDAC1, YWHAE, DCTN2, PPP2R1B, PSAT1, RTCB, EFR3A, EIF4G2, MAPRE2, METAP2, IDH2, HNRNPA2B1, HUWE1, NUCB1, TXNDC5, SRSF7, HSPA4L, TOLLIP, CAPN1, AARS1, VASP, CTSA, STRAP, PSMA3, EIF3I, DYNLRB1, SH3GL1, YWHAZ, ESYT1, ARHGDIA, MAP4K4, PITRM1, CD151, DCTN1, CDC42, AHSA1, RPS3A, GLIPR2, PFKL, ARPCIA, GSS, PDLIM5, RPS16, SFPQ, MYO9B, HNRNPUL2, SLC9A1, PCYOX1, PPP1CA, RPL4, TUBB6, CAVIN2, TXNDC17, SRPK1, PSMD13, GNB2, HNRNPF, PSMD1, DAAM1, NAPA, MYO1C, EIF4G1, CYCS, FBLN5, DNAJB1, RAB1A, LCAT, PURB, PRDX5, RPS23, EHD2, EHD1, EHD3, CD34, THBS1, RAB1B, HSPA9, AHCY, ACOT7, RPL9, C4B, UGGT1, PEF1, SDCBP, RPSA, LIMA1, APOB, SCFD1, G3BP1, GFUS, H1-4, PSMA1, VPS4B, FDPS, COL4A2, RAB5C, GLOD4, FGA, PSMB6, RNH1, TRIO, ATL3, FGG, PEBP1, AK2, ARPC2, PAICS, SNX6, RELN, TFRC, ARHGDIB, RPS18, LPL, LAP3, EIF4A1, NME1, BIRC6, LSM4, B4GALT1, PSMB4, MYO1B, PAFAH1B1, ALDOA, RAB11B, SND1, LOXL3, HSD17B4, COPE, HLA-B, ATIC, GJA1, DDAH2, GBA1, ADRM1, LTA4H, HS6ST1, ANXA11, RPL8, PITPNA, NUTF2, ILF2, H3C1, XRN2, ACTC1, EXT2, MDH2, TF, PFKP, PDIA6, SARS1, COPG1, RPL35A, PKM, RPL18, LAMP2, ELAVL1, ACTR3, CTSD, KARS1, RUVBL1, PLTP, PARP1, ILF3, LRP1, GSPT1, FARSA, TALDO1, KHSRP, GOT1, SRSF2, UBE2D3, ATP5F1A, JAK1, RBBP7, THBS3, WARS1, EEF1A1, BMP1, UNC5B, QSOX1, FBN1, LIPA, EIF6, CARS1, ALDH2, AKR1B1, SET, CCT6A, SNRPD1, NACA, GSTP1, HNRNPUL1, CDK4, PREP, SLC3A2, FLII, BANF1, OLA1, PHGDH, DDX39B, NPEPPS, MBP, BTF3, HNRNPH1, DSP, AXL, RAB5B, ADSL, PPP2CB, CTNNA1, RPL19, RPS19, MYO18A, HNRNPU, ANXA8L1, CAPRIN1, PARK7, DARS1, GMPPB, MEMO1, PDLIM1, ABCF2, HNRNPA3, APEH, DPYSL2, ACAT2, HSPA4, PSMA6, RPL13, ATP6V1E1, CSNK2A1, COPA, COL6A1, RPS9, GRN, SNRPB, IGF2R, EIF3L, TSG101, CLTC, IQGAP1, EIF2S1, PRPS1, EIF3A, MTHFD1, APRT, RPS7, GMPS, STX12, WDR1, LAMP1, PTBP1, PSMB3, DPP3, CKB, ITIH2, SNX2, HSP90ABI, LAMB2, SLIT3, HDLBP, GGH, BLVRA, ALDH16A1, HNRNPL, POSTN, YARS1, C5, COL4A1, IDE, COL18A1, CAT, GNPDA1, SERPINA10, TWF1, HYOU1, VPS35, SNRPD3, EIF3B, VPS29, DNM2, RPL17, P4HB, MAP2K4, PSMD6, ARSA, RPL27A, ITGAV, PSMA2, HSPA8, S100A6, CDC37, NRP1, MACF1, PRCP, CLUH, COL12A1, HTRA1, SRM, SRC, CALU, ALDH7A1, CORO1C, CAD, UGP2, MAPK1, LGALS3BP, GSN, TXNL1, ALB, IGFALS, MTAP, PSMB2, TXN, RPS25, COLEC10, SLK, PLXDC2, CANX, PPP2R1A, RNASE4, FABP5, ATP6V1B2, RPL29, PSME2, RPL7A, AKR1B10, ELOB, STIP1, IMPA1, TLL1, IMPDH2, IGF2, CAPNS1, TPP1, USP24, ENO1, ANXA4, ASS1, NONO, CYB5R3, ASAH1, RPL14, EZR, TPI1, IGFBP6, DAZAP1, MYL12B, S100A10, RPLP0, CALM2, AP2A1, BRD4, AP2A2, ANXA5, KPNB1, RPS14, PCDH19, PRKAR2A, CSTB, TGFBI, ACTR1A, HEXB, CS, LDLR, SERPINF1, F13A1, HSP90B1, CCT3, GANAB, BCLAF1, NSFL1C, PRDX1, DYNLL1, PEPD, TXNRD1, SSC5D, B2M, NOP58, HARS1, FBLN1, ANXA7, DOCK7, VPS36, RAN, LAMC1, JUP, DLG1, PPP2R2A, CNN2, UBA1, SVEP1, HNRNPA1, RAI14, RPL11, DDX6, NOTCH3, CTNND1, VAT1, ATP6V1A, FTH1, PTX3, USP5, ANXA6, COPB1, COL1A2, UGDH, ATP5F1B, AK1, PA2G4, SRSF1, PSMD14, LMNA, EIF4E, CP, GARS1, PABPC1, MASP1, HSPA1A, C8B, DDX3X, PSMB8, PXDN, SRSF6, COL6A2, CAP1, DDX1, PDIA4, LSM2, GALK1, BGN, PSMB7, ACACA, RACK1, SEC23B, CIS, H4C1, HSPA5, LGMN, TGFB1, MMP2, UMPS, PLCD1, WASHC2C, EIF3E, HGFAC, RAB6A, NARS1, SEC61B, S100A4, EIF5A, VCP, SDCBP2, RPS2, YWHAB, RPL30, NUP93, C1QTNF3, PLP2, CAPZA1, CCT2, PNP, HSPG2, PYGB, VARS1, RPL7, RAC1, MAP4, ARPC3, WASF2, RAD21, USO1, RPL26, PSMD3, MAN2A1, ACO1, ANXA3, GDI2, NME2, ACO2, DNAJA1, ATRX, NCAM1, YES1, DYNC1LI1, DYNC1H1, IDH1, CPNE1, HNRNPD, RPL3, ACIN1, S100A11, CCT8, RARS1, CBR1, RAB7A, PGAM1, FASN, RUVBL2, PWP1, CCT4, MGP, HNRNPK, FN1, RPL6, CAPZA2, ACTR2, PRKAR1A, NOTCH2, NUDC, PCBP1, PSMC4, EFEMP1, ABCE1, SLC25A5, SPTAN1, UBE2N, RAB2A, SNRPD2, NEO1, RTN4, COPB2, NAGA, XPNPEP1, TKT, ARCN1, LGALS3, NID1, PDCD6, PSMD11, FARSB, DOCK1, EIF3K, HSPD1, PPIB, HSPA2, MYG1, EPRS1, APOM, TNPO1, GIPC1, NIBAN2, GPI, PSMC3, HSD17B12, PSMD7, ABI1, MPP7, LDHA, SEPTIN2, PPA1, PRDX4, FUBP1, EIF3C, HSPB1, QARS1, RPS3, PGK1, EIF5B, PSMB5, CCT5, EEF1D, PTPRF, RAB14, DDX5, RPS4X, MAN1A1, RPS8, DSTN, RPS15A, PCSK9, ARPC4, ANXA1, EEF1G, G6PD, H3-3A, NDRG1, YWHAQ, TSKU, MVD, AMY1A, ACLY, CSE1L, TPT1, NCL, SEPHS1, ADAMTS5, HMCN1, SNRPG, YWHAH, PSMC1, SERPIND1, CLU, TSPAN14, ANXA2, SEPTIN9, ECM1, GNS, FLNB, SEC23IP, GSTO1, TUFM, GCN1, EEF1E1, TAGLN2, BLMH, GDI1, TARS1, ESD, KIF5B, RPL22, GAPDH, COL2A1, IL6ST, PSMC6, TCP1, PGM2, HSP90AA1, ME1, MDH1, MFGE8, TNC, GET3, ENO3, RPLP1, PCOLCE, RCN1, HNRNPM, HGS, PSMB1, VCAN, EEF2, LAMA2, PLEC, EEF1B2, RPS11, PDCD6IP, GLUD1, UCHL3, CAPG, COL1A1, CHMP4B, DDX17, MAPRE1, BZW1, CYFIP1, UBE2V1, CCT7, PDIA3, CTTN, ACTN4, ACTN1, HSPH1, YWHAG, RPL5, PSMA5, or a combination thereof. Also provided is a biomarker panel, comprising two or more biomarker proteins selected from List-A2. In some embodiments, the biomarker panel comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or 25 or more biomarker proteins selected from List-A2.

The biomarker protein may be selected from List-A3, wherein List-A3 is a group consisting of the proteins USP7, TARDBP, APIB1, ZYX, RHOA, PI4KA, PSMC5, PCMT1, HNRNPC, GART, MAPK3, IP09, JAM3, CRYAB, STOM, RPS13, DLST, PRPSAP2, WDR26, PSME4, LIPG, NDNF, RAP2B, TLN1, FLNA, MRC2, SLC2A1, RPL10, PTMA, BZW2, LRRC59, RPL13A, ACAA1, H1-5, FSCN1, PSMD12, MARS1, TLN2, ARL8B, EHD4, TUBB1, MYH9, TGFB2, PITPNB, RBM3, ANO6, VCL, FBL, MYL6, CARM1, CST3, RALY, EIF2S3, TIMP3, ITGB3, ITGA6, SNAP23, DBNL, PFN1, EIF2A, EMILIN1, PRMT5, HINT1, ATP6V1H, RPS17, HECTD1, DNAJA2, EML2, EIF5, ICAM1, OSBP, CAND1, PSMD2, MACROH2A1, TMED10, PSAP, PALLD, SUMO2, DPP7, CMPK1, PDGFRB, RPL23, MIF, WIPI2, SYNCRIP, PCBP2, LGALS1, PFAS, EXOC1, EFEMP2, VAPA, RAP1B, RPL24, CACNA2D1, LLGL1, TJP2, MED23, AGRN, NRAS, CORO1B, RPS21, PRDX2, CD81, ARPC5, CTNNB1, TUBA4A, EIF3F, PGD, NAPG, AIMP1, TTLL12, FCSK, PSME3, NAP1L1, IARS1, SUB1, SRSF3, CAPN2, SERPINH1, TPM4, MYOSA, PPP1CB, PSMD4, RPLP2, ATP6AP2, TRIM28, RPS26, RPS27A, DYNC112, PLCB3, RALB, RALA, SEPTIN7, DDB1, ALCAM, RAB10, SPARC, FTL, DLAT, CRABP2, ARF5, DNAJB11, APP, USP14, IP07, PLXNA1, CDH2, KHDRBS1, RPL31, NAMPT, RPL23A, GPX1, HSPE1, PSMA4, TUBB, CTSB, SRI, ACTR10, NPM1, RTRAF, RPL18A, VIM, EIF3D, NSF, LIMS1, ACTB, USP15, CFL1, PPIA, RHOG, AIMP2, RPS20, ETF1, CD44, ADAM10, NUDT21, ATRN, AKR1A1, RPL15, FLNC, SOD1, RPS6, CALR, ITGB1, VDAC1, YWHAE, PPP2R1B, PSAT1, RTCB, EFR3A, EIF4G2, MAPRE2, METAP2, HNRNPA2B1, HUWE1, NUCB1, TXNDC5, SRSF7, HSPA4L, TOLLIP, CAPN1, AARS1, VASP, CTSA, STRAP, PSMA3, EIF3I, DYNLRB1, SH3GL1, YWHAZ, ESYT1, ARHGDIA, MAP4K4, PITRM1, CD151, DCTN1, CDC42, AHSA1, RPS3A, GLIPR2, PFKL, ARPCIA, GSS, PDLIM5, RPS16, SFPQ, MYO9B, HNRNPUL2, SLC9A1, PCYOX1, PPP1CA, RPL4, TUBB6, CAVIN2, TXNDC17, SRPK1, PSMD13, GNB2, DAAM1, NAPA, MYO1C, EIF4G1, CYCS, FBLN5, DNAJB1, RAB1A, LCAT, PURB, PRDX5, RPS23, EHD2, EHD1, EHD3, CD34, THBS1, RAB1B, HSPA9, AHCY, ACOT7, RPL9, C4B, UGGT1, PEF1, SDCBP, RPSA, LIMA1, APOB, SCFD1, G3BP1, GFUS, H1-4, PSMA1, VPS4B, FDPS, COL4A2, RAB5C, GLOD4, FGA, PSMB6, RNH1, TRIO, ATL3, FGG, PEBP1, AK2, ARPC2, PAICS, SNX6, RELN, TFRC, ARHGDIB, RPS18, LPL, LAP3, EIF4A1, NME1, BIRC6, LSM4, B4GALT1, PSMB4, MYO1B, PAFAHIB1, ALDOA, RAB11B, SND1, LOXL3, HSD17B4, COPE, HLA-B, ATIC, GJA1, DDAH2, GBA1, ADRM1, LTA4H, HS6ST1, ANXA11, RPL8, PITPNA, NUTF2, ILF2, H3C1, XRN2, ACTC1, EXT2, MDH2, TF, PFKP, PDIA6, SARS1, COPG1, RPL35A, PKM, RPL18, LAMP2, ELAVL1, ACTR3, CTSD, KARS1, RUVBL1, PLTP, PARP1, ILF3, LRP1, GSPT1, FARSA, TALDO1, KHSRP, GOT1, SRSF2, UBE2D3, ATP5F1A, JAK1, RBBP7, THBS3, WARS1, EEF1A1, BMP1, UNC5B, QSOX1, FBN1, LIPA, EIF6, CARS1, SET, CCT6A, SNRPD1, NACA, GSTP1, HNRNPUL1, CDK4, PREP, SLC3A2, FLII, BANF1, OLA1, PHGDH, DDX39B, NPEPPS, MBP, BTF3, HNRNPH1, DSP, AXL, RAB5B, ADSL, PPP2CB, CTNNA1, RPL19, RPS19, MYO18A, HNRNPU, ANXA8L1, CAPRIN1, PARK7, DARS1, GMPPB, MEMO1, PDLIM1, ABCF2, HNRNPA3, APEH, DPYSL2, ACAT2, HSPA4, PSMA6, RPL13, ATP6V1E1, CSNK2A1, COPA, COL6A1, RPS9, GRN, SNRPB, IGF2R, EIF3L, TSG101, CLTC, IQGAP1, EIF2S1, PRPS1, EIF3A, MTHFD1, APRT, RPS7, GMPS, STX12, WDR1, LAMP1, PTBP1, PSMB3, DPP3, CKB, ITIH2, SNX2, HSP90AB1, LAMB2, SLIT3, HDLBP, GGH, BLVRA, ALDH16A1, HNRNPL, POSTN, YARS1, C5, COL4A1, IDE, COL18A1, CAT, GNPDA1, SERPINA10, TWF1, HYOU1, VPS35, SNRPD3, EIF3B, VPS29, DNM2, RPL17, P4HB, MAP2K4, PSMD6, ARSA, RPL27A, ITGAV, PSMA2, HSPA8, S100A6, CDC37, NRP1, MACF1, PRCP, CLUH, COL12A1, HTRA1, SRM, SRC, CALU, ALDH7A1, CORO1C, CAD, UGP2, MAPK1, LGALS3BP, GSN, TXNL1, ALB, IGFALS, MTAP, PSMB2, TXN, RPS25, COLEC10, SLK, PLXDC2, CANX, PPP2R1A, RNASE4, FABP5, ATP6V1B2, RPL29, PSME2, RPL7A, AKR1B10, ELOB, STIP1, IMPA1, TLL1, IMPDH2, IGF2, CAPNS1, TPP1, USP24, ENO1, ANXA4, ASS1, NONO, CYB5R3, ASAH1, RPL14, EZR, TPI1, IGFBP6, DAZAP1, MYL12B, S100A10, RPLP0, CALM2, AP2A1, BRD4, AP2A2, ANXA5, KPNB1, RPS14, PCDH19, PRKAR2A, CSTB, TGFBI, ACTR1A, HEXB, CS, LDLR, SERPINF1, F13A1, HSP90B1, CCT3, GANAB, BCLAF1, NSFL1C, PRDX1, DYNLL1, PEPD, TXNRD1, SSC5D, B2M, NOP58, HARS1, FBLN1, ANXA7, DOCK7, VPS36, RAN, LAMC1, JUP, DLG1, PPP2R2A, CNN2, UBA1, SVEP1, HNRNPA1, RAI14, RPL11, DDX6, NOTCH3, CTNND1, VAT1, ATP6V1A, FTH1, PTX3, USP5, ANXA6, COPB1, COL1A2, UGDH, ATP5F1B, AK1, PA2G4, SRSF1, PSMD14, LMNA, EIF4E, CP, GARS1, PABPC1, MASP1, HSPA1A, C8B, DDX3X, PSMB8, PXDN, SRSF6, COL6A2, CAP1, DDX1, PDIA4, LSM2, GALK1, BGN, PSMB7, ACACA, RACK1, SEC23B, CIS, H4C1, HSPA5, LGMN, TGFB1, MMP2, UMPS, PLCD1, WASHC2C, EIF3E, HGFAC, RAB6A, NARS1, SEC61B, S100A4, EIF5A, VCP, SDCBP2, RPS2, YWHAB, RPL30, NUP93, C1QTNF3, PLP2, CAPZA1, CCT2, PNP, HSPG2, PYGB, VARS1, RPL7, RAC1, MAP4, ARPC3, WASF2, RAD21, USO1, RPL26, PSMD3, MAN2A1, ACO1, ANXA3, GDI2, NME2, ACO2, DNAJA1, ATRX, NCAM1, YES1, DYNC1LI1, DYNC1H1, IDH1, CPNE1, HNRNPD, RPL3, AC1N1, S100A11, CCT8, RARS1, CBR1, RAB7A, PGAM1, FASN, RUVBL2, PWP1, CCT4, MGP, HNRNPK, FN1, RPL6, CAPZA2, ACTR2, PRKAR1A, NOTCH2, NUDC, PCBP1, PSMC4, EFEMP1, ABCE1, SLC25A5, SPTAN1, UBE2N, RAB2A, SNRPD2, NEO1, RTN4, COPB2, NAGA, XPNPEP1, TKT, ARCN1, LGALS3, NID1, PDCD6, PSMD11, FARSB, DOCK1, EIF3K, HSPD1, PPIB, HSPA2, MYG1, EPRS1, APOM, TNPO1, GIPC1, NIBAN2, GPI, PSMC3, HSD17B12, PSMD7, ABI1, MPP7, LDHA, SEPTIN2, PPA1, PRDX4, FUBP1, EIF3C, HSPB1, QARS1, RPS3, PGK1, EIF5B, PSMB5, CCT5, EEF1D, PTPRF, RAB14, DDX5, RPS4X, MAN1A1, RPS8, DSTN, RPS15A, PCSK9, ARPC4, ANXA1, EEF1G, G6PD, H3-3A, NDRG1, YWHAQ, TSKU, MVD, AMY1A, ACLY, CSE1L, TPT1, NCL, SEPHS1, ADAMTS5, HMCN1, SNRPG, YWHAH, PSMC1, SERPIND1, CLU, TSPAN14, ANXA2, SEPTIN9, ECM1, GNS, FLNB, SEC23IP, GSTO1, TUFM, GCN1, EEF1E1, TAGLN2, BLMH, GDI1, TARS1, ESD, KIF5B, RPL22, GAPDH, COL2A1, IL6ST, PSMC6, TCP1, PGM2, HSP90AA1, ME1, MDH1, MFGE8, TNC, GET3, ENO3, RPLP1, PCOLCE, RCN1, HNRNPM, HGS, PSMB1, VCAN, EEF2, LAMA2, PLEC, EEF1B2, RPS11, PDCD6IP, GLUD1, UCHL3, CAPG, COL1A1, CHMP4B, DDX17, MAPRE1, BZW1, CYFIP1, UBE2V1, CCT7, PDIA3, CTTN, ACTN4, ACTN1, HSPH1, YWHAG, RPL5, PSMA5, or a combination thereof. Also provided is a biomarker panel, comprising two or more biomarker proteins selected from List-A3. In some embodiments, the biomarker panel comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or 25 or more biomarker proteins selected from List-A3.

The biomarker protein may be selected from List-A4, wherein List-A4 is a group consisting of the proteins ZYX, RHOA, PI4KA, PSMC5, PCMT1, HNRNPC, GART, MAPK3, IP09, JAM3, STOM, RPS13, DLST, PRPSAP2, NDNF, RAP2B, TLN1, FLNA, MRC2, SLC2A1, RPL10, PTMA, BZW2, LRRC59, RPL13A, ACAA1, H1-5, FSCN1, PSMD12, MARS1, TLN2, ARL8B, EHD4, TUBB1, MYH9, TGFB2, PITPNB, RBM3, ANO6, VCL, FBL, MYL6, CST3, RALY, EIF2S3, ITGA6, SNAP23, DBNL, PFN1, EIF2A, EMILIN1, PRMT5, HINT1, RPS17, HECTD1, DNAJA2, EML2, EIF5, ICAM1, OSBP, CAND1, PSMD2, MACROH2A1, TMED10, PSAP, PALLD, SUMO2, DPP7, CMPK1, PDGFRB, RPL23, MIF, WIPI2, SYNCRIP, PCBP2, LGALS1, PFAS, EXOC1, EFEMP2, RAP1B, RPL24, CACNA2D1, LLGL1, TJP2, AGRN, NRAS, CORO1B, RPS21, PRDX2, CD81, ARPC5, CTNNB1, TUBA4A, EIF3F, PGD, NAPG, AIMP1, TTLL12, FCSK, PSME3, NAPIL1, IARS1, SUB1, SRSF3, CAPN2, SERPINH1, TPM4, PPP1CB, RPLP2, ATP6AP2, TRIM28, RPS26, RPS27A, DYNC112, PLCB3, RALB, RALA, SEPTIN7, DDB1, ALCAM, RAB10, SPARC, FTL, DLAT, CRABP2, ARF5, DNAJB11, APP, USP14, PLXNA1, CDH2, KHDRBS1, RPL31, NAMPT, RPL23A, GPX1, HSPE1, PSMA4, TUBB, CTSB, SRI, ACTR10, NPM1, RPL18A, VIM, EIF3D, NSF, LIMS1, ACTB, USP15, CFL1, PPIA, RHOG, AIMP2, RPS20, ETF1, CD44, ADAM10, NUDT21, ATRN, AKR1A1, RPL15, FLNC, SOD1, RPS6, CALR, ITGB1, VDAC1, YWHAE, PPP2R1B, PSAT1, RTCB, EFR3A, MAPRE2, METAP2, HNRNPA2B1, NUCB1, TXNDC5, HSPA4L, TOLLIP, CAPN1, AARS1, VASP, CTSA, STRAP, PSMA3, EIF3I, DYNLRB1, SH3GL1, YWHAZ, ARHGDIA, PITRM1, CD151, DCTN1, CDC42, AHSA1, RPS3A, GLIPR2, PFKL, ARPCIA, PDLIM5, RPS16, SFPQ, MYO9B, HNRNPUL2, PCYOX1, PPP1CA, RPL4, TUBB6, CAVIN2, TXNDC17, SRPK1, PSMD13, GNB2, DAAM1, NAPA, MYO1C, EIF4G1, CYCS, FBLN5, DNAJB1, RAB1A, LCAT, PURB, PRDX5, RPS23, EHD2, EHD1, EHD3, CD34, THBS1, HSPA9, AHCY, ACOT7, RPL9, C4B, UGGT1, PEF1, SDCBP, RPSA, LIMA1, APOB, G3BP1, GFUS, H1-4, PSMA1, VPS4B, FDPS, COL4A2, RAB5C, GLOD4, FGA, PSMB6, RNH1, TRIO, ATL3, FGG, PEBP1, AK2, ARPC2, PAICS, SNX6, RELN, TFRC, ARHGDIB, RPS18, LPL, LAP3, EIF4A1, NME1, BIRC6, B4GALT1, PSMB4, MYO1B, PAFAH1B1, ALDOA, RAB11B, SND1, LOXL3, HLA-B, ATIC, GJA1, DDAH2, GBA1, ADRM1, LTA4H, HS6ST1, ANXA11, RPL8, PITPNA, NUTF2, H3C1, XRN2, ACTC1, EXT2, MDH2, TF, PFKP, SARS1, COPG1, RPL35A, PKM, RPL18, LAMP2, ELAVL1, ACTR3, CTSD, KARS1, RUVBL1, PLTP, PARP1, LRP1, GSPT1, FARSA, TALDO1, KHSRP, GOT1, SRSF2, UBE2D3, ATP5F1A, JAK1, RBBP7, THBS3, WARS1, EEF1A1, BMP1, UNC5B, QSOX1, FBN1, LIPA, EIF6, CARS1, SET, CCT6A, SNRPD1, NACA, GSTP1, HNRNPUL1, CDK4, SLC3A2, FLII, BANF1, OLA1, PHGDH, NPEPPS, BTF3, DSP, AXL, RAB5B, ADSL, PPP2CB, CTNNA1, RPL19, RPS19, HNRNPU, ANXA8L1, CAPRIN1, PARK7, DARS1, MEMO1, PDLIM1, ABCF2, HNRNPA3, APEH, DPYSL2, ACAT2, HSPA4, PSMA6, RPL13, ATP6V1E1, CSNK2A1, COPA, RPS9, GRN, IGF2R, EIF3L, TSG101, CLTC, IQGAP1, EIF2S1, PRPS1, EIF3A, MTHFD1, APRT, RPS7, GMPS, WDR1, LAMP1, PTBP1, PSMB3, CKB, ITIH2, SNX2, HSP90AB1, LAMB2, SLIT3, GGH, BLVRA, ALDH16A1, POSTN, YARS1, C5, COL4A1, IDE, COL18A1, CAT, GNPDA1, SERPINA10, TWF1, HYOU1, VPS35, SNRPD3, EIF3B, VPS29, DNM2, RPL17, P4HB, PSMD6, ARSA, RPL27A, ITGAV, PSMA2, HSPA8, S100A6, CDC37, MACF1, PRCP, CLUH, COL12A1, HTRA1, SRM, SRC, CALU, ALDH7A1, CORO1C, CAD, UGP2, MAPK1, LGALS3BP, GSN, ALB, IGFALS, PSMB2, TXN, RPS25, COLEC10, SLK, PLXDC2, PPP2R1A, RNASE4, FABP5, ATP6V1B2, RPL29, PSME2, RPL7A, AKRIB10, STIP1, TLL1, IMPDH2, IGF2, CAPNS1, TPP1, ENO1, ANXA4, ASS1, NONO, ASAH1, RPL14, EZR, TPI1, IGFBP6, DAZAP1, MYL12B, S100A10, RPLP0, CALM2, AP2A1, AP2A2, ANXA5, KPNB1, RPS14, PRKAR2A, CSTB, TGFBI, HEXB, LDLR, HSP90B1, CCT3, GANAB, BCLAF1, NSFL1C, PRDX1, DYNLL1, PEPD, TXNRD1, SSC5D, B2M, HARS1, FBLN1, ANXA7, DOCK7, VPS36, RAN, LAMC1, JUP, DLG1, CNN2, UBA1, HNRNPA1, RAI14, RPL11, CTNND1, VAT1, ATP6V1A, PTX3, USP5, ANXA6, COPB1, COL1A2, UGDH, ATP5F1B, AK1, PA2G4, SRSF1, PSMD14, EIF4E, CP, GARS1, PABPC1, MASP1, HSPAIA, C8B, PSMB8, PXDN, SRSF6, COL6A2, CAP1, DDX1, LSM2, GALK1, BGN, PSMB7, RACK1, SEC23B, CIS, H4C1, HSPA5, LGMN, TGFB1, MMP2, UMPS, PLCD1, WASHC2C, EIF3E, HGFAC, NARS1, S100A4, EIF5A, VCP, SDCBP2, RPS2, YWHAB, RPL30, PLP2, CAPZA1, CCT2, HSPG2, PYGB, VARS1, RPL7, RAC1, MAP4, ARPC3, WASF2, RPL26, PSMD3, MAN2A1, ANXA3, GDI2, NME2, ACO2, DNAJA1, ATRX, NCAM1, YES1, DYNC1LI1, DYNC1H1, IDH1, CPNE1, HNRNPD, RPL3, ACIN1, CCT8, RARS1, CBR1, RAB7A, PGAM1, FASN, RUVBL2, CCT4, MGP, HNRNPK, FN1, RPL6, CAPZA2, ACTR2, PRKAR1A, NOTCH2, NUDC, PCBP1, PSMC4, RAB2A, SNRPD2, NEO1, RTN4, NAGA, XPNPEP1, TKT, ARCN1, LGALS3, NID1, PDCD6, PSMD11, FARSB, DOCK1, EIF3K, HSPD1, PPIB, HSPA2, MYG1, EPRS1, TNPO1, NIBAN2, GPI, PSMC3, HSD17B12, PSMD7, ABI1, MPP7, LDHA, SEPTIN2, PPA1, PRDX4, FUBP1, EIF3C, HSPB1, QARS1, RPS3, PGK1, EIF5B, PSMB5, CCT5, EEF1D, PTPRF, RAB14, RPS4X, MAN1A1, RPS8, DSTN, RPS15A, PCSK9, ARPC4, ANXA1, EEF1G, G6PD, H3-3A, YWHAQ, TSKU, AMY1A, ACLY, CSE1L, TPT1, NCL, SEPHS1, YWHAH, PSMC1, SERPIND1, CLU, TSPANI4, ANXA2, SEPTIN9, ECM1, GNS, FLNB, TUFM, GCN1, TAGLN2, BLMH, GDI1, TARS1, ESD, KIF5B, GAPDH, COL2A1, IL6ST, PSMC6, TCP1, PGM2, HSP90AA1, ME1, MDH1, MFGE8, TNC, GET3, ENO3, RPLP1, PCOLCE, RCN1, HNRNPM, HGS, PSMB1, VCAN, EEF2, LAMA2, PLEC, EEFIB2, RPS11, PDCD6IP, GLUD1, UCHL3, COL1A1, CHMP4B, DDX17, MAPRE1, BZW1, CYFIP1, UBE2V1, CCT7, CTTN, ACTN4, ACTN1, HSPH1, YWHAG, RPL5, PSMA5, or a combination thereof. Also provided is a biomarker panel, comprising two or more biomarker proteins selected from List-A4. In some embodiments, the biomarker panel comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or 25 or more biomarker proteins selected from List-A4.

A biomarker protein may be a human plasma derived biomarker, optionally having a p-value of less than 0.05 in human derived EVs by genotype. The biomarker may be determined by a method of identifying a candidate biomarker protein as described herein (e.g., (e.g., p<0.05 human derived EV biomarker by genotype or for DYT1 status). The biomarker protein may have a fold change of at least ±0.5, at least ±1.0, at least ±1.5, at least ±2.0, at least ±3.0, or at least ±4.0 relative to a reference level, as described herein. The biomarker protein may be a high priority candidate biomarker, as described herein. The biomarker protein may be selected from List-B1, wherein List-B1 is a group consisting of the proteins USP7, SEC31A, TARDBP, AP1B1, ZYX, RHOA, PI4KA, PSMC5, PCMT1, HNRNPC, GART, MAPK3, IP09, JAM3, CRYAB, STOM, RPS13, DLST, PRPSAP2, WDR26, PSME4, LIPG, NDNF, RAP2B, TLN1, FLNA, MRC2, SLC2A1, RPL10, PTMA, BZW2, LRRC59, RPL13A, ACAA1, H1-5, FSCN1, PSMD12, MARS1, TLN2, ARL8B, EHD4, TUBB1, MYH9, TGFB2, PITPNB, RBM3, or ANO6. Also provided is a biomarker panel, comprising two or more biomarker proteins selected from List-B1. In some embodiments, the biomarker panel comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or 25 or more biomarker proteins selected from List-B1. The biomarker protein may selected from List-B2, wherein List-B2 is a group consisting of the proteins USP7, TARDBP, APIB1, ZYX, RHOA, PI4KA, PSMC5, PCMT1, HNRNPC, GART, MAPK3, IP09, JAM3, CRYAB, STOM, RPS13, DLST, PRPSAP2, WDR26, PSME4, LIPG, NDNF, RAP2B, TLN1, FLNA, MRC2, SLC2A1, RPL10, PTMA, BZW2, LRRC59, RPL13A, ACAA1, H1-5, FSCN1, PSMD12, MARS1, TLN2, ARL8B, EHD4, TUBB1, MYH9, TGFB2, PITPNB, RBM3, ANO6, or a combination thereof. Also provided is a biomarker panel, comprising two or more biomarker proteins selected from List-B2. In some embodiments, the biomarker panel comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or 25 or more biomarker proteins selected from List-B2. The biomarker may be selected from List-B3, wherein List-B3 is a group consisting of the proteins ZYX, RHOA, PI4KA, PSMC5, PCMT1, HNRNPC, GART, MAPK3, IP09, JAM3, STOM, RPS13, DLST, PRPSAP2, NDNF, RAP2B, TLN1, FLNA, MRC2, SLC2A1, RPL10, PTMA, BZW2, LRRC59, RPL13A, ACAA1, H1-5, FSCN1, PSMD12, MARS1, TLN2, ARL8B, EHD4, TUBB1, MYH9, TGFB2, PITPNB, RBM3, ANO6, or a combination thereof. Also provided is a biomarker panel, comprising two or more biomarker proteins selected from List-B3. In some embodiments, the biomarker panel comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or 25 or more biomarker proteins selected from List-B3.

A biomarker protein may be a human plasma derived biomarker, which has a p-value of less than 0.05 in human derived EVs by genotype, and a fold change of at least ±0.5 relative to a reference level, as described herein. The biomarker may be determined by a method of identifying a candidate biomarker protein as described herein (e.g., p<0.05 human derived EV biomarker by genotype for DYT1 status). The biomarker protein may have a fold change of at least ±1.0, at least ±1.5, at least ±2.0, at least ±3.0, or at least ±4.0 relative to a reference level. The biomarker protein may be selected from List-C1, wherein List-C1 is a group consisting ofthe proteins KCNB2, SLC4A1, FAM131C, RNF216, NECTIN1, OBP2A, HBA1, NDNF, MPIG6B, BRD9, STOM, ITGA2B, NAA20, CEP170, FAM177A1, PLEK, TUBB1, TLN1, ILK, FLNA, TUBAIB, WDR26, MYH9, RHOA, RPS13, NEK9, PSMC5, ATRIP, DDOST, PCMT1, LMNB2, FGL1, MUCL1, RPL13A, VWF, ATP5F1C, ACAA1, WNK1, H1-5, DLD, PSMD12, PITPNB, LAMA1, TRIM16, DLST, CRYAB, SLPI, PRPSAP2, F8, SEC31A, RBM3, LYPLA1, PTGDR2, ARL8B, USP2, TUBAIC, SETD2, WFDC12, JAM3, SOGA1, TFB1M, RAP2B, HBD, ADAMTS19, GP9, C16orf86, GALNTL6, MFAP5, GP1BB, ANK1, HLA-C, SPTB, MRC2, SLC2A1, EHD4, PAFAH1B2, ZYX, RHOC, GOLGA4, SLC25A6, ACOX3, RPL10, USP7, PTMA, RNF39, HNRNPC, DNAH2, ATP6V1G1, BZW2, LRRC59, PSME4, LORICRIN, GART, TRAPPC9, MAPK3, LIPG, KRT33A, FSCN1, TGFB2, SELENOF, IP09, MARS1, TARDBP, TLN2, GPR17, IGHV8-51-1, SP110, PCDHB15, SLC25A11, AP1B1, ANO6, FAM98A, ABCB9, MK167, LCK, FYN, YES1, SRC, EHD1, EHD3, EHD2 or a combination thereof. Also provided is a biomarker panel, comprising two or more biomarker proteins selected from List-C1. In some embodiments, the biomarker panel comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or 25 or more biomarker proteins selected from List-C1. The biomarker protein may be selected from List-C2, wherein List-C2 is a group consisting of the proteins KCNB2, SLC4A1, FAM131C, RNF216, NECTIN1, OBP2A, HBA1, NDNF, MPIG6B, BRD9, STOM, ITGA2B, NAA20, CEP170, FAM177A1, PLEK, TUBB1, TLN1, ILK, FLNA, TUBAIB, WDR26, MYH9, RHOA, RPS13, NEK9, PSMC5, ATRIP, DDOST, PCMT1, LMNB2, FGL1, MUCL1, RPL13A, VWF, ATP5F1C, ACAA1, WNK1, H1-5, DLD, PSMD12, PITPNB, LAMA1, TRIM16, DLST, CRYAB, SLPI, PRPSAP2, F8, RBM3, LYPLA1, PTGDR2, ARL8B, USP2, TUBAIC, SETD2, WFDC12, JAM3, SOGA1, TFB1M, RAP2B, HBD, ADAMTS19, GP9, C16orf86, GALNTL6, MFAP5, GP1BB, ANK1, HLA-C, SPTB, MRC2, SLC2A1, EHD4, PAFAH1B2, ZYX, RHOC, GOLGA4, SLC25A6, ACOX3, RPL10, USP7, PTMA, RNF39, HNRNPC, DNAH2, ATP6V1G1, BZW2, LRRC59, PSME4, LORICRIN, GART, TRAPPC9, MAPK3, LIPG, KRT33A, FSCN1, TGFB2, SELENOF, IP09, MARS1, TARDBP, TLN2, GPR17, IGHV8-51-1, SP110, PCDHB15, SLC25A11, AP1B1, ANO6, FAM98A, ABCB9, MKI67, LCK, FYN, YES1, SRC, EHD1, EHD3, EHD2 or a combination thereof. Also provided is a biomarker panel, comprising two or more biomarker proteins selected from List-C2. In some embodiments, the biomarker panel comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or 25 or more biomarker proteins selected from List-C2. The biomarker protein may be selected from List-C3, wherein List-C3 is a group consisting of the proteins KCNB2, SLC4A1, FAM131C, RNF216, NECTIN1, OBP2A, HBA1, NDNF, MPIG6B, BRD9, STOM, ITGA2B, NAA20, CEP170, FAM177A1, PLEK, TUBB1, TLN1, ILK, FLNA, TUBAIB, MYH9, RHOA, RPS13, NEK9, PSMC5, ATRIP, DDOST, PCMT1, LMNB2, FGL1, MUCL1, RPL13A, VWF, ATP5F1C, ACAA1, WNK1, H1-5, DLD, PSMD12, PITPNB, LAMA1, TRIM16, DLST, SLPI, PRPSAP2, F8, RBM3, LYPLA1, PTGDR2, ARL8B, USP2, TUBAIC, SETD2, WFDC12, JAM3, SOGA1, TFB1M, RAP2B, HBD, ADAMTS19, GP9, C16orf86, GALNTL6, MFAP5, GP1BB, ANK1, HLA-C, SPTB, MRC2, SLC2A1, EHD4, PAFAH1B2, ZYX, RHOC, GOLGA4, SLC25A6, ACOX3, RPL10, PTMA, RNF39, HNRNPC, DNAH2, ATP6V1G1, BZW2, LRRC59, LORICRIN, GART, TRAPPC9, MAPK3, KRT33A, FSCN1, TGFB2, SELENOF, IP09, MARS1, TLN2, GPR17, IGHV8-51-1, SP110, PCDHB15, SLC25A11, ANO6, FAM98A, ABCB9, MK167, LCK, FYN, YES1, SRC, EHD1, EHD3, EHD2 or a combination thereof. Also provided is a biomarker panel, comprising two or more biomarker proteins selected from List-C3. In some embodiments, the biomarker panel comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or 25 or more biomarker proteins selected from List-C3.

A biomarker protein may be a human plasma derived biomarker, optionally an EV biomarker, which has a p-value of less than 0.05 in human derived EVs by genotype. A biomarker protein may have a p-value of less than 0.05, such as in a method of identifying a candidate biomarker protein as described herein (e.g., p<0.05 human derived EV biomarker for DYT1 status and common with an MEF derived EV biomarker). The biomarker protein may have a fold change of at least ±0.5, at least ±1.0, at least ±1.5, at least ±2.0, at least ±3.0, or at least ±4.0 relative to a reference level, as described herein. The biomarker protein may be selected from List-D1, wherein List-D1 is a group consisting of the proteins USP7, SEC31A, TARDBP, AP1B1, ZYX, PSMC5, PCMT1, GART, MAPK3, IP09, CRYAB, RPS13, PSME4, LIPG, MRC2, SLC2A1, RPL10, BZW2, LRRC59, RPL13A, ACAA1, H1-5, FSCN1, PSMD12, MARS1, TLN2, ARL8B, EHD4, TGFB2, RBM3 or a combination thereof. Also provided is a biomarker panel, comprising two or more biomarker proteins selected from List-D1. In some embodiments, the biomarker panel comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or 25 or more biomarker proteins selected from List-D1. The biomarker protein may be selected from List-D2, wherein List-D2 is a group consisting of the proteins USP7, TARDBP, AP1B1, ZYX, PSMC5, PCMT1, GART, MAPK3, IP09, CRYAB, RPS13, PSME4, LIPG, MRC2, SLC2A1, RPL10, BZW2, LRRC59, RPL13A, ACAA1, H1-5, FSCN1, PSMD12, MARS1, TLN2, ARL8B, EHD4, TGFB2, RBM3 or a combination thereof. Also provided is a biomarker panel, comprising two or more biomarker proteins selected from List-D2. In some embodiments, the biomarker panel comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or 25 or more biomarker proteins selected from List-D2. The biomarker protein may be selected from List-D3, wherein List-D3 is a group consisting of the proteins ZYX, PSMC5, PCMT1, GART, MAPK3, IP09, RPS13, MRC2, SLC2A1, RPL10, BZW2, LRRC59, RPL13A, ACAA1, H1-5, FSCN1, PSMD12, MARS1, TLN2, ARL8B, EHD4, TGFB2, RBM3 or a combination thereof. Also provided is a biomarker panel, comprising two or more biomarker proteins selected from List-D3. In some embodiments, the biomarker panel comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, or 22 or more biomarker proteins selected from List-D3,

A biomarker protein may be a human plasma derived EV biomarker, which is corrected following treatment using an agent as described herein (e.g., ritonavir), optionally wherein the biomarker has a p-value of less than 0.05 in human derived EVs by genotype. The biomarker may be determined by a method of identifying a candidate biomarker protein as described herein (e.g., identifying a human derived EV dystonia protein biomarker that corrects its aberrant value following treatment with an agent as described herein, such as for ritonavir in MEF EVs). The biomarker protein may have a fold change of at least ±0.5, at least ±1.0, at least ±1.5, at least ±2.0, at least ±3.0, or at least ±4.0 relative to a reference level, as described herein. The biomarker protein may be selected from List-E1, wherein List-E1 is agroup consisting of the proteins USP7, SEC31A, TARDBP, AP1B1, ZYX, RHOA, PI4KA, PSMC5, PCMT1, HNRNPC, GART, MAPK3, IP09, JAM3, CRYAB, STOM, RPS13, DLST, PRPSAP2, WDR26, PSME4, LIPG, NDNF, RAP2B, TLN1, FLNA, MRC2, SLC2A1, RPL10, PTMA, BZW2, LRRC59, RPL13A, ACAA1, H1-5, FSCN1, PSMD12, MARS1, TLN2, ARL8B, EHD4 or a combination thereof. Also provided is a biomarker panel, comprising two or more biomarker proteins selected from List-E1. In some embodiments, the biomarker panel comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or 25 or more biomarker proteins selected from List-E1. The biomarker protein may be selected from List-E2, wherein List-E2 is a group consisting of the proteins USP7, TARDBP, AP1B1, ZYX, RHOA, PI4KA, PSMC5, PCMT1, HNRNPC, GART, MAPK3, IP09, JAM3, CRYAB, STOM, RPS13, DLST, PRPSAP2, WDR26, PSME4, LIPG, NDNF, RAP2B, TLN1, FLNA, MRC2, SLC2A1, RPL10, PTMA, BZW2, LRRC59, RPL13A, ACAA1, H1-5, FSCN1, PSMD12, MARS1, TLN2, ARL8B, EHD4 or a combination thereof. Also provided is a biomarker panel, comprising two or more biomarker proteins selected from List-E2. In some embodiments, the biomarker panel comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or 25 or more biomarker proteins selected from List-E2. The biomarker may be selected from List-E3, wherein List-E3 is a group consisting of the proteins ZYX, RHOA, PI4KA, PSMC5, PCMT1, HNRNPC, GART, MAPK3, IP09, JAM3, STOM, RPS13, DLST, PRPSAP2, NDNF, RAP2B, TLN1, FLNA, MRC2, SLC2A1, RPL10, PTMA, BZW2, LRRC59, RPL13A, ACAA1, H1-5, FSCN1, PSMD12, MARS1, TLN2, ARL8B, EHD4 or a combination thereof. Also provided is a biomarker panel, comprising two or more biomarker proteins selected from List-E3. In some embodiments, the biomarker panel comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or 25 or more biomarker proteins selected from List-E3.

A biomarker protein may be a human plasma derived EV biomarker, which is corrected following treatment using an agent as described herein (e.g., salurbinal), optionally wherein the biomarker has a p-value of less than 0.05 in human derived EVs by genotype. The biomarker may be determined by a method of identifying a candidate biomarker protein as described herein (e.g., identifying a human derived EV dystonia protein biomarker that corrects its aberrant value following treatment with an agent as described herein, such as for salurbinal in MEF EVs). The biomarker protein may have a fold change of at least ±0.5, at least ±1.0, at least ±1.5, at least ±2.0, at least ±3.0, or at least ±4.0 relative to a reference level, as described herein. The biomarker protein may be selected from List-F1, wherein List-F1 is a group consisting of the proteins USP7, SEC31A, TARDBP, JAM3, PSMC5, PCMT1, HNRNPC, CRYAB, STOM, RPS13, AP1B1, ZYX, RHOA, GART, MAPK3, IP09, PRPSAP2, DLST, PI4KA or a combination thereof. Also provided is a biomarker panel, comprising two or more biomarker proteins selected from List-F1. In some embodiments, the biomarker panel comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, or 18 or more biomarker proteins selected from List-F1. The biomarker protein may be selected from List-F2, wherein List-F2 is a group consisting of the proteins USP7, TARDBP, JAM3, PSMC5, PCMT1, HNRNPC, CRYAB, STOM, RPS13, AP1B1, ZYX, RHOA, GART, MAPK3, IP09, PRPSAP2, DLST, PI4KA or a combination thereof. Also provided is a biomarker panel, comprising two or more biomarker proteins selected from List-F2. In some embodiments, the biomarker panel comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, or 17 or more biomarker proteins selected from List-F2. The biomarker may be selected from List-F3, wherein List-F3 is a group consisting of the proteins JAM3, PSMC5, PCMT1, HNRNPC, STOM, RPS13, ZYX, RHOA, GART, MAPK3, IP09, PRPSAP2, DLST, PI4KA or a combination thereof. Also provided is a biomarker panel, comprising two or more biomarker proteins selected from List-F3. In some embodiments, the biomarker panel comprises 2 or more, 3 or more, 4 or more, 5 or more, 6 or more, 7 or more, 8 or more, 9 or more, 10 or more, 11 or more, 12 or more, or 13 or more biomarker proteins selected from List-F3.

A biomarker protein may be a human plasma derived EV biomarker, for which treatment with an integrated stress response inhibitor replicates DYT1 state (e.g., as demonstrated in MEF EVs as described herein), optionally wherein the biomarker has a p-value of less than 0.05 in human derived EVs by genotype. The biomarker may be determined by a method of identifying a candidate biomarker protein as described herein (e.g., identifying a human derived EV dystonia protein biomarker that shows replication of DYT1 state following treatment with an integrated stress response inhibitor, as described herein, such as for ISRIB in MEF EVs). The biomarker protein may have a fold change of at least ±0.5, at least ±1.0, at least ±1.5, at least ±2.0, at least ±3.0, or at least ±4.0 relative to a reference level, as described herein. The biomarker protein may be selected from List-G1, wherein List-G1 a group consisting of the proteins USP7, SEC31A, TARDBP, AP1B1, ZYX, RHOA, HNRNPC, GART, MAPK3, IP09, JAM3, WDR26, RAP2B, TLN1, FLNA, SLC2A1, PTMA, ACAA1, MARS1, TLN2, ARL8B, EHD4, TGFB2, PITPNB, PI4KA, PSMC5, PCMT1, H1-5, FSCN1, PSMD12, PSME4, LIPG, NDNF, ANO6 or a combination thereof. Also provided is a biomarker panel, comprising two or more biomarker proteins selected from List-G1. In some embodiments, the biomarker panel comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, or 17 or more biomarker proteins selected from List-G1. The biomarker protein may be selected from List-G2, wherein List-G2 a group consisting of the proteins USP7, TARDBP, AP1B1, ZYX, RHOA, HNRNPC, GART, MAPK3, IP09, JAM3, WDR26, RAP2B, TLN1, FLNA, SLC2A1, PTMA, ACAA1, MARS1, TLN2, ARL8B, EHD4, TGFB2, PITPNB, PI4KA, PSMC5, PCMT1, H1-5, FSCN1, PSMD12, PSME4, LIPG, NDNF, ANO6 or a combination thereof. Also provided is a biomarker panel, comprising two or more biomarker proteins selected from List-G2. In some embodiments, the biomarker panel comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, or 17 or more biomarker proteins selected from List-G2. The biomarker may be selected from List-G3, wherein List-G3 a group consisting of the proteins ZYX, RHOA, HNRNPC, GART, MAPK3, IP09, JAM3, RAP2B, TLN1, FLNA, SLC2A1, PTMA, ACAA1, MARS1, TLN2, ARL8B, EHD4, TGFB2, PITPNB, PI4KA, PSMC5, PCMT1, H1-5, FSCN1, PSMD12, NDNF, ANO6 or a combination thereof. Also provided is a biomarker panel, comprising two or more biomarker proteins selected from List-G3. In some embodiments, the biomarker panel comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, or 17 or more biomarker proteins selected from List-G3.

Biomarker miRNA

A dystonia biomarker may be an miRNA molecule, as described herein. Such microRNAs may bind to messenger RNA targets involved in dystonia pathogenesis and are indicative of disease state. In one aspect, the miRNA biomarker is selected from List-MR1, wherein List-MR1 is a group consisting of the miRNA molecules hsa-miR-1.3p, hsa-miR-6770-5p, hsa-miR-9-5p, hsa-miR-1275, hsa-miR-3200-5p, hsa-miR-5010-3p, hsa-let-7d-5p, hsa-miR-7.5p, hsa-let-7b-5p, hsa-miR-98-5p, hsa-let-7g-5p, hsa-miR-32-5p, hsa-miR-203a-3p, hsa-miR-99a-3p, or a combination thereof. Also provided is a biomarker panel, comprising two or more biomarker miRNA molecules selected from List-G1. In some embodiments, the biomarker panel comprises 2 or more, 3 or more, 4 or more, 5 or more, 6 or more, 7 or more, 8 or more, 9 or more, 10 or more, 11 or more, 12 or more, or 13 or more biomarker miRNA molecules selected from List-G1.

In some embodiments, the nucleic acid sequence of the List-MR1 miRNAs comprises or consists of the mature form nucleic acid sequences set forth in TABLE 2. As a skilled artisan would recognize, other related forms of the List-MR1 miRNA molecules that are not mature may also be suitable as a biomarkers.

Table 2: Dystonia biomarker miRNA molecules and mature sequence

TABLE 2
Dystonia biomarker miRNA molecules and mature sequence

| miRNA Name | Mature Sequence |
|---|---|
| hsa-let-7b-5p | UGAGGUAGUAGGUUGUGUGGUU (SEQ ID NO: 1) |
| hsa-let-7d-5p | AGAGGUAGUAGGUUGCAUAGUU (SEQ ID NO: 2) |
| hsa-let-7g-5p | UGAGGUAGUAGUUUGUACAGUU (SEQ ID NO: 3) |
| hsa-miR-1275 | GUGGGGGAGAGGCUGUC (SEQ ID NO: 4) |
| hsa-miR-1-3p | UGGAAUGUAAAGAAGUAUGUAU (SEQ ID NO: 5) |
| hsa-miR-203a-3p | GUGAAAUGUUUAGGACCACUAG (SEQ ID NO: 6) |
| hsa-miR-3200-5p | AAUCUGAGAAGGCGCACAAGGU (SEQ ID NO: 7) |
| hsa-miR-32-5p | UAUUGCACAUUACUAAGUUGCA (SEQ ID NO: 8) |
| hsa-miR-5010-3p | UUUUGUGUCUCCCAUUCCCCAG (SEQ ID NO: 9) |
| hsa-miR-6770-5p | UGAGAAGGCACAGCUUGCACGUGA (SEQ ID NO: 10) |
| hsa-miR-7-5p | UGGAAGACUAGUGAUUUUGUUGUU (SEQ ID NO: 11) |
| hsa-miR-9-5p | UCUUUGGUUAUCUAGCUGUAUGA (SEQ ID NO: 12) |
| hsa-miR-98-5p | UGAGGUAGUAAGUUGUAUUGUU (SEQ ID NO: 13) |
| hsa-miR-99a-3p | CAAGCUCGCUUCUAUGGGUCUG (SEQ ID NO: 14) |

### Biomarker Metabolites

A dystonia biomarker may be a metabolite molecule, as described herein. Such metabolites may be involved in dystonia pathogenesis in the subject and are indicative of disease state. In one aspect, the metabolite biomarker is selected from an acylcamitine, amino acid, bile acid, ceramide, cholesterol ester, diacylglycerol, fatty acid, glycerophospholipid, glycosylceramide, triacylglycerol, or a sphingolipid. In one aspect, the metabolite biomarker is selected from List-ML1, wherein List-ML1 is a group consisting of the metabolite molecules PC aa C24:0_Glycerophospholipids, 5-AVA_Aminoacids Related, Cystine_Aminoacids Related, Cer(d16:1/20:0)_Ceramides, lysoPC a C26:0_Glycerophospholipids, Asp_Aminoacids, Cys_Aminoacids, Tyr_Aminoacids, PC aa C26:0_Glycerophospholipids, lysoPC a C28:0_Glycerophospholipids, C18:2_Acylcamitines, Cer(d18:2/18:1)_Ceramides, Cer(d16:1/18:0)_Ceramides, p-Cresol-SO4_Cresols, PC aa C40:6_Glycerophospholipids, C16-OH_Acylcarnitines, Gly_Aminoacids, Hypoxanthine_Nucleobases Related, EPA_Fatty Acids, C14:2-OH_Acylcamitines, or a combination thereof. Also provided is a biomarker panel, comprising two or more biomarker metabolite molecules selected from List-ML1. In some embodiments, the biomarker panel comprises 2 or more, 3 or more, 4 or more, 5 or more, 6 or more, 7 or more, 8 or more, 9 or more, 10 or more, 11 or more, 12 or more, or 13 or more biomarker metabolite molecules selected from List-ML1.

### G. Methods of Use

In one aspect, a dystonia biomarker is selected from a protein, miRNA, or metabolite implicated in dystonia disease state as described herein may be used in any of the contemplated methods. A dystonia biomarker of a method as described herein may be a protein selected from List-A1, List-A2, List-A3, List-A4, List-B1, List-B2, List-B3, List-C1, List-C2, List-C3, List-D1, List-D2, List-D3, List-E1, List-E2, List-E3, List-F1, List-F2, List-F3, List-G1, List-G2, List-G3, TABLE 4, TABLE 5, TABLE 6, TABLE 7, TABLE 8, TABLE 9, TABLE S1, TABLE S3, TABLE S4, TABLE S5, TABLE S6, TABLE S7, or a combination thereof. A panel of dystonia biomarkers may comprise one or more of any one of the proteins selected from List-A1, List-A2, List-A3, List-A4, List-B1, List-B2, List-B3, List-C1, List-C2, List-C3, List-D1, List-D2, List-D3, List-E1, List-E2, List-E3, List-F1, List-F2, List-F3, List-G1, List-G2, List-G3, TABLE 4, TABLE 5, TABLE 6, TABLE 7, TABLE 8, TABLE 9, TABLE S1, TABLE S3, TABLE S4, TABLE S5, TABLE S6, TABLE S7, or a combination thereof. In one aspect, a dystonia biomarker is selected from an miRNA molecule selected from List-MR1, TABLE 2, TABLE 3, or a combination thereof. A panel of dystonia biomarkers may comprise one or more of any one of the miRNA molecules selected from List-MR1, TABLE 2, TABLE 3, or a combination thereof. In one aspect, a dystonia biomarker is selected from an metabolite molecule selected from List-ML1, or a combination thereof. A panel of dystonia biomarkers may comprise one or more of any one of the metabolite molecules selected from List-ML1, or a combination thereof.

### Methods of Treating Dystonia

Disclosed herein is method of treating dystonia in a subject in need thereof, the method comprising administering to the subject one or more therapeutic agents when one or more dystonia biomarkers in the subject is aberrant compared to a reference level. The dystonia biomarker may be obtained from a biosample of the subject.

Disclosed herein is a method of treating a subject having a dystonia, the method comprising obtaining a biosample from a subject after treatment; determining the level (e.g., expression level) of one or more dystonia biomarker (e.g., a protein, miRNA, or metabolite as in the subject described herein) in the post-treatment biosample, wherein: if the post-treatment level represents an improvement or correction over a pre-treatment level of the one or more dystonia biomarker, or if the post-treatment level is within an acceptable range of a reference level, then continuing to administer the treatment. The dystonia biomarker monitors pharmacodynamic response to treatment with a therapeutic agent. In some embodiments, said level of a dystonia biomarker is its expression level in the subject.

Disclosed herein is a method of treating dystonia in a subject, comprising administering to the subject an amount of one or more therapeutic agents, wherein the amount of the one or more therapeutic agents is increased or reduced from a preliminary dosage amount when one or more dystonia biomarkers in the subject is aberrant compared to a reference level. The preliminary dosage amount may an initial starting dosage of the therapeutic compound. For instance, the dosage amount may set forth in a Food and Drug Administration (FDA) or European Medical Agency (EMA) approved pharmaceutical product label for the therapeutic agent. Alternatively, the preliminary dosage may be starting dosage of the therapeutic agent for a subject in a clinical trial.

In an aspect, a disclosed method can comprise obtaining a first biosample from the subject prior to treatment and detecting the level of one or more dystonia biomarker (e.g., a protein, miRNA, or metabolite as described herein) in the first (e.g., pre-treatment) biosample. In another aspect, a disclosed method can comprise processing a biosample previously obtained from the subject. In an aspect, a disclosed method can comprise obtaining a further (e.g., second, third, fourth, or subsequent) biosample from the subject during or after treatment and detecting the level of one or more proteins in the first (e.g., pre-treatment) biosample.

In an aspect, determining the level of one or more proteins or metabolites in a biosample as a dystonia biomarker can comprise liquid chromatography with tandem mass spectrometry (LC-MS-MS), parallel reaction monitoring (PRM), or multiple reaction monitoring (MRM). In an aspect, determining the level of one or more miRNAs in a biosample as a dystonia biomarker can comprise using RNASeq or RT-qPCR. In an aspect, determining the level of one or more miRNAs can comprise a commercial assay (e.g., NanoString nCounter® assays).

In an aspect, a disclosed reference level (e.g., expression level) can comprise an level from a dystonia biomarker obtained from a biosample from a subject not having a dystonia. In an aspect, a disclosed reference level can comprise a reference level (e.g., expression level) comprising an aggregate level (e.g., expression level) obtained from biosamples of subjects not having a dystonia. In an aspect, a reference biosample can comprise a biosample from a subject not having a dystonia or an aggregate of biosamples from subjects not having a dystonia.

In an aspect, obtaining a reference biosample can comprise obtaining a biosample from a subject not having a dystonia and determining the level (e.g., expression level) of one or more dystonia biomarker (e.g., a protein, miRNA, or metabolite as described herein) in the biosample. In an aspect, obtaining a reference biosample can comprise obtaining a biosample from subjects not having a dystonia and determining the level (e.g., expression level) of one or more dystonia biomarker (e.g., a protein, miRNA, or metabolite as described herein) in the biosamples.

In an aspect, the post-treatment level (e.g., expression level) of the one or more dystonia biomarker (e.g., a protein, miRNA, or metabolite as described herein) can represent an improvement or correction over a pre-treatment level when the post-treatment level is more similar to a reference level than to the pre-treatment level.

In an aspect, a biosample can comprise tissues, cells, biopsies, blood, lymph, CFS, serum, plasma, urine, saliva, mucus, tears, or a combination thereof. In an aspect, a biosample can comprise extracellular vesicles or extracellular vesicles collected from cultured patient-derived cells. In an aspect, cultured patient-derived cells can comprise primary cells, immortalized cells, iPSC cells, or any combination thereof.

In an aspect, one or more disclosed dystonia biomarker (e.g., a protein, miRNA, or metabolite as described herein) can be those biomarkers described in Section F herein.

In an aspect, one or more dystonia biomarker (e.g., a protein, miRNA, or metabolite as described herein) can be associated with dysfunction in phosphor-eIF2α signaling. In an aspect, the one or more dystonia biomarker (e.g., a protein, miRNA, or metabolite as described herein) can be associated with dysfunction in the integrated stress response.

In an aspect, a dystonia can be focal dystonia, blepharospasm, cervical dystonia, oromandibular dystonia, task-specific or occupational dystonia, spasmodic dysphonia, generalized dystonia, segmental dystonia, DYT1-related dystonia, DYT6-related dystonia, DYT28-related dystonia, dopa-responsive dystonia, myoclonic dystonia, X-linked dystonia-Parkinsonism, rapid-onset dystonia-Parkinsonism, paroxysmal dystonia choreoathetosis, paroxysmal kinesigenic dystonia, paroxysmal nonkinesigenic dyskinesia, paroxysmal exertion-induced dyskinesia, primary dystonia, acquired dystonia, tardive dyskinesia, or tardive dystonia.

In an aspect of a disclosed method, treatment can comprise administering to the subject one or more agents that modulate the level (e.g., expression level) of one or more aberrant biomarker. In an aspect, modulating the level (e.g., expression level) can comprise increasing the level, decreasing the level, or both. In an aspect, the level (e.g., expression level) of one or more of the differentially expressed proteins can be increased or decreased.

In an aspect, a disclosed method can comprise treating a subject. In an aspect, treating a subject can comprise administering one or more agents that target eIF2α signaling, such as, for example, contributing to the phosphorylation or de-phosphorylation of eIF2α.

In an aspect, a disclosed agent can be ritonavir, nelfinavir, lopinavir, saquinavir, deshydroxy-lopinavir, cobicistat, deshydroxy-ritonavir, or any combination thereof. In an aspect, a disclosed agent can be ritonavir.

In an aspect, a disclosed method can comprise repeating one or more steps of the disclosed method. In an aspect, a disclosed method can comprise modifying an administering step.

In an aspect, an aberrant biomarker (e.g., differentially expressed biomarker) may comprise differential expression comprising at least a 2-fold change, at least at 5-fold change, at least a 7-fold change, at least a 10-fold change, or more than a 10-fold change between the two biosamples. In an aspect, aberrant biomarker (e.g., differentially expressed biomarker) can comprise at least a +1, +2, +3, −1, −2, or −3 Z-score. In an aspect, d aberrant biomarker (e.g., differentially expressed biomarker) can comprise at least a Cohen's d effect size of at least 0.2, at least 0.5, at least 0.8, or greater than 0.8. In an aspect, aberrant biomarker (e.g., differentially expressed biomarker) can comprise at least a Cohen's d effect size of at least 1, at least 2, at least 3, or greater than 3.

In an aspect, an aberrant dystonia biomarker may be below its reference level, or above its reference level. When reviewing multiple dystonia biomarkers, one or more aberrant dystonia biomarkers may be below its reference level and at least one of the one or more aberrant dystonia biomarkers is above its reference level. A dystonia biomarker may be aberrant by at least ±0.5 fold, at least ±1.0 fold, at least ±1.5 fold, at least ±2.0 fold, at least ±3.0 fold, or at least ±4.0 fold relative to a reference level. A reference level may be a control value determined from a subject or group of subjects, each of which does not have dystonia; or a normal or desired value, optionally wherein the normal or desired value is according to a Diagnostic and Statistical Manual (DSM).

In an aspect, a disclosed method can comprise generating a proteomic profile for one or both biosamples. Individual components of the proteomic profile include but are not limited to those members described in Section F herein. For the purposes of the present invention the proteomic profile comprises from least two to all of the proteins listed in Section F herein.

In an aspect, a disclosed method can comprise administering one or more additional therapeutic agents. One or more additional therapeutic agents can comprise any therapeutic agents disclosed herein.

### Methods of Selecting a Subject for a Dystonia Clinical Trial

Disclosed herein is a method of selecting a subject for a clinical trial, the method comprising measuring one or more dystonia biomarkers biomarker (e.g., a protein, miRNA, or metabolite as in the subject described herein) in the subject from a biological sample obtained from the subject, and enrolling the subject in the clinical trial when the one or more dystonia biomarkers in the subject is aberrant compared to a reference level, or not enrolling the subject in the clinical trial when the one or more dystonia biomarkers in the subject is not aberrant compared to a reference level. The level of the dystonia biomarker may be an inclusion criteria in a clinical trial. The level of the dystonia biomarker may be an exclusion criteria in a clinical trial. The dystonia biomarker may be for enrichment of recruitment into a clinical trial for treatment of dystonia, for example to screen for subjects most likely to respond to a given intervention within an arm of the clinical trial.

aspect, a disclosed method can comprise obtaining a first biosample from the subject prior to treatment and detecting the level of one or more dystonia biomarker (e.g., a protein, miRNA, or metabolite as described herein) in the first (e.g., pre-treatment) biosample. In another aspect, a disclosed method can comprise processing a biosample previously obtained from the subject. In an aspect, a disclosed method can comprise obtaining a further (e.g., second, third, fourth, or subsequent) biosample from the subject during or after treatment and detecting the level of one or more proteins in the first (e.g., pre-treatment) biosample.

In an aspect, determining the level of one or more proteins or metabolites in a biosample as a dystonia biomarker can comprise liquid chromatography with tandem mass spectrometry (LC-MS-MS), parallel reaction monitoring (PRM), or multiple reaction monitoring (MRM). In an aspect, determining the level of one or more miRNAs in a biosample as a dystonia biomarker can comprise using RNASeq or RT-qPCR. In an aspect, determining the level of one or more miRNAs can comprise a commercial assay (e.g., NanoString nCounter® assays).

In an aspect, a disclosed reference level (e.g., expression level) can comprise an level from a dystonia biomarker obtained from a biosample from a subject not having a dystonia. In an aspect, a disclosed reference level can comprise a reference level (e.g., expression level) comprising an aggregate level (e.g., expression level) obtained from biosamples of subjects not having a dystonia. In an aspect, a reference biosample can comprise a biosample from a subject not having a dystonia or an aggregate of biosamples from subjects not having a dystonia.

In an aspect, obtaining a reference biosample can comprise obtaining a biosample from a subject not having a dystonia and determining the level (e.g., expression level) of one or more dystonia biomarker (e.g., a protein, miRNA, or metabolite as described herein) in the biosample. In an aspect, obtaining a reference biosample can comprise obtaining a biosample from subjects not having a dystonia and determining the level (e.g., expression level) of one or more dystonia biomarker (e.g., a protein, miRNA, or metabolite as described herein) in the biosamples.

In an aspect, the post-treatment level (e.g., expression level) of the one or more dystonia biomarker (e.g., a protein, miRNA, or metabolite as described herein) can represent an improvement or correction over a pre-treatment level when the post-treatment level is more similar to a reference level than to the pre-treatment level.

In an aspect, a biosample can comprise tissues, cells, biopsies, blood, lymph, CFS, serum, plasma, urine, saliva, mucus, tears, or a combination thereof. In an aspect, a biosample can comprise extracellular vesicles or extracellular vesicles collected from cultured patient-derived cells. In an aspect, cultured patient-derived cells can comprise primary cells, immortalized cells, iPSC cells, or any combination thereof.

In an aspect, one or more disclosed dystonia biomarker (e.g., a protein, miRNA, or metabolite as described herein) can be those biomarkers described in Section F herein.

In an aspect, one or more dystonia biomarker (e.g., a protein, miRNA, or metabolite as described herein) can be associated with dysfunction in phosphor-eIF2α signaling. In an aspect, the one or more dystonia biomarker (e.g., a protein, miRNA, or metabolite as described herein) can be associated with dysfunction in the integrated stress response.

In an aspect, a dystonia can be focal dystonia, blepharospasm, cervical dystonia, oromandibular dystonia, task-specific or occupational dystonia, spasmodic dysphonia, generalized dystonia, segmental dystonia, DYT1-related dystonia, DYT6-related dystonia, DYT28-related dystonia, dopa-responsive dystonia, myoclonic dystonia, X-linked dystonia-Parkinsonism, rapid-onset dystonia-Parkinsonism, paroxysmal dystonia choreoathetosis, paroxysmal kinesigenic dystonia, paroxysmal nonkinesigenic dyskinesia, paroxysmal exertion-induced dyskinesia, primary dystonia, acquired dystonia, tardive dyskinesia, or tardive dystonia.

In an aspect of a disclosed method, treatment can comprise administering to the subject one or more agents that modulate the level (e.g., expression level) of one or more aberrant biomarker. In an aspect, modulating the level (e.g., expression level) can comprise increasing the level, decreasing the level, or both. In an aspect, the level (e.g., expression level) of one or more of the differentially expressed proteins can be increased or decreased.

In an aspect, a disclosed method can comprise treating a subject. In an aspect, treating a subject can comprise administering one or more agents that target eIF2α signaling, such as, for example, contributing to the phosphorylation or de-phosphorylation of eIF2α.

In an aspect, a disclosed agent can be ritonavir, nelfinavir, lopinavir, saquinavir, deshydroxy-lopinavir, cobicistat, deshydroxy-ritonavir, or any combination thereof. In an aspect, a disclosed agent can be ritonavir.

In an aspect, a disclosed method can comprise repeating one or more steps of the disclosed method. In an aspect, a disclosed method can comprise modifying an administering step.

In an aspect, an aberrant biomarker (e.g., differentially expressed biomarker) may comprise differential expression comprising at least a 2-fold change, at least at 5-fold change, at least a 7-fold change, at least a 10-fold change, or more than a 10-fold change between the two biosamples. In an aspect, aberrant biomarker (e.g., differentially expressed biomarker) can comprise at least a +1, +2, +3, −1, −2, or −3 Z-score. In an aspect, d aberrant biomarker (e.g., differentially expressed biomarker) can comprise at least a Cohen's d effect size of at least 0.2, at least 0.5, at least 0.8, or greater than 0.8. In an aspect, aberrant biomarker (e.g., differentially expressed biomarker) can comprise at least a Cohen's d effect size of at least 1, at least 2, at least 3, or greater than 3.

In an aspect, an aberrant dystonia biomarker may be below its reference level, or above its reference level. When reviewing multiple dystonia biomarkers, one or more aberrant dystonia biomarkers may be below its reference level and at least one of the one or more aberrant dystonia biomarkers is above its reference level. A dystonia biomarker may be aberrant by at least ±0.5 fold, at least ±1.0 fold, at least ±1.5 fold, at least ±2.0 fold, at least ±3.0 fold, or at least ±4.0 fold relative to a reference level. A reference level may be a control value determined from a subject or group of subjects, each of which does not have dystonia; or a normal or desired value, optionally wherein the normal or desired value is according to a Diagnostic and Statistical Manual (DSM).

In an aspect, a disclosed method can comprise generating a proteomic profile for one or both biosamples. Individual components of the proteomic profile include but are not limited to those members described in Section F herein. For the purposes of the present invention the proteomic profile comprises from least two to all of the proteins listed in Section F herein.

### Methods of Selecting a Therapeutic Agent for Treatment of Dystonia

Disclosed herein is a method of selecting a therapeutic agent for treatment of dystonia in a subject, the method comprising measuring one or more dystonia biomarkers (e.g., a protein, miRNA, or metabolite as in the subject described herein) in the subject from a biological sample obtained from the subject, and selecting the therapeutic agent when the one or more dystonia biomarkers in the subject is aberrant compared to a reference level; or not aberrant compared to a reference level. The dystonia biomarker may be improved or corrected (e.g., increased or decreased expression of the biomarker) after treatment with the therapeutic agent.

In an aspect, a disclosed method can comprise obtaining a first biosample from the subject prior to treatment and detecting the level of one or more dystonia biomarker (e.g., a protein, miRNA, or metabolite as described herein) in the first (e.g., pre-treatment) biosample. In another aspect, a disclosed method can comprise processing a biosample previously obtained from the subject. In an aspect, a disclosed method can comprise obtaining a further (e.g., second, third, fourth, or subsequent) biosample from the subject during or after treatment and detecting the level of one or more proteins in the first (e.g., pre-treatment) biosample.

In an aspect, determining the level of one or more proteins or metabolites in a biosample as a dystonia biomarker can comprise liquid chromatography with tandem mass spectrometry (LC-MS-MS), parallel reaction monitoring (PRM), or multiple reaction monitoring (MRM). In an aspect, determining the level of one or more miRNAs in a biosample as a dystonia biomarker can comprise using RNASeq or RT-qPCR. In an aspect, determining the level of one or more miRNAs can comprise a commercial assay (e.g., NanoString nCounter® assays).

In an aspect, a disclosed reference level (e.g., expression level) can comprise an level from a dystonia biomarker obtained from a biosample from a subject not having a dystonia. In an aspect, a disclosed reference level can comprise a reference level (e.g., expression level) comprising an aggregate level (e.g., expression level) obtained from biosamples of subjects not having a dystonia. In an aspect, a reference biosample can comprise a biosample from a subject not having a dystonia or an aggregate of biosamples from subjects not having a dystonia.

In an aspect, obtaining a reference biosample can comprise obtaining a biosample from a subject not having a dystonia and determining the level (e.g., expression level) of one or more dystonia biomarker (e.g., a protein, miRNA, or metabolite as described herein) in the biosample. In an aspect, obtaining a reference biosample can comprise obtaining a biosample from subjects not having a dystonia and determining the level (e.g., expression level) of one or more dystonia biomarker (e.g., a protein, miRNA, or metabolite as described herein) in the biosamples.

In an aspect, the post-treatment level (e.g., expression level) of the one or more dystonia biomarker (e.g., a protein, miRNA, or metabolite as described herein) can represent an improvement or correction over a pre-treatment level when the post-treatment level is more similar to a reference level than to the pre-treatment level.

In an aspect, a biosample can comprise tissues, cells, biopsies, blood, lymph, CFS, serum, plasma, urine, saliva, mucus, tears, or a combination thereof. In an aspect, a biosample can comprise extracellular vesicles or extracellular vesicles collected from cultured patient-derived cells. In an aspect, cultured patient-derived cells can comprise primary cells, immortalized cells, iPSC cells, or any combination thereof.

In an aspect, one or more disclosed dystonia biomarker (e.g., a protein, miRNA, or metabolite as described herein) can be those biomarkers described in Section F herein.

In an aspect, one or more dystonia biomarker (e.g., a protein, miRNA, or metabolite as described herein) can be associated with dysfunction in phosphor-eIF2α signaling. In an aspect, the one or more dystonia biomarker (e.g., a protein, miRNA, or metabolite as described herein) can be associated with dysfunction in the integrated stress response.

In an aspect, a dystonia can be focal dystonia, blepharospasm, cervical dystonia, oromandibular dystonia, task-specific or occupational dystonia, spasmodic dysphonia, generalized dystonia, segmental dystonia, DYT1-related dystonia, DYT6-related dystonia, DYT28-related dystonia, dopa-responsive dystonia, myoclonic dystonia, X-linked dystonia-Parkinsonism, rapid-onset dystonia-Parkinsonism, paroxysmal dystonia choreoathetosis, paroxysmal kinesigenic dystonia, paroxysmal nonkinesigenic dyskinesia, paroxysmal exertion-induced dyskinesia, primary dystonia, acquired dystonia, tardive dyskinesia, or tardive dystonia.

In an aspect of a disclosed method, treatment can comprise administering to the subject one or more agents that modulate the level (e.g., expression level) of one or more aberrant biomarker. In an aspect, modulating the level (e.g., expression level) can comprise increasing the level, decreasing the level, or both. In an aspect, the level (e.g., expression level) of one or more of the differentially expressed proteins can be increased or decreased.

In an aspect, a disclosed method can comprise treating a subject. In an aspect, treating a subject can comprise administering one or more agents that target eIF2α signaling, such as, for example, contributing to the phosphorylation or de-phosphorylation of eIF2α.

In an aspect, a disclosed agent can be ritonavir, nelfinavir, lopinavir, saquinavir, deshydroxy-lopinavir, cobicistat, deshydroxy-ritonavir, or any combination thereof. In an aspect, a disclosed agent can be ritonavir.

In an aspect, a disclosed method can comprise repeating one or more steps of the disclosed method. In an aspect, a disclosed method can comprise modifying an administering step.

In an aspect, an aberrant biomarker (e.g., differentially expressed biomarker) may comprise differential expression comprising at least a 2-fold change, at least at 5-fold change, at least a 7-fold change, at least a 10-fold change, or more than a 10-fold change between the two biosamples. In an aspect, aberrant biomarker (e.g., differentially expressed biomarker) can comprise at least a +1, +2, +3, −1, −2, or −3 Z-score. In an aspect, d aberrant biomarker (e.g., differentially expressed biomarker) can comprise at least a Cohen's d effect size of at least 0.2, at least 0.5, at least 0.8, or greater than 0.8. In an aspect, aberrant biomarker (e.g., differentially expressed biomarker) can comprise at least a Cohen's d effect size of at least 1, at least 2, at least 3, or greater than 3.

In an aspect, an aberrant dystonia biomarker may be below its reference level, or above its reference level. When reviewing multiple dystonia biomarkers, one or more aberrant dystonia biomarkers may be below its reference level and at least one of the one or more aberrant dystonia biomarkers is above its reference level. A dystonia biomarker may be aberrant by at least ±0.5 fold, at least ±1.0 fold, at least ±1.5 fold, at least ±2.0 fold, at least ±3.0 fold, or at least ±4.0 fold relative to a reference level. A reference level may be a control value determined from a subject or group of subjects, each of which does not have dystonia; or a normal or desired value, optionally wherein the normal or desired value is according to a Diagnostic and Statistical Manual (DSM).

In an aspect, a disclosed method can comprise generating a proteomic profile for one or both biosamples. Individual components of the proteomic profile include but are not limited to those members described in Section F herein. For the purposes of the present invention the proteomic profile comprises from least two to all of the proteins listed in Section F herein.

### Methods of Predicting Responsiveness to a Treatment for Dystonia

Disclosed herein is a method of predicting responsiveness to a treatment for dystonia, the method comprising obtaining a biosample from a subject having a dystonia; determining the expression level of one or more dystonia biomarkers (e.g., a protein, miRNA, or metabolite) in the biosample to create a proteomic profile; comparing the subject's proteomic profile to a proteomic profile of a treatment-responsive subject; predicting that the subject having a dystonia will be responsive to the treatment when the proteomic profiles are similar; or predicting that the subject having a dystonia will not be responsive to the treatment when the proteomic profiles are aberrant.

In an aspect, a disclosed method can comprise obtaining a first biosample from the subject prior to treatment and detecting the level of one or more dystonia biomarker (e.g., a protein, miRNA, or metabolite as described herein) in the first (e.g., pre-treatment) biosample. In another aspect, a disclosed method can comprise processing a biosample previously obtained from the subject. In an aspect, a disclosed method can comprise obtaining a further (e.g., second, third, fourth, or subsequent) biosample from the subject during or after treatment and detecting the level of one or more proteins in the first (e.g., pre-treatment) biosample.

In an aspect, determining the level of one or more proteins or metabolites in a biosample as a dystonia biomarker can comprise liquid chromatography with tandem mass spectrometry (LC-MS-MS), parallel reaction monitoring (PRM), or multiple reaction monitoring (MRM). In an aspect, determining the level of one or more miRNAs in a biosample as a dystonia biomarker can comprise using RNASeq or RT-qPCR. In an aspect, determining the level of one or more miRNAs can comprise a commercial assay (e.g., NanoString nCounter® assays).

In an aspect, a disclosed reference level (e.g., expression level) can comprise an level from a dystonia biomarker obtained from a biosample from a subject not having a dystonia. In an aspect, a disclosed reference level can comprise a reference level (e.g., expression level) comprising an aggregate level (e.g., expression level) obtained from biosamples of subjects not having a dystonia. In an aspect, a reference biosample can comprise a biosample from a subject not having a dystonia or an aggregate of biosamples from subjects not having a dystonia.

In an aspect, obtaining a reference biosample can comprise obtaining a biosample from a subject not having a dystonia and determining the level (e.g., expression level) of one or more dystonia biomarker (e.g., a protein, miRNA, or metabolite as described herein) in the biosample. In an aspect, obtaining a reference biosample can comprise obtaining a biosample from subjects not having a dystonia and determining the level (e.g., expression level) of one or more dystonia biomarker (e.g., a protein, miRNA, or metabolite as described herein) in the biosamples.

In an aspect, the post-treatment level (e.g., expression level) of the one or more dystonia biomarker (e.g., a protein, miRNA, or metabolite as described herein) can represent an improvement or correction over a pre-treatment level when the post-treatment level is more similar to a reference level than to the pre-treatment level.

In an aspect, a biosample can comprise tissues, cells, biopsies, blood, lymph, CFS, serum, plasma, urine, saliva, mucus, tears, or a combination thereof. In an aspect, a biosample can comprise extracellular vesicles or extracellular vesicles collected from cultured patient-derived cells. In an aspect, cultured patient-derived cells can comprise primary cells, immortalized cells, iPSC cells, or any combination thereof.

In an aspect, one or more disclosed dystonia biomarker (e.g., a protein, miRNA, or metabolite as described herein) can be those biomarkers described in Section F herein.

In an aspect, one or more dystonia biomarker (e.g., a protein, miRNA, or metabolite as described herein) can be associated with dysfunction in phosphor-eIF2α signaling. In an aspect, the one or more dystonia biomarker (e.g., a protein, miRNA, or metabolite as described herein) can be associated with dysfunction in the integrated stress response.

In an aspect, a dystonia can be focal dystonia, blepharospasm, cervical dystonia, oromandibular dystonia, task-specific or occupational dystonia, spasmodic dysphonia, generalized dystonia, segmental dystonia, DYT1-related dystonia, DYT6-related dystonia, DYT28-related dystonia, dopa-responsive dystonia, myoclonic dystonia, X-linked dystonia-Parkinsonism, rapid-onset dystonia-Parkinsonism, paroxysmal dystonia choreoathetosis, paroxysmal kinesigenic dystonia, paroxysmal nonkinesigenic dyskinesia, paroxysmal exertion-induced dyskinesia, primary dystonia, acquired dystonia, tardive dyskinesia, or tardive dystonia.

In an aspect of a disclosed method, treatment can comprise administering to the subject one or more agents that modulate the level (e.g., expression level) of one or more aberrant biomarker. In an aspect, modulating the level (e.g., expression level) can comprise increasing the level, decreasing the level, or both. In an aspect, the level (e.g., expression level) of one or more of the differentially expressed proteins can be increased or decreased.

In an aspect, a disclosed method can comprise treating a subject. In an aspect, treating a subject can comprise administering one or more agents that target eIF2α signaling, such as, for example, contributing to the phosphorylation or de-phosphorylation of eIF2α.

In an aspect, a disclosed agent can be ritonavir, nelfinavir, lopinavir, saquinavir, deshydroxy-lopinavir, cobicistat, deshydroxy-ritonavir, or any combination thereof. In an aspect, a disclosed agent can be ritonavir.

In an aspect, a disclosed method can comprise repeating one or more steps of the disclosed method. In an aspect, a disclosed method can comprise modifying an administering step.

In an aspect, an aberrant biomarker (e.g., differentially expressed biomarker) may comprise differential expression comprising at least a 2-fold change, at least at 5-fold change, at least a 7-fold change, at least a 10-fold change, or more than a 10-fold change between the two biosamples. In an aspect, aberrant biomarker (e.g., differentially expressed biomarker) can comprise at least a +1, +2, +3, −1, −2, or −3 Z-score. In an aspect, d aberrant biomarker (e.g., differentially expressed biomarker) can comprise at least a Cohen's d effect size of at least 0.2, at least 0.5, at least 0.8, or greater than 0.8. In an aspect, aberrant biomarker (e.g., differentially expressed biomarker) can comprise at least a Cohen's d effect size of at least 1, at least 2, at least 3, or greater than 3.

In an aspect, an aberrant dystonia biomarker may be below its reference level, or above its reference level. When reviewing multiple dystonia biomarkers, one or more aberrant dystonia biomarkers may be below its reference level and at least one of the one or more aberrant dystonia biomarkers is above its reference level. A dystonia biomarker may be aberrant by at least ±0.5 fold, at least ±1.0 fold, at least ±1.5 fold, at least ±2.0 fold, at least ±3.0 fold, or at least ±4.0 fold relative to a reference level. A reference level may be a control value determined from a subject or group of subjects, each of which does not have dystonia; or a normal or desired value, optionally wherein the normal or desired value is according to a Diagnostic and Statistical Manual (DSM).

In an aspect, a disclosed method can comprise generating a proteomic profile for one or both biosamples. Individual components of the proteomic profile include but are not limited to those members described in Section F herein. For the purposes of the present invention the proteomic profile comprises from least two to all of the proteins listed in Section F herein.

In an aspect, a disclosed method can comprise administering one or more additional therapeutic agents. One or more additional therapeutic agents can comprise any therapeutic agents disclosed herein.

In another embodiment, the dystonia comprises focal dystonia, blepharospasm, cervical dystonia, oromandibular dystonia, task-specific or occupational dystonia, spasmodic dysphonia, generalized dystonia, segmental dystonia, DYT1-related dystonia, DYT6-related dystonia, DYT28-related dystonia, dopa-responsive dystonia, myoclonic dystonia, X-linked dystonia-Parkinsonism, rapid-onset dystonia-Parkinsonism, paroxysmal dystonia choreoathetosis, paroxysmal kinesigenic dystonia, paroxysmal nonkinesigenic dyskinesia, paroxysmal exertion-induced dyskinesia, primary dystonia, acquired dystonia, tardive dyskinesia, or tardive dystonia.

Another aspect of the present disclosure provides treating the subject having a dystonia, wherein treating the subject comprises, consists of, or consists essentially of administering one or more agents that modulate the expression level of one or more differentially expressed miRNAs. “Agents” and “Therapeutic Agents” are known to the art and are described supra. In an aspect, the one or more agents can treat, prevent, inhibit, and/or ameliorate one or more comorbidities in a subject. In an aspect, one or more therapeutic agents can treat, inhibit, prevent, and/or ameliorate a dystonia symptom or a dystonia related complication. In one embodiment, the one or more agents comprise ritonavir.

### Methods of Classifying a Subject Having Dystonia

Disclosed herein is a method classifying a subject having dystonia, the method comprising measuring one or more dystonia biomarkers (e.g., a protein, miRNA, or metabolite as in the subject described herein) in the subject from a biological sample obtained from the subject, and classifying the subject as having the one or more dystonia biomarkers when the one or more dystonia biomarkers in the subject is aberrant compared to a reference level, or not having the one or more dystonia biomarker when the one or more dystonia biomarkers in the subject is not aberrant compared to a reference level.

In an aspect, a disclosed method can comprise obtaining a first biosample from the subject prior to treatment and detecting the level of one or more dystonia biomarker (e.g., a protein, miRNA, or metabolite as described herein) in the first (e.g., pre-treatment) biosample. In another aspect, a disclosed method can comprise processing a biosample previously obtained from the subject. In an aspect, a disclosed method can comprise obtaining a further (e.g., second, third, fourth, or subsequent) biosample from the subject during or after treatment and detecting the level of one or more proteins in the first (e.g., pre-treatment) biosample.

In an aspect, determining the level of one or more proteins or metabolites in a biosample as a dystonia biomarker can comprise liquid chromatography with tandem mass spectrometry (LC-MS-MS), parallel reaction monitoring (PRM), or multiple reaction monitoring (MRM). In an aspect, determining the level of one or more miRNAs in a biosample as a dystonia biomarker can comprise using RNASeq or RT-qPCR. In an aspect, determining the level of one or more miRNAs can comprise a commercial assay (e.g., NanoString nCounter® assays).

In an aspect, a disclosed reference level (e.g., expression level) can comprise an level from a dystonia biomarker obtained from a biosample from a subject not having a dystonia. In an aspect, a disclosed reference level can comprise a reference level (e.g., expression level) comprising an aggregate level (e.g., expression level) obtained from biosamples of subjects not having a dystonia. In an aspect, a reference biosample can comprise a biosample from a subject not having a dystonia or an aggregate of biosamples from subjects not having a dystonia.

In an aspect, obtaining a reference biosample can comprise obtaining a biosample from a subject not having a dystonia and determining the level (e.g., expression level) of one or more dystonia biomarker (e.g., a protein, miRNA, or metabolite as described herein) in the biosample. In an aspect, obtaining a reference biosample can comprise obtaining a biosample from subjects not having a dystonia and determining the level (e.g., expression level) of one or more dystonia biomarker (e.g., a protein, miRNA, or metabolite as described herein) in the biosamples.

In an aspect, the post-treatment level (e.g., expression level) of the one or more dystonia biomarker (e.g., a protein, miRNA, or metabolite as described herein) can represent an improvement or correction over a pre-treatment level when the post-treatment level is more similar to a reference level than to the pre-treatment level.

In an aspect, a biosample can comprise tissues, cells, biopsies, blood, lymph, CFS, serum, plasma, urine, saliva, mucus, tears, or a combination thereof. In an aspect, a biosample can comprise extracellular vesicles or extracellular vesicles collected from cultured patient-derived cells. In an aspect, cultured patient-derived cells can comprise primary cells, immortalized cells, iPSC cells, or any combination thereof.

In an aspect, one or more disclosed dystonia biomarker (e.g., a protein, miRNA, or metabolite as described herein) can be those biomarkers described in Section F herein.

In an aspect, one or more dystonia biomarker (e.g., a protein, miRNA, or metabolite as described herein) can be associated with dysfunction in phosphor-eIF2α signaling. In an aspect, the one or more dystonia biomarker (e.g., a protein, miRNA, or metabolite as described herein) can be associated with dysfunction in the integrated stress response.

In an aspect, a dystonia can be focal dystonia, blepharospasm, cervical dystonia, oromandibular dystonia, task-specific or occupational dystonia, spasmodic dysphonia, generalized dystonia, segmental dystonia, DYT1-related dystonia, DYT6-related dystonia, DYT28-related dystonia, dopa-responsive dystonia, myoclonic dystonia, X-linked dystonia-Parkinsonism, rapid-onset dystonia-Parkinsonism, paroxysmal dystonia choreoathetosis, paroxysmal kinesigenic dystonia, paroxysmal nonkinesigenic dyskinesia, paroxysmal exertion-induced dyskinesia, primary dystonia, acquired dystonia, tardive dyskinesia, or tardive dystonia.

In an aspect of a disclosed method, treatment can comprise administering to the subject one or more agents that modulate the level (e.g., expression level) of one or more aberrant biomarker. In an aspect, modulating the level (e.g., expression level) can comprise increasing the level, decreasing the level, or both. In an aspect, the level (e.g., expression level) of one or more of the differentially expressed proteins can be increased or decreased.

In an aspect, a disclosed method can comprise treating a subject. In an aspect, treating a subject can comprise administering one or more agents that target eIF2α signaling, such as, for example, contributing to the phosphorylation or de-phosphorylation of eIF2α.

In an aspect, a disclosed agent can be ritonavir, nelfinavir, lopinavir, saquinavir, deshydroxy-lopinavir, cobicistat, deshydroxy-ritonavir, or any combination thereof. In an aspect, a disclosed agent can be ritonavir.

In an aspect, a disclosed method can comprise repeating one or more steps of the disclosed method. In an aspect, a disclosed method can comprise modifying an administering step.

In an aspect, an aberrant biomarker (e.g., differentially expressed biomarker) may comprise differential expression comprising at least a 2-fold change, at least at 5-fold change, at least a 7-fold change, at least a 10-fold change, or more than a 10-fold change between the two biosamples. In an aspect, aberrant biomarker (e.g., differentially expressed biomarker) can comprise at least a +1, +2, +3, −1, −2, or −3 Z-score. In an aspect, d aberrant biomarker (e.g., differentially expressed biomarker) can comprise at least a Cohen's d effect size of at least 0.2, at least 0.5, at least 0.8, or greater than 0.8. In an aspect, aberrant biomarker (e.g., differentially expressed biomarker) can comprise at least a Cohen's d effect size of at least 1, at least 2, at least 3, or greater than 3.

In an aspect, an aberrant dystonia biomarker may be below its reference level, or above its reference level. When reviewing multiple dystonia biomarkers, one or more aberrant dystonia biomarkers may be below its reference level and at least one of the one or more aberrant dystonia biomarkers is above its reference level. A dystonia biomarker may be aberrant by at least ±0.5 fold, at least ±1.0 fold, at least ±1.5 fold, at least ±2.0 fold, at least ±3.0 fold, or at least ±4.0 fold relative to a reference level. A reference level may be a control value determined from a subject or group of subjects, each of which does not have dystonia; or a normal or desired value, optionally wherein the normal or desired value is according to a Diagnostic and Statistical Manual (DSM).

In an aspect, a disclosed method can comprise generating a proteomic profile for one or both biosamples. Individual components of the proteomic profile include but are not limited to those members described in Section F herein. For the purposes of the present invention the proteomic profile comprises from least two to all of the proteins listed in Section F herein.

In an aspect, a disclosed method can comprise administering one or more additional therapeutic agents. One or more additional therapeutic agents can comprise any therapeutic agents disclosed herein.

### Methods of Identifying a Dystonia Biomarker

A method of identifying a dystonia biomarker in a subject, the method comprising: obtaining a biosample from a subject having a dystonia, obtaining a biosample from a subject not having a dystonia, determining the reference level of one or more proteins, miRNAs, and/or metabolites in the biosample from a subject having a dystonia, and identifying those proteins, miRNAs, and/or metabolites that are aberrant in the biosample obtained from the subject having a dystonia when compared to the biosample from the subject not having a dystonia; wherein those aberrant proteins, miRNAs, and/or metabolites are biomarkers of dystonia.

In an aspect, a disclosed method can comprise obtaining a first biosample from the subject prior to treatment and detecting the level of one or more dystonia biomarker (e.g., a protein, miRNA, or metabolite as described herein) in the first (e.g., pre-treatment) biosample. In another aspect, a disclosed method can comprise processing a biosample previously obtained from the subject. In an aspect, a disclosed method can comprise obtaining a further (e.g., second, third, fourth, or subsequent) biosample from the subject during or after treatment and detecting the level of one or more proteins in the first (e.g., pre-treatment) biosample.

In an aspect, determining the level of one or more proteins or metabolites in a biosample as a dystonia biomarker can comprise liquid chromatography with tandem mass spectrometry (LC-MS-MS), parallel reaction monitoring (PRM), or multiple reaction monitoring (MRM). In an aspect, determining the level of one or more miRNAs in a biosample as a dystonia biomarker can comprise using RNASeq or RT-qPCR. In an aspect, determining the level of one or more miRNAs can comprise a commercial assay (e.g., NanoString nCounter® assays).

In an aspect, a disclosed reference level (e.g., expression level) can comprise an level from a dystonia biomarker obtained from a biosample from a subject not having a dystonia. In an aspect, a disclosed reference level can comprise a reference level (e.g., expression level) comprising an aggregate level (e.g., expression level) obtained from biosamples of subjects not having a dystonia. In an aspect, a reference biosample can comprise a biosample from a subject not having a dystonia or an aggregate of biosamples from subjects not having a dystonia.

In an aspect, obtaining a reference biosample can comprise obtaining a biosample from a subject not having a dystonia and determining the level (e.g., expression level) of one or more dystonia biomarker (e.g., a protein, miRNA, or metabolite as described herein) in the biosample. In an aspect, obtaining a reference biosample can comprise obtaining a biosample from subjects not having a dystonia and determining the level (e.g., expression level) of one or more dystonia biomarker (e.g., a protein, miRNA, or metabolite as described herein) in the biosamples.

In an aspect, the post-treatment level (e.g., expression level) of the one or more dystonia biomarker (e.g., a protein, miRNA, or metabolite as described herein) can represent an improvement or correction over a pre-treatment level when the post-treatment level is more similar to a reference level than to the pre-treatment level.

In an aspect, a biosample can comprise tissues, cells, biopsies, blood, lymph, CFS, serum, plasma, urine, saliva, mucus, tears, or a combination thereof. In an aspect, a biosample can comprise extracellular vesicles or extracellular vesicles collected from cultured patient-derived cells. In an aspect, cultured patient-derived cells can comprise primary cells, immortalized cells, iPSC cells, or any combination thereof.

In an aspect, one or more disclosed dystonia biomarker (e.g., a protein, miRNA, or metabolite as described herein) can be those biomarkers described in Section F herein.

In an aspect, one or more dystonia biomarker (e.g., a protein, miRNA, or metabolite as described herein) can be associated with dysfunction in phosphor-eIF2α signaling. In an aspect, the one or more dystonia biomarker (e.g., a protein, miRNA, or metabolite as described herein) can be associated with dysfunction in the integrated stress response.

In an aspect, a dystonia can be focal dystonia, blepharospasm, cervical dystonia, oromandibular dystonia, task-specific or occupational dystonia, spasmodic dysphonia, generalized dystonia, segmental dystonia, DYT1-related dystonia, DYT6-related dystonia, DYT28-related dystonia, dopa-responsive dystonia, myoclonic dystonia, X-linked dystonia-Parkinsonism, rapid-onset dystonia-Parkinsonism, paroxysmal dystonia choreoathetosis, paroxysmal kinesigenic dystonia, paroxysmal nonkinesigenic dyskinesia, paroxysmal exertion-induced dyskinesia, primary dystonia, acquired dystonia, tardive dyskinesia, or tardive dystonia.

In an aspect of a disclosed method, treatment can comprise administering to the subject one or more agents that modulate the level (e.g., expression level) of one or more aberrant biomarker. In an aspect, modulating the level (e.g., expression level) can comprise increasing the level, decreasing the level, or both. In an aspect, the level (e.g., expression level) of one or more of the differentially expressed proteins can be increased or decreased.

In an aspect, a disclosed method can comprise treating a subject. In an aspect, treating a subject can comprise administering one or more agents that target eIF2α signaling, such as, for example, contributing to the phosphorylation or de-phosphorylation of eIF2α.

In an aspect, a disclosed agent can be ritonavir, nelfinavir, lopinavir, saquinavir, deshydroxy-lopinavir, cobicistat, deshydroxy-ritonavir, or any combination thereof. In an aspect, a disclosed agent can be ritonavir.

In an aspect, a disclosed method can comprise repeating one or more steps of the disclosed method. In an aspect, a disclosed method can comprise modifying an administering step.

In an aspect, an aberrant biomarker (e.g., differentially expressed biomarker) may comprise differential expression comprising at least a 2-fold change, at least at 5-fold change, at least a 7-fold change, at least a 10-fold change, or more than a 10-fold change between the two biosamples. In an aspect, aberrant biomarker (e.g., differentially expressed biomarker) can comprise at least a +1, +2, +3, −1, −2, or −3 Z-score. In an aspect, d aberrant biomarker (e.g., differentially expressed biomarker) can comprise at least a Cohen's d effect size of at least 0.2, at least 0.5, at least 0.8, or greater than 0.8. In an aspect, aberrant biomarker (e.g., differentially expressed biomarker) can comprise at least a Cohen's d effect size of at least 1, at least 2, at least 3, or greater than 3.

In an aspect, an aberrant dystonia biomarker may be below its reference level, or above its reference level. When reviewing multiple dystonia biomarkers, one or more aberrant dystonia biomarkers may be below its reference level and at least one of the one or more aberrant dystonia biomarkers is above its reference level. A dystonia biomarker may be aberrant by at least ±0.5 fold, at least ±1.0 fold, at least ±1.5 fold, at least ±2.0 fold, at least ±3.0 fold, or at least ±4.0 fold relative to a reference level. A reference level may be a control value determined from a subject or group of subjects, each of which does not have dystonia; or a normal or desired value, optionally wherein the normal or desired value is according to a Diagnostic and Statistical Manual (DSM).

In an aspect, a disclosed method can comprise generating a proteomic profile for one or both biosamples. Individual components of the proteomic profile include but are not limited to those members described in Section F herein. For the purposes of the present invention the proteomic profile comprises from least two to all of the proteins listed in Section F herein.

In an aspect, a disclosed method can comprise administering one or more additional therapeutic agents. One or more additional therapeutic agents can comprise any therapeutic agents disclosed herein.

In another embodiment, the dystonia comprises focal dystonia, blepharospasm, cervical dystonia, oromandibular dystonia, task-specific or occupational dystonia, spasmodic dysphonia, generalized dystonia, segmental dystonia, DYT1-related dystonia, DYT6-related dystonia, DYT28-related dystonia, dopa-responsive dystonia, myoclonic dystonia, X-linked dystonia-Parkinsonism, rapid-onset dystonia-Parkinsonism, paroxysmal dystonia choreoathetosis, paroxysmal kinesigenic dystonia, paroxysmal nonkinesigenic dyskinesia, paroxysmal exertion-induced dyskinesia, primary dystonia, acquired dystonia, tardive dyskinesia, or tardive dystonia.

Another aspect of the present disclosure provides treating the subject having a dystonia, wherein treating the subject comprises, consists of, or consists essentially of administering one or more agents that modulate the expression level of one or more differentially expressed miRNAs. “Agents” and “Therapeutic Agents” are known to the art and are described supra. In an aspect, the one or more agents can treat, prevent, inhibit, and/or ameliorate one or more comorbidities in a subject. In an aspect, one or more therapeutic agents can treat, inhibit, prevent, and/or ameliorate a dystonia symptom or a dystonia related complication. In one embodiment, the one or more agents comprise ritonavir.

### H. Kits

Disclosed herein is a kit comprising a disclosed pharmaceutical formulation with our without additional therapeutic agents to treat, prevent, inhibit, and/or ameliorate one or more symptoms or complications associated with a dystonia. Disclosed herein is a kit comprising the reagents necessary to perform one or more of the disclosed methods, such as, for example, PRM, MRM, or LC/MS/MS to detect one or more biomarkers of a dystonia.

Disclosed herein is a kit comprising the reagents necessary to perform one or more of the disclosed methods, such as, for example, RT-qPCR, RNAseq, a commercial assay (e.g., NanoString nCounter® assays), isothermal amplification-based assays, oligonucleotide-templated reactions, nanobead-based systems, and microfluidic-based assays for miRNA capture from biosamples and detection, and any combination thereof.

In an aspect, a disclosed kit can comprise a protein biomarker panel. A disclosed biomarker panel can detect 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or 25 or more, of the disclosed protein biomarker.

In an aspect, a disclosed kit can comprise a miRNA biomarker panel. A disclosed biomarker panel can detect 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or 25 or more, of the disclosed miRNA biomarkers.

In an aspect, a disclosed kit can comprise a metabolite biomarker panel. A disclosed biomarker panel can detect 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, or 25 or more, of the disclosed metabolite biomarkers.

In an aspect, a disclosed kit can comprise at least two components constituting the kit. Together, the components constitute a functional unit for a given purpose (such as, for example, treating a subject diagnosed with or suspected of having a dystonia). Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation. In an aspect, a kit for use in a disclosed method can comprise one or more containers holding a disclosed pharmaceutical formulation, a disclosed therapeutic agent, a disclosed reagent, or a combination thereof, and a label or package insert with instructions for use. In an aspect, suitable containers include, for example, bottles, vials, syringes, blister pack, etc. The containers can be formed from a variety of materials such as glass or plastic. The container can hold, for example, a disclosed pharmaceutical formulation and/or a disclosed therapeutic agent and can have a sterile access port (for example the container may be an intravenous solution bag or a vial having a stopper pierceable by a hypodermic injection needle). The label or package insert can indicate that a disclosed pharmaceutical formulation and/or a disclosed therapeutic agent can be used for treating, preventing, inhibiting, and/or ameliorating a dystonia or complications and/or symptoms associated with a dystonia. In an aspect, a disclosed kit can comprise additional components necessary for administration such as, for example, other buffers, diluents, filters, needles, and syringes.

In an aspect, a disclosed kit can comprise one or more therapeutic agents that target eIF2α signaling or target ISR dysregulation.

In an aspect, one or more disclosed agents in a disclosed kit can modulate the expression level of one or more proteins in Section F herein.

Another aspect of the present disclosure provides all that is described and illustrated herein.

The following Examples are provided by way of illustration and not by way of limitation.

### EXAMPLES

A series of translational studies of whole plasma and extracellular vesicles (EVs) were performed to support biomarker discovery in human subjects afflicted with dystonia. EVs have been found to circulate through many different body fluids including blood and urine. The isolation of EVs results in a large enrichment of low-abundant molecules that have pathophysiological significance. EV cargo composition changes between cell types and physiological states as composition determines EV secretion and function. An important breakthrough was the discovery of nucleic acids in EVs such as mRNA and miRNA. RNA molecules present in EVs seem to follow selective incorporation as evidence points to their enrichment relative to the RNA profiles of the secreting cells. Interestingly, several studies have shown that EV-associated mRNAs and miRNAs can be functionally transferred to recipient cells. A physiological relevance for the presence of mRNAs and miRNAs in EVs including immunological and vascularization functions among others have already been reported.

Early identification of the factors driving dystonia can provide a treatment path to intervene before disease onset. EVs are suitable to identify biomarkers for dystonias and to monitor translational state integrity. Identifying biomarkers in DYT1 and other dystonias represents an opportunity to improve diagnosis, predict disease progression, and/or track the efficacy of treatment. The Examples set forth herein provide a panel of biomarkers (e.g., proteins, microRNAs, and metabolites) for a dystonia (e.g., DYT1). The work described herein also classified these biomarkers as responsive to a putative therapeutic drug and as indicators of dysfunction in the “integrated stress response”, a pathway known to be involved in multiple forms of dystonia and other neurological diseases. Because DYT1 dystonia is part of a group of dystonias with ISR dysregulation, the biomarker panels described herein can be used to define subsets of biochemically similar dystonias when applied to individuals with sporadic and all other forms of dystonia. Besides diagnostic value for disease states, individuals with this biomarker panel would be candidates for particular therapeutic interventions. Details of candidate biomarker identification and selection are described below.

### Example 1

### DYT-TOR1A Genotype Alter Extracellular Vesicle Composition in Murine Cell Model and Showed Potential for Biomarker Discovery

Introduction: Biomarkers that can be used to identify patient subgroups with shared pathophysiology, or that can be used as pharmacodynamic readouts of disease state are valuable assets for successful clinical trial design. In translational research for brain diseases, extracellular vesicles (EVs) have become a high-priority target for biomarker discovery because of their ubiquity in peripheral biofluids and potential to indicate brain state. Here it is shown that a murine cell model demonstrates proof-of-concept that DYT1 genotype disrupts EV protein composition and shows corrective effects of ritonavir treatment.

Abbreviated Materials and Methods: Unbiased quantitative proteomics were performed on EVs isolated from DYT-TOR1A knockin mouse embryonic fibroblasts and littermate controls to discover candidates for protein biomarkers. The response of genotype perturbations to drug treatment conditions was examined to determine pharmacodynamic properties.

Results: Many DYT-TOR1A MEF EV differences were significantly corrected by ritonavir, a drug recently shown to correct DYT-TOR1A phenotypes in cell and mouse disease models. Tool compounds were used to explore the effect of the integrated stress response (ISR), which regulates protein synthesis and is implicated in dystonia pathogenesis. ISR inhibition in WT cells partially phenocopied the effects of DYT-TOR1A on EV proteome composition, and ISR potentiation in DYT-TOR1A caused changes that paralleled ritonavir treatment.

These results collectively show that DYT-TOR1A genotype alters EV protein composition, and these changes can be dynamically modulated by a candidate therapeutic drug and ISR activity state. These mouse model findings provide proof-of-concept that EVs may be a useful source of biomarkers in human populations and further suggest specific homologs to evaluate in cross species validation.

Dystonia is a movement disorder characterized by sustained muscle contractions with abnormal twisting movements (Balint et al., Nat Rev Dis Prim., 2018, 4(1):25). DYT-TOR1A is a rare inherited dystonia caused by a mutation in TOR1A (n. delGAG, p. ΔE) leading to a childhood onset form of the disease that often involves most of the body (e.g., early-onset, generalized dystonia) (Ozelius L J, et al. Nat Genet., 1997, 17(1):40-8). Currently, there is substantial unmet clinical need for DYT-TOR1A dystonia treatment. Oral medications are limited by narrow therapeutic windows and side effects, typically leaving deep brain stimulation surgery as the major alternative treatment option (Lungu C et al., Neurology, 2020, 94(12):526-37). To fill these treatment gaps, drug discovery efforts are underway to identify highly effective, well tolerated, and orally bioavailable small molecules. It was previously demonstrated that ritonavir, an HIV protease inhibitor, rescues diverse disease phenotypes in DYT-TOR1A preclinical models (Caffall Z F et al., Sci Transl Med., 2021, 13(607):1-14). However, translating effective treatments from the bench into the clinic is especially difficult for neurological diseases, which have a below average success rate in all clinical trial phases compared to other body systems (PharmaIntelligence Rep., 2020; Takebe T et al., Clin Transl Sci., 2018, 11(6):597-606). One strategy to improve clinical trial design is identifying and measuring biomarkers before and during the treatment intervention. Biomarkers have multiple classifications depending on their clinical context of use. These include predictive biomarkers, which can be used to stratify patient subpopulations and enrich recruitment for subjects most likely to respond to the given intervention, and pharmacodynamic/response biomarkers, which track physiological changes throughout treatment to assess successful target engagement (BEST (Biomarkers, EndpointS, and other Tools) Resource, 2021; Califf R M, Exp Biol Med., 2018, 243(3):213-21). In diseases of the CNS like dystonia, peripheral biomarkers for brain state are challenging to isolate because of the blood brain barrier (BBB). Extracellular vesicles (EVs) have been found to be a promising source for CNS disease biomarkers, since they can cross the BBB and carry protein and RNA cargo secreted by brain cells (Hornung S et al., Front Mol Neurosci. 2020, 13). As one example, neurofilament light chain in plasma EVs has been studied in X-linked dystonia-parkinsonism and other neurodegenerative diseases as a biomarker for brain axonal degeneration (Al Ali J et al., 2021, 36(1):206-15; Gaetani L et al., J Neurol Neurosurg Psychiatry, 2019, 90(8):870-81). EVs provide a view into the physiological state of their cells of origin.

In this study, it was sought to determine whether the DYT-TOR1A genotype altered EV composition, a finding that would open the possibility to use EVs as biomarkers in this disease. Focus was on obtaining proof-of-concept in cell lines which also secrete EVs because DYT-TOR1A is a rare genetic disease with geographically isolated human subject populations (Defazio G et al., Lancet Neurol., 2004, 3(11):673-8; Ozelius L et al., GeneReviews®, University of Washington, Seattle, 2016). While DYT1 patient-derived cell lines were considered (Al Ali J et al., 2021, 36(1):206-15; Cruz L et al., Mol Ther Nucleic Acids, 2020, 4; 21:1-12), the use murine of embryonic fibroblasts (MEFs) derived from the Tor1aΔGAG/+ knockin mouse model of DYT-TOR1A (Goodchild R E et al., Neuron, 2005, 48(6):923-32) was chosen because of its construct validity and that it provides a uniform genetic background to reduce variability in a proof-of-concept experiment. The effects of DYT-TOR1A on EV protein composition was examined using quantitative LC-MS/MS proteomics. Once putative genotype-modified candidates were identified, their behavior was explored in response to pharmacological manipulations: therapeutic treatment with a candidate dystonia drug, ritonavir, and modulation of a conserved signaling pathway perturbed in multiple dystonias, the integrated stress response (ISR) (Rittiner J E et al., Neuron, 2016, 92(6):1238-51). Lastly, experimental observations were combined with pragmatic criteria for ideal clinical biomarkers to put forth candidates with the highest potential for future tests of DYT-TOR1A EV biomarkers in human subjects.

### Results

### DYT-TOR1A MEF EVs Show Altered Protein Composition.

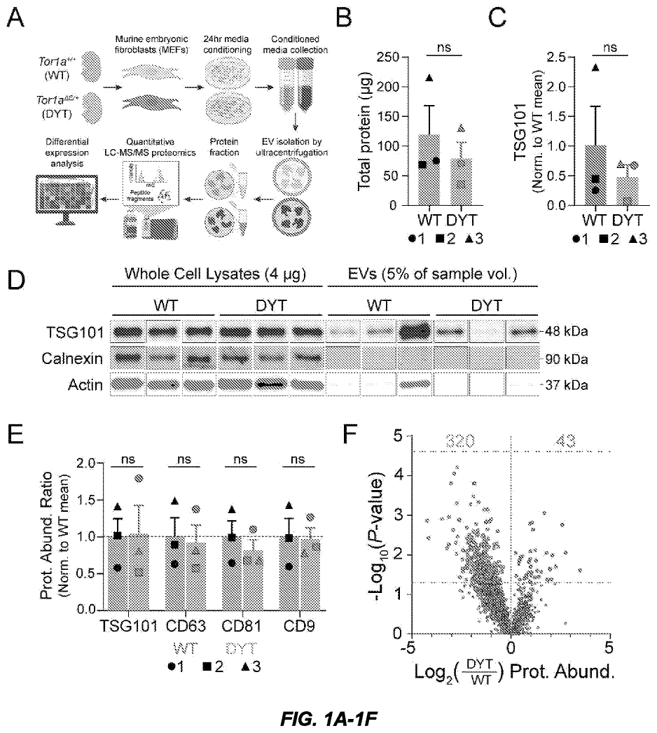

Immortalized murine embryonic fibroblast (MEF) cell lines were prepared from heterozygous knockin mice bearing the DYT-TOR1A mutation (Tor1aΔGAG/+ genotype hereafter abbreviated as DYT-TOR1A or DYT) (Goodchild R E et al., Neuron, 2005, 48(6):923-32) and wildtype (WT) littermate embryos according to standard methodology (see Materials and Methods). Three independent cell lines for each genotype were used. Genotype and all drug treatment conditions were tested in a blinded experimental design and in parallel by splitting the parental cell line flask into separate flasks for each condition (see FIG. 1A). EVs produced during the 24-hour period following media exchange with an EV-depleted media were isolated from the conditioned media by ultracentrifugation (Théry C et al., Curr Protoc Cell Biol., 2006, 1-29). Protein was isolated from the resultant EV pellet. DYT-TOR1A did not significantly modify recovery of total protein (see FIG. 1B) or amount of the constitutive EV marker, TSG101 (see FIG. 1C). Specific EV enrichment was confirmed by western blot for TSG101 compared to non-EV markers (calnexin, actin) (see FIG. 1D) (Théry C et al., J Extracell Vesicles, 2018, 7). Samples were then subjected to unbiased, quantitative LC-MS/MS proteomics analysis. Quantitative proteomic measurements also demonstrated that EV protein abundances of classic EV markers (TSG101 and the tetraspanins CD9, CD81, and CD63) were not modified by genotype (see FIG. 1E).

The EV proteome was characterized to identify genotype-dependent changes in EV protein abundances between DYT-TOR1A and WT EV samples. Following alignment of peptide signals to unique identifying peptides (UIPs) and removal of proteins with fewer than two detected UIPs, 1974 proteins were detected across all cell lines. Using a Bonferroni-adjusted p-value threshold for multiple hypothesis testing (p<2.5e-5) (Armstrong R A. Ophthalmic Physiol Opt., 2014, 34(5):502-8), no significant genotype effects were identified. This discovery dataset was used to identify putative DYT biomarkers for testing in follow-on experiments. Using an uncorrected p-value cutoff of less than 0.05, 363 of 1974 proteins were identified with significantly different abundances in DYT-TOR1A versus WT EVs (see FIG. 1F). This differential subset of 363 is more than 3.5 times larger than would be predicted by chance (e.g., 99 proteins from the total of 1974, based on the expected proportion α=0.05).

Among the 363 differential proteins, there was an asymmetric distribution of genotype effects. The DYT-TOR1A effects showed a bias towards decreased abundances, with 320 proteins being significantly less abundant compared to only 43 being more abundant in DYT-TOR1A relative to WT (two-tailed binomial sign test, p<0.0001). This skewed distribution was also maintained across all EV proteins (1491 less, 483 more; two-tailed binomial sign test, p<0.0001).

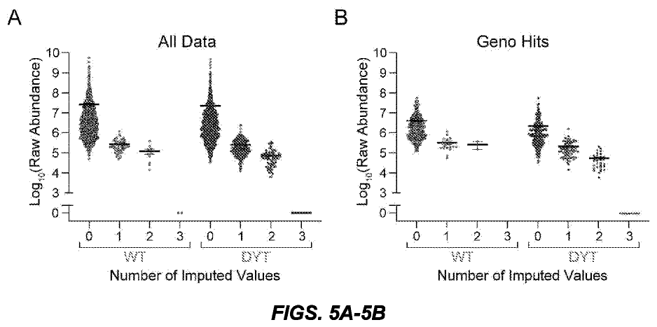

Technical reasons that could artifactually cause such a distribution bias were considered, e.g. lower EV yields and/or detection thresholds not being met preferentially in DYT samples. As FIG. 1E demonstrates, there were no significant genotype-dependent differences in abundance of EV constituents detected in the LC-MS/MS data. Secondly, when a protein is not detected in a sample, an imputed value is given as described in Materials and Methods prior to sample loading normalization. It was examined whether the DYT genotype effects came preferentially from proteins with multiple imputed values. Instead, hits were observed to be distributed proportionally across proteins with 0, 1, 2 or 3 inputted values and the vast majority of hits came from proteins with no imputed values (see FIGS. 5A-5B). These observations rule out LC-MS/MS detection thresholds as a systematic confound. In summary, proteomic analysis was performed on MEF culture-derived EV preparations and 363 candidate proteins were identified for DYT-TOR1A genotype biomarkers.

### Ritonavir Shows Corrective Effects on DYT-TOR1A EV Protein Composition

Recent studies have shown corrective effects of the HIV protease inhibitor ritonavir on cell and brain phenotypes in DYT-TOR1A preclinical models (Caffall Z F et al., Sci Transl Med., 2021, 13(607):1-14). To explore the potential for the DYT-TOR1A genotype-associated EV changes that were identified to be used as pharmacodynamic biomarkers of disease state, DYT-TOR1A MEF cultures were exposed to 20 μM ritonavir throughout the 24 hours of media conditioning preceding EV isolation. EV protein fractions were analyzed by quantitative LC-MS/MS proteomics performed in the same batch run as all conditions reported in this study.

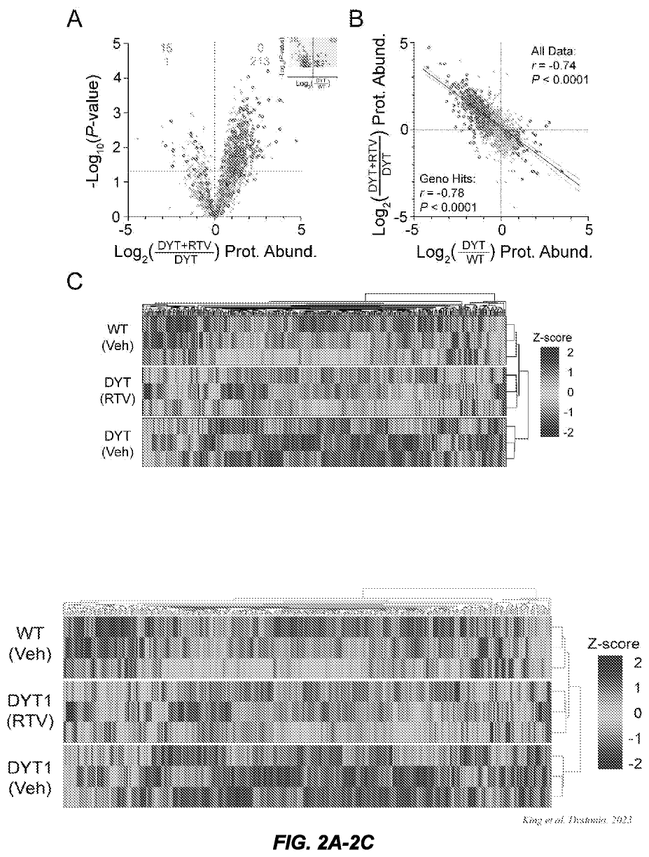

Of the subset of 363 proteins significantly disrupted by DYT-TOR1A genotype basally, we found that >60% (230/363) had significant changes in abundance following ritonavir treatment at a threshold of p≤0.05. This number of hits is 12 times greater than would be predicted by chance if ritonavir had no true effect on the genotype-dependent hits (18.15 proteins by α=0.05). When examining the behavior of the 363 putative DYT biomarkers independent of p-value, the overwhelming majority of proteins showed ritonavir effects on protein abundance that were in the corrective direction (344/363) (see FIG. 2A). The putative DYT biomarker subset of proteins also showed strong and inverse correlations between genotype and ritonavir effects (Pearson's r=−0.78, p<0.0001) (see FIG. 2B). Noticing the very large number of proteins modified by ritonavir, the relationship between DYT genotype disruptions and DYT+RTV effects across the entire proteome was examined, which showed that the strong inverse correlation was maintained (n=1974, Pearson's r=−0.74, p<0.0001) (see FIG. 2B). Proteome-wide, ritonavir significantly modified 29% of the DYT EV proteome (uncorrected p<0.05, 582/1974) in a direction that was opposite to the genotype effect, with an asymmetric distribution toward increasing abundances for both significant and non-significant abundance changes (two-tailed binomial sign test: 508/582, with log 2 fold change>0, p<0.0001; 1372/1974 with log 2 fold change>0, p<0.0001). Lastly, hierarchical clustering was utilized to evaluate ritonavir's effects on the putative DYT-TOR1A genotype biomarker proteins (n=363). This analysis showed that ritonavir-treated DYT-TOR1A EV samples clustered more closely with WT than DYT-TOR1A samples (see FIG. 2C).

In summary, DYT-TOR1A genotype disruptions of EV protein composition show potential as pharmacodynamic markers of disease state. Ritonavir treatment acutely modified a substantial fraction of DYT-TOR1A genotype-dependent protein disruptions (95%) and caused dendrogram clustering of EV proteome to become more closely related to WT samples than the DYT-TOR1A genotype.

### Influence of the Integrated Stress Response Pathway on EV Composition in WT and DYT-TOR1A

DYT-TOR1A and other dystonias show dysfunction in a biochemical pathway, the integrated stress response (ISR), that has wide-reaching effects on the proteome because it regulates global protein synthesis (Rittiner J E et al., Neuron., 2016, 92(6):1238-51). This prompted the question of how the broad EV compositional differences observed in the previous two experiments were related to ISR pathway effects.

ISR tool compounds were used to modify ISR activity. Prior studies established the corrective directionality of the eIF2α phosphatase inhibitor salubrinal in DYT-TOR1A cell and mouse model phenotypes and sufficiency of the ISR inhibitor ISRIB to mimic DYT-TOR1A phenotypes (Caffall Z F et al., Sci Transl Med., 2021, 13(607):1-14; Rittiner J E et al., Neuron., 2016, 92(6):1238-51; Helseth A R et al., Science, 2021, 372(6540)). It was therefore hypothesized that ISRIB-induced EV composition changes in WT MEF EVs would reproduce DYT-TOR1A genotype differences that were related to ISR dysregulation and that salubrinal treatment of DYT-TOR1A MEF EVs would cause normalizing shifts in genotype differences if they were related to ISR dysregulation.

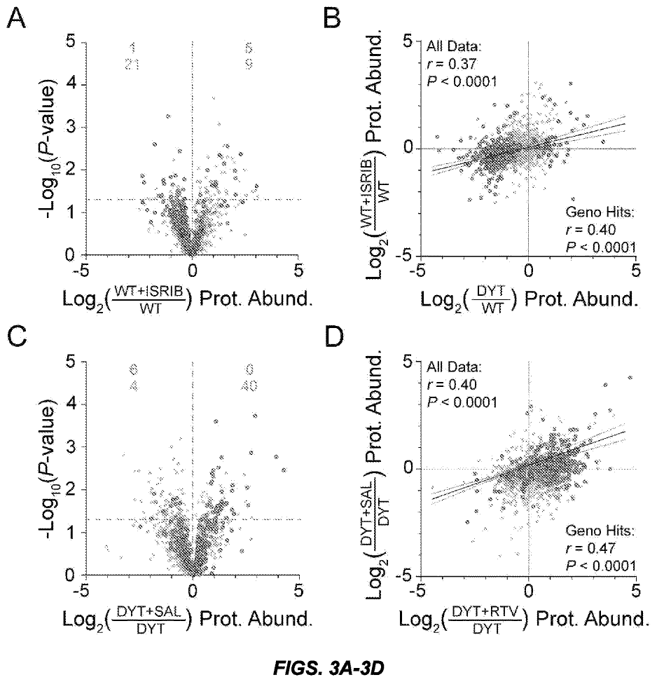

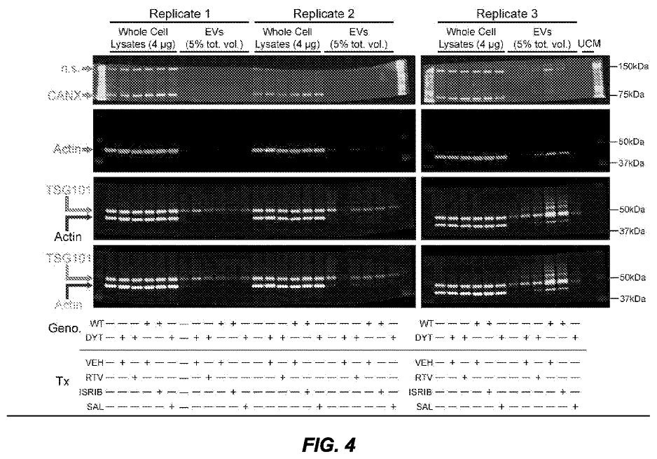

WT MEFs were treated with 50 nM ISRIB to inhibit ISR pathway output for 24 hours prior to EV harvest from the conditioned media. ISRIB treatment of WT cells disrupted fewer proteins at the statistical threshold of p<0.05 than were observed between DYT and WT samples (103/1974 (5%) vs. 363/1974 (18%)) and only 7% of the genotype-disrupted proteins (26/363) were reproduced by ISRIB at the statistical threshold (p<0.05). However, an examination of proteome-wide effects independent of p-value thresholds showed protein abundance directionality (greater or lesser) to be non-randomly distributed (Fisher's exact test, p<0.0001) and in a directionality similar to the DYT genotype effects (see FIG. 3A). A Pearson's correlation analysis showed a positive correlation between DYT genotype effects and ISRIB effects, supporting the hypothesis that ISRIB treatment of WT cells mimics DYT genotype effects (Pearson r=0.40, p<0.0001) (see FIG. 3B). Interestingly, as was observed with ritonavir effects, this correlation was also maintained when the entire proteome was evaluated (Pearson r=0.37, p<0.0001).

To augment ISR activity in DYT-TOR1A MEFs, cell cultures were treated with 20 μM salubrinal during the 24-hour conditioning period prior to EV harvest from the media. Salubrinal is a specific inhibitor of eIF2α phosphatases, CReP and GADD34 (Boyce M, et al. Science, 2005, 307(5711):935-9). Salubrinal treatment of DYT samples significantly modified 9% of the total proteins (169/1974) and caused significant corrective effects on 13% of DYT disrupted proteins (46/363) (see FIG. 3C). Like ISRIB, secondary analyses of effects independent of p-value thresholds showed that DYT disrupted proteins were not randomly distributed (Fisher's exact test, p<0.0001) and showed directionality biases supporting the hypothesis that salubrinal has corrective effects on DYT disruptions (see FIG. 3C). Lastly, the concordance of drug effects between salubrinal and ritonavir on DYT-TOR1A MEF EV protein abundances was examined, given that both drugs augment ISR activity (Caffall Z F et al., Sci Transl Med., 2021, 13(607):1-14; Boyce M et al., Science, 2005, 307(5711):935-9; De Gassart A et al., Proc Natl Acad Sci USA, 2016, 113(2):E117-26). Ritonavir and salubrinal effects on the putative DYT-TOR1A biomarker proteins were positively correlated (Pearson r=0.47, p<0.0001) (FIG. 3D). This result is consistent with a degree of shared mechanism of action between salubrinal and ritonavir.

In summary, ISR tool compound experiments demonstrate that ISR activity effects correlate with DYT-TOR1A genotype disruptions and ritonavir corrective effects on MEF EV protein composition. These results support the hypothesis that DYT-TOR1A genotype disruptions of MEF EV protein abundances and the corrective effects of ritonavir treatment are related, at least in part, to ISR pathway activity.

### Stratification of EV Components for Biomarker Potential in Human Samples

This study takes advantage of the benefits of control over biological variables that an animal model system affords to generate initial proteomic discovery datasets for putative biomarkers of DYT-TOR1A. To guide translation to dystonia biomarker discovery in future patient-derived cell line or human plasma and CSF samples, we considered the results from our three experimental tests alongside human biospecimen datasets to prioritize candidates with the greatest potential.

The stratification process considered the following features. First, protein candidates were identified that have been previously detected in human plasma (Schwenk J M et al., J Proteome Res., 2017, 16(12):4299-310). This criterion identified 164 of the 363 genotype-disrupted proteins. Second, candidates where identified that showed conserved directionality of effects across two drug perturbations, independent of effect size or p-value (Ritonavir-DYT, ISRIB-WT). This criterion identified 121 of 164 proteins. Then, a composite score of genotype and ritonavir effect sizes was created by summing the absolute value of their respective Cohen's d score. The results of this analysis are compiled in TABLE S1 (see Absolute Cohen's d Sum (Geno+RTV)). Overall, a third of the DYT-TOR1A genotype disrupted proteins show favorable characteristics according to these prioritizations.

A discovery proteomics approach was used to determine whether DYT-TOR1A alters EV composition by comparing EVs isolated from DYT-TOR1A heterozygous knockin MEF cultures to those from wildtype littermate controls. A subset of 363 proteins were identified with significant genotype effects. Pharmacodynamic responsivity to candidate drugs was tested and ritonavir was found to have a broad corrective effect on the EV proteome, and at least a subset of these changes further correlates with ISR activity. Altogether, these results provide preclinical proof-of-principle for the potential to use EVs in DYT-TOR1A for predictive and pharmacodynamic biomarker applications and define a prioritized list of candidate biomarkers based on follow-on testing and human bioinformatic data.

A significant takeaway is that in DYT-TOR1A, rather than identifying one or a handful of candidate biomarkers, broad proteome-wide disruptions and corrections were found. At least 3 potential mechanisms were identified that could cause the widespread EV composition disturbances observed in DYT-TOR1A. First, in previous in vitro studies of DYT-TOR1A, patient-derived dermal fibroblasts exhibit secretion deficits through the ER-to-Golgi secretory pathway (Rittiner J E et al., Neuron, 2016, 92(6):1238-51; Hewett J W et al., Proc Natl Acad Sci., 2007, 104(17):7271-6), which regulates trafficking to a variety of intracellular locations prior to extracellular release (Pettersen Hessvik N et al., Cell Mol Life Sci., 2018, 75:193-208). EVs are a heterogeneous population of vesicles produced by distinct biogenesis mechanisms—exosomes form as intraluminal vesicles within late endosomes and are released when these multivesicular bodies fuse with the plasma membrane, while microvesicles arise from direct outward budding of the plasma membrane (Pettersen Hessvik N et al., Cell Mol Life Sci., 2018, 75:193-208; Yáñez-Mó M et al., J Extracell Vesicles, 2015, 4(1):27066; Tricarico C et al., Small GTPases, 2017, 8(4):220-32). However, both carry cargo sorted and transported by the ER-to-Golgi pathway, and the ultracentrifugation EV isolation method used in this study likely includes a mixed EV population (Zhang Q et al., Cell Rep., 2019, 27(3):940-954; Théry C et al. Current Protocols in Cell Biology., 2006, 30(1):3.22.1-3.22.29). Broad-based changes in DYT-TOR1A EV composition may reflect upstream disruptions in these intracellular trafficking pathways. Second, ΔE-TorsinA abnormally localizes to the nuclear envelope relative to TorsinA's usual predominance in the ER, and this mislocalization is likely to influence trafficking through the nuclear envelope (Goodchild R E et al., Neuron., 2005, 48(6):923-32; Naismith T V et al., Proc Natl Acad Sci., 2004, 101(20):7612-7; Jokhi V et al., Cell Rep., 2013, 3(4):988-95; Rampello A J et al., J Cell Biol., 2020, 219(6); Shroff K et al., Neurobiol Dis., 2021, 158). A third mechanism that could cause broad EV compositional changes is the influence of ISR dysregulation on protein synthesis in DYT-TOR1A. ISR dysfunction is implicated in the pathogenesis of DYT-TOR1A and other dystonias (Rittiner J E et al., Neuron., 2016, 92(6):1238-51). The ISR regulates mRNA translation at the level of translation initiation (Pakos-Zebrucka et al., EMBO Rep., 2016, 17(10):1374-95). ISR activity markedly and globally reconfigures which proteins are translated (Pakos-Zebrucka et al., EMBO Rep., 2016, 17(10):1374-95; Vattem K M et al., Proc Natl Acad Sci., 2004, 101(31):11269-74; Wek R C, Cold Spring Harb Perspect Biol., 2018, 10(7)). In addition, HIV protease inhibitors (including ritonavir) activate the ISR (Caffall Z F et al., Sci Transl Med., 2021, 13(607):1-14; De Gassart A et al., Proc Natl Acad Sci USA, 2016, 113(2):E117-26) and show corrective effects on several DYT-TOR1A phenotypes (Caffall Z F et al., Sci Transl Med., 2021, 13(607):1-14). Therefore, the influence of the ISR on global proteostasis could contribute to the EV proteome genotype effects and ritonavir effects we observed. Although EV cargo loading is a regulated process, rather than a simple stochastic loading of nearby proteins (Tricarico C et al., Small GTPases., 2017, 8(4):220-32; Villarroya-Beltri C et al., Semin Cancer Biol., 2014, 28:3-13.), a sufficiently large change in proteostasis could be reflected across multiple subcellular compartments, including EVs. Future studies examining the intracellular dynamics of the DYT-TOR1A candidate biomarkers identified in this study could further test these three candidate pathophysiological mechanisms.

It was striking that the strongest and broadest drug effect identified in this study was caused by ritonavir and not ISR-targeting tool compounds. In measuring the pharmacodynamic responsivity of DYT-TOR1A genotype-disrupted proteins to ritonavir, it was found that 63% of these proteins (230/363) were significantly different in DYT-TOR1A following ritonavir treatment and 95% of these changes were in the corrective direction toward WT. This is best illustrated by unsupervised hierarchical clustering of each sample showing that all three ritonavir-treated DYT-TOR1A samples cluster closer to WT cell lines than their DYT vehicle-treated corresponding cell lines. These results identify a proteomic “signature” that could be used as a measure of pharmacodynamic response.

Many of the differentially abundant EV proteins identified in this study show strong cross species homology and are detected in human plasma. Since similar mouse-to-human predictive approaches have proven useful in other diseases (Eninger T et al., Proc Natl Acad Sci., 2022, 119(24)), generation of a prioritized candidate biomarker set using the advantages of the mouse model system to guide pharmacodynamic biomarker discovery in human patients with this rare disease was emphasized.

Identifying a DYT-TOR1A biomarker signature also has implications for other forms of dystonia beyond DYT-TOR1A that may benefit from predictive biomarkers. While DYT-TOR1A has a recognizable clinical manifestation and is readily diagnosed by genotype testing, sporadic dystonias with no known genetic etiology are the most common form of dystonia. It was previously shown that ~4% of sporadic cervical dystonia patients had mutations in ATF4, the main effector protein of the ISR, and several other inherited dystonias also have ISR involvement (Rittiner J E et al., Neuron., 2016, 92(6):1238-51; Musacchio T et al., Ann Neurol., 2021, 89(6):1257-8; Kuipers D J S et al., Ann Neurol., 2021, 89(3):485-97; Burnett S B et al., Neurobiol Dis., 2020, 146; Zakirova Z, PLOS Genet., 2018, 14(1):e1007169). It was therefore anticipated that EV biomarkers may be useful not only for pharmacodynamic monitoring but also for identifying dystonia subpopulations with shared pathophysiology. Such predictive biomarkers could help identify sporadic dystonia patients who are most likely to respond to ritonavir or other ISR-modifying treatments in future clinical trials. Finally, a common but poorly understood feature of many inherited dystonias is that they show reduced penetrance. Current DYT-TOR1A genetic mouse models are not suited to address whether EV biomarkers may also have prognostic value because the model does not reproduce the dystonia phenotype. Future human studies will be needed to determine whether DYT-TOR1A EV biomarkers vary based on symptom manifestation and can be used to predict disease penetrance. Our results provide proof-of-concept that DYT-TOR1A genotype disrupts EV composition and its pharmacodynamic responsiveness under the more optimal homogenous conditions afforded by mouse models. We hope that these findings will accelerate future biomarker discovery.

### Materials and Methods

### Experimental Blinding, Power, and Statistical Approach

Sample size was arbitrarily set a priori at three samples per group. Experimenters were blinded to MEF cell line genotype and drug treatment prior to cell culture experiments, and proteomics were performed on these blinded sample groups. Experimenters were unblinded after initial differential abundance analyses were completed. For differences in protein abundances, statistical testing used unpaired Student's t tests between n=3 WT and n=3 DYT samples without correction for multiple hypothesis testing or with Bonferroni correction where noted (Armstrong R A, Ophthalmic Physiol Opt., 2014, 34(5):502-8). Two-tailed binomial sign test was performed using a null probability P=0.5. Fisher's Exact Test was performed on contingency tables for overlaps of the 363 significantly different proteins between conditions using abundances greater than or less than zero log 2 fold change.

### Animals

ΔE Torsin1a knockin (IMSR_JAX:025637) (Goodchild R E et al., Neuron, 2005, 48(6):923-32) mice on C57BL/6 background were bred in standard housing conditions with food and water provided ad libitum. All procedures were approved by the Duke University Institutional Animal Care and Use Committee (IACUC).

### Cell Lines and Cell Culture

Mouse embryonic fibroblasts were harvested as previously described (Shroff K et al., Neurobiol Dis., 2021, 158) from E14 TOR1AΔE/+ mice and immortalized via SV40 transfection. MEFs were maintained in sterile-filtered MEF media [DMEM (Thermo Fisher Scientific, #11995-065)+10% fetal bovine serum (Hyclone, #SH0071.03)+lx GlutaMAX (Gibco, #35050-061)+1% penicillin/streptomycin/amphotericin (Gibco, #15240062)+1% Non-Essential Amino Acids (Gibco, #11140050)+55 nM β-Mercaptoethanol (Gibco, #21985023)] at 37° C./5% CO2.

### MEF EV-Conditioned Media Collection

EV-depleted (dEV) media was prepared by spinning 10% FBS MEF media for 18 hours at 100,000×g (Beckman L8-55M ultracentrifuge; SW27 rotor; 23,600 rpm; 4° C.) (Guha D et al., J Neuroinflammation, 2019, 16(1):254). MEFs were seeded at 5.8×105 cells into one 15 cm dish per line. At 90% confluence, cells were passaged 1:10 into four 15 cm dishes per line and when each line reached ~50% confluence, media was exchanged for dEV media containing 1% dEV FBS and the given drug treatment. Ritonavir (Tocris Biosciences, #5856), ISRIB (Sigma, #SML0843), and salubrinal (Tocris Biosciences, #2347) were dissolved in DMSO (100 mg/ml) and frozen in aliquots at −20° C. On the day of each treatment, these aliquots were thawed and added to dEV media containing 1% FBS to final concentrations (0.04% DMSO vehicle, 50 nM ISRIB, 20 μM ritonavir, or 20 μM salubrinal). After 24 hours in dEV media, the EV-conditioned media and cells were collected separately.

### EV-Protein Isolation

EV-conditioned media was centrifuged at 4° C. for 20 min at 2000×g (Sorvall HS-4, 3500 rpm). Supernatant was transferred to a new tube and centrifuged at 4° C. for 30 min at 8000×g (Sorvall HS-4, 6500 rpm). Final clarified supernatant was stored at −80° C. Media was thawed in room temperature water bath and 36 mL per sample was ultra-centrifuged in Ultra-Clear tubes (Beckman Coulter, #344058) for 16 hours at 100,000×g (Beckman L8-55M ultracentrifuge; SW27 rotor; 23,600 rpm; 4° C.) to isolate EVs (21). The supernatant was discarded and protein was extracted from the pellet. Protein was extracted by adding 50 μL modified RIPA buffer [1% Triton X-100, 0.5% SDS, 0.5% deoxycholic acid, 50 mM NaPO4 at pH 7.4, 150 mM NaCl, 2 mM EDTA, 50 mM NaF, 10 mM sodium pyrophosphate, 1 mM sodium orthovanadate, and protease inhibitor cocktail (Roche, #04693159001)], vortexing on low speed for 15 see, and shaking on an orbital shaker for 1 hour at 4° C. Cell lysates were prepared in 1 mL modified RIPA buffer by rotating on a Nutator for 2 hours. Lysates were then sonicated and centrifuged for 10 min at 10,000×g to remove insoluble material, and the supernatant was taken as the whole cell lysate protein fraction.

### Immunoblotting

Total EV protein concentrations were quantified using a Micro BCA™ Protein Assay Kit (Thermo Fisher Scientific, #23235) and cell lysate protein concentrations were quantified by Pierce™ BCA assay (Thermo Fisher Scientific, #23225). Proteins were resolved on 4-15% TGX gels (BioRad, #5671085), transferred to nitrocellulose membrane, blocked in TBS-T (0.1% Tween-20) with 5% BSA, and probed as indicated. Densitometry was quantified using ImageJ (Schneider C A et al., Nat Methods., 2012, 9(7):671-5). The following primary antibodies and dilution ratios were used for immunoblotting experiments: anti-Actin—1:5000 (Millipore, #MAB1501); anti-TSG101—1:1000 (Abcam, #ab30871); anti-calnexin—1:1000 (Proteintech, #10427-2-AP).

### Quantitative Mass Spectrometry Proteomics

Sample Preparation: The Duke Proteomics and Metabolomics Core Facility (DPMCF) received 18 samples (3 biological replicates each of six conditions). Methods are as described in (Shroff K et al., Neurobiol Dis., 2021, 158:105464.) with minor modifications and restated here for convenience: “Samples were first normalized to 20 μg and spiked with undigested casein at a total of 40, 80, or 160 fmol/μg, then reduced with 10 mM dithiothreitol for 30 min at 80° C. and alkylated with 20 mM iodoacetamide for 30 min at room temperature. Next, they were supplemented with a final concentration of 1.2% phosphoric acid and 741 μL of S-Trap (Protifi) binding buffer (90% MeOH/100 mM triethylammonium bicarbonate). Proteins were trapped on the S-Trap, digested using 20 ng/μl sequencing grade trypsin (Promega) for 1 hr at 47° C., and eluted using 50 mM triethylammonium bicarbonate, followed by 0.2% formic acid, and lastly using 50% acetonitrile/0.2% formic acid All samples were then lyophilized to dryness and resuspended in 40 μL 1% trifluoracetic acid/2% acetonitrile containing 12.5 fmol/μL yeast alcohol dehydrogenase (ADH YEAST). A Sample Pool QC (SPQC) was created from 3 μL of each sample. SPQCs were run periodically throughout the acquisition period.

Quantitative Analysis Methods: Quantitative LC-MS/MS was performed on 2 μL of each sample, using a nanoAcquity UPLC system (Waters Corp) coupled to a Thermo Orbitrap Fusion Lumos high resolution accurate mass tandem mass spectrometer (Thermo) via a nano-electrospray ionization source. Briefly, the sample was first trapped on a Symmetry C18 20 mm×180 μm trapping column (5 μl/min at 99.9/0.1 v/v water/acetonitrile), after which the analytical separation was performed using a 1.8 μm Acquity HSS T3 C18 75 μm×250 mm column (Waters Corp.) with a 90-min linear gradient of 5 to 30% acetonitrile with 0.10% formic acid at a flow rate of 400 nL/min with a column temperature of 55 C. Data collection on the Fusion Lumos mass spectrometer was performed in a data-dependent acquisition (DDA) mode of acquisition with a r=120,000 (@m/z 200) full MS scan from m/z 375-1500 with a target automatic gain control (AGC) value of 2e5 ions. MS/MS scans were acquired at Rapid scan rate (Ion Trap) with an AGC target of 5e3 ions and a max injection time of 25 ms. The total cycle time between full MS scans was 2 sec. A 20 s dynamic exclusion was employed to increase depth of coverage.

Proteomics Data Analysis: Following 22 total UPLC-MS/MS analyses (including 4 SPQC injections) were imported into Proteome Discoverer 2.3 (Thermo Scientific Inc.), and analyses were aligned based on the accurate mass and retention time of detected ions (“features”) using Minora Feature Detector algorithm in Proteome Discoverer. Relative peptide abundance was calculated based on area-under-the-curve (AUC) of the selected ion chromatograms of the aligned features across all runs. The MS/MS data was searched against the SwissProt M. musculus database, SwissProt bovine database (downloaded September 2019) and an equal number of reversed sequence “decoys” for false discovery rate determination. Mascot Distiller and Mascot Server (v 2.5, Matrix Sciences) were utilized to produce fragment ion spectra and to perform the database searches. Database search parameters included fixed modification on Cys (carbamidomethyl) and variable modifications on Meth (oxidation) and Asn and Gln (deamidation). Full trypsin enzyme rules were selected with 2 ppm precursor and 0.8 Da product ion mass tolerances. Peptide Validator and Protein FDR Validator nodes in Proteome Discoverer were used to annotate the data at a maximum 1% protein false discovery rate.

Following data alignment and AUC quantitation, missing values were imputed in the following manner. If less than half of the values are missing within any one treatment group, values are imputed with an intensity derived from a normal distribution defined by measured values within the same intensity range (20 bins). If greater than half values are missing for a peptide in a group and a peptide intensity is >5e6, then it was concluded that peptide was misaligned and its measured intensity is set to 0. All remaining missing values are imputed with the lowest 5% of all detected values. These data were then subjected to a sample loading normalization in which the total signals were summed and those summed values were used as normalizing factors across all samples. All peptide AUCs belonging to the same protein were then summed together to generate a protein level intensity.” (Shroff K et al., Neurobiol Dis., 2021, 158:105464).

Data were analyzed using GraphPad Prism v9 and R v4.2.0. Hierarchical clustering was performed in R using Euclidean distance measures and average-linkage clustering (Crameri F., Zenodo, 2021, 12). All raw data and Protein Discoverer results files that support this study are publicly available in MassIVE.ucsd.edu under the identifier MSV000090835.

### Potential Candidate Biomarker Criteria

For proteins, our DYT-TOR1A genotype-dependent subset of 363 differential proteins were annotated as ‘In Human Plasma’ based on their presence in a public database, the Human Plasma Proteome Project (HPPP) (Ignjatovic V et al., J Proteome Res., 2019, 18(12):4085-4097; Deutsch E W et al., J Proteome Res., 2021, 20(12):5241-63). The HPPP is a set of >3500 proteins that have been detected with varying degrees of evidence in different mass spectrometry studies. We focused on HPPP proteins that were detected in a minimum of 3 distinct studies. This criterion identified 164 of the 363 genotype-disrupted proteins.

We next used Cohen's d as a standardized effect size for each genotype and drug treatment condition. This was calculated using the formulas below (Lee D K, Korean J Anesthesiol., 2016, 69(6):555-62), where n1 and n2 are group sample sizes, s1 and s2 are group standard deviations, and s²pooled is a pooled variance calculated using both groups' features. Calculation of Cohen's d and s²pooled are shown below in Equations 1 and 2, respectively.

$${Cohen}^{′} s d = \frac{{\overline{x}}_{Exp} - {\overline{x}}_{Control}}{{s}_{pooled}} \qquad Equation 1$$

$${s}_{pooled}^{2} = \sqrt{\frac{( {n}_{1} - 1 ) {s}_{1}^{2} + ( {n}_{2} - 1 ) {s}_{2}^{2}}{{n}_{1} + {n}_{2} - 2}} \qquad Equation 2$$

To rank candidate proteins by their genotype and ritonavir-treatment effect sizes, the absolute value of Cohen's d for each condition was summed to make a combined score, ‘Absolute Cohen's d Sum (Geno+RTV)’. Candidate biomarkers were filtered based on the directionality of their pharmacodynamic response to ritonavir and ISRIB being concordant with genotype predictions (ritonavir opposing genotype directionality, ISRIB reproducing genotype directionality). These criteria were then combined to stratify biomarker subsets as displayed in TABLE S1.

### Example 2

Small RNAseq Analysis of Secreted EVs from RNA Isolated from the DYT1 Vs WT MEF

RNA was isolated from the DYT1 vs WT mouse embryonic fibroblast (MEF) secreted EVs, using the same cultures used in proteomic experiments. Small RNA sequencing was performed using a Illumina Novoseq 500. A total of 694 total miRNAs were detected.

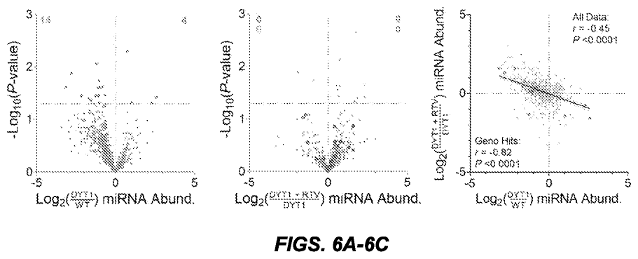

Studies demonstrated genotype and ritonavir pharmacodynamic effects. Results for the genotype effects on EV miRNAs are shown in FIG. 6A. Genotype Z scores (uncorrected p values) ranged from −5.2 to 14. The number of independent culture preparations per genotype was three (N=3). The EV fraction was divided and separately analyzed for proteins and RNAs. An uncorrelated P value threshold of 0.05 was used to identify genotype effects. The results of Ritronavir effects on DYT1 EV miRNA disruptions following 24 hours of treatment are shown in FIG. 6B. Genotype-disrupted miRNAs and genotype-directionality as identified in FIG. 6A are indicated by blue and red dots in FIG. 6B. The correlation of corrective effects on genotype-disrupted set of miRNAs is shown in FIG. 6C, where ritonavir showed a strong correlation of corrective effects on genotype-disrupted subset of miRNAs. Blue and red data points of FIG. 6C identify genotype effect and directionality originally as in FIG. 6A.

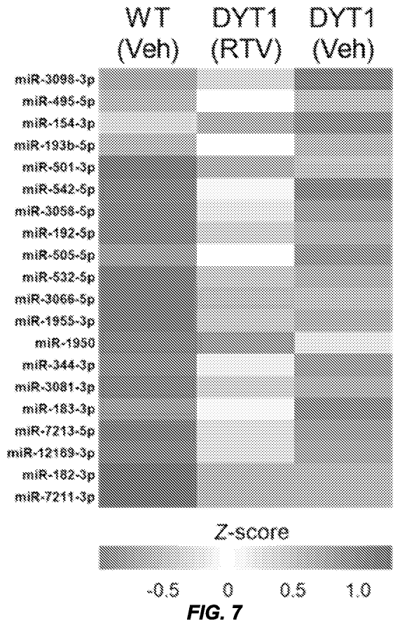

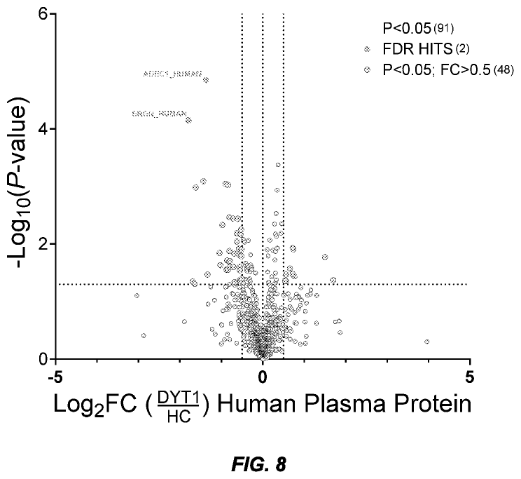

Results showing a heat map of Z-score from 3 conditions (WT vehicle, DYT1 RTV, and DYT1 vehicle) for the genotype-disrupted miRNAs are provided in FIG. 7.

The disclosed murine studies support potential for both protein and miRNA biofluid signatures in DYT1. Murine studies document pharmacodynamic responsiveness of biomarkers to drug treatment that activates the ISR pathway.

### Example 3

Candidate Human DYT1 Dystonia Protein Biomarkers from Whole Plasma

Preliminary analysis of biomarkers using whole plasma samples (N=14 DYT1 Patients and 27 age-matched Healthy Controls) was performed. Duke Proteomics and Metabolomics Core Facility (DPMCF) was used for analysis of proteins from 41 plasma samples using Quantitative data-independent acquisition (DIA) LC-MS/MS Analysis. Tryptic digests were analyzed by microflow LC-MS/MS using an ACQUITY UPLC (Waters Corporation) interfaced to an Exploris 480 Orbitrap (ThermoFisher Scientific) using an Optamax NG source with heated electrospray probe. Raw MS Data was processed in Spectronaut 15 with the Pulsar search engine using the Homo sapiens database, downloaded from Uniprot. Here, 17,163 peptides were quantified, representing 759 proteins in the plasma samples. Of the 759 proteins represented, 91 proteins were significantly differentially abundant (p<0.05) in DYT1 patient plasma as compared to plasma from age/sex-matched Healthy Control subjects. Further analysis found that two proteins (ADEC1 and SRGN) were significantly differential after FDR correction (see FIG. 8). 48 proteins were >±1.0 fold different from Healthy Controls

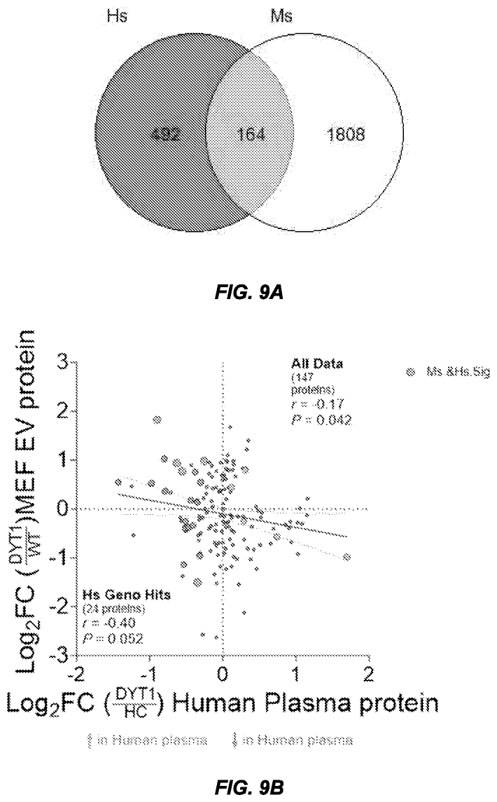

When reviewing for cross-species proteomic results, the human plasma proteome had little overlap with mouse EV proteome (see FIG. 9A), in contrast to relationships between human EV and mouse EV proteomes (as shown below). There were 147 proteins in the MEF data hits that have homolog detected in human total plasma proteomics (see FIG. 9B).

This pilot human plasma study showed biomarker potential in plasma, for protein differences between DYT1 and HC. Some preservation of cross-species relationships helps guide biomarker selection to those with pharmacodynamic responses shown in mice.

### Example 4

Candidate Human DYT1 Dystonia miRNA Biomarkers from Whole Plasma.

Plasma was collected form 13 DYT1 dystonia patients and 27 age and sex-matches healthy volunteers across 3 collaborating centers (Duke, MHG, UF). DYT genotype was confirmed for all samples. microRNAs were isolated from whole plasma using the Qiagen miRNAeasy serum/plasma isolation kit with Spike-Ins. Library generation was performed according to standard protocols using the Qiagen miRNA Library kit.

Samples were subjected to smRNA-seq using an Illumina NovaSeq 6000. RNAseq data was processed using the Qiagen CLC Genomics Workbench and data are presented as normalized UMI counters per million read (CPM)

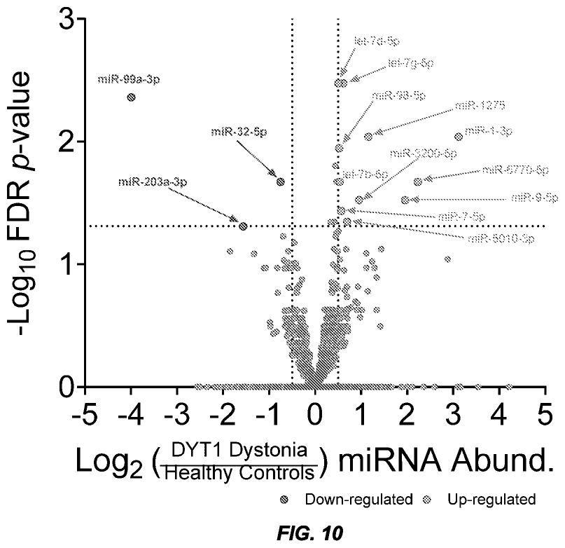

Results: MicroRNAs isolated from whole plasma from healthy volunteers and DYT-TOR1A dystonia subjects were subjected to smRNA-seq using an Illumina NovaSeq 600. Data are presented as normalized UMI counts per million read (CPM). 1634 total miRNAs were detected. A total of 14 miRNAs passed FDR correction p-value thresholds for significance (3 downregulated; 11 upregulated) (adj. P values ranging from 0.049-0.003) and showed effect sizes ranging from 1.5 to more than 4-fold differences. Z-scores for 14 genotype-disrupted miRNAs shown for all samples. Genotype Z scores range from −4.99 to 4.11 (see FIG. 10, and summarized in TABLE 3 below).

TABLE 3
Candidate Human DYTI Dystonia miRNA
Biomarkers from Whole Plasma
(14 miRNAs; FC >1.5(+/-); FDR adj. P < 0.05)

| MicroRNA | Fold change | FDR p-value | P-value |
|---|---|---|---|
| hsa-miR-1.3p | 4.12 | 0.0092 | 0.000067 |
| hsa-miR-6770-5p | 3.23 | 0.021 | 0.00031 |
| hsa-miR-9-5p | 2.95 | 0.03 | 0.00057 |
| hsa-miR-1275 | 2.15 | 0.0092 | 0.000064 |
| hsa-miR-3200-5p | 1.95 | 0.03 | 0.00054 |
| hsa-miR-5010-3p | 1.69 | 0.045 | 0.00098 |
| hsa-let-7d-5p | 1.61 | 0.0033 | 0.00001 |
| hsa-miR-7-5p | 1.57 | 0.037 | 0.00075 |
| hsa-let-7b-5p | 1.53 | 0.021 | 0.00027 |
| hsa-miR-98-5p | 1.52 | 0.011 | 0.000099 |
| hsa-let-7g-5p | 1.5 | 0.0033 | 0.0000059 |
| hsa-miR-32-5p | 1.75 | 0.021 | 0.00034 |
| hsa-miR-203a-3p | -2.56 | 0.049 | 0.0013 |
| hsa-miR-99a-3p | -4.99 | 0.0044 | 0.000019 |

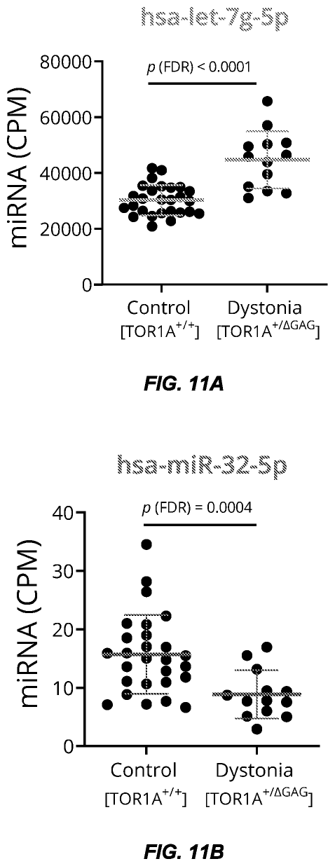

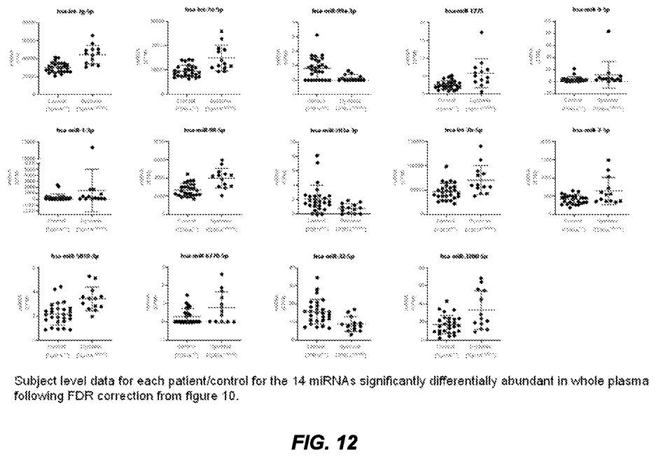

Subject-level data examples for 1 each of an up-regulated and down-regulated miRNA are shown in FIGS. 11A-11B. Subject level data for each patient/control for the 14 miRNAs significantly differentially abundant in whole plasma following FDR correction from FIG. 12.

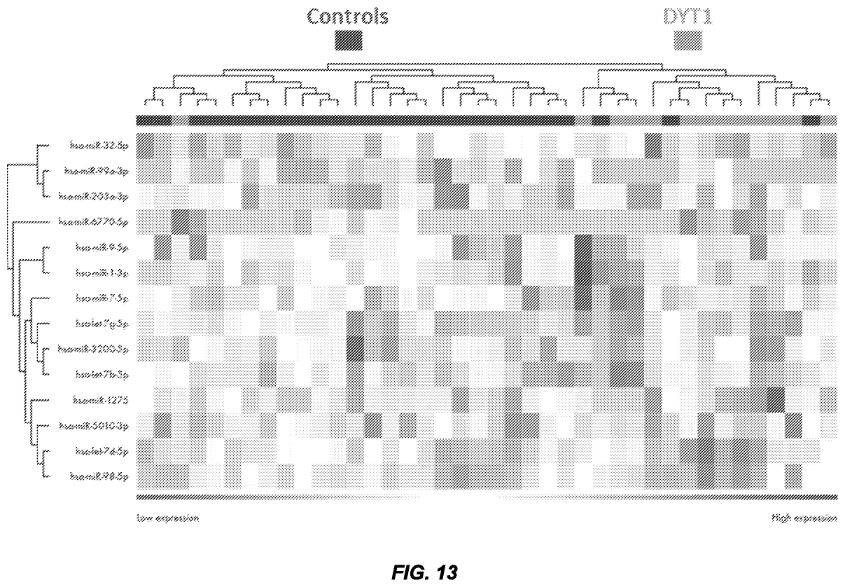

Unsupervised hierarchical heat map clustering of human miRNA levels in plasma shows separation of DYT1 from controls (see FIG. 13).

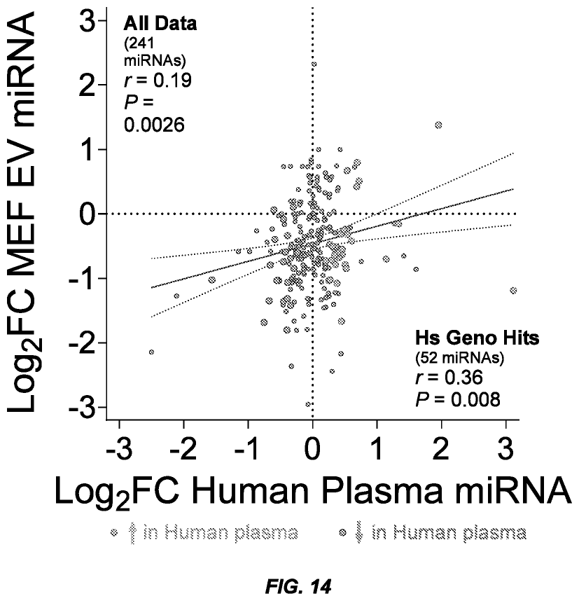

Cross-species comparison of DYT1-genotype effects on miRNAs with sequence homology (241 miRNAs) are provided in FIG. 14.

Pilot human plasma study shows biomarker potential in miRNA differences between DYT1 and HC. Some preservation of cross-species relationships helps guide biomarker selection to those with pharmacodynamic responses shown in mice.

### Example 5

Candidate Metabolites from Whole Plasma Metabolomics

Duke Proteomics and Metabolomics Core Facility (DPMCF) also analyzed metabolites from 41 plasma samples using the Biocrates MxP Quant 500 Kit. Plasma from 14 subjects with DYT1 and 27 Healthy Controls was utilized. Within these samples, 478 metabolites were quantified, twenty of which were significantly differentially abundant (p<0.05) in DYT1 patient plasma as compared to plasma from age/sex-matched Healthy Control subjects. Further analysis lead to the find of seven metabolites having greater than ±0.5 fold different from Healthy Controls and none were >±1.0 fold different.

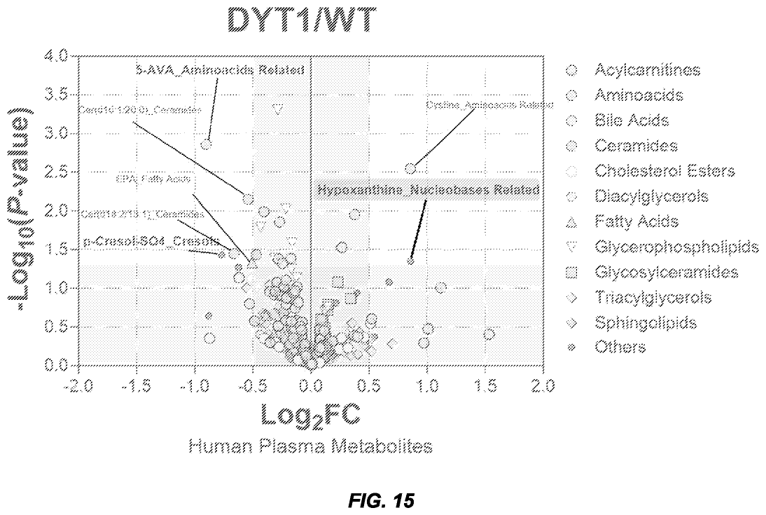

478 metabolites were quantified using a Biocrates MxP® Quant 500 Kit, with results shown in FIG. 15 and details provided in TABLE S2. 20 metabolites were significantly differentially abundant (p<0.05) in DYT1 patient plasma as compared to plasma from age/sex matched Healthy Control subjects. These 20 metabolites were PC aa C24:0_Glycerophospholipids, 5-AVA_Aminoacids Related, Cystine_Aminoacids Related, Cer(d16:1/20:0)_Ceramides, lysoPC a C26:0_Glycerophospholipids, Asp_Aminoacids, Cys_Aminoacids, Tyr_Aminoacids, PC aa C26:0_Glycerophospholipids, lysoPC a C28:0_Glycerophospholipids, C18:2_Acylcarnitines, Cer(d18:2/18:1)_Ceramides, Cer(d16:1/18:0)_Ceramides, p-Cresol-S04_Cresols, PC aa C40:6_Glycerophospholipids, C16-OH_Acylcarnitines, Gly_Aminoacids, Hypoxanthine_Nucleobases Related, EPA_Fatty Acids, C14:2-OH_Acylcarnitines. Of those, 7 greater than +0.5 fold different from Healthy Controls and none were >±1.0 fold different

### Example 6

Candidate Human DYT1 Dystonia Protein from Plasma-Derived EVs

For a pilot human study evaluation, analysis of plasma EV proteomics was performed using plasma from 14 subjects with DYT1 and 27 Healthy Controls. Data was obtained for extracellular vesicles using previously acquired samples. The ExoQuick® ULTRA EV Isolation System was used to fractionate and enrich EVs from plasma. Isolated plasma EV samples were then submitted to proteomic analysis by the Duke Proteomics and Metabolomics Core Facility (DPMCF). At the DPMCF, quantitative (LC/MS/MS) was performed on 500 ng of each sample, using an EvoSep One UPLC system coupled to a Thermo Orbitrap Astral high-resolution accurate mass tandem mass spectrometer (Thermo). Briefly, the sample was eluted directly from an Evotip which was subjected to a low organic wash and then subjected to an analytical separation using a 1.7 μm EvoSep 150 um ID×15 cm endurance (EveoSep) column running an SPD30 gradient from approximately 5 to 30% acetonitrile with 0.1% formic acid with a column temperature of 50 C. Data collection on the Orbitrap Astral mass spectrometer was performed in a data-independent acquisition (DIA) mode of acquisition with a r=240,000 (@m/z 200) full MS scan from m/z 380-980 with a target AGC value of 4e5 ions. Fixed DIA, windows of 4 m/z from m/z 380 to 980 DIA MS/MS scans were acquired in the Astral with a target AGC value of 5e4 and a max fill time of 6 ms. HCD collision energy setting of 27% was used for all MS2 scans.

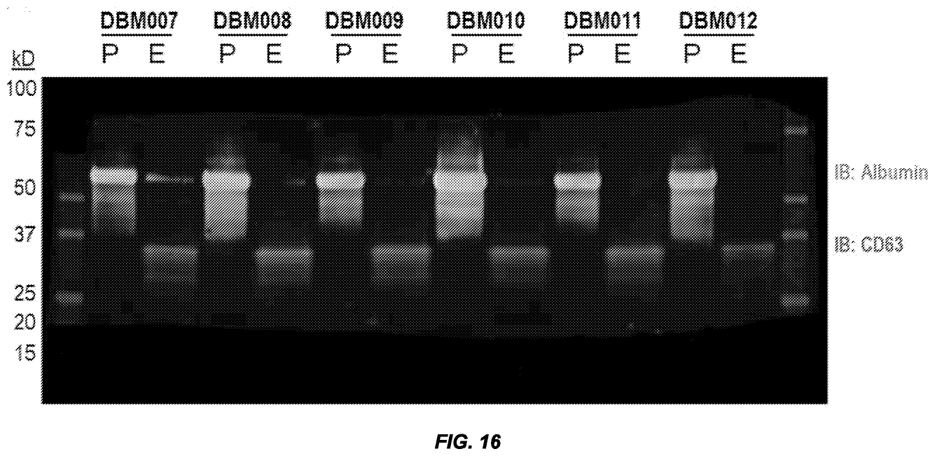

The protein component of plasma derived extracellular vesicles were differentially abundant in DYT1 patients as compared to healthy controls. Extracellular vesicles isolated from human plasma using ExoQuick ULTRA EV isolation system were enriched in exosome marker CD63 and depleted of plasma protein albumin. See FIG. 16, which shows 6 separate plasma sample enrichments.

30,601 peptides were quantified representing 2455 proteins in the plasma samples. Results for 123 proteins that were significantly differentially abundant (p<0.05) in DYT1 patient plasma as compared to plasma from age/sex matched Healthy Control subjects were identified as candidate biomarkers, and are shown below in TABLE 4.

TABLE 4
123 candidate biomarker proteins significantly differentially abundant (p < 0.05)
in DYTI patients as compared to healthy controls.
ADAP2, PTMA, PRSS1, WFDC12, EHD4, SLC4A1, MPIG6B, GALNTL6, HNRNPC, MFAP5, LCK; FYN; YES1; SRC,
PI4KA, RAP2B, USP2, AP1B1, PCDHB15, TRAPPC9, KCNB2, GP9, SELENOF, TUBB1, ZYX, DNAH2, RBM3, ILK,
ANK1, ITGA2B, PRSS3, TGFB2, OBP2A, STOM, BRD9, DLST, KRT33A, SP110, ABCB9, ATP6V1G1, ARL8B,
SEC31A, NAA20, FGL1, TFB1M, IPO9, SPTB, WDR26, FLNA, WNK1, C16orf86, NEK9, MARS1, SLPI, SETD2, LAMA1,
DDOST, SOGA1, MAPK3, FAM131C, RHOA, LORICRIN, IGHV8-51-1, JAM3, SLC2A1, PRPSAP2, PLEK, PSMC5,
MKI67, RPL10, CLK3, LRRC59, CRYAB, PITPNB, TRIM16, LYPLA1, F8, EHD1; EHD3; EHD2, LMNB2, VWF, GPR17,
TLN1, PCMT1, DLD, HLA-C, GP1BB, SLC25A11, RPL13A, NECTIN1, RNF216, ADAMTS19, ANO6, PSMD12, MRC2,
TLN2, FAM177A1, RHOC, HBA1, ACAA1, ATRIP, PTGDR2, PAFAH1B2, USP7, SLC25A6, ATP5F1C, TUBA1C,
ACOX3, PSME4, FSCN1, HBD, H1-5, BZW2, GOLGA4, AGL, FAM98A, TUBA1B, TARDBP, RNF39, RPS13, CEP170,
TOR3A, GART, MYH9, NDNF, LIPG, MUCL1,

Additional details for these 123 proteins (e.g., mean, standard deviation, number of samples, P-values, and Cohen's D values) from the study can be found in TABLE S3-S7. The remaining 2,332 proteins tested had P-values>0.05 and omitted for brevity. 116 proteins were also >±0.5 fold different from Healthy Controls (see TABLE 5).

Table 5: Human Plasma derived EVs Biomarker Candidates for DYT1 Patients Status (p<0.05, FC±0.5)

TABLE 5
Human Plasma derived EVs Biomarker Candidates for DYT1 Patients
Status (p < 0.05; FC ± 0.5)

| KCNB2 | STOM | TUBA1B | LMNB2 | PSMD12 | RBM3 |
|---|---|---|---|---|---|
| SLC4A1 | ITGA2B | WDR26 | FGL1 | PITPNB | LYPLA1 |
| FAM131C | NAA20 | MYH9 | MUCL1 | LAMA1 | PTGDR2 |
| RNF216 | CEP170 | RHOA | RPL13A | TRIM16 | ARL8B |
| NECTIN1 | FAM177A1 | RPS13 | VWF | DLST | USP2 |
| OBP2A | PLEK | NEK9 | ATP5F1C | CRYAB | TUBA1C |
| HBA1 | TUBB1 | PSMC5 | ACAA1 | SLPI | SETD2 |
| NDNF | TLN1 | ATRIP | WNK1 | PRPSAP2 | WFDC12 |
| MPIG6B | ILK | DDOST | H1-5 | F8 | JAM3 |
| BRD9 | FLNA | PCMT1 | DLD | SEC31A | SOGA1 |
| TFB1M | HLA-C | ACOX3 | PSME4 | IPO9 | ANO6 |
| RAP2B | SPTB | RPL10 | LORICRIN | MARS1 | FAM98A |
| HBD | MRC2 | USP7 | GART | TARDBP | ABCB9 |
| ADAMTS19 | SLC2A1 | PTMA | TRAPPC9 | TLN2 | MKI67 |
| GP9 | EHD4 | RNF39 | MAPK3 | GPR17 | LCK; FYN; |
| C16orf86 | PAFAH1B2 | HNRNPC | LIPG | IGHV8- | YES1; SRC |
| GALNTL6 | ZYX | DNAH2 | KRT33A | 51-1 | EHD1; EHD3; |
| MFAP5 | RHOC | ATP6V1G1 | FSCN1 | SP110 | EHD2 |
| GP1BB | GOLGA4 | BZW2 | TGFB2 | PCDHB15 | |
| ANK1 | SLC25A6 | LRRC59 | SELENOF | SLC25A11 | |
| | | | | AP1B1 | |

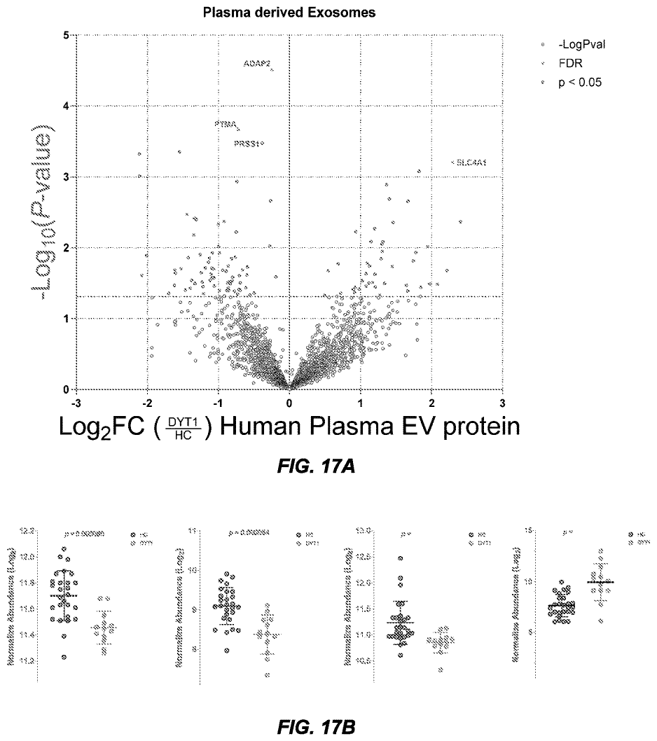

A volcano plot showing differentially expressed proteins (DEPs) in DYT1 patient plasma derived EVs as compared to Health Controls is provided in FIG. 17A. 71 proteins were >±1.0 fold different from Healthy Controls (blue). 4 proteins were significantly differential after FDR correction. (red) (ADAP2; PTMA; PRSS1; SLC4A1). Subject level data for each patient/control for the 4 DEP significant following FDR correction are shown in FIG. 17B.

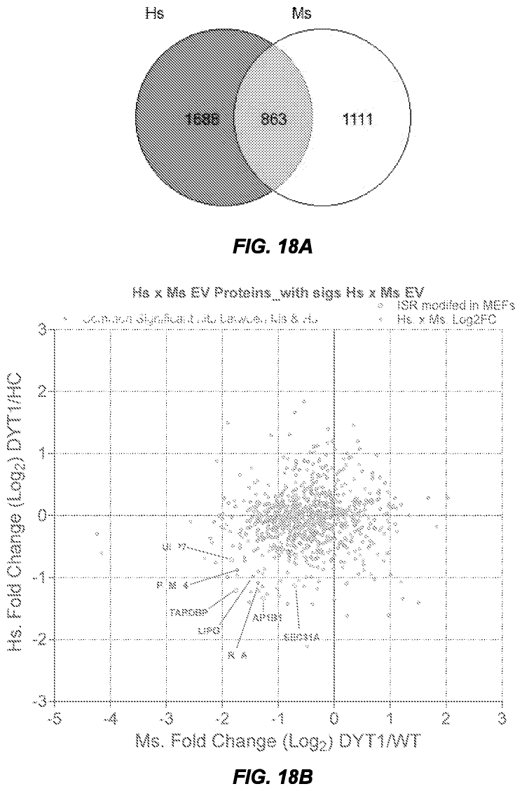

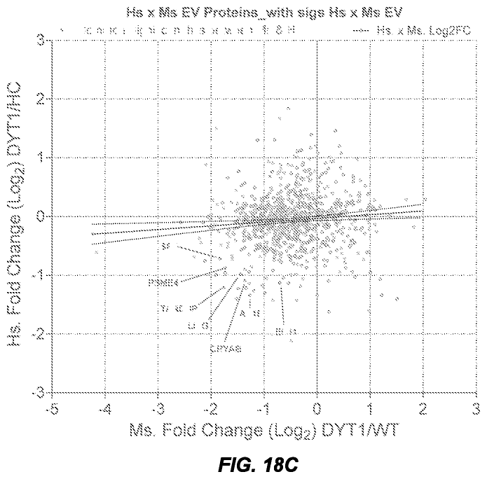

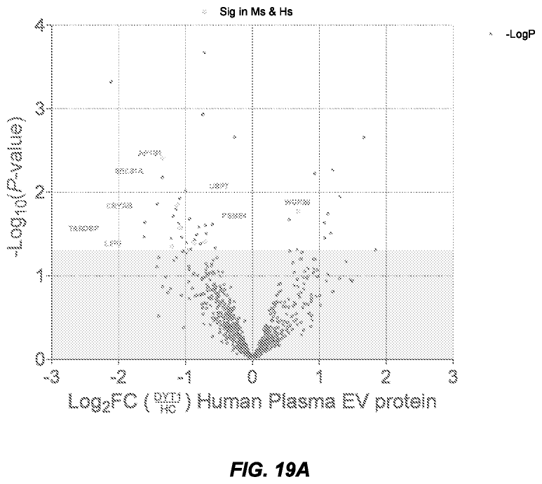

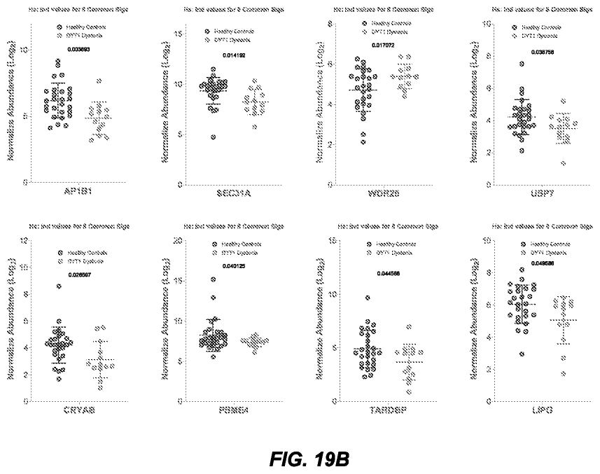

When reviewing for cross-species proteomic results, 863 proteins in MEF data hits were found to have a homolog detected in human plasma derived EV proteomics (see FIG. 18A). Seven proteins were significantly different in both mouse and human EVs compared to WT (HC) and in the same direction (lower) (USP7, PSME4, TARDBP, LIPG, CRYAB, APIB1, SEC31A). The seven proteins were significantly reduced (uncorr p<0.05) in both the mouse and human EV datasets (see FIGS. 18B-18C)

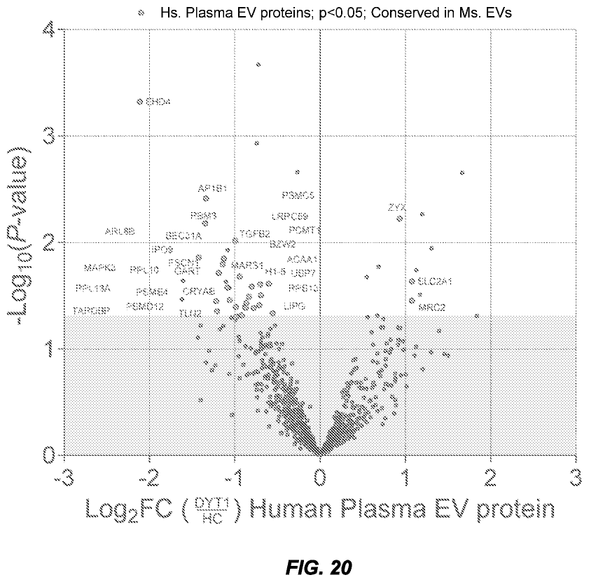

Of these 863 human plasma EV proteins, those that were significantly different by DYT1 genotype (p<0.05) and whose levels are regulated in a similar direction in mouse DYT1 EV experiment (MEF) are presented in FIG. 20 (fuchsia symbols) and summarized in TABLE 6 below.

TABLE 6
Human Plasma derived EVs Biomarker Candidates for
DYT1 Patients Status Shared with MEF derived EVs (p < 0.05)

| | | | | | |
|---|---|---|---|---|---|
| USP7 | PSMC5 | CRYAB | SLC2A1 | ACAA1 | TLN2 |
| SEC31A | PCMT1 | RPS13 | RPL10 | H1-5 | ARL8B |
| TARDBP | GART | PSME4 | BZW2 | FSCN1 | EHD4 |
| AP1B1 | MAPK3 | LIPG | LRRC59 | PSMD12 | TGFB2 |
| ZYX | IPO9 | MRC2 | RPL13A | MARS1 | RBM3 |

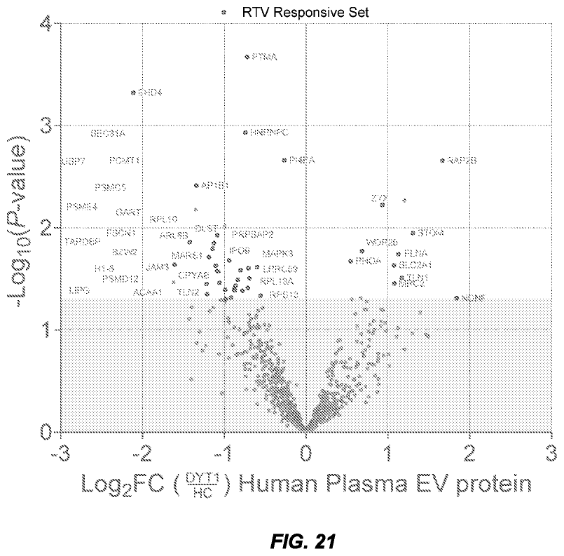

Human Plasma EV proteins that are significantly different by DYT1 genotype and whose levels are modulated by ritonavir in a corrective direction in mouse DYT1 EV experiment (MEF) are presented in FIG. 21 (red symbols) and summarized in TABLE 7 below. An additional 687 proteins were modulated by ritonavir in a corrective direction in mouse DYT1 EV experiment (MEF), but did not reach significance (p<0.05)

### Candidate Biomarkers for Tracking Ritonavir Treatment

TABLE 7
Human Plasma derived EVs Biomarker Candidates:
Corrected by Ritonavir in MEF EVs

| | | | | | |
|---|---|---|---|---|---|
| USP7 | PSMC5 | CRYAB | LIPG | RPL10 | FSCN1 |
| SEC31A | PCMT1 | STOM | NDNF | PTMA | PSMD12 |
| TARDBP | HNRNPC | RPS13 | RAP2B | BZW2 | MARS1 |
| AP1B1 | GART | DLST | TLN1 | LRRC59 | TLN2 |
| ZYX | MAPK3 | PRPSAP2 | FLNA | RPL13A | ARL8B |
| RHOA | IPO9 | WDR26 | MRC2 | ACAA1 | EHD4 |
| PI4KA | JAM3 | PSME4 | SLC2A1 | H1-5 | |

Of those, bidirectional ISR tool compound manipulations in mouse cells show results supporting that ISR pathway activity regulates 4 of the 7 proteins (abundance modulated in DYT1 cells toward correction with salubrinal and in WT cells toward DYT1-like with ISRIB) supporting that they are indicators of DYT1 dystonia genotype that, at least these, are modulated by the ISR. This further argues that ISR-boosting directionality would be corrective.

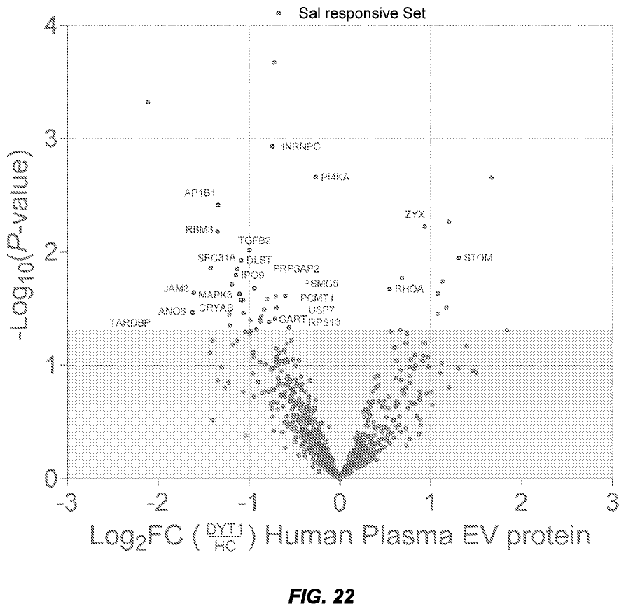

To identify human plasma markers predictive of ISR state, the human DYT1 biomarkers with mouse data showing ISR compound sensitivity in mouse experiments were illustrated. Human data highlighting homologs of EV Proteins normalized by salubrinal treatment in DYT1 MEF EVs are presented in FIG. 22 and summarized in TABLE 8 below, showing candidate biomarkers.

TABLE 8
Human Plasma derived EVs Biomarker Candidates:
DYT1 Corrected by Salubrinal in MEF EVs

| | | | | | |
|---|---|---|---|---|---|
| USP7 | PSMC5 | CRYAB | AP1B1 | GART | PRPSAP2 |
| SEC31A | PCMT1 | STOM | ZYX | MAPK3 | DLST |
| TARDBP | HNRNPC | RPS13 | RHOA | IPO9 | PI4KA |
| JAM3 | | | | | |

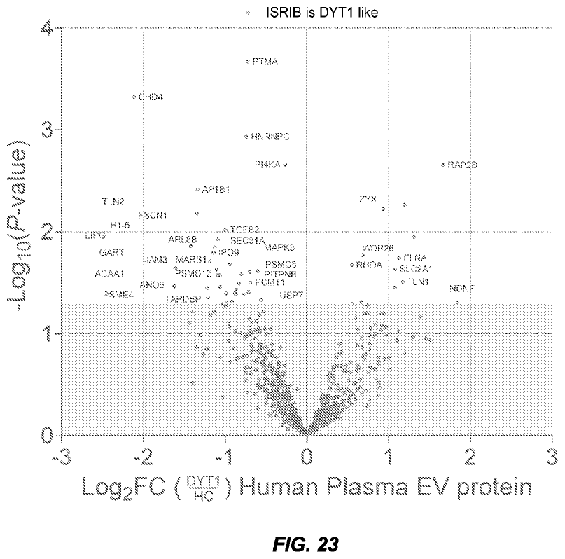

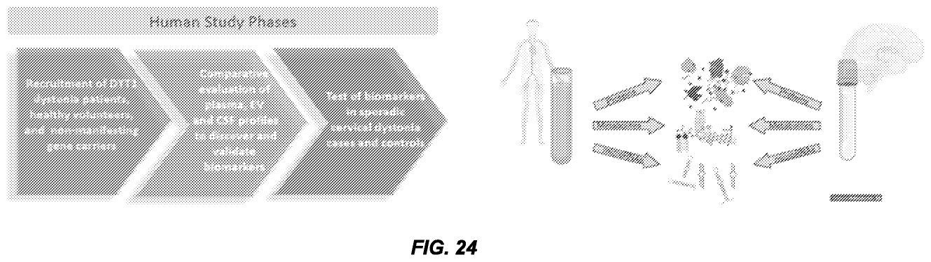

Human data highlighting homologs of EV Proteins made disease-like in WT by ISRIB treatment in DYT1 MEF EVs presented in FIG. 23 and summarized in TABLE 9 below, showing candidate biomarkers.

TABLE 9
Human Plasma derived EVs Biomarker Candidates:
ISRIB replicated DYT1 state in MEF EVs

| | | | | | |
|---|---|---|---|---|---|
| USP7 | HNRNPC | RAP2B | MARS1 | PI4KA | PSME4 |
| SEC31A | GART | TLN1 | TLN2 | PSMC5 | LIPG |
| TARDBP | MAPK3 | FLNA | ARL8B | PCMT1 | NDNF |
| AP1B1 | IPO9 | SLC2A1 | EHD4 | H1-5 | ANO6 |
| ZYX | JAM3 | PTMA | TGFB2 | FSCN1 | |
| RHOA | WDR26 | ACAA1 | PITPNB | PSMD12 | |

TABLE S1
List of 363 genotype-disrupted proteins (e.g., as described in Example 1)

| | | | | | ISRIB | Absolute | | DYT + | WT + |
|---|---|---|---|---|---|---|---|---|---|
| | | | | | and | Cohen's d | DYT/ | RTV/ | ISRIB/ |
| Accession | | | In | RTV | DYT | Sum | WT | DYT | WT |
| (Mus | | | Human | Corrective | Parallel | (Geno + | Cohen's | Cohen's | Cohen's |
| musculus) | GN | Description | Plasma | Direction | Direction | RTV) | d | d | d |
| P54071 | Idh2 | Isocitrate dehydrogenase [NADP], mitochondrial | YES | YES | YES | 26.73 | −3.12 | 23.61 | −2.01 |
| P47738 | Aldh2 | Aldehyde dehydrogenase, mitochondrial | YES | YES | YES | 25.29 | −7.08 | 18.21 | −7.25 |
| Q9D892 | Itpa | Inosine triphosphate pyrophosphatase | YES | YES | YES | 23.68 | −10.88 | 12.80 | −1.25 |
| Q9D1M4 | Eef1e1 | Eukaryotic translation elongation factor 1 | YES | YES | YES | 20.67 | −3.77 | 16.90 | −3.65 |
| | | epsilon-1 | | | | | | | |
| Q60972 | Rbbp4 | Histone-binding protein RBBP4 | YES | YES | YES | 18.10 | −4.22 | 13.88 | −1.73 |
| O89051 | Itm2b | Integral membrane protein 2B | YES | YES | YES | 17.16 | −5.63 | 11.53 | −2.13 |
| Q9Z1N5 | Ddx39b | Spliceosome RNA helicase Ddx39b | YES | YES | YES | 16.28 | −2.49 | 13.79 | −1.12 |
| Q8C878 | Uba3 | NEDD8-activating enzyme E1 catalytic subunit | YES | YES | YES | 14.97 | −4.95 | 10.01 | −1.68 |
| O35382 | Exoc4 | Exocyst complex component 4 | YES | YES | YES | 14.59 | −6.89 | 7.70 | −1.51 |
| O35343 | Kpna4 | Importin subunit alpha-3 | YES | YES | YES | 14.28 | -11.90 | 2.38 | −3.18 |
| Q99KJ8 | Dctn2 | Dynactin subunit 2 | YES | YES | YES | 13.94 | −8.43 | 5.51 | −0.53 |
| Q8K2V6 | Ipo11 | Importin-11 | YES | YES | YES | 13.53 | −3.78 | 9.75 | −1.97 |
| Q01405 | Sec23a | Protein transport protein Sec23A | YES | YES | YES | 13.20 | −5.06 | 8.14 | −0.95 |
| Q8BML9 | Qars | Glutamine--tRNA ligase | YES | YES | YES | 13.17 | −5.96 | 7.21 | −2.14 |
| P09528 | Fth1 | Ferritin heavy chain | YES | YES | YES | 12.58 | -12.27 | 0.31 | −3.46 |
| P27612 | Plaa | Phospholipase A-2-activating protein | YES | YES | YES | 12.45 | −3.77 | 8.69 | −2.39 |
| Q6ZQ08 | Cnot1 | CCR4-NOT transcription complex subunit 1 | YES | YES | YES | 12.30 | −6.76 | 5.54 | −2.56 |
| Q9CYR0 | Ssbp1 | Single-stranded DNA-binding protein, | YES | YES | YES | 12.21 | −8.91 | 3.30 | −1.49 |
| | | mitochondrial | | | | | | | |
| Q922B2 | Dars | Aspartate--tRNA ligase, cytoplasmic | YES | YES | YES | 12.17 | −2.85 | 9.32 | −1.13 |
| Q9Z2X1 | Hnrnpf | Heterogeneous nuclear ribonucleoprotein F | YES | YES | YES | 12.02 | −3.98 | 8.04 | −0.18 |
| P68433 | Hist1h3a | Histone H3.1 | YES | YES | YES | 11.98 | −3.88 | 8.09 | −0.97 |
| Q9CZN7 | Shmt2 | Serine hydroxymethyltransferase, mitochondrial | YES | YES | YES | 11.93 | −2.95 | 8.98 | −1.44 |
| P10923 | Spp1 | Osteopontin | YES | YES | YES | 11.35 | 4.42 | −6.93 | 2.67 |
| Q99PV0 | Prpf8 | Pre-mRNA-processing-splicing factor 8 | YES | YES | YES | 11.34 | −3.39 | 7.95 | −0.01 |
| Q7TQI3 | Otub1 | Ubiquitin thioesterase OTUB1 | YES | YES | YES | 11.30 | −5.45 | 5.84 | −2.26 |
| Q9WVG5 | Lipg | Endothelial lipase | YES | YES | YES | 11.27 | −8.39 | 2.88 | −2.47 |
| Q9D379 | Ephx1 | Epoxide hydrolase 1 | YES | YES | YES | 11.03 | −9.55 | 1.48 | −0.32 |
| Q7TMY8 | Huwe1 | E3 ubiquitin-protein ligase HUWE1 | YES | YES | YES | 10.96 | −5.92 | 5.04 | −3.93 |
| P28352 | Apex1 | DNA-(apurinic or apyrimidinic site) lyase | YES | YES | YES | 10.71 | −2.65 | 8.06 | −0.67 |
| O08999 | Ltbp2 | Latent-transforming growth factor beta-binding | YES | YES | YES | 10.68 | 4.96 | −5.72 | 0.70 |
| | | protein 2 | | | | | | | |
| Q99L27 | Gmpr2 | GMP reductase 2 | YES | YES | YES | 10.50 | −5.40 | 5.10 | −2.98 |
| Q99LC8 | Eif2b1 | Translation initiation factor elF-2B subunit alpha | YES | YES | YES | 10.22 | −5.19 | 5.03 | −0.70 |
| Q6A4J8 | Usp7 | Ubiquitin carboxyl-terminal hydrolase 7 | YES | YES | YES | 10.10 | −3.20 | 6.89 | −1.90 |
| O89079 | Cope | Coatomer subunit epsilon | YES | YES | YES | 10.07 | −2.83 | 7.24 | −1.30 |
| Q8JZK9 | Hmgcs1 | Hydroxymethylglutaryl-CoA synthase, | YES | YES | YES | 10.01 | −2.75 | 7.26 | −0.06 |
| | | cytoplasmic | | | | | | | |
| P51855 | Gss | Glutathione synthetase | YES | YES | YES | 10.00 | 4.34 | 5.66 | −3.22 |
| Q64105 | Spr | Sepiapterin reductase | YES | YES | YES | 9.99 | −6.30 | 3.69 | −3.64 |
| Q6P9R2 | Oxsr1 | Serine/threonine-protein kinase OSR1 | YES | YES | YES | 9.84 | −3.96 | 5.88 | −0.72 |
| Q8BTZ7 | Gmppb | Mannose-1-phosphate guanyltransferase beta | YES | YES | YES | 9.76 | −4.38 | 5.38 | −2.41 |
| Q9D0R2 | Tars | Threonine--tRNA ligase, cytoplasmic | YES | YES | YES | 9.50 | −3.21 | 6.28 | −1.16 |
| Q8BMG7 | Rab3gap2 | Rab3 GTPase-activating protein non-catalytic | YES | YES | YES | 9.42 | −4.72 | 4.70 | −0.43 |
| | | subunit | | | | | | | |
| O55029 | Copb2 | Coatomer subunit beta' | YES | YES | YES | 9.40 | −3.26 | 6.13 | −1.11 |
| O35643 | Ap1b1 | AP-1 complex subunit beta-1 | YES | YES | YES | 9.37 | −4.84 | 4.53 | −1.08 |
| Q99KK7 | Dpp3 | Dipeptidyl peptidase 3 | YES | YES | YES | 9.32 | −4.18 | 5.13 | −1.56 |
| B0V2N1 | Ptprs | Receptor-type tyrosine-protein phosphatase S | YES | YES | YES | 9.28 | 3.88 | −5.40 | 0.13 |
| Q8R081 | Hnrnpl | Heterogeneous nuclear ribonucleoprotein L | YES | YES | YES | 9.12 | −2.95 | 6.17 | −0.32 |
| Q9D0B6 | Pbdc1 | Protein PBDC1 | YES | YES | YES | 9.08 | −5.71 | 3.36 | −1.06 |
| Q9EPU0 | Upf1 | Regulator of nonsense transcripts 1 | YES | YES | YES | 9.02 | −4.78 | 4.24 | −1.84 |
| O70133 | Dhx9 | ATP-dependent RNA helicase A | YES | YES | YES | 9.00 | −2.77 | 6.23 | −1.27 |
| Q62448 | Eif4g2 | Eukaryotic translation initiation factor 4 gamma | YES | YES | YES | 8.90 | −6.11 | 2.79 | −3.04 |
| | | 2 | | | | | | | |
| O70309 | Itgb5 | Integrin beta-5 | YES | YES | YES | 8.87 | 4.18 | −4.69 | 1.22 |
| Q8BVE3 | Atp6v1h | V-type proton ATPase subunit H | YES | YES | YES | 8.73 | −3.91 | 4.82 | −0.66 |
| Q9QUR6 | Prep | Prolyl endopeptidase | YES | YES | YES | 8.56 | −3.15 | 5.40 | −1.11 |
| Q9CQT1 | Mri1 | Methylthioribose-1-phosphate isomerase | YES | YES | YES | 8.46 | −2.6' | 5.85 | −0.01 |
| P45376 | Akr1b1 | Aldose reductase | YES | YES | YES | 8.44 | −3.12 | 5.32 | −0.22 |
| Q3UPL0 | Sec31a | Protein transport protein Sec31A | YES | YES | YES | 8.41 | −2.91 | 5.50 | −1.34 |
| Q8BPB5 | Efemp1 | EGF-containing fibulin-like extracellular matrix | YES | YES | YES | 8.34 | 3.74 | −4.60 | 0.07 |
| | | protein 1 | | | | | | | |
| P51660 | Hsd17b4 | Peroxisomal multifunctional enzyme type 2 | YES | YES | YES | 8.33 | −4.32 | 4.01 | −2.20 |
| Q9CQ65 | Mtap | S-methyl-5'-thioadenosine phosphorylase | YES | YES | YES | 8.12 | −4.62 | 3.50 | −0.85 |
| Q8BRF7 | Scfd1 | Sec1 family domain-containing protein 1 | YES | YES | YES | 8.08 | −3.65 | 4.43 | −2.48 |
| Q61656 | Ddx5 | Probable ATP-dependent RNA helicase DDX5 | YES | YES | YES | 8.05 | −3.57 | 4.48 | −0.92 |
| P61164 | Actr1a | Alpha-centractin | YES | YES | YES | 7.99 | −3.69 | 4.29 | −1.18 |
| Q8K124 | Plekho2 | Pleckstrin homology domain-containing family O | YES | YES | YES | 7.93 | 2.90 | −5.03 | 0.14 |
| | | member 2 | | | | | | | |
| Q9CZU6 | Cs | Citrate synthase, mitochondrial | YES | YES | YES | 7.88 | −3.19 | 4.69 | −1.59 |
| Q9QXS6 | Dbn1 | Drebrin | YES | YES | YES | 7.87 | −3.29 | 4.58 | −3.18 |
| Q3TXS7 | Psmd1 | 26S proteasome non-ATPase regulatory subunit | YES | YES | YES | 7.84 | −3.50 | 4.33 | −0.65 |
| | | 1 | | | | | | | |
| Q9QYC0 | Add1 | Alpha-adducin | YES | YES | YES | 7.76 | −3.46 | 4.30 | 0.00 |
| P24452 | Capg | Macrophage-capping protein | YES | YES | YES | 7.70 | −4.13 | 3.57 | −0.61 |
| P97310 | Mcm2 | DNA replication licensing factor MCM2 | YES | YES | YES | 7.66 | −3.70 | 3.96 | −2.10 |
| Q62371 | Ddr2 | Discoidin domain-containing receptor 2 | YES | YES | YES | 7.66 | −6.73 | 0.93 | −7.89 |
| Q8BMA6 | Srp68 | Signal recognition particle subunit SRP68 | YES | YES | YES | 7.60 | −4.90 | 2.70 | −1.21 |
| Q922R8 | Pdia6 | Protein disulfide-isomerase A6 | YES | YES | YES | 7.51 | −4.24 | 3.27 | −1.84 |
| P28271 | Aco1 | Cytoplasmic aconitate hydratase | YES | YES | YES | 7.49 | −4.16 | 3.33 | −1.03 |
| P48678 | Lmna | Prelamin-A/C | YES | YES | YES | 7.44 | −3.47 | 3.97 | −0.19 |
| Q8VE98 | Cd276 | CD276 antigen | YES | YES | YES | 7.44 | −5.68 | 1.75 | −2.07 |
| Q99JF5 | Mvd | Diphosphomevalonate decarboxylase | YES | YES | YES | 7.34 | −2.96 | 4.38 | −0.34 |
| Q69ZU6 | Thsd7a | Thrombospondin type-1 domain-containing | YES | YES | YES | 7.28 | 6.27 | −1.01 | 1.92 |
| | | protein 7A | | | | | | | |
| O35286 | Dhx15 | Pre-mRNA-splicing factor ATP-dependent RNA | YES | YES | YES | 7.27 | −2.69 | 4.59 | −1.19 |
| | | helicase DHX15 | | | | | | | |
| O54774 | Ap3d1 | AP-3 complex subunit delta-1 | YES | YES | YES | 7.27 | −4.00 | 3.27 | −0.19 |
| Q05306 | Col10a1 | Collagen alpha-1(X) chain | YES | YES | YES | 7.24 | 3.83 | −3.41 | 0.36 |
| Q9QXK3 | Copg2 | Coatomer subunit gamma-2 | YES | YES | YES | 7.09 | −4.04 | 3.05 | −0.44 |
| Q7M6Y3 | Picalm | Phosphatidylinositol-binding clathrin assembly | YES | YES | YES | 7.04 | −4.19 | 2.85 | −1.01 |
| | | protein | | | | | | | |
| P36536 | Sar1a | GTP-binding protein SAR1a | YES | YES | YES | 7.03 | −2.63 | 4.40 | −1.23 |
| Q6DIB5 | Megf10 | Multiple epidermal growth factor-like domains | YES | YES | YES | 6.86 | 4.06 | −2.80 | 1.37 |
| | | protein 10 | | | | | | | |
| P32921 | Wars | Tryptophan--tRNA ligase, cytoplasmic | YES | YES | YES | 6.84 | −2.46 | 4.38 | −1.03 |
| Q8C0E2 | Vps26b | Vacuolar protein sorting-associated protein 26B | YES | YES | YES | 6.38 | −3.37 | 3.01 | −1.43 |
| Q62261 | Sptbn1 | Spectrin beta chain, non-erythrocytic 1 | YES | YES | YES | 6.23 | −2.95 | 3.27 | −0.82 |
| Q810B6 | Ankfy1 | Rabankyrin-5 | YES | YES | YES | 6.11 | −3.26 | 2.86 | −1.45 |
| P32507 | Nectin2 | Nectin-2 | YES | YES | YES | 6.05 | −2.73 | 3.32 | −1.19 |
| Q8CG19 | Ltbp1 | Latent-transforming growth factor beta-binding | YES | YES | YES | 6.03 | 2.96 | −3.08 | 2.17 |
| | | protein 1 | | | | | | | |
| P16546 | Sptan1 | Spectrin alpha chain, non-erythrocytic 1 | YES | YES | YES | 6.02 | −2.81 | 3.22 | −0.40 |
| Q9EQ80 | Nif3l1 | NIF3-like protein 1 | YES | YES | YES | 5.96 | −4.38 | 1.58 | −3.93 |
| Q8CHP8 | Pgp | Glycerol-3-phosphate phosphatase | YES | YES | YES | 5.94 | −3.66 | 2.28 | −0.86 |
| Q8BH61 | F13a1 | Coagulation factor XIII A chain | YES | YES | YES | 5.81 | 4.15 | −1.66 | 1.21 |
| Q8CI59 | Steap3 | Metalloreductase STEAP3 | YES | YES | YES | 5.70 | −3.89 | 1.81 | −3.91 |
| Q61165 | Slc9a1 | Sodium/hydrogen exchanger 1 | YES | YES | YES | 5.62 | 3.02 | −2.60 | 0.44 |
| Q9JLV5 | Cul3 | Cullin-3 | YES | YES | YES | 5.58 | −3.22 | 2.36 | −0.66 |
| O88207 | Col5a1 | Collagen alpha-1(V) chain | YES | YES | YES | 5.55 | 4.66 | −0.89 | 0.06 |
| Q9R1E6 | Enpp2 | Ectonucleotide | YES | YES | YES | 5.51 | −5.08 | 0.43 | −3.03 |
| | | pyrophosphatase/phosphodiesterase family | | | | | | | |
| | | member 2 | | | | | | | |
| Q8CDN6 | Txnl1 | Thioredoxin-like protein 1 | YES | YES | YES | 5.33 | −3.34 | 1.99 | −0.66 |
| P16092 | Fgfr1 | Fibroblast growth factor receptor 1 | YES | YES | YES | 5.30 | 2.49 | −2.81 | 5.66 |
| Q04857 | Col6a1 | Collagen alpha-1(VI) chain | YES | YES | YES | 5.26 | 3.31 | −1.96 | 0.14 |
| Q8BGD9 | Eif4b | Eukaryotic translation initiation factor 4B | YES | YES | YES | 5.16 | 3.43 | −1.72 | 3.88 |
| Q921F2 | Tardbp | TAR DNA-binding protein 43 | YES | YES | YES | 5.14 | −4.35 | 0.80 | 0.28 |
| Q9D1G1 | Rab1b | Ras-related protein Rab-1B | YES | YES | YES | 5.10 | −3.95 | 1.15 | −2.54 |
| Q62167 | Ddx3x | ATP-dependent RNA helicase DDX3X | YES | YES | YES | 5.09 | −2.85 | 2.24 | −1.08 |
| Q9CXY6 | Ilf2 | Interleukin enhancer-binding factor 2 | YES | YES | YES | 5.07 | −2.60 | 2.47 | −1.37 |
| P23492 | Pnp | Purine nucleoside phosphorylase | YES | YES | YES | 4.95 | −3.24 | 1.71 | −0.76 |
| O35226 | Psmd4 | 26S proteasome non-ATPase regulatory subunit | YES | YES | YES | 4.91 | 4.31 | −0.60 | 0.71 |
| | | 4 | | | | | | | |
| Q61982 | Notch3 | Neurogenic locus notch homolog protein 3 | YES | YES | YES | 4.77 | 2.55 | −2.22 | 0.52 |
| Q64324 | Stxbp2 | Syntaxin-binding protein 2 | YES | YES | YES | 4.72 | −2.90 | 1.82 | −0.96 |
| P97807 | Fh | Fumarate hydratase, mitochondrial | YES | YES | YES | 4.51 | −2.39 | 2.12 | −0.28 |
| Q9DCN2 | Cyb5r3 | NADH-cytochrome b5 reductase 3 | YES | YES | YES | 4.25 | −3.61 | 0.64 | 0.64 |
| P10518 | Alad | Delta-aminolevulinic acid dehydratase | YES | YES | YES | 3.96 | −2.39 | 1.57 | −1.50 |
| Q9D906 | Atg7 | Ubiquitin-like modifier-activating enzyme ATG7 | YES | YES | YES | 3.89 | −3.27 | 0.61 | −2.35 |
| P31786 | Dbi | Acyl-CoA-binding protein | YES | YES | YES | 3.83 | −2.92 | 0.91 | −2.44 |
| P27773 | Pdia3 | Protein disulfide-isomerase A3 | YES | YES | YES | 3.53 | −3.17 | 0.37 | 0.39 |
| Q9R001 | Adamts5 | A disintegrin and metalloproteinase with | YES | YES | YES | 3.17 | −2.42 | 0.76 | −2.06 |
| | | thrombospondin motifs 5 | | | | | | | |
| P51859 | Hdgf | Hepatoma-derived growth factor | YES | YES | YES | 3.05 | 2.89 | −0.15 | 1.97 |
| P34152 | Ptk2 | Focal adhesion kinase 1 | YES | YES | YES | 3.03 | −2.93 | 0.10 | −3.26 |
| Q9ES89 | Extl2 | Exostosin-like 2 | YES | YES | YES | 2.57 | −2.45 | 0.12 | −1.42 |
| Q78ZA7 | Nap1l4 | Nucleosome assembly protein 1-like 4 | YES | YES | NO | 50.77 | -45.04 | 5.74 | 2.43 |
| P97792 | Cxadr | Coxsackievirus and adenovirus receptor | YES | YES | NO | 16.73 | -12.85 | 3.88 | 1.72 |
| | | homolog | | | | | | | |
| P56959 | Fus | RNA-binding protein FUS | YES | YES | NO | 14.88 | −2.94 | 11.94 | 0.85 |
| O55023 | Impa1 | Inositol monophosphatase 1 | YES | YES | NO | 14.51 | −3.62 | 10.89 | 1.73 |
| Q9DAW9 | Cnn3 | Calponin-3 | YES | YES | NO | 13.28 | −8.50 | 4.78 | 2.76 |
| Q9Z1Z0 | Uso1 | General vesicular transport factor p115 | YES | YES | NO | 12.14 | −3.02 | 9.12 | 2.44 |
| D3YXG0 | Hmcn1 | Hemicentin-1 | YES | YES | NO | 11.51 | 7.93 | −3.58 | −2.25 |
| Q99J45 | Nrbp1 | Nuclear receptor-binding protein | YES | YES | NO | 9.54 | −3.20 | 6.35 | 0.18 |
| Q9WV55 | Vapa | Vesicle-associated membrane protein- | YES | YES | NO | 8.93 | −7.02 | 1.91 | 3.18 |
| | | associated protein A | | | | | | | |
| P61089 | Ube2n | Ubiquitin-conjugating enzyme E2 N | YES | YES | NO | 8.62 | −2.60 | 6.02 | 0.41 |
| O35737 | Hnrnph1 | Heterogeneous nuclear ribonucleoprotein H | YES | YES | NO | 8.31 | −4.08 | 4.23 | 2.47 |
| P50543 | S100a11 | Protein S100-A11 | YES | YES | NO | 8.18 | −3.69 | 4.49 | 0.33 |
| O09131 | Gsto1 | Glutathione S-transferase omega-1 | YES | YES | NO | 8.09 | −4.63 | 3.45 | 0.04 |
| P12025 | Mdk | Midkine | YES | YES | NO | 7.88 | −4.24 | 3.64 | 1.33 |
| Q9JMH9 | Myo18a | Unconventional myosin-XVIIIa | YES | YES | NO | 7.78 | −2.87 | 4.91 | 2.27 |
| P67984 | Rpl22 | 60S ribosomal protein L22 | YES | YES | NO | 7.66 | −6.07 | 1.59 | 2.38 |
| O35405 | Pld3 | Phospholipase D3 | YES | YES | NO | 7.47 | −6.86 | 0.61 | 1.18 |
| Q9ES30 | C1qtnf3 | Complement C1q tumor necrosis factor-related | YES | YES | NO | 6.95 | 3.71 | −3.24 | −0.18 |
| | | protein 3 | | | | | | | |
| P35279 | Rab6a | Ras-related protein Rab-6A | YES | YES | NO | 6.80 | −3.66 | 3.14 | 0.44 |
| P62996 | Tra2b | Transformer-2 protein homolog beta | YES | YES | NO | 6.63 | −3.35 | 3.28 | 0.54 |
| P42669 | Pura | Transcriptional activator protein Pur-alpha | YES | YES | NO | 6.04 | −3.68 | 2.36 | 0.52 |
| P08003 | Pdia4 | Protein disulfide-isomerase A4 | YES | YES | NO | 5.92 | −3.67 | 2.25 | 1.37 |
| Q8BL97 | Srsf7 | Serine/arginine-rich splicing factor 7 | YES | YES | NO | 5.72 | −2.81 | 2.91 | 0.13 |
| P32067 | Ssb | Lupus La protein homolog | YES | YES | NO | 5.45 | −2.66 | 2.79 | 0.41 |
| Q9EPL8 | Ipo7 | Importin-7 | YES | YES | NO | 5.25 | −3.01 | 2.25 | 0.30 |
| P23927 | Cryab | Alpha-crystallin B chain | YES | YES | NO | 5.18 | −4.08 | 1.11 | 0.39 |
| O54890 | Itgb3 | Integrin beta-3 | YES | YES | NO | 4.87 | −2.91 | 1.96 | 0.14 |
| Q60751 | lgf1r | Insulin-like growth factor 1 receptor | YES | YES | NO | 4.86 | −4.23 | 0.63 | 0.75 |
| Q3U7R1 | Esyt1 | Extended synaptotagmin-1 | YES | YES | NO | 4.61 | −3.36 | 1.25 | 0.18 |
| P22005 | Penk | Proenkephalin-A | YES | YES | NO | 4.31 | 2.02 | −2.29 | −0.11 |
| O35114 | Scarb2 | Lysosome membrane protein 2 | YES | YES | NO | 3.91 | −3.66 | 0.24 | 0.09 |
| Q6X893 | Slc44a1 | Choline transporter-like protein 1 | YES | YES | NO | 3.67 | −2.83 | 0.84 | 0.80 |
| A2AVA0 | Svep1 | Sushi, von Willebrand factor type A, EGF and | YES | YES | NO | 2.64 | 2.25 | −0.40 | −0.50 |
| | | pentraxin domain-containing protein 1 | | | | | | | |
| Q9Z1X4 | Ilf3 | Interleukin enhancer-binding factor 3 | YES | NO | YES | 7.19 | −5.96 | −1.23 | −4.89 |
| Q9Z1R3 | Apom | Apolipoprotein M | YES | NO | YES | 6.89 | 6.49 | 0.40 | 2.53 |
| P51881 | Slc25a5 | ADP/ATP translocase 2 | YES | NO | YES | 5.11 | −4.33 | −0.78 | −0.54 |
| Q6P5H2 | Nes | Nestin | YES | NO | YES | 4.89 | 4.52 | 0.37 | 2.29 |
| P97333 | Nrp | Neuropilin-1 | YES | NO | YES | 4.87 | −3.52 | −1.35 | −0.07 |
| P97765 | Wbp2 | WW domain-binding protein 2 | YES | NO | YES | 4.40 | −3.50 | −0.89 | −1.50 |
| Q8C129 | Lnpep | Leucyl-cystinyl aminopeptidase | YES | NO | YES | 3.10 | −2.70 | −0.39 | −1.24 |
| P97298 | Serpinf1 | Pigment epithelium-derived factor | YES | NO | NO | 16.52 | 15.05 | 1.47 | −1.83 |
| P48441 | Idua | Alpha-L-iduronidase | YES | NO | NO | 5.77 | −5.69 | −0.08 | 0.52 |
| P35564 | Canx | Calnexin | YES | NO | NO | 2.95 | −2.79 | −0.16 | 0.64 |
| Q9D5V5 | Cul5 | Cullin-5 | NO | YES | YES | 40.19 | -27.15 | 13.04 | −3.39 |
| Q9CX56 | Psmd8 | 26S proteasome non-ATPase regulatory subunit | NO | YES | YES | 34.01 | −10.56 | 23.45 | −3.31 |
| | | 8 | | | | | | | |
| P97760 | Polr2c | DNA-directed RNA polymerase II subunit RPB3 | NO | YES | YES | 25.31 | -18.09 | 7.22 | −0.89 |
| Q3UYV9 | Ncbp1 | Nuclear cap-binding protein subunit 1 | NO | YES | YES | 23.68 | −2.53 | 21.14 | −1.51 |
| Q9QY36 | Naa10 | N-alpha-acetyltransferase 10 | NO | YES | YES | 21.98 | -16.00 | 5.98 | −4.08 |
| Q00612 | G6pdx | Glucose-6-phosphate 1-dehydrogenase X | NO | YES | YES | 20.45 | 3.70 | 16.75 | −0.43 |
| Q923G2 | Polr2h | DNA-directed RNA polymerases I, II, and III | NO | YES | YES | 19.58 | −10.14 | 9.44 | −4.14 |
| | | subunit RPABC3 | | | | | | | |
| Q60766 | Irgm1 | Immunity-related GTPase family M protein 1 | NO | YES | YES | 19.48 | -16.10 | 3.38 | −1.23 |
| Q60996 | Ppp2r5c | Serine/threonine-protein phosphatase 2A 56 | NO | YES | YES | 19.42 | −3.08 | 16.33 | −1.19 |
| | | kDa regulatory subunit gamma isoform | | | | | | | |
| P49717 | Mcm4 | DNA replication licensing factor MCM4 | NO | YES | YES | 19.23 | −10.89 | 8.34 | −4.74 |
| Q9D4H8 | Cul2 | Cullin-2 | NO | YES | YES | 18.93 | -11.48 | 7.44 | −3.59 |
| A2A6Q5 | Cdc27 | Cell division cycle protein 27 homolog | NO | YES | YES | 18.65 | -11.34 | 7.31 | −1.81 |
| Q8C052 | Map1s | Microtubule-associated protein 1S | NO | YES | YES | 18.41 | -14.53 | 3.88 | −5.93 |
| Q9QXB9 | Drg2 | Developmentally-regulated GTP-binding protein | NO | YES | YES | 17.11 | 3.20 | 13.91 | −1.00 |
| | | 2 | | | | | | | |
| Q91ZW3 | Smarca5 | SWI/SNF-related matrix-associated actin- | NO | YES | YES | 16.86 | −7.95 | 8.91 | −1.07 |
| | | dependent regulator of chromatin subfamily A | | | | | | | |
| | | member 5 | | | | | | | |
| E9Q555 | Rnf213 | E3 ubiquitin-protein ligase RNF213 | NO | YES | YES | 16.14 | −7.05 | 9.09 | −3.97 |
| Q8CBY8 | Dctn4 | Dynactin subunit 4 | NO | YES | YES | 15.84 | −7.29 | 8.55 | −2.05 |
| Q61550 | Rad21 | Double-strand-break repair protein rad21 | NO | YES | YES | 15.76 | −10.85 | 4.91 | −0.66 |
| | | homolog | | | | | | | |
| Q6PIP5 | Nudcd1 | NudC domain-containing protein 1 | NO | YES | YES | 15.34 | −8.12 | 7.22 | −2.33 |
| Q80YQ2 | Med23 | Mediator of RNA polymerase II transcription | NO | YES | YES | 14.51 | −4.92 | 9.59 | −2.09 |
| | | subunit 23 | | | | | | | |
| Q8K4B0 | Mta1 | Metastasis-associated protein MTA1 | NO | YES | YES | 14.49 | −3.61 | 10.88 | −0.75 |
| P53995 | Anapc1 | Anaphase-promoting complex subunit 1 | NO | YES | YES | 13.40 | −8.58 | 4.82 | −1.53 |
| Q9WV80 | Snx1 | Sorting nexin-1 | NO | YES | YES | 13.25 | −7.53 | 5.71 | −0.37 |
| Q5PRF0 | Heatr5a | HEAT repeat-containing protein 5A | NO | YES | YES | 13.15 | −6.31 | 6.85 | −1.63 |
| P70398 | Usp9x | Probable ubiquitin carboxyl-terminal hydrolase | NO | YES | YES | 13.09 | −7.23 | 5.86 | −2.17 |
| | | FAF-X | | | | | | | |
| Q9WTL7 | Lypla2 | Acyl-protein thioesterase 2 | NO | YES | YES | 13.08 | −8.45 | 4.63 | −1.34 |
| Q8R349 | Cdc16 | Cell division cycle protein 16 homolog | NO | YES | YES | 13.07 | −2.55 | 10.52 | −0.03 |
| Q80YQ8 | Rmnd5a | Protein RMD5 homolog A | NO | YES | YES | 12.66 | −7.25 | 5.41 | −5.34 |
| Q8VE73 | Cul7 | Cullin-7 | NO | YES | YES | 12.62 | −9.84 | 2.79 | −1.50 |
| Q9CRT8 | Xpot | Exportin-T | NO | YES | YES | 12.41 | −7.80 | 4.61 | −5.96 |
| Q3V1L4 | Nt5c2 | Cytosolic purine 5′-nucleotidase | NO | YES | YES | 12.32 | −8.22 | 4.10 | −1.39 |
| Q8VDW0 | Ddx39a | ATP-dependent RNA helicase DDX39A | NO | YES | YES | 12.20 | −3.23 | 8.96 | −1.53 |
| Q6VN19 | Ranbp10 | Ran-binding protein 10 | NO | YES | YES | 12.16 | −5.29 | 6.87 | −0.86 |
| O08810 | Eftud2 | 116 kDa U5 small nuclear ribonucleoprotein | NO | YES | YES | 12.16 | −4.16 | 8.00 | −0.06 |
| | | component | | | | | | | |
| P62869 | Elob | Elongin-B | NO | YES | YES | 11.88 | −4.49 | 7.39 | −2.06 |
| Q7TMC8 | Fuk | L-fucose kinase | NO | YES | YES | 11.70 | −4.94 | 6.76 | −1.63 |
| Q80UW8 | Polr2e | DNA-directed RNA polymerases I, II, and III | NO | YES | YES | 11.60 | −4.55 | 7.06 | −2.64 |
| | | subunit RPABC1 | | | | | | | |
| Q9WVG6 | Carm1 | Histone-arginine methyltransferase CARM1 | NO | YES | YES | 11.51 | −2.94 | 8.58 | −1.04 |
| Q9JKC8 | Ap3m1 | AP-3 complex subunit mu-1 | NO | YES | YES | 11.48 | −4.88 | 6.60 | −1.11 |
| Q99JX3 | Gorasp2 | Golgi reassembly-stacking protein 2 | NO | YES | YES | 11.15 | −3.45 | 7.69 | −1.02 |
| B1AZI6 | Thoc2 | THO complex subunit 2 | NO | YES | YES | 11.15 | −2.83 | 8.32 | −0.41 |
| Q8BZQ7 | Anapc2 | Anaphase-promoting complex subunit 2 | NO | YES | YES | 11.12 | −4.40 | 6.72 | −0.42 |
| Q921G8 | Tubgcp2 | Gamma-tubulin complex component 2 | NO | YES | YES | 11.11 | −6.58 | 4.54 | −2.53 |
| Q9EQQ9 | Mgea5 | Protein O-GlcNAcase | NO | YES | YES | 11.09 | −8.62 | 2.47 | 0.76 |
| Q8BJ71 | Nup93 | Nuclear pore complex protein Nup93 | NO | YES | YES | 10.91 | −3.44 | 7.48 | −1.34 |
| Q8CFI7 | Polr2b | DNA-directed RNA polymerase II subunit RPB2 | NO | YES | YES | 10.81 | −3.17 | 7.64 | −1.23 |
| Q6ZQ88 | Kdm1a | Lysine-specific histone demethylase 1A | NO | YES | YES | 10.76 | −7.50 | 3.26 | −5.21 |
| Q61881 | Mcm7 | DNA replication licensing factor MCM7 | NO | YES | YES | 10.48 | −3.12 | 7.36 | −2.31 |
| P37913 | Lig1 | DNA ligase 1 | NO | YES | YES | 10.42 | −3.00 | 7.42 | −0.03 |
| Q8QZY9 | Sf3b4 | Splicing factor 3B subunit 4 | NO | YES | YES | 10.35 | −6.59 | 3.76 | −0.09 |
| Q9JIG7 | Ccdc22 | Coiled-coil domain-containing protein 22 | NO | YES | YES | 10.32 | −5.86 | 4.47 | −2.17 |
| Q80WQ2 | Vac14 | Protein VAC14 homolog | NO | YES | YES | 10.30 | −3.87 | 6.42 | −1.46 |
| Q9D0M1 | Prpsap1 | Phosphoribosyl pyrophosphate synthase- | NO | YES | YES | 10.23 | −5.71 | 4.52 | −1.69 |
| | | associated protein 1 | | | | | | | |
| Q9ERF3 | Wdr61 | WD repeat-containing protein 61 | NO | YES | YES | 10.09 | −3.06 | 7.03 | −1.03 |
| Q9R190 | Mta2 | Metastasis-associated protein MTA2 | NO | YES | YES | 9.96 | −2.92 | 7.04 | −1.01 |
| A2AN08 | Ubr4 | E3 ubiquitin-protein ligase UBR4 | NO | YES | YES | 9.85 | −3.29 | 6.56 | −1.11 |
| Q9JJ80 | Rpf2 | Ribosome production factor 2 homolog | NO | YES | YES | 9.76 | −4.79 | 4.97 | −0.12 |
| P13864 | Dnmt1 | DNA (cytosine-5)-methyltransferase 1 | NO | YES | YES | 9.73 | −3.40 | 6.33 | −0.74 |
| Q9CXK8 | Nip7 | 60S ribosome subunit biogenesis protein NIP7 | NO | YES | YES | 9.72 | −3.52 | 6.20 | −0.78 |
| | | homolog | | | | | | | |
| P08775 | Polr2a | DNA-directed RNA polymerase II subunit RPB1 | NO | YES | YES | 9.69 | −4.65 | 5.04 | −1.44 |
| P49718 | Mcm5 | DNA replication licensing factor MCM5 | NO | YES | YES | 9.62 | −4.16 | 5.46 | −1.12 |
| P70700 | Polr1b | DNA-directed RNA polymerase I subunit RPA2 | NO | YES | YES | 9.56 | −6.97 | 2.59 | −2.12 |
| P47809 | Map2k4 | Dual specificity mitogen-activated protein kinase | NO | YES | YES | 9.52 | −3.59 | 5.93 | −2.03 |
| | | 4 | | | | | | | |
| Q8CG48 | Smc2 | Structural maintenance of chromosomes protein | NO | YES | YES | 9.42 | −6.31 | 3.12 | −2.04 |
| | | 2 | | | | | | | |
| Q8BTZ4 | Anapc5 | Anaphase-promoting complex subunit 5 | NO | YES | YES | 9.33 | −3.85 | 5.48 | −1.38 |
| Q9JHJ3 | Glmp | Glycosylated lysosomal membrane protein | NO | YES | YES | 8.94 | −2.22 | 6.71 | −0.51 |
| Q9DCE5 | Pak1ip1 | p21-activated protein kinase-interacting protein | NO | YES | YES | 8.83 | −3.59 | 5.24 | −0.90 |
| | | 1 | | | | | | | |
| P97452 | Bop1 | Ribosome biogenesis protein BOP1 | NO | YES | YES | 8.81 | −4.64 | 4.17 | −1.74 |
| Q91W96 | Anapc4 | Anaphase-promoting complex subunit 4 | NO | YES | YES | 8.75 | −3.84 | 4.91 | −0.87 |
| P61222 | Abce1 | ATP-binding cassette sub-family E member 1 | NO | YES | YES | 8.70 | −4.42 | 4.28 | −2.14 |
| Q5SWU9 | Acaca | Acetyl-CoA carboxylase 1 | NO | YES | YES | 8.58 | −3.23 | 5.34 | −1.07 |
| P61082 | Ube2m | NEDD8-conjugating enzyme Ubc12 | NO | YES | YES | 8.51 | −3.01 | 5.50 | −1.81 |
| Q8C7V3 | Utp15 | U3 small nucleolar RNA-associated protein 15 | NO | YES | YES | 8.48 | −5.18 | 3.30 | −1.59 |
| | | homolog | | | | | | | |
| Q6PE01 | Snrnp40 | U5 small nuclear ribonucleoprotein 40 kDa | NO | YES | YES | 8.47 | −3.01 | 5.46 | −0.74 |
| | | protein | | | | | | | |
| Q9CSH3 | Dis3 | Exosome complex exonuclease RRP44 | NO | YES | YES | 8.26 | −3.18 | 5.08 | −1.83 |
| Q9WVM3 | Anapc7 | Anaphase-promoting complex subunit 7 | NO | YES | YES | 8.16 | −4.34 | 3.82 | −2.19 |
| Q9Z2D8 | Mbd3 | Methyl-CpG-binding domain protein 3 | NO | YES | YES | 8.13 | −3.83 | 4.30 | −3.71 |
| O70310 | Nmt1 | Glycylpeptide N-tetradecanoyltransferase 1 | NO | YES | YES | 8.11 | −2.70 | 5.42 | −0.90 |
| Q9CQR6 | Ppp6c | Serine/threonine-protein phosphatase 6 catalytic | NO | YES | YES | 8.11 | −3.11 | 5.00 | −0.86 |
| | | subunit | | | | | | | |
| Q6P2B1 | Tnpo3 | Transportin-3 | NO | YES | YES | 7.97 | −2.76 | 5.22 | −0.26 |
| Q3U2P1 | Sec24a | Protein transport protein Sec24A | NO | YES | YES | 7.85 | −3.37 | 4.48 | −1.19 |
| Q8C2E7 | Washc5 | WASH complex subunit 5 | NO | YES | YES | 7.85 | −3.4' | 4.44 | −1.11 |
| Q922H1 | Prmt3 | Protein arginine N-methyltransferase 3 | NO | YES | YES | 7.77 | −2.90 | 4.87 | −1.43 |
| Q6P1F6 | Ppp2r2a | Serine/threonine-protein phosphatase 2A 55 | NO | YES | YES | 7.76 | −2.84 | 4.92 | −0.78 |
| | | kDa regulatory subunit B alpha isoform | | | | | | | |
| P52432 | Polr1c | DNA-directed RNA polymerases I and III subunit | NO | YES | YES | 7.73 | −3.02 | 4.71 | −0.55 |
| | | RPAC1 | | | | | | | |
| P97311 | Mcm6 | DNA replication licensing factor MCM6 | NO | YES | YES | 7.62 | −2.85 | 4.76 | −1.57 |
| Q8C6G8 | Wdr26 | WD repeat-containing protein 26 | NO | YES | YES | 7.53 | −2.96 | 4.57 | −0.27 |
| Q9D2R0 | Aacs | Acetoacetyl-CoA synthetase | NO | YES | YES | 7.50 | −3.63 | 3.87 | −1.12 |
| Q3UMB9 | Washc4 | WASH complex subunit 4 | NO | YES | YES | 7.45 | −3.92 | 3.53 | −0.51 |
| Q6NZC7 | Sec23ip | SEC23-interacting protein | NO | YES | YES | 7.32 | −2.37 | 4.95 | −0.61 |
| P27048 | Snrpb | Small nuclear ribonucleoprotein-associated | NO | YES | YES | 7.21 | −3.43 | 3.77 | −1.36 |
| | | protein B | | | | | | | |
| Q6ZQL4 | Wdr43 | WD repeat-containing protein 43 | NO | YES | YES | 7.09 | −4.08 | 3.01 | −0.12 |
| Q61009 | Scarb1 | Scavenger receptor class B member 1 | NO | YES | YES | 7.03 | −2.59 | 4.45 | −0.52 |
| Q80UP5 | Ankrd13a | Ankyrin repeat domain-containing protein 13A | NO | YES | YES | 6.96 | −4.84 | 2.12 | −1.95 |
| Q6PDI5 | Ecpas | Proteasome adapter and scaffold protein | NO | YES | YES | 6.95 | −3.05 | 3.90 | −1.29 |
| | | ECM29 | | | | | | | |
| O70311 | Nmt2 | Glycylpeptide N-tetradecanoyltransferase 2 | NO | YES | YES | 6.88 | −2.56 | 4.32 | −2.23 |
| P62746 | Rhob | Rho-related GTP-binding protein RhoB | NO | YES | YES | 6.87 | −3.89 | 2.99 | −3.04 |
| Q8VHK9 | Dhx36 | ATP-dependent RNA helicase DHX36 | NO | YES | YES | 6.86 | −3.24 | 3.62 | −1.16 |
| Q9ES00 | Ube4b | Ubiquitin conjugation factor E4 B | NO | YES | YES | 6.80 | −3.18 | 3.61 | −1.51 |
| Q2YDW2 | Msto1 | Protein misato homolog 1 | NO | YES | YES | 6.77 | −2.94 | 3.83 | −2.14 |
| Q5SSW2 | Psme4 | Proteasome activator complex subunit 4 | NO | YES | YES | 6.60 | −3.57 | 3.02 | −2.28 |
| Q99J09 | Wdr77 | Methylosome protein 50 | NO | YES | YES | 6.43 | −3.12 | 3.32 | −0.95 |
| Q8VHR5 | Gatad2b | Transcriptional repressor p66-beta | NO | YES | YES | 6.38 | −3.54 | 2.85 | −3.53 |
| Q8R2N2 | Utp4 | U3 small nucleolar RNA-associated protein 4 | NO | YES | YES | 6.28 | −3.01 | 3.27 | −0.61 |
| | | homolog | | | | | | | |
| Q8CA71 | Shisa4 | Protein shisa-4 | NO | YES | YES | 6.26 | 5.65 | −0.60 | 1.17 |
| Q8VI75 | Ipo4 | Importin-4 | NO | YES | YES | 6.14 | −3.81 | 2.33 | −0.59 |
| Q3UKC1 | Tax1bp1 | Tax1-binding protein 1 homolog | NO | YES | YES | 6.10 | −3.59 | 2.51 | −2.19 |
| Q8K2Z4 | Ncapd2 | Condensin complex subunit 1 | NO | YES | YES | 6.04 | −4.67 | 1.37 | −1.17 |
| Q9CX34 | Sugt1 | Protein SGT1 homolog | NO | YES | YES | 6.00 | −3.70 | 2.30 | −1.34 |
| P70180 | Npr3 | Atrial natriuretic peptide receptor 3 | NO | YES | YES | 5.96 | 5.51 | −0.45 | 1.25 |
| Q8BKX1 | Baiap2 | Brain-specific angiogenesis inhibitor 1- | NO | YES | YES | 5.89 | 3.04 | −2.86 | 0.92 |
| | | associated protein 2 | | | | | | | |
| Q99K70 | Rragc | Ras-related GTP-binding protein C | NO | YES | YES | 5.86 | −3.32 | 2.54 | −1.22 |
| P61588 | Rnd3 | Rho-related GTP-binding protein RhoE | NO | YES | YES | 5.85 | −5.49 | 0.36 | −0.02 |
| Q8CFE3 | Rcor1 | REST corepressor 1 | NO | YES | YES | 5.77 | −3.19 | 2.57 | −2.32 |
| Q8VDJ3 | Hdlbp | Vigilin | NO | YES | YES | 5.44 | −3.09 | 2.35 | −2.62 |
| Q9CQE8 | RTRAF | RNA transcription, translation and transport | NO | YES | YES | 5.39 | −2.98 | 2.41 | −1.36 |
| | | factor protein | | | | | | | |
| Q9WUP7 | Uchl5 | Ubiquitin carboxyl-terminal hydrolase isozyme | NO | YES | YES | 5.30 | −3.12 | 2.18 | −0.87 |
| | | L5 | | | | | | | |
| Q8CE96 | Trmt6 | tRNA (adenine(58)-N(1))-methyltransferase | NO | YES | YES | 5.23 | −2.79 | 2.4 | −1.15 |
| | | non-catalytic subunit TRM6 | | | | | | | |
| P54823 | Ddx6 | Probable ATP-dependent RNA helicase DDX6 | NO | YES | YES | 5.19 | −2.94 | 2.26 | −1.64 |
| P62309 | Snrpg | Small nuclear ribonucleoprotein G | NO | YES | YES | 5.11 | −2.61 | 2.50 | −1.07 |
| Q3UVG3 | Fam91a1 | Protein FAM91A1 | NO | YES | YES | 5.07 | −4.04 | 1.03 | −2.48 |
| Q3TJ91 | Llgl2 | Lethal(2) giant larvae protein homolog 2 | NO | YES | YES | 5.04 | −3.08 | 1.96 | −0.47 |
| Q8BHL3 | Tbc1d10b | TBC1 domain family member 10B | NO | YES | YES | 5.03 | 3.17 | −1.86 | 4.94 |
| Q9EPU4 | Cpsf1 | Cleavage and polyadenylation specificity factor | NO | YES | YES | 4.98 | −3.14 | 1.84 | −2.92 |
| | | subunit 1 | | | | | | | |
| P97820 | Map4k4 | Mitogen-activated protein kinase 4 | NO | YES | YES | 4.91 | −3.00 | 1.90 | −0.20 |
| P25976 | Ubtf | Nucleolar transcription factor 1 | NO | YES | YES | 4.89 | −3.93 | 0.97 | −0.25 |
| Q8CGB6 | Tns2 | Tensin-2 | NO | YES | YES | 4.77 | −4.49 | 0.28 | −1.94 |
| Q99104 | Myo5a | Unconventional myosin-Va | NO | YES | YES | 4.75 | −4.50 | 0.25 | −2.16 |
| P29391 | Ftl1 | Ferritin light chain 1 | NO | YES | YES | 4.60 | −3.92 | 0.68 | −1.53 |
| Q9CZX7 | Pip4p2 | Type 2 phosphatidylinositol 4,5-bisphosphate 4- | NO | YES | YES | 4.56 | −3.35 | 1.21 | −2.0 |
| | | phosphatase | | | | | | | |
| Q9ER00 | Stx12 | Syntaxin-12 | NO | YES | YES | 4.55 | −3.97 | 0.57 | −1.97 |
| Q9DC48 | Cdc40 | Pre-mRNA-processing factor 17 | NO | YES | YES | 4.54 | 2.94 | −1.60 | 1.89 |
| Q9JI33 | Ntn4 | Netrin-4 | NO | YES | YES | 4.44 | −3.53 | 0.91 | −0.52 |
| B1AY13 | Usp24 | Ubiquitin carboxyl-terminal hydrolase 24 | NO | YES | YES | 4.34 | −2.84 | 1.50 | −1.19 |
| Q80YF6 | Upk3b | Uroplakin-3b | NO | YES | YES | 4.22 | 3.65 | −0.57 | 1.87 |
| P04370 | Mbp | Myelin basic protein | NO | YES | YES | 4.14 | 2.30 | −1.84 | 1.49 |
| Q91XU3 | Pip4k2c | Phosphatidylinositol 5-phosphate 4-kinase type- | NO | YES | YES | 4.13 | 3.31 | −0.82 | 1.23 |
| | | 2 gamma | | | | | | | |
| Q64008 | Rab34 | Ras-related protein Rab-34 | NO | YES | YES | 4.01 | 2.97 | −1.04 | 1.01 |
| P97496 | Smarcc1 | SWI/SNF complex subunit SMARCC1 | NO | YES | YES | 3.92 | −2.41 | 1.51 | −0.79 |
| P11157 | Rrm2 | Ribonucleoside-diphosphate reductase subunit | NO | YES | YES | 3.83 | −2.87 | 0.96 | −0.46 |
| | | M2 | | | | | | | |
| O35379 | Abcc1 | Multidrug resistance-associated protein 1 | NO | YES | YES | 3.72 | 3.36 | −0.36 | 2.39 |
| Q9WVD4 | Clcn5 | H(+)/CI(−) exchange transporter 5 | NO | YES | YES | 3.21 | −3.13 | 0.08 | −0.81 |
| Q3V1G4 | Olfml2b | Olfactomedin-like protein 2B | NO | YES | YES | 2.53 | −2.32 | 0.21 | −0.89 |
| Q6Q899 | Ddx58 | Probable ATP-dependent RNA helicase DDX58 | NO | YES | NO | 22.79 | -14.23 | 8.56 | 1.08 |
| Q9D6Z1 | Nop56 | Nucleolar protein 56 | NO | YES | NO | 20.15 | −5.02 | 15.13 | 0.16 |
| Q810A7 | Ddx42 | ATP-dependent RNA helicase DDX42 | NO | YES | NO | 19.21 | −8.07 | 11.14 | 1.21 |
| Q9Z0G0 | Gipc1 | PDZ domain-containing protein GIPC1 | NO | YES | NO | 16.91 | 5.56 | -11.35 | −1.04 |
| Q91YN9 | Bag2 | BAG family molecular chaperone regulator 2 | NO | YES | NO | 15.32 | −3.31 | 12.01 | 0.21 |
| P61211 | Arl1 | ADP-ribosylation factor-like protein 1 | NO | YES | NO | 15.23 | −2.71 | 12.52 | 0.42 |
| Q9CU62 | Smc1a | Structural maintenance of chromosomes protein | NO | YES | NO | 14.90 | −4.00 | 10.90 | 0.19 |
| | | 1A | | | | | | | |
| Q6DFW4 | Nop58 | Nucleolar protein 58 | NO | YES | NO | 14.12 | −2.89 | 11.23 | 1.62 |
| A2AQ19 | Rtf1 | RNA polymerase-associated protein RTF1 | NO | YES | NO | 13.74 | −4.59 | 9.15 | 2.04 |
| | | homolog | | | | | | | |
| Q99LD9 | Eif2b2 | Translation initiation factor elF-2B subunit beta | NO | YES | NO | 12.40 | −5.60 | 6.79 | 3.14 |
| Q9DBD5 | Pelp1 | Proline-, glutamic acid- and leucine-rich protein | NO | YES | NO | 11.96 | −7.8 | 4.15 | 1.12 |
| | | 1 | | | | | | | |
| Q9R0X0 | Med20 | Mediator of RNA polymerase II transcription | NO | YES | NO | 11.89 | −5.88 | 6.01 | 0.06 |
| | | subunit 20 | | | | | | | |
| Q8CG85 | Mamdc2 | MAM domain-containing protein 2 | NO | YES | NO | 11.03 | 8.05 | −2.97 | −2.66 |
| Q8K2T8 | Paf1 | RNA polymerase II-associated factor 1 homolog | NO | YES | NO | 10.93 | −5.79 | 5.14 | 6.09 |
| Q9R1C7 | Prpf40a | Pre-mRNA-processing factor 40 homolog A | NO | YES | NO | 10.68 | −3.27 | 7.41 | 0.20 |
| Q99LU0 | Chmp1b1 | Charged multivesicular body protein 1b-1 | NO | YES | NO | 9.82 | 4.63 | −5.19 | −3.45 |
| Q9JLI8 | Sart3 | Squamous cell carcinoma antigen recognized | NO | YES | NO | 9.08 | −3.65 | 5.43 | 0.90 |
| | | by T-cells 3 | | | | | | | |
| Q7JJ13 | Brd2 | Bromodomain-containing protein 2 | NO | YES | NO | 8.95 | −3.71 | 5.24 | 0.05 |
| Q61235 | Sntb2 | Beta-2-syntrophin | NO | YES | NO | 8.73 | −5.47 | 3.26 | 4.44 |
| Q8CI95 | Osbpl11 | Oxysterol-binding protein-related protein 11 | NO | YES | NO | 8.58 | −3.95 | 4.62 | 0.12 |
| Q99LL5 | Pwp1 | Periodic tryptophan protein 1 homolog | NO | YES | NO | 8.39 | −2.58 | 5.81 | 1.11 |
| Q921F4 | Hnrnpll | Heterogeneous nuclear ribonucleoprotein L-like | NO | YES | NO | 8.38 | −5.13 | 3.25 | 0.76 |
| Q922J6 | Tspan2 | Tetraspanin-2 | NO | YES | NO | 8.34 | −5.72 | 2.61 | 0.62 |
| Q810D6 | Grwd1 | Glutamate-rich WD repeat-containing protein 1 | NO | YES | NO | 8.06 | 5.41 | 2.65 | 4.92 |
| Q8BTI8 | Srrm2 | Serine/arginine repetitive matrix protein 2 | NO | YES | NO | 7.87 | −4.36 | 3.51 | 0.17 |
| Q8JZX4 | Rbm17 | Splicing factor 45 | NO | YES | NO | 7.77 | −5.40 | 2.38 | 0.36 |
| Q8N7N5 | Dcaf8 | DDB1- and CUL4-associated factor 8 | NO | YES | NO | 7.70 | −4.22 | 3.49 | 0.84 |
| O35474 | Edil3 | EGF-like repeat and discoidin I-like domain- | NO | YES | NO | 7.67 | −3.16 | 4.51 | 3.24 |
| | | containing protein 3 | | | | | | | |
| P39876 | Timp3 | Metalloproteinase inhibitor 3 | NO | YES | NO | 7.42 | 4.49 | −2.93 | −3.42 |
| Q8BQM4 | Heatr3 | HEAT repeat-containing protein 3 | NO | YES | NO | 7.08 | −3.98 | 3.09 | 1.40 |
| Q9ESU6 | Brd4 | Bromodomain-containing protein 4 | NO | YES | NO | 6.97 | −3.64 | 3.33 | 0.37 |
| Q8K2B0 | P3h4 | Endoplasmic reticulum protein SC65 | NO | YES | NO | 6.88 | −5.26 | 1.62 | 1.07 |
| Q99JB8 | Pacsin3 | Protein kinase C and casein kinase II substrate | NO | YES | NO | 6.45 | 3.04 | −3.41 | −2.60 |
| | | protein 3 | | | | | | | |
| Q6PGB6 | Naa50 | N-alpha-acetyltransferase 50 | NO | YES | NO | 6.3 | −4.36 | 1.96 | 1.03 |
| Q8BMC0 | Lpar6 | Lysophosphatidic acid receptor 6 | NO | YES | NO | 6.22 | 3.20 | −3.03 | −1.25 |
| Q62433 | Ndrg1 | Protein NDRG1 | NO | YES | NO | 6.19 | −3.50 | 2.70 | 1.14 |
| Q8C3Y4 | Kntc1 | Kinetochore-associated protein 1 | NO | YES | NO | 6.09 | −3.61 | 2.48 | 0.31 |
| P24788 | Cdk11b | Cyclin-dependent kinase 11B | NO | YES | NO | 6.07 | −3.23 | 2.84 | 4.28 |
| P63166 | Sumo1 | Small ubiquitin-related modifier 1 | NO | YES | NO | 5.99 | −3.71 | 2.28 | 5.82 |
| Q9QXA5 | Lsm4 | U6 snRNA-associated Sm-like protein LSm4 | NO | YES | NO | 5.40 | −2.91 | 2.49 | 0.61 |
| P52927 | Hmga2 | High mobility group protein HMGI-C | NO | YES | NO | 5.34 | −3.36 | 1.97 | 0.21 |
| Q99LC2 | Cstf1 | Cleavage stimulation factor subunit 1 | NO | YES | NO | 4.79 | −2.99 | 1.81 | 1.34 |
| P47226 | Tes | Testin | NO | YES | NO | 4.69 | −4.49 | 0.20 | 1.50 |
| Q689Z5 | Sbno1 | Protein strawberry notch homolog 1 | NO | YES | NO | 4.67 | −3.37 | 1.29 | 0.31 |
| Q8C4B4 | Unc119b | Protein unc-119 homolog B | NO | YES | NO | 4.63 | −2.42 | 2.22 | 0.17 |
| O88271 | Cfdp1 | Craniofacial development protein 1 | NO | YES | NO | 4.50 | −2.62 | 1.87 | 8.58 |
| Q9CQS8 | Sec61b | Protein transport protein Sec61 subunit beta | NO | YES | NO | 3.57 | −3.29 | 0.27 | 3.71 |
| P58466 | Ctdsp1 | Carboxy-terminal domain RNA polymerase II | NO | NO | YES | 9.37 | −9.09 | −0.28 | −10.61 |
| | | polypeptide A small phosphatase 1 | | | | | | | |
| Q80TF3 | Pcdh19 | Protocadherin-19 | NO | NO | YES | 7.74 | −7.44 | −0.30 | −2.26 |
| Q62283 | Tspan7 | Tetraspanin-7 | NO | NO | YES | 7.37 | −4.48 | −2.89 | −0.21 |
| E9Q634 | Myo1e | Unconventional myosin-le | NO | NO | YES | 5.88 | −4.42 | −1.46 | −1.56 |
| Q99JY8 | Plpp3 | Phospholipid phosphatase 3 | NO | NO | YES | 4.97 | 4.46 | 0.51 | 4.49 |
| Q921I6 | Sh3bp4 | SH3 domain-binding protein 4 | NO | NO | YES | 4.75 | −3.84 | −0.91 | −0.29 |
| Q9QYI3 | Dnajc7 | DnaJ homolog subfamily C member 7 | NO | NO | YES | 4.50 | −3.83 | −0.67 | −2.37 |
| P32233 | Drg1 | Developmentally-regulated GTP-binding protein | NO | NO | YES | 4.38 | −3.32 | −1.06 | −1.58 |
| | | 1 | | | | | | | |
| Q4VC33 | Maea | Macrophage erythroblast attacher | NO | NO | YES | 3.96 | −3.58 | −0.37 | −2.82 |

Table S1 Key:
Column 1: Accession (Mus musculus)
Column 2: GN gene name
Column 3: Description, protein name
Column 4: In Human Plasma (YES/NO); see methods for datasets and criteria
Column 5: RTV Corrective Direction (YES = protein abundance effect opposite to DYT/WT)
Column 6: ISRIB and DYT Parallel Direction (YES = protein abundance effect similar to DYT/WT)
Column 7: Absolute Cohen's d Sum (Geno + RTV)
Column 8: DYT/WT Cohen's d
Column 9: DYT + RTV/DYT Cohen's d
Column 10: WT + ISRIB/WT Cohen's d

TABLE S2
List of 478 Metabolites Quantified in Human DYT1 Subject and Healthy Controls

| | | Mean of | Mean of | | | |
|---|---|---|---|---|---|---|
| | | Healthy | DYT1- | | SE of | |
| Metabolite | P value | Control | Dystonia | Difference | difference | t ratio |
| PC aa C24:0_ | 0.0004937 | 0.2419 | 0.1986 | 0.04331 | 0.01134 | 3.819 |
| Glycerophospholipids | | | | | | |
| 5-AVA_Aminoacids Related | 0.0013736 | 0.1206 | 0.06457 | 0.05599 | 0.0162 | 3.457 |
| Cystine_Aminoacids Related | 0.00284633 | 135.9 | 246.2 | −110.2 | 34.52 | 3.193 |
| Cer(d16:1/20:0)_Ceramides | 0.00709256 | 0.3731 | 0.2566 | 0.1165 | 0.04081 | 2.855 |
| lysoPC a C26:0_ | 0.00939202 | 0.8514 | 0.734 | 0.1174 | 0.04265 | 2.752 |
| Glycerophospholipids | | | | | | |
| Asp_Aminoacids | 0.01023977 | 8.883 | 6.715 | 2.169 | 0.8018 | 2.705 |
| Cys_Aminoacids | 0.01111543 | 102.9 | 133.6 | −30.77 | 11.3 | 2.723 |
| Tyr_Aminoacids | 0.01392985 | 100.3 | 83.3 | 17.03 | 6.585 | 2.586 |
| PC aa C26:0_ | 0.01625462 | 1.837 | 1.361 | 0.4759 | 0.1853 | 2.568 |
| Glycerophospholipids | | | | | | |
| lysoPC a C28:0_ | 0.02599999 | 1.123 | 1.004 | 0.1196 | 0.05096 | 2.347 |
| Glycerophospholipids | | | | | | |
| C18:2_Acylcarnitines | 0.02946179 | 0.0672 | 0.08082 | −0.01362 | 0.005793 | 2.351 |
| Cer(d18:2/18:1)_Ceramides | 0.03571867 | 0.0585 | 0.03697 | 0.02152 | 0.009761 | 2.205 |
| Cer(d16:1/18:0)_Ceramides | 0.03682499 | 0.3344 | 0.2412 | 0.09316 | 0.04283 | 2.175 |
| p-Cresol-SO4_Cresols | 0.03691433 | 12.21 | 7.167 | 5.042 | 2.311 | 2.181 |
| PC aa C40:6_ | 0.0408721 | 21.85 | 17.57 | 4.274 | 2.002 | 2.135 |
| Glycerophospholipids | | | | | | |
| C16-OH_Acylcarnitines | 0.04121264 | 0.08047 | 0.0714 | 0.009074 | 0.00423 | 2.145 |
| Gly_Aminoacids | 0.04170703 | 406.3 | 335.3 | 70.99 | 33.61 | 2.112 |
| Hypoxanthine_ | 0.04480802 | 1.661 | 3.012 | 1.351 | 0.6166 | 2.191 |
| Nucleobases Related | | | | | | |
| EPA_Fatty Acids | 0.0467916 | 0.2286 | 0.161 | 0.06759 | 0.03268 | 2.068 |
| C14:2-OH_Acylcarnitines | 0.04762799 | 0.03848 | 0.03255 | 0.005932 | 0.002822 | 2.102 |
| 3-IPA_Indoles Derivatives | 0.05359111 | 0.4884 | 0.3176 | 0.1708 | 0.08573 | 1.992 |
| lysoPC a C26:1_ | 0.05671659 | 1.139 | 1.014 | 0.1258 | 0.06267 | 2.007 |
| Glycerophospholipids | | | | | | |
| C5-M-DC_Acylcarnitines | 0.07277922 | 0.02859 | 0.01861 | 0.009978 | 0.005397 | 1.849 |
| CE(18:1)_Cholesterol Esters | 0.07282126 | 200.1 | 170.1 | 30.03 | 16.13 | 1.862 |
| lysoPC a C24:0_ | 0.07318874 | 0.5954 | 0.5465 | 0.04883 | 0.02603 | 1.876 |
| Glycerophospholipids | | | | | | |
| CE(14:0)_Cholesterol Esters | 0.07410013 | 13.39 | 9.408 | 3.979 | 2.127 | 1.87 |
| C4_Acylcarnitines | 0.07822723 | 0.318 | 0.2738 | 0.04427 | 0.02433 | 1.819 |
| DHEAS_Hormones | 0.08296974 | 4.405 | 7.027 | −2.622 | 1.448 | 1.811 |
| HexCer(d18:1/24:0)_ | 0.08321631 | 1.93 | 2.265 | −0.3358 | 0.1854 | 1.811 |
| Glycosylceramides | | | | | | |
| Pro_Aminoacids | 0.08433788 | 353.8 | 289.2 | 64.55 | 36.31 | 1.778 |
| TG(18:1_38:7)_Triacylglycerols | 0.09032914 | 0.3121 | 0.2432 | 0.06884 | 0.03914 | 1.759 |
| lysoPC a C28:1_ | 0.09290439 | 1.245 | 1.096 | 0.1488 | 0.08519 | 1.747 |
| Glycerophospholipids | | | | | | |
| CE(20:5)_Cholesterol Esters | 0.09320374 | 39.04 | 28.29 | 10.75 | 6.235 | 1.723 |
| AABA_Aminoacids Related | 0.0951793 | 40.71 | 34.66 | 6.05 | 3.526 | 1.716 |
| C3-OH_Acylcarnitines | 0.0963099 | 0.53 | 0.4878 | 0.04212 | 0.02446 | 1.722 |
| Sarcosine_Aminoacids Related | 0.0984212 | 7.123 | 15.48 | −8.353 | 4.67 | 1.789 |
| TG(20:5_36:2)_Triacylglycerols | 0.09849772 | 0.8493 | 0.5787 | 0.2705 | 0.1597 | 1.694 |
| CE(16:1)_Cholesterol Esters | 0.0997108 | 35.17 | 25.84 | 9.332 | 5.487 | 1.701 |
| PC aa C38:6_ | 0.10092202 | 67.7 | 57.1 | 10.6 | 6.248 | 1.697 |
| Glycerophospholipids | | | | | | |
| C18:1-OH_Acylcarnitines | 0.10772731 | 0.1393 | 0.1268 | 0.0125 | 0.007503 | 1.666 |
| BABA_Aminoacids Related | 0.10974376 | 0.102 | 0.08245 | 0.01955 | 0.01185 | 1.651 |
| Cer(d18:1/18:1)_Ceramides | 0.11071854 | 0.05443 | 0.04262 | 0.01181 | 0.007156 | 1.65 |
| Cer(d18:1/25:0)_Ceramides | 0.11248157 | 0.534 | 0.4637 | 0.07031 | 0.04298 | 1.636 |
| Choline_Vitamins & Cofactors | 0.11313504 | 8.648 | 7.47 | 1.178 | 0.7232 | 1.629 |
| C14:1-OH_Acylcarnitines | 0.11467449 | 0.07579 | 0.06832 | 0.00747 | 0.004577 | 1.632 |
| Cortisol_Hormones | 0.11513914 | 0.2928 | 0.3855 | −0.09268 | 0.05547 | 1.671 |
| Cer(d16:1/23:0)_Ceramides | 0.12159238 | 0.2945 | 0.2366 | 0.05792 | 0.03635 | 1.594 |
| Thr_Aminoacids | 0.12309177 | 203.6 | 171.3 | 32.25 | 20.44 | 1.578 |
| Met_Aminoacids | 0.12580453 | 41.71 | 34.78 | 6.934 | 4.429 | 1.566 |
| PC ae C30:2_ | 0.13036954 | 0.3444 | 0.3138 | 0.03064 | 0.01956 | 1.566 |
| Glycerophospholipids | | | | | | |
| Arg_Aminoacids | 0.13102482 | 132.7 | 114.7 | 17.97 | 11.57 | 1.553 |
| C7-DC_Acylcarnitines | 0.13185662 | 0.07007 | 0.06058 | 0.009482 | 0.006139 | 1.545 |
| HexCer(d18:2/23:0)_ | 0.13471686 | 0.4383 | 0.5558 | −0.1175 | 0.07585 | 1.55 |
| Glycosylceramides | | | | | | |
| Ser_Aminoacids | 0.13541578 | 150.2 | 131.8 | 18.37 | 12.02 | 1.528 |
| GABA_Biogenic Amines | 0.13808184 | 0.2762 | 0.2481 | 0.02811 | 0.01843 | 1.525 |
| CE(22:6)_Cholesterol Esters | 0.14767415 | 16.89 | 13.68 | 3.217 | 2.142 | 1.502 |
| C3:1_Acylcarnitines | 0.15135385 | 0.281 | 0.2605 | 0.02054 | 0.0139 | 1.478 |
| Xanthine_Nucleobases Related | 0.1552735 | 1.503 | 1.745 | −0.2421 | 0.1669 | 1.451 |
| CE(16:0)_Cholesterol Esters | 0.15552086 | 125.2 | 108.6 | 16.58 | 11.3 | 1.466 |
| alpha-AAA_Aminoacids | 0.15852728 | 1.734 | 1.201 | 0.5321 | 0.3686 | 1.444 |
| Related | | | | | | |
| Ile_Aminoacids | 0.16258427 | 119.4 | 103.1 | 16.33 | 11.47 | 1.424 |
| HexCer(d18:1/23:0)_ | 0.16340278 | 2.09 | 2.318 | −0.2285 | 0.16 | 1.428 |
| Glycosylceramides | | | | | | |
| CE(18:2)_Cholesterol Esters | 0.16830894 | 766.7 | 673.9 | 92.82 | 65.48 | 1.417 |
| PC ae C44:5_ | 0.17542089 | 1.5 | 1.658 | −0.1582 | 0.1141 | 1.387 |
| Glycerophospholipids | | | | | | |
| PC ae C42:4_ | 0.1762454 | 0.9637 | 1.074 | −0.1106 | 0.0788 | 1.404 |
| Glycerophospholipids | | | | | | |
| HexCer(d18:1/18:0)_ | 0.17925038 | 0.3861 | 0.3239 | 0.06226 | 0.04498 | 1.384 |
| Glycosylceramides | | | | | | |
| Phe_Aminoacids | 0.18074027 | 86.47 | 78.71 | 7.766 | 5.676 | 1.368 |
| PC aa C36:5_ | 0.19007727 | 22.04 | 18.13 | 3.901 | 2.921 | 1.336 |
| Glycerophospholipids | | | | | | |
| HexCer(d18:1/22:0)_ | 0.19132596 | 3.855 | 4.265 | −0.4102 | 0.3053 | 1.343 |
| Glycosylceramides | | | | | | |
| PC ae C44:4_ | 0.1978505 | 0.3837 | 0.4166 | −0.03295 | 0.02482 | 1.328 |
| Glycerophospholipids | | | | | | |
| SM C20:2_Sphingolipids | 0.19921854 | 0.4429 | 0.3817 | 0.06123 | 0.0463 | 1.323 |
| Cer(d16:1/22:0)_Ceramides | 0.20855702 | 0.6015 | 0.5108 | 0.09075 | 0.07066 | 1.284 |
| TG(20:2_32:1)_ | 0.21091182 | 0.448 | 0.3367 | 0.1114 | 0.08678 | 1.283 |
| Triacylglycerols | | | | | | |
| Cer(d18:1/26:0)_Ceramides | 0.21438557 | 0.1104 | 0.08885 | 0.02156 | 0.01695 | 1.272 |
| PC ae C38:0_ | 0.21454829 | 1.861 | 1.664 | 0.1973 | 0.1559 | 1.266 |
| Glycerophospholipids | | | | | | |
| TG(20:5_36:3)_Triacylglycerols | 0.21835934 | 0.7199 | 0.5505 | 0.1695 | 0.1354 | 1.252 |
| DHA_Fatty Acids | 0.22399217 | 1.338 | 1.024 | 0.3141 | 0.2538 | 1.237 |
| beta-Ala_Biogenic Amines | 0.22704621 | 10.25 | 5.569 | 4.684 | 3.8 | 1.232 |
| PC aa C28:1_ | 0.24357293 | 5.107 | 4.525 | 0.5824 | 0.49 | 1.189 |
| Glycerophospholipids | | | | | | |
| TrpBetaine_Aminoacids | 0.24767082 | 0.3341 | 0.4805 | −0.1464 | 0.1224 | 1.196 |
| Related | | | | | | |
| HexCer(d18:1/24:1)_ | 0.25140277 | 6.228 | 6.615 | −0.3876 | 0.3309 | 1.171 |
| Glycosylceramides | | | | | | |
| Hex2Cer(d18:1/16:0)_ | 0.25519864 | 5.137 | 5.511 | −0.3741 | 0.3211 | 1.165 |
| Glycosylceramides | | | | | | |
| SDMA_Aminoacids Related | 0.259729 | 0.6799 | 0.6239 | 0.05593 | 0.04868 | 1.149 |
| Betaine_Aminoacids Related | 0.25994187 | 77 | 66.16 | 10.83 | 9.443 | 1.147 |
| 3-Met-His_Aminoacids | 0.26329356 | 17.34 | 12.4 | 4.94 | 4.35 | 1.136 |
| Related | | | | | | |
| H1_Sugars | 0.26508696 | 6903 | 6188 | 714.6 | 631.3 | 1.132 |
| TG(22:6_34:1)_ | 0.2652582 | 2.495 | 1.846 | 0.6493 | 0.5742 | 1.131 |
| Triacylglycerols | | | | | | |
| C5_Acylcarnitines | 0.26792606 | 0.1821 | 0.1623 | 0.0198 | 0.01748 | 1.133 |
| PC aa C34:1_ | 0.27426963 | 210.3 | 190.8 | 19.45 | 17.5 | 1.111 |
| Glycerophospholipids | | | | | | |
| C5-OH (C3-DC-M)_ | 0.27523111 | 0.09701 | 0.09204 | 0.004969 | 0.004453 | 1.116 |
| Acylcarnitines | | | | | | |
| TG(18:1_30:2)_Triacylglycerols | 0.2818155 | 1.412 | 1.803 | −0.3919 | 0.3549 | 1.104 |
| GCA_Bile Acids | 0.2838924 | 0.1104 | 0.1578 | −0.04737 | 0.04301 | 1.101 |
| TG(22:6_32:1)_Triacylglycerols | 0.28895988 | 0.6868 | 0.5255 | 0.1612 | 0.1499 | 1.076 |
| PC ae C36:1_ | 0.29383811 | 9.002 | 8.249 | 0.753 | 0.7053 | 1.068 |
| Glycerophospholipids | | | | | | |
| Cer(d18:1/14:0)_Ceramides | 0.30293271 | 0.186 | 0.1654 | 0.02054 | 0.01956 | 1.05 |
| Glu_Aminoacids | 0.30628312 | 64.99 | 53.47 | 11.52 | 11.05 | 1.042 |
| PC aa C32:1_ | 0.31028521 | 17.06 | 14.26 | 2.794 | 2.71 | 1.031 |
| Glycerophospholipids | | | | | | |
| C16:1-OH_Acylcarnitines | 0.31240248 | 0.0369 | 0.03463 | 0.002266 | 0.002193 | 1.033 |
| PC aa C38:5_ | 0.31402909 | 53.16 | 48.02 | 5.136 | 4.992 | 1.029 |
| Glycerophospholipids | | | | | | |
| PC aa C36:2_ | 0.32311658 | 271.3 | 254.9 | 16.34 | 16.23 | 1.006 |
| Glycerophospholipids | | | | | | |
| Hex2Cer(d18:1/24:1)_ | 0.32564671 | 0.7214 | 0.7839 | −0.06251 | 0.06251 | 0.9999 |
| Glycosylceramides | | | | | | |
| CE(20:4)_Cholesterol Esters | 0.32675523 | 133 | 118 | 14.94 | 14.93 | 1.001 |
| PC ae C44:6_ | 0.32756429 | 1.328 | 1.433 | −0.1053 | 0.1052 | 1.002 |
| Glycerophospholipids | | | | | | |
| Ala_Aminoacids | 0.33107852 | 587.1 | 523.2 | 63.96 | 64.35 | 0.994 |
| 1-Met-His_Aminoacids Related | 0.33227678 | 7.108 | 6.473 | 0.6347 | 0.6463 | 0.9821 |
| PC aa C38:0_ | 0.333542 | 2.58 | 2.36 | 0.2196 | 0.2221 | 0.989 |
| Glycerophospholipids | | | | | | |
| C10_Acylcarnitines | 0.33427609 | 0.23 | 0.4625 | −0.2325 | 0.2313 | 1.005 |
| C3_Acylcarnitines | 0.33615105 | 0.6129 | 0.5561 | 0.05677 | 0.05773 | 0.9835 |
| Asn_Aminoacids | 0.34044973 | 84.92 | 78.14 | 6.784 | 7.005 | 0.9684 |
| Cer(d18:1/16:0)_Ceramides | 0.34246215 | 0.8588 | 0.9077 | −0.04891 | 0.05085 | 0.9619 |
| Lys_Aminoacids | 0.34357378 | 302.3 | 279.8 | 22.53 | 23.47 | 0.9598 |
| C16:2-OH_Acylcarnitines | 0.34665201 | 0.0286 | 0.02704 | 0.001565 | 0.00163 | 0.9605 |
| HexCer(d18:1/16:0)_ | 0.35057415 | 1.721 | 1.816 | −0.09526 | 0.1003 | 0.9498 |
| Glycosylceramides | | | | | | |
| C18:1_Acylcarnitines | 0.35587106 | 0.1216 | 0.1317 | −0.0101 | 0.01069 | 0.9449 |
| lysoPC a C20:3_ | 0.3601367 | 3.143 | 3.437 | −0.294 | 0.3141 | 0.936 |
| Glycerophospholipids | | | | | | |
| C2_Acylcarnitines | 0.36130537 | 6.598 | 5.944 | 0.6539 | 0.7047 | 0.9279 |
| TG(18:1_28:1)_Triacylglycerols | 0.36750797 | 1.002 | 1.34 | −0.338 | 0.3648 | 0.9264 |
| TG(20:3_36:3)_Triacylglycerols | 0.3771626 | 0.748 | 0.6515 | 0.09653 | 0.1067 | 0.9048 |
| PC ae C36:3_ | 0.38116658 | 8.277 | 8.829 | −0.5526 | 0.6219 | 0.8885 |
| Glycerophospholipids | | | | | | |
| TG(18:1_38:6)_Triacylglycerols | 0.38611609 | 1.2 | 1.054 | 0.1465 | 0.1666 | 0.8795 |
| TG(18:2_30:1)_Triacylglycerols | 0.38717978 | 2.454 | 3.06 | −0.6061 | 0.6854 | 0.8843 |
| FA(18:2)_Fatty Acids | 0.38849989 | 198.2 | 159.1 | 39.14 | 44.73 | 0.875 |
| CE(20:3)_Cholesterol Esters | 0.39007969 | 11.34 | 10.23 | 1.113 | 1.269 | 0.877 |
| DG(18:1_18:4)_Diacylglycerols | 0.39023699 | 0.1504 | 0.1255 | 0.02491 | 0.02857 | 0.8719 |
| C8_Acylcarnitines | 0.39212873 | 0.08601 | 0.2491 | 0.1631 | 0.1839 | 0.8872 |
| Kynurenine_Aminoacids Related | 0.39225703 | 2.489 | 2.278 | 0.2116 | 0.2436 | 0.8687 |
| TG(22:6_34:2)_Triacylglycerols | 0.39473079 | 1.931 | 1.5 | 0.4315 | 0.5012 | 0.8608 |
| DG(18:1_18:3)_Diacylglycerols | 0.39633743 | 0.1465 | 0.1084 | 0.03815 | 0.04416 | 0.8638 |
| Lac_Carboxylic Acids | 0.3963394 | 1552 | 1715 | −162.8 | 189.8 | 0.8581 |
| TG(18:2_28:0)_Triacylglycerols | 0.39751243 | 1.156 | 1.497 | −0.3407 | 0.3935 | 0.8659 |
| TG(16:0_40:8)_Triacylglycerols | 0.40115243 | 0.5599 | 0.4568 | 0.103 | 0.1214 | 0.8491 |
| Leu_Aminoacids | 0.40151755 | 186.9 | 172 | 14.93 | 17.59 | 0.8484 |
| Val_Aminoacids | 0.40291747 | 323.8 | 306.3 | 17.51 | 20.68 | 0.8466 |
| C6 (C4:1-DC)_Acylcarnitines | 0.40611818 | 0.1396 | 0.1807 | −0.04114 | 0.04783 | 0.8601 |
| SM (OH) C14:1_Sphingolipids | 0.41540662 | 6.732 | 6.173 | 0.5591 | 0.6744 | 0.8291 |
| C12_Acylcarnitines | 0.42394343 | 0.09644 | 0.1275 | −0.03109 | 0.03764 | 0.8259 |
| PC ae C40:6_Glycerophospholipids | 0.42461691 | 4.066 | 3.838 | 0.2278 | 0.281 | 0.8109 |
| PC ae C40:4_Glycerophospholipids | 0.42573162 | 2.391 | 2.532 | −0.1416 | 0.1747 | 0.8102 |
| 3-IAA_Indoles Derivatives | 0.42618354 | 6.364 | 9.304 | −2.939 | 3.579 | 0.8214 |
| PC ae C40:1_Glycerophospholipids | 0.42719584 | 1.375 | 1.292 | 0.08327 | 0.1033 | 0.8058 |
| DCA_Bile Acids | 0.42803207 | 0.3154 | 0.4329 | −0.1175 | 0.1446 | 0.8127 |
| HexCer(d18:1/18:1)_ | 0.43038207 | 0.3345 | 0.3061 | 0.02844 | 0.03549 | 0.8014 |
| Glycosylceramides | | | | | | |
| HipAcid_Carboxylic Acids | 0.43125735 | 4.806 | 3.592 | 1.214 | 1.522 | 0.7975 |
| TG(14:0_35:1)_Triacylglycerols | 0.43152584 | 0.4155 | 0.3323 | 0.08314 | 0.1035 | 0.8033 |
| HexCer(d16:1/22:0)_ | 0.43262937 | 0.1105 | 0.0953 | 0.01524 | 0.01909 | 0.7984 |
| Glycosylceramides | | | | | | |
| Cer(d18:1/23:0)_Ceramides | 0.43513287 | 1.384 | 1.286 | 0.09777 | 0.1237 | 0.7902 |
| GCDCA_Bile Acids | 0.43701858 | 3.892 | 4.677 | −0.7857 | 0.9911 | 0.7927 |
| C3-DC (C4-OH)_Acylcarnitines | 0.43756841 | 0.1605 | 0.1445 | 0.01599 | 0.0201 | 0.7954 |
| Cer(d18:2/24:0)_Ceramides | 0.44052546 | 0.6849 | 0.7476 | −0.06276 | 0.08017 | 0.7828 |
| CA_Bile Acids | 0.44144884 | 0.3005 | 0.1642 | 0.1363 | 0.1751 | 0.7784 |
| GLCA_Bile Acids | 0.44432966 | 0.0786 | 0.05948 | 0.01912 | 0.02469 | 0.7744 |
| TG(22:5_34:1)_Triacylglycerols | 0.44708394 | 1.71 | 1.463 | 0.2472 | 0.3196 | 0.7732 |
| TG(18:1_26:0)_Triacylglycerols | 0.4478696 | 0.9689 | 1.393 | −0.4243 | 0.5437 | 0.7805 |
| TG(22:2_32:4)_Triacylglycerols | 0.45238817 | 0.1405 | 0.1201 | 0.02036 | 0.02663 | 0.7644 |
| Creatinine_Aminoacids Related | 0.45319089 | 96.53 | 102 | −5.466 | 7.196 | 0.7596 |
| AA_Fatty Acids | 0.45998508 | 0.5078 | 0.4471 | 0.0607 | 0.08117 | 0.7478 |
| PC aa C36:6_ | 0.46148379 | 0.974 | 0.9006 | 0.07342 | 0.09853 | 0.7452 |
| Glycerophospholipids | | | | | | |
| Trp_Aminoacids | 0.46291036 | 74.56 | 78.72 | −4.164 | 5.606 | 0.7427 |
| TG(22:6_32:0)_Triacylglycerols | 0.46350399 | 0.649 | 0.5178 | 0.1312 | 0.1766 | 0.7426 |
| PC ae C36:4_ | 0.46409452 | 19.71 | 20.92 | −1.215 | 1.641 | 0.7402 |
| Glycerophospholipids | | | | | | |
| PC aa C38:4_ | 0.46460909 | 118.6 | 109.8 | 8.809 | 11.86 | 0.743 |
| Glycerophospholipids | | | | | | |
| TG(20:4_34:2)_Triacylglycerols | 0.46490763 | 3.614 | 4.377 | −0.7625 | 1.018 | 0.7488 |
| TG(14:0_36:4)_Triacylglycerols | 0.4737379 | 2.467 | 2.905 | −0.4376 | 0.5972 | 0.7329 |
| TG(20:4_32:2)_Triacylglycerols | 0.47452746 | 0.505 | 0.6227 | −0.1177 | 0.1608 | 0.7315 |
| PC ae C38:2_ | 0.47496485 | 2.35 | 2.236 | 0.1135 | 0.1567 | 0.7243 |
| Glycerophospholipids | | | | | | |
| C14:2_Acylcarnitines | 0.47896714 | 0.09509 | 0.108 | −0.01291 | 0.01778 | 0.7261 |
| TG(17:1_36:4)_Triacylglycerols | 0.47910644 | 0.3132 | 0.3575 | −0.04423 | 0.06129 | 0.7216 |
| TG(22:4_32:2)_Triacylglycerols | 0.48619227 | 0.1828 | 0.1531 | 0.02963 | 0.04194 | 0.7067 |
| SM C18:0_Sphingolipids | 0.48877281 | 22.13 | 20.69 | 1.434 | 2.04 | 0.7028 |
| Cer(d18:2/18:0)_Ceramides | 0.49210665 | 0.1683 | 0.1542 | 0.0141 | 0.02023 | 0.6973 |
| TMAO_Amine Oxides | 0.49284692 | 6.719 | 9.295 | −2.576 | 3.668 | 0.7022 |
| HexCer(d18:2/24:0)_ | 0.49348745 | 1.521 | 1.404 | 0.1166 | 0.1673 | 0.697 |
| Glycosylceramides | | | | | | |
| SM (OH) C24:1_Sphingolipids | 0.49448678 | 1.045 | 0.9876 | 0.05709 | 0.08235 | 0.6933 |
| TCDCA_Bile Acids | 0.49679077 | 0.1773 | 0.1437 | 0.03368 | 0.04906 | 0.6864 |
| TG(18:2_32:2)_Triacylglycerols | 0.49983743 | 4.25 | 5.025 | −0.7746 | 1.121 | 0.6907 |
| t4-OH-Pro_Aminoacids Related | 0.50222066 | 24.88 | 19.52 | 5.369 | 7.91 | 0.6788 |
| Cer(d16:1/24:0)_Ceramides | 0.50391621 | 0.3881 | 0.3528 | 0.03534 | 0.05217 | 0.6774 |
| PC aa C30:0_ | 0.50883025 | 4.989 | 4.652 | 0.337 | 0.502 | 0.6712 |
| Glycerophospholipids | | | | | | |
| Cer(d18:2/22:0)_Ceramides | 0.5091775 | 0.4196 | 0.4497 | −0.03014 | 0.04498 | 0.67 |
| TG(20:3_34:0)_Triacylglycerols | 0.50917821 | 0.3702 | 0.3235 | 0.04676 | 0.06976 | 0.6703 |
| ProBetaine_Aminoacids Related | 0.50942311 | 11.4 | 22.35 | −10.95 | 16.15 | 0.6779 |
| TG(18:1_33:2)_Triacylglycerols | 0.51271159 | 1.504 | 1.333 | 0.1715 | 0.2581 | 0.6642 |
| TG(18:3_38:5)_Triacylglycerols | 0.51618693 | 0.3599 | 0.3218 | 0.03803 | 0.05771 | 0.6589 |
| TG(16:1_28:0)_Triacylglycerols | 0.5202445 | 0.1913 | 0.3113 | −0.1199 | 0.1821 | 0.6587 |
| Cer(d18:2/24:1)_Ceramides | 0.52066074 | 0.3033 | 0.3192 | −0.0159 | 0.02442 | 0.651 |
| TG(20:0_32:4)_Triacylglycerols | 0.52419613 | 0.2601 | 0.2975 | −0.03742 | 0.05757 | 0.65 |
| HArg_Aminoacids Related | 0.52671841 | 2.28 | 2.502 | −0.2218 | 0.3461 | 0.6408 |
| Cer(d18:1/26:1)_Ceramides | 0.5299612 | 0.04227 | 0.03727 | 0.005004 | 0.007822 | 0.6397 |
| C5-DC (C6-OH)_Acylcarnitines | 0.53013289 | 0.1302 | 0.1258 | 0.004402 | 0.006891 | 0.6388 |
| TG(18:2_34:2)_Triacylglycerols | 0.53855149 | 39.98 | 46.05 | −6.076 | 9.658 | 0.6291 |
| TG(16:0_40:7)_Triacylglycerols | 0.53866804 | 0.8249 | 0.689 | 0.1359 | 0.2185 | 0.6219 |
| PC ae C40:2_ | 0.54036068 | 1.814 | 1.718 | 0.09638 | 0.1554 | 0.6201 |
| Glycerophospholipids | | | | | | |
| TG(16:0_28:1)_Triacylglycerols | 0.54467042 | 0.8932 | 1.229 | −0.3358 | 0.5419 | 0.6197 |
| TG(18:2_35:3)_Triacylglycerols | 0.54861123 | 0.4432 | 0.5008 | −0.0576 | 0.09412 | 0.6119 |
| PC aa C36:1_ | 0.55012037 | 48.71 | 46.18 | 2.523 | 4.166 | 0.6056 |
| Glycerophospholipids | | | | | | |
| Taurine_Aminoacids Related | 0.55047118 | 63.49 | 67.96 | −4.476 | 7.37 | 0.6074 |
| TG(18:2_38:6)_Triacylglycerols | 0.55316255 | 0.8552 | 0.7681 | 0.08715 | 0.1451 | 0.6006 |
| DG(18:2_18:2)_Diacylglycerols | 0.55360864 | 1.816 | 2.047 | −0.231 | 0.3822 | 0.6043 |
| TG(16:1_36:4)_Triacylglycerols | 0.55656369 | 3.07 | 3.543 | −0.4734 | 0.7868 | 0.6017 |
| TG(18:3_34:2)_Triacylglycerols | 0.55672012 | 4.993 | 5.738 | −0.7451 | 1.243 | 0.5997 |
| CE(18:3)_Cholesterol Esters | 0.55769348 | 36.81 | 33.3 | 3.515 | 5.894 | 0.5963 |
| PC ae C32:2_ | 0.55846787 | 1.046 | 1.112 | −0.06586 | 0.1104 | 0.5967 |
| Glycerophospholipids | | | | | | |
| PC ae C42:5_ | 0.55964019 | 2.065 | 2.138 | −0.07328 | 0.1242 | 0.5898 |
| Glycerophospholipids | | | | | | |
| PC ae C32:1_ | 0.56181319 | 3.132 | 3.268 | −0.1366 | 0.232 | 0.5888 |
| Glycerophospholipids | | | | | | |
| TG(16:0_36:4)_Triacylglycerols | 0.56211365 | 26.86 | 30.64 | −3.785 | 6.392 | 0.5922 |
| lysoPC a C16:0_ | 0.5639664 | 129.6 | 136.1 | −6.572 | 11.21 | 0.5865 |
| Glycerophospholipids | | | | | | |
| C16:1_Acylcarnitines | 0.5652941 | 0.06829 | 0.07458 | −0.006288 | 0.01067 | 0.5894 |
| TG(18:2_30:0)_Triacylglycerols | 0.56567306 | 4.217 | 5.015 | −0.7988 | 1.366 | 0.5845 |
| TG(18:2_34:4)_Triacylglycerols | 0.56635771 | 0.7763 | 0.8818 | −0.1054 | 0.1802 | 0.5852 |
| Gln_Aminoacids | 0.56747564 | 922.9 | 890.1 | 32.81 | 56.57 | 0.58 |
| TG(18:2_34:3)_Triacylglycerols | 0.5690527 | 6.353 | 7.278 | −0.9255 | 1.591 | 0.5817 |
| TLCA_Bile Acids | 0.57022912 | 0.003919 | 0.003238 | 0.0006803 | 0.001181 | 0.5759 |
| PC ae C34:2_ | 0.57032105 | 13.38 | 13.99 | −0.6097 | 1.06 | 0.5751 |
| Glycerophospholipids | | | | | | |
| TG(18:1_32:3)_Triacylglycerols | 0.5792813 | 1.162 | 1.307 | −0.1451 | 0.2569 | 0.5647 |
| TG(20:3_32:0)_Triacylglycerols | 0.57930855 | 0.5721 | 0.4758 | 0.09624 | 0.171 | 0.5629 |
| C16:2_Acylcarnitines | 0.58164132 | 0.03797 | 0.04063 | −0.002664 | 0.004718 | 0.5647 |
| TG(18:1_33:3)_Triacylglycerols | 0.58190183 | 0.4357 | 0.3843 | 0.05139 | 0.09188 | 0.5594 |
| C18_Acylcarnitines | 0.58206619 | 0.05027 | 0.05309 | −0.002826 | 0.005051 | 0.5595 |
| TDCA_Bile Acids | 0.5843783 | 0.1637 | 0.2044 | −0.04063 | 0.07272 | 0.5587 |
| TG(14:0_32:2)_Triacylglycerols | 0.58471278 | 0.807 | 0.9812 | −0.1742 | 0.3126 | 0.5572 |
| PC aa C42:5_ | 0.58495666 | 0.3532 | 0.3378 | 0.01545 | 0.02785 | 0.5548 |
| Glycerophospholipids | | | | | | |
| TG(18:0_38:6)_Triacylglycerols | 0.58612367 | 0.5128 | 0.4637 | 0.04911 | 0.08882 | 0.553 |
| lysoPC a C14:0_ | 0.5886235 | 2.391 | 2.571 | −0.18 | 0.3269 | 0.5508 |
| Glycerophospholipids | | | | | | |
| TG(20:4_32:0)_Triacylglycerols | 0.59033423 | 1.299 | 1.598 | −0.2987 | 0.5455 | 0.5476 |
| TG(20:5_34:1)_Triacylglycerols | 0.59132434 | 1.206 | 1.025 | 0.1807 | 0.3329 | 0.5428 |
| TG(16:0_28:2)_Triacylglycerols | 0.59475371 | 0.537 | 0.6652 | −0.1282 | 0.2364 | 0.5425 |
| PC ae C34:3_ | 0.59651379 | 10.01 | 10.61 | −0.6022 | 1.123 | 0.5364 |
| Glycerophospholipids | | | | | | |
| TG(18:1_33:1)_Triacylglycerols | 0.59741251 | 3.429 | 3.066 | 0.3637 | 0.6806 | 0.5344 |
| TG(17:0_36:3)_Triacylglycerols | 0.59844683 | 1.353 | 1.245 | 0.108 | 0.2024 | 0.5337 |
| TG(18:3_32:1)_Triacylglycerols | 0.60058149 | 1.857 | 2.152 | −0.2947 | 0.5531 | 0.5328 |
| Cer(d18:2/20:0)_Ceramides | 0.60307017 | 0.1512 | 0.1435 | 0.007757 | 0.01476 | 0.5254 |
| SM (OH) C16:1_Sphingolipids | 0.60470195 | 3.414 | 3.238 | 0.1764 | 0.3356 | 0.5256 |
| PC ae C38:4_ | 0.60656125 | 13.37 | 13.92 | −0.5436 | 1.044 | 0.5207 |
| Glycerophospholipids | | | | | | |
| TG(20:1_34:1)_Triacylglycerols | 0.60785704 | 0.5873 | 0.5324 | 0.05486 | 0.1054 | 0.5206 |
| C14_Acylcarnitines | 0.61017656 | 0.05075 | 0.05534 | −0.004587 | 0.00879 | 0.5218 |
| TG(18:3_30:0)_Triacylglycerols | 0.61139418 | 0.6635 | 0.7995 | 0.136 | 0.2627 | 0.5177 |
| PC aa C40:5_ | 0.61153706 | 8.676 | 8.028 | 0.6482 | 1.259 | 0.5149 |
| Glycerophospholipids | | | | | | |
| PC aa C34:2_ | 0.61173639 | 450.4 | 431.8 | 18.64 | 36.24 | 0.5143 |
| Glycerophospholipids | | | | | | |
| TG(17:2_34:3)_Triacylglycerols | 0.61174997 | 0.2274 | 0.2023 | 0.02504 | 0.0486 | 0.5151 |
| PC aa C34:3_ | 0.61219417 | 18.26 | 17.31 | 0.9439 | 1.839 | 0.5132 |
| Glycerophospholipids | | | | | | |
| TG(17:0_34:2)_Triacylglycerols | 0.61454209 | 0.901 | 0.81 | 0.09096 | 0.1783 | 0.5101 |
| GDCA_Bile Acids | 0.6161905 | 0.2832 | 0.336 | −0.05284 | 0.1035 | 0.5106 |
| PC aa C36:4_ | 0.6164944 | 206.6 | 197.2 | 9.439 | 18.61 | 0.5072 |
| Glycerophospholipids | | | | | | |
| TG(17:0_34:3)_Triacylglycerols | 0.61665702 | 0.4311 | 0.3774 | 0.05368 | 0.1057 | 0.5077 |
| PC aa C34:4_ | 0.61690175 | 2.089 | 2.23 | −0.141 | 0.2787 | 0.5062 |
| Glycerophospholipids | | | | | | |
| TG(17:2_36:2)_ | 0.61690981 | 0.2698 | 0.2476 | 0.0222 | 0.04385 | 0.5063 |
| Triacylglycerols | | | | | | |
| PC aa C42:6_ | 0.61850018 | 0.5042 | 0.4829 | 0.0213 | 0.04218 | 0.5049 |
| Glycerophospholipids | | | | | | |
| TG(18:3_32:0)_ | 0.618888 | 1.679 | 1.987 | −0.3082 | 0.6084 | 0.5065 |
| Triacylglycerols | | | | | | |
| TG(16:0_34:4)_Triacylglycerols | 0.61905331 | 1.155 | 1.353 | −0.1975 | 0.3904 | 0.506 |
| SM C26:0_Sphingolipids | 0.62346748 | 0.1699 | 0.1643 | 0.005544 | 0.01116 | 0.4968 |
| HexCer(d18:1/20:0)_ | 0.62410189 | 0.5806 | 0.5336 | 0.04701 | 0.09466 | 0.4966 |
| Glycosylceramides | | | | | | |
| TG(18:2_36:3)_Triacylglycerols | 0.62536882 | 16.74 | 18.79 | −2.055 | 4.14 | 0.4963 |
| TG(20:1_30:1)_Triacylglycerols | 0.62623564 | 0.2102 | 0.1922 | 0.01805 | 0.03672 | 0.4916 |
| TG(20:4_32:1)_Triacylglycerols | 0.62797387 | 1.328 | 1.575 | −0.2473 | 0.5016 | 0.493 |
| TG(18:2_32:0)_Triacylglycerols | 0.62916088 | 13.72 | 15.81 | −2.099 | 4.269 | 0.4916 |
| TG(16:0_36:5)_Triacylglycerols | 0.63100425 | 3.875 | 4.341 | −0.4661 | 0.9536 | 0.4887 |
| TG(16:1_38:5)_Triacylglycerols | 0.63174476 | 0.4947 | 0.4483 | 0.04644 | 0.09531 | 0.4873 |
| Cit_Aminoacids Related | 0.63264913 | 46.21 | 44.18 | 2.03 | 4.213 | 0.4819 |
| TG(16:0_30:2)_Triacylglycerols | 0.63495806 | 0.9849 | 1.14 | −0.155 | 0.3216 | 0.4821 |
| SM (OH) C22:1_Sphingolipids | 0.636067 | 11.01 | 10.58 | 0.429 | 0.8966 | 0.4785 |
| HCys_Aminoacids Related | 0.64221016 | 5.729 | 6.024 | −0.2953 | 0.627 | 0.471 |
| PC ae C38:6_ | 0.64302264 | 7.1 | 6.83 | 0.2704 | 0.5768 | 0.4687 |
| Glycerophospholipids | | | | | | |
| PC ae C42:3_Glycerophospholipids | 0.64317712 | 0.8254 | 0.8522 | −0.02671 | 0.05684 | 0.4699 |
| TG(18:1_30:1)_Triacylglycerols | 0.64818706 | 4.713 | 5.323 | −0.6105 | 1.318 | 0.4631 |
| TG(18:0_32:0)_Triacylglycerols | 0.64928683 | 1.088 | 1.561 | −0.4735 | 1.02 | 0.4641 |
| PC ae C36:5_Glycerophospholipids | 0.65003285 | 12.97 | 13.56 | −0.5891 | 1.284 | 0.4587 |
| TG(16:0_34:3)_Triacylglycerols | 0.65172903 | 9.629 | 11.08 | −1.456 | 3.169 | 0.4595 |
| TG(20:1_32:0)_Triacylglycerols | 0.65239947 | 0.09793 | 0.1081 | −0.01018 | 0.02227 | 0.4572 |
| TG(17:2_36:4)_Triacylglycerols | 0.65414177 | 0.2485 | 0.23 | 0.01846 | 0.04063 | 0.4543 |
| TG(14:0_34:2)_Triacylglycerols | 0.65655708 | 4.1 | 4.686 | −0.586 | 1.297 | 0.4517 |
| TG(18:2_36:2)_Triacylglycerols | 0.6581013 | 20.21 | 22.21 | −2.006 | 4.456 | 0.4501 |
| SM C24:0_Sphingolipids | 0.6581447 | 17.03 | 17.59 | −0.5645 | 1.264 | 0.4466 |
| PC aa C32:0_Glycerophospholipids | 0.65816722 | 14.71 | 14.24 | 0.4757 | 1.06 | 0.4486 |
| PC ae C34:1_Glycerophospholipids | 0.65876966 | 10.55 | 10.21 | 0.3453 | 0.773 | 0.4467 |
| PC ae C38:1_Glycerophospholipids | 0.65942422 | 0.6126 | 0.5875 | 0.02509 | 0.05623 | 0.4463 |
| TG(18:1_36:3)_Triacylglycerols | 0.6615903 | 35.46 | 39.07 | −3.614 | 8.118 | 0.4451 |
| SM C26:1_Sphingolipids | 0.6623757 | 0.268 | 0.2583 | 0.009729 | 0.022 | 0.4422 |
| His_Aminoacids | 0.66379327 | 118 | 114.6 | 3.389 | 7.724 | 0.4387 |
| TG(14:0_36:3)_Triacylglycerols | 0.66419007 | 6.104 | 6.687 | −0.5825 | 1.321 | 0.4411 |
| TG(17:1_34:1)_Triacylglycerols | 0.66457301 | 1.118 | 0.996 | 0.1221 | 0.2781 | 0.4391 |
| TG(18:3_34:3)_Triacylglycerols | 0.665489 | 0.9329 | 1.035 | −0.102 | 0.2319 | 0.44 |
| TG(22:4_34:2)_Triacylglycerols | 0.66555957 | 0.503 | 0.5544 | −0.05139 | 0.1168 | 0.4398 |
| TG(14:0_34:3)_Triacylglycerols | 0.66607075 | 1.195 | 1.356 | −0.1611 | 0.3671 | 0.4388 |
| Cer(d18:1/18:0)_Ceramides | 0.66967654 | 0.2839 | 0.2952 | −0.01126 | 0.02612 | 0.4314 |
| TG(16:1_32:2)_Triacylglycerols | 0.67144155 | 0.769 | 0.942 | −0.173 | 0.4004 | 0.432 |
| PC aa C36:3_ | 0.67175584 | 143.9 | 139.3 | 4.642 | 10.86 | 0.4275 |
| Glycerophospholipids | | | | | | |
| Hex3Cer(d18:1/16:0)_ | 0.67208119 | 2.343 | 2.417 | −0.07433 | 0.1734 | 0.4288 |
| Glycosylceramides | | | | | | |
| Cer(d18:1/24:0)_Ceramides | 0.67383479 | 3.255 | 3.389 | −0.134 | 0.315 | 0.4256 |
| TG(18:3_34:0)_Triacylglycerols | 0.67558841 | 1.111 | 1.237 | −0.1259 | 0.2958 | 0.4257 |
| TG(20:4_36:4)_Triacylglycerols | 0.68166532 | 1.063 | 1.138 | −0.0751 | 0.1802 | 0.4168 |
| PC aa C42:1_ | 0.68277744 | 0.3697 | 0.3606 | 0.009088 | 0.02196 | 0.4139 |
| Glycerophospholipids | | | | | | |
| TG(16:0_32:3)_Triacylglycerols | 0.68279103 | 1.154 | 1.323 | −0.1692 | 0.4074 | 0.4153 |
| TG(18:2_32:1)_Triacylglycerols | 0.6834659 | 13.46 | 14.96 | −1.501 | 3.62 | 0.4147 |
| C9_Acylcarnitines | 0.68371315 | 0.09554 | 0.0928 | 0.002737 | 0.006629 | 0.4129 |
| lysoPC a C18:2_ | 0.6854984 | 56.74 | 60.32 | −3.571 | 8.697 | 0.4106 |
| Glycerophospholipids | | | | | | |
| TG(20:1_32:1)_Triacylglycerols | 0.68647266 | 0.2927 | 0.2643 | 0.02841 | 0.06937 | 0.4096 |
| TG(18:1_35:2)_Triacylglycerols | 0.68752848 | 2.067 | 1.936 | 0.1311 | 0.3222 | 0.4071 |
| TG(16:1_36:1)_Triacylglycerols | 0.68813737 | 1.556 | 1.428 | 0.1282 | 0.3151 | 0.4068 |
| TG(20:4_34:1)_Triacylglycerols | 0.68838507 | 4.712 | 5.197 | −0.4847 | 1.189 | 0.4075 |
| TG(16:0_32:2)_Triacylglycerols | 0.6884385 | 5.333 | 6.094 | −0.7612 | 1.87 | 0.407 |
| PC aa C42:4_ | 0.68877151 | 0.2668 | 0.2598 | 0.006969 | 0.01719 | 0.4054 |
| Glycerophospholipids | | | | | | |
| TG(18:3_34:1)_Triacylglycerols | 0.68884408 | 7.746 | 8.471 | −0.7245 | 1.78 | 0.407 |
| TG(20:5_34:2)_Triacylglycerols | 0.6905542 | 1.01 | 0.8951 | 0.1145 | 0.285 | 0.4018 |
| AconAcid_Carboxylic Acids | 0.69194766 | 5.724 | 5.532 | 0.1925 | 0.4801 | 0.401 |
| Cer(d18:2/16:0)_Ceramides | 0.69357775 | 0.2084 | 0.2158 | −0.007362 | 0.01844 | 0.3992 |
| TG(16:0_38:5)_Triacylglycerols | 0.69358399 | 2.066 | 2.279 | −0.2122 | 0.5299 | 0.4004 |
| TG(20:3_34:2)_Triacylglycerols | 0.69514666 | 1.222 | 1.333 | −0.1108 | 0.2784 | 0.398 |
| SM C18:1_Sphingolipids | 0.69746508 | 10.15 | 9.728 | 0.4245 | 1.075 | 0.3948 |
| TG(18:1_36:2)_Triacylglycerols | 0.69803182 | 36.84 | 40.42 | −3.575 | 9.067 | 0.3943 |
| PC ae C40:5_ | 0.69813774 | 3.569 | 3.661 | −0.09225 | 0.2354 | 0.3919 |
| Glycerophospholipids | | | | | | |
| SM C24:1_Sphingolipids | 0.69914705 | 34.1 | 35.18 | −1.081 | 2.764 | 0.391 |
| TG(18:1_38:5)_Triacylglycerols | 0.699999 | 2.159 | 2.042 | 0.1174 | 0.3002 | 0.3909 |
| PC ae C42:1_ | 0.70888131 | 0.4695 | 0.4568 | 0.01264 | 0.03342 | 0.3781 |
| Glycerophospholipids | | | | | | |
| TG(18:1_36:1)_Triacylglycerols | 0.71526119 | 10.49 | 11.28 | −0.7861 | 2.124 | 0.3702 |
| TG(14:0_34:0)_Triacylglycerols | 0.71633161 | 0.6692 | 0.8841 | −0.2148 | 0.5797 | 0.3706 |
| TG(18:0_36:1)_Triacylglycerols | 0.71869038 | 1.378 | 1.566 | −0.1876 | 0.5114 | 0.3668 |
| TG(16:1_36:5)_Triacylglycerols | 0.71946051 | 0.6268 | 0.6825 | −0.05568 | 0.1523 | 0.3657 |
| TG(16:1_34:3)_Triacylglycerols | 0.72251959 | 1.703 | 1.914 | −0.2117 | 0.5856 | 0.3615 |
| TG(18:1_31:0)_Triacylglycerols | 0.72352751 | 1.436 | 1.268 | 0.1679 | 0.4682 | 0.3585 |
| TG(18:1_36:4)_Triacylglycerols | 0.72368886 | 14.26 | 15.36 | −1.096 | 3.058 | 0.3585 |
| TG(18:1_36:0)_Triacylglycerols | 0.72688371 | 1.663 | 1.814 | −0.1511 | 0.4256 | 0.3551 |
| TG(16:0_35:2)_Triacylglycerols | 0.72735213 | 1.783 | 1.629 | 0.1547 | 0.4382 | 0.353 |
| DG(16:0_18:1)_Diacylglycerols | 0.73191908 | 0.6331 | 0.686 | −0.05288 | 0.1523 | 0.3473 |
| PC ae C30:0_ | 0.73326865 | 0.5774 | 0.5945 | −0.01705 | 0.04943 | 0.345 |
| Glycerophospholipids | | | | | | |
| TG(16:0_32:0)_Triacylglycerols | 0.7338751 | 4.533 | 5.744 | −1.211 | 3.509 | 0.3451 |
| lysoPC a C20:4_ | 0.73785932 | 9.843 | 10.18 | −0.3343 | 0.9891 | 0.338 |
| Glycerophospholipids | | | | | | |
| PC aa C40:2_ | 0.74489373 | 0.354 | 0.3464 | 0.007615 | 0.02315 | 0.329 |
| Glycerophospholipids | | | | | | |
| C14:1_Acylcarnitines | 0.74556657 | 0.119 | 0.1155 | 0.003432 | 0.01043 | 0.3292 |
| TG(18:2_36:5)_Triacylglycerols | 0.74671273 | 2.061 | 1.866 | 0.1951 | 0.5997 | 0.3253 |
| Ind-SO4_Indoles Derivatives | 0.74701451 | 1.87 | 1.788 | 0.0819 | 0.2519 | 0.3251 |
| TG(18:0_36:5)_Triacylglycerols | 0.74915504 | 0.5009 | 0.4738 | 0.02716 | 0.08404 | 0.3232 |
| FA(18:1)_Fatty Acids | 0.75457537 | 115.5 | 125.3 | −9.792 | 30.97 | 0.3161 |
| TG(16:0_38:4)_Triacylglycerols | 0.75567069 | 1.754 | 1.881 | −0.1276 | 0.4043 | 0.3157 |
| PC aa C32:2_ | 0.75701177 | 4.694 | 4.91 | 0.2159 | 0.6888 | 0.3135 |
| Glycerophospholipids | | | | | | |
| TG(17:0_34:1)_Triacylglycerols | 0.75904377 | 1.026 | 0.9419 | 0.08363 | 0.2691 | 0.3108 |
| TG(20:4_33:2)_Triacylglycerols | 0.76400742 | 0.3239 | 0.3443 | −0.02037 | 0.06691 | 0.3044 |
| Putrescine_Biogenic Amines | 0.765083 | 0.1141 | 0.1204 | −0.006234 | 0.02066 | 0.3018 |
| TG(16:1_34:2)_Triacylglycerols | 0.76574569 | 7.771 | 8.549 | 0.7785 | 2.571 | 0.3028 |
| TG(18:2_35:2)_Triacylglycerols | 0.76790038 | 1.182 | 1.249 | −0.06705 | 0.2238 | 0.2996 |
| TG(14:0_34:1)_Triacylglycerols | 0.76850061 | 5.579 | 6.168 | −0.5893 | 1.973 | 0.2987 |
| Cer(d18:1/22:0)_Ceramides | 0.76856693 | 1.204 | 1.234 | −0.02982 | 0.1003 | 0.2972 |
| TG(20:3_36:4)_Triacylglycerols | 0.77204674 | 0.4412 | 0.4241 | 0.01713 | 0.0583 | 0.2939 |
| TG(17:0_36:4)_Triacylglycerols | 0.7723352 | 0.6179 | 0.6499 | −0.03207 | 0.1093 | 0.2933 |
| PC aa C36:0_ | 0.77382882 | 1.349 | 1.297 | 0.05261 | 0.1805 | 0.2915 |
| Glycerophospholipids | | | | | | |
| TG(18:3_36:4)_Triacylglycerols | 0.77777585 | 1.574 | 1.457 | 0.1177 | 0.4141 | 0.2842 |
| TG(18:0_36:2)_Triacylglycerols | 0.77859522 | 4.612 | 4.89 | −0.2778 | 0.9743 | 0.2851 |
| TG(18:1_34:1)_Triacylglycerols | 0.77934351 | 105.2 | 100 | 5.174 | 18.25 | 0.2835 |
| FA(20:3)_Fatty Acids | 0.77935417 | 0.4599 | 0.4876 | −0.02769 | 0.09772 | 0.2834 |
| PC ae C40:3_ | 0.78167884 | 1.133 | 1.153 | −0.02037 | 0.07276 | 0.28 |
| Glycerophospholipids | | | | | | |
| TG(16:1_32:1)_Triacylglycerols | 0.78296359 | 2.245 | 2.574 | −0.3292 | 1.177 | 0.2797 |
| lysoPC a C17:0_ | 0.78299085 | 2.9 | 2.98 | 0.08 | 0.2871 | 0.2787 |
| Glycerophospholipids | | | | | | |
| TG(17:0_32:1)_Triacylglycerols | 0.78360573 | 0.375 | 0.3314 | 0.04362 | 0.1568 | 0.2781 |
| PC ae C34:0_ | 0.78388427 | 1.537 | 1.497 | 0.04065 | 0.1466 | 0.2772 |
| Glycerophospholipids | | | | | | |
| TG(18:2_36:1)_Triacylglycerols | 0.78411605 | 6.506 | 6.845 | −0.3398 | 1.222 | 0.278 |
| TG(20:4_30:0)_Triacylglycerols | 0.7851462 | 0.575 | 0.6363 | −0.06121 | 0.2216 | 0.2762 |
| TG(16:0_38:6)_Triacylglycerols | 0.78563831 | 1.299 | 1.395 | −0.09565 | 0.3467 | 0.2759 |
| PC ae C36:2_ | 0.78634649 | 16.12 | 15.79 | 0.3303 | 1.206 | 0.2739 |
| Glycerophospholipids | | | | | | |
| DG(16:1_18:1)_Diacylglycerols | 0.78841294 | 0.3203 | 0.2973 | 0.02305 | 0.08454 | 0.2726 |
| TG(18:1_32:2)_Triacylglycerols | 0.78892704 | 8.108 | 8.576 | −0.468 | 1.724 | 0.2715 |
| PC aa C40:4_ | 0.79277799 | 3.27 | 3.402 | −0.1327 | 0.4982 | 0.2663 |
| Glycerophospholipids | | | | | | |
| TG(20:0_34:1)_Triacylglycerols | 0.79480794 | 0.1477 | 0.1392 | 0.008509 | 0.03233 | 0.2632 |
| TG(16:0_38:7)_Triacylglycerols | 0.79611963 | 0.2832 | 0.2532 | 0.02996 | 0.1145 | 0.2617 |
| TG(17:1_36:5)_Triacylglycerols | 0.79664328 | 0.1827 | 0.1943 | −0.01159 | 0.04441 | 0.2611 |
| TG(20:3_32:2)_Triacylglycerols | 0.79713024 | 0.2553 | 0.2379 | 0.0174 | 0.06685 | 0.2602 |
| TG(16:1_36:2)_Triacylglycerols | 0.79719128 | 7.844 | 7.463 | 0.381 | 1.462 | 0.2606 |
| TG(18:1_34:4)_Triacylglycerols | 0.7976553 | 1.388 | 1.466 | −0.07842 | 0.301 | 0.2605 |
| DG(14:0_14:0)_Diacylglycerols | 0.79838848 | 7.26 | 7.385 | −0.1242 | 0.48 | 0.2588 |
| PC aa C42:2_ | 0.80164365 | 0.2848 | 0.2898 | −0.005031 | 0.01976 | 0.2546 |
| Glycerophospholipids | | | | | | |
| TG(18:1_30:0)_Triacylglycerols | 0.80298447 | 7.771 | 8.468 | −0.6967 | 2.753 | 0.2531 |
| TG(18:1_33:0)_Triacylglycerols | 0.80308961 | 1.357 | 1.259 | 0.09799 | 0.3879 | 0.2526 |
| Cer(d18:1/20:0)_Ceramides | 0.81250627 | 0.2108 | 0.206 | 0.004778 | 0.01991 | 0.2399 |
| PC ae C36:0_ | 0.813619 | 0.7989 | 0.7848 | 0.01401 | 0.05878 | 0.2383 |
| Glycerophospholipids | | | | | | |
| TG(18:0_36:4)_Triacylglycerols | 0.81400636 | 2.796 | 2.94 | −0.1445 | 0.6051 | 0.2389 |
| TG(16:0_34:0)_Triacylglycerols | 0.81637992 | 4.606 | 5.148 | −0.5416 | 2.297 | 0.2357 |
| TG(18:0_30:1)_Triacylglycerols | 0.81704823 | 0.5087 | 0.5698 | −0.06115 | 0.2598 | 0.2353 |
| TG(16:0_36:3)_Triacylglycerols | 0.82396403 | 69.79 | 72.9 | −3.111 | 13.78 | 0.2258 |
| TG(18:2_38:5)_Triacylglycerols | 0.82399481 | 1.332 | 1.391 | −0.05957 | 0.2636 | 0.226 |
| TG(16:0_34:2)_Triacylglycerols | 0.8255593 | 32.41 | 34.52 | −2.116 | 9.467 | 0.2235 |
| TG(16:0_40:6)_Triacylglycerols | 0.83107358 | 1.073 | 1.124 | −0.0511 | 0.2363 | 0.2162 |
| TG(20:4_36:3)_Triacylglycerols | 0.83110566 | 2.314 | 2.242 | 0.07186 | 0.3321 | 0.2164 |
| TG(16:0_35:3)_Triacylglycerols | 0.83139273 | 0.8655 | 0.8215 | 0.04394 | 0.204 | 0.2154 |
| TG(20:4_36:2)_Triacylglycerols | 0.83351093 | 3.141 | 3.047 | 0.09456 | 0.4436 | 0.2132 |
| PC ae C38:3_ | 0.83357275 | 4.351 | 4.422 | −0.07083 | 0.3344 | 0.2118 |
| Glycerophospholipids | | | | | | |
| TG(18:2_34:1)_Triacylglycerols | 0.83466491 | 63.09 | 65.76 | −2.669 | 12.6 | 0.2118 |
| ADMA_Aminoacids Related | 0.8351315 | 0.5681 | 0.5582 | 0.009957 | 0.04727 | 0.2106 |
| TG(18:2_33:0)_Triacylglycerols | 0.83528013 | 0.8778 | 0.8337 | 0.04409 | 0.2094 | 0.2105 |
| TG(16:0_38:3)_Triacylglycerols | 0.83551969 | 1.349 | 1.298 | 0.05114 | 0.2433 | 0.2102 |
| TG(18:3_33:2)_Triacylglycerols | 0.83624436 | 0.3233 | 0.3089 | 0.01432 | 0.06849 | 0.2091 |
| TG(16:0_35:1)_Triacylglycerols | 0.83758557 | 1.23 | 1.151 | 0.07851 | 0.378 | 0.2077 |
| TG(16:1_36:3)_Triacylglycerols | 0.83809709 | 7.694 | 8.038 | −0.344 | 1.657 | 0.2077 |
| TG(18:2_34:0)_Triacylglycerols | 0.83814467 | 8.317 | 8.688 | −0.3703 | 1.788 | 0.2071 |
| Cer(d18:1/24:1)_Ceramides | 0.83933334 | 1.397 | 1.418 | −0.02106 | 0.1031 | 0.2042 |
| DG(18:1_18:2)_Diacylglycerols | 0.84094536 | 2.884 | 2.989 | −0.1048 | 0.5151 | 0.2034 |
| GUDCA_Bile Acids | 0.84295082 | 0.07787 | 0.07218 | 0.005686 | 0.0285 | 0.1995 |
| SM (OH) C22:2_Sphingolipids | 0.84538591 | 8.319 | 8.155 | 0.1631 | 0.8244 | 0.1979 |
| PC aa C42:0_ | 0.84697728 | 0.5769 | 0.5691 | 0.007849 | 0.04021 | 0.1952 |
| Glycerophospholipids | | | | | | |
| TG(16:0_36:2)_Triacylglycerols | 0.84795528 | 63.09 | 60.92 | 2.177 | 11.22 | 0.194 |
| TG(20:0_32:3)_Triacylglycerols | 0.84843059 | 0.2877 | 0.2781 | 0.009533 | 0.04928 | 0.1934 |
| GLCAS_Bile Acids | 0.84848781 | 1.151 | 1.067 | 0.08451 | 0.435 | 0.1943 |
| TG(18:2_38:4)_Triacylglycerols | 0.85067213 | 1.056 | 1.096 | −0.04034 | 0.2109 | 0.1913 |
| TG(18:2_35:1)_Triacylglycerols | 0.85353636 | 1.361 | 1.317 | 0.0434 | 0.2325 | 0.1867 |
| TG(18:0_36:3)_Triacylglycerols | 0.86338724 | 5.828 | 6.015 | 0.1872 | 1.074 | 0.1744 |
| HexCer(d18:2/22:0)_ | 0.86553249 | 1.135 | 1.108 | 0.02763 | 0.1614 | 0.1712 |
| Glycosylceramides | | | | | | |
| PC aa C40:3_ | 0.86665656 | 0.5404 | 0.5467 | −0.006285 | 0.03706 | 0.1696 |
| Glycerophospholipids | | | | | | |
| TG(18:2_36:4)_Triacylglycerols | 0.86904945 | 8.259 | 8.646 | −0.3876 | 2.331 | 0.1663 |
| Hex2Cer(d18:1/14:0)_ | 0.8708036 | 0.6371 | 0.6228 | 0.01432 | 0.08718 | 0.1642 |
| Glycosylceramides | | | | | | |
| TG(20:2_34:3)_Triacylglycerols | 0.87108704 | 0.3025 | 0.2934 | 0.009089 | 0.05539 | 0.1641 |
| TG(22:5_34:2)_Triacylglycerols | 0.87306079 | 1.284 | 1.331 | −0.04698 | 0.29 | 0.162 |
| TG(18:1_35:3)_Triacylglycerols | 0.87538402 | 0.7351 | 0.7153 | 0.01977 | 0.1244 | 0.1589 |
| Hex3Cer(d18:1/24:1)_ | 0.87585981 | 1.19 | 1.215 | −0.02535 | 0.1601 | 0.1583 |
| Glycosylceramides | | | | | | |
| TG(18:2_31:0)_Triacylglycerols | 0.87846314 | 0.6919 | 0.6631 | 0.02873 | 0.1857 | 0.1547 |
| TG(14:0_36:1)_Triacylglycerols | 0.87978323 | 1.355 | 1.424 | −0.06933 | 0.4514 | 0.1536 |
| TG(16:0_33:2)_Triacylglycerols | 0.88047544 | 0.8797 | 0.84 | 0.03973 | 0.2611 | 0.1522 |
| TG(16:0_33:1)_Triacylglycerols | 0.88348937 | 1.58 | 1.496 | 0.08384 | 0.565 | 0.1484 |
| TG(16:0_32:1)_Triacylglycerols | 0.88416371 | 10.22 | 10.79 | −0.5732 | 3.884 | 0.1476 |
| TG(18:1_34:2)_Triacylglycerols | 0.88738142 | 73.83 | 75.87 | −2.043 | 14.22 | 0.1436 |
| TG(18:1_32:0)_Triacylglycerols | 0.88757129 | 22.87 | 23.85 | −0.9822 | 6.856 | 0.1433 |
| PC ae C44:3_ | 0.88999041 | 0.2536 | 0.2554 | −0.001829 | 0.01309 | 0.1397 |
| Glycerophospholipids | | | | | | |
| PC aa C32:3_ | 0.89143426 | 0.7669 | 0.777 | −0.01007 | 0.07283 | 0.1383 |
| Glycerophospholipids | | | | | | |
| TG(16:1_32:0)_Triacylglycerols | 0.89850095 | 1.676 | 1.79 | −0.1138 | 0.8811 | 0.1291 |
| TG(18:1_34:3)_Triacylglycerols | 0.90061587 | 12.53 | 12.85 | −0.3201 | 2.526 | 0.1267 |
| DG(16:0_18:2)_Diacylglycerols | 0.9057745 | 0.6169 | 0.6331 | −0.01616 | 0.1349 | 0.1198 |
| lysoPC a C18:0_ | 0.90762245 | 42.96 | 43.45 | −0.4983 | 4.242 | 0.1175 |
| Glycerophospholipids | | | | | | |
| TG(14:0_38:5)_Triacylglycerols | 0.91115043 | 0.4287 | 0.4179 | 0.01081 | 0.09556 | 0.1131 |
| TMCA_Bile Acids | 0.91144034 | 0.01782 | 0.01875 | −0.0009316 | 0.008251 | 0.1129 |
| TG(20:3_34:3)_Triacylglycerols | 0.91521292 | 0.3683 | 0.3596 | 0.008658 | 0.08001 | 0.1082 |
| TG(18:3_35:2)_Triacylglycerols | 0.91946647 | 0.3557 | 0.3636 | −0.007972 | 0.07785 | 0.1024 |
| lysoPC a C16:1_ | 0.92028915 | 3.742 | 3.698 | 0.04453 | 0.4393 | 0.1014 |
| Glycerophospholipids | | | | | | |
| TG(20:2_34:4)_Triacylglycerols | 0.92177398 | 0.03683 | 0.03759 | −0.0007615 | 0.007684 | 0.0991 |
| CE(15:0)_Cholesterol Esters | 0.92234867 | 3.017 | 3.043 | −0.02641 | 0.2685 | 0.09837 |
| TG(20:2_34:2)_Triacylglycerols | 0.92237287 | 0.6371 | 0.6255 | 0.01154 | 0.1168 | 0.09878 |
| TG(18:2_36:0)_Triacylglycerols | 0.92294452 | 0.8953 | 0.9133 | −0.01801 | 0.1838 | 0.09802 |
| TG(18:2_33:1)_Triacylglycerols | 0.93039354 | 2.023 | 1.987 | 0.03581 | 0.4052 | 0.0884 |
| TG(20:4_34:0)_Triacylglycerols | 0.93081513 | 0.7918 | 0.81 | −0.01824 | 0.2074 | 0.08796 |
| TG(20:2_34:1)_Triacylglycerols | 0.93230958 | 0.8117 | 0.7991 | 0.01259 | 0.1464 | 0.08598 |
| TG(18:3_36:2)_Triacylglycerols | 0.93737746 | 3.484 | 3.435 | 0.04946 | 0.6236 | 0.07932 |
| TG(17:1_32:1)_Triacylglycerols | 0.94179347 | 0.3759 | 0.3662 | 0.009725 | 0.1316 | 0.07391 |
| Orn_Aminoacids Related | 0.9452308 | 105.1 | 105.9 | −0.7467 | 10.76 | 0.06943 |
| C16_Acylcarnitines | 0.94644983 | 0.1199 | 0.1206 | −0.0007048 | 0.01038 | 0.06789 |
| PC ae C30:1_ | 0.94839164 | 2.5 | 2.485 | 0.01425 | 0.2177 | 0.06544 |
| Glycerophospholipids | | | | | | |
| TG(14:0_36:2)_Triacylglycerols | 0.94911061 | 6.104 | 6.024 | 0.0806 | 1.249 | 0.06455 |
| TG(18:1_32:1)_Triacylglycerols | 0.95067706 | 25.44 | 25.06 | 0.3817 | 6.096 | 0.06261 |
| TG(22:4_32:0)_Triacylglycerols | 0.95288001 | 0.2572 | 0.2621 | −0.004948 | 0.0827 | 0.05984 |
| TG(18:2_33:2)_Triacylglycerols | 0.95361177 | 0.911 | 0.9219 | −0.01089 | 0.1846 | 0.05897 |
| TG(18:0_32:1)_Triacylglycerols | 0.95447339 | 1.474 | 1.512 | −0.03864 | 0.6674 | 0.0579 |
| TG(16:0_36:6)_Triacylglycerols | 0.9562618 | 0.4665 | 0.4588 | 0.007672 | 0.1381 | 0.05556 |
| TG(18:0_32:2)_Triacylglycerols | 0.95800766 | 0.7045 | 0.7178 | −0.01329 | 0.249 | 0.05339 |
| TG(17:1_36:3)_Triacylglycerols | 0.96081868 | 0.5337 | 0.5385 | −0.004872 | 0.09789 | 0.04977 |
| TG(17:2_38:5)_Triacylglycerols | 0.96350116 | 0.0925 | 0.09329 | −0.000787 | 0.01702 | 0.04625 |
| TG(20:4_34:3)_Triacylglycerols | 0.96376957 | 0.7436 | 0.7516 | −0.007986 | 0.1733 | 0.04609 |
| CO_Acylcarnitines | 0.96496978 | 46.67 | 46.51 | 0.159 | 3.587 | 0.04433 |
| TG(18:3_36:3)_Triacylglycerols | 0.96572795 | 3.351 | 3.382 | −0.03157 | 0.7282 | 0.04335 |
| PC ae C38:5_ | 0.96908435 | 18.6 | 18.65 | −0.05014 | 1.284 | 0.03904 |
| Glycerophospholipids | | | | | | |
| DG(18:1_18:1)_Diacylglycerols | 0.96983419 | 0.8598 | 0.8652 | −0.005453 | 0.1425 | 0.03826 |
| Cer(d18:2/23:0)_Ceramides | 0.96987045 | 0.2019 | 0.2027 | −0.000796 | 0.02089 | 0.03811 |
| TG(17:1_34:2)_Triacylglycerols | 0.97160143 | 0.7855 | 0.7788 | 0.006714 | 0.1863 | 0.03603 |
| TG(16:0_34:1)_Triacylglycerols | 0.97173615 | 36.43 | 36.82 | −0.3856 | 10.75 | 0.03586 |
| PC ae C42:2_ | 0.9721665 | 0.5696 | 0.5711 | −0.001447 | 0.04106 | 0.03524 |
| Glycerophospholipids | | | | | | |
| TG(20:3_32:1)_Triacylglycerols | 0.97248067 | 0.5801 | 0.5748 | 0.005295 | 0.1517 | 0.0349 |
| TG(16:1_34:1)_Triacylglycerols | 0.97559164 | 11.41 | 11.3 | 0.105 | 3.388 | 0.03099 |
| TG(16:0_37:3)_Triacylglycerols | 0.97756503 | 0.3814 | 0.3792 | 0.002201 | 0.07742 | 0.02843 |
| TG(18:1_36:5)_Triacylglycerols | 0.97826188 | 3.256 | 3.238 | 0.01807 | 0.6572 | 0.02749 |
| TG(18:0_34:2)_Triacylglycerols | 0.97855206 | 3.762 | 3.733 | 0.02915 | 1.071 | 0.0272 |
| TG(20:1_34:2)_Triacylglycerols | 0.97922508 | 0.4732 | 0.4712 | 0.001943 | 0.07394 | 0.02628 |
| Trigonelline_Alkaloids | 0.97978234 | 5.157 | 5.24 | −0.08345 | 3.234 | 0.0258 |
| PC aa C38:3_ | 0.9822057 | 46.51 | 46.38 | 0.1302 | 5.776 | 0.02254 |
| Glycerophospholipids | | | | | | |
| TG(16:1_34:0)_Triacylglycerols | 0.98406994 | 1.546 | 1.536 | 0.01043 | 0.516 | 0.02021 |
| TG(16:0_38:2)_Triacylglycerols | 0.98416882 | 0.8391 | 0.8422 | −0.003157 | 0.1572 | 0.02008 |
| TG(20:3_34:1)_Triacylglycerols | 0.98425331 | 1.627 | 1.62 | 0.006595 | 0.3303 | 0.01997 |
| SM C16:0_Sphingolipids | 0.985115 | 109.4 | 109.3 | 0.1607 | 8.511 | 0.01888 |
| TG(18:0_34:3)_Triacylglycerols | 0.98596643 | 0.8686 | 0.8648 | 0.003746 | 0.2106 | 0.01779 |
| TG(22:5_32:1)_Triacylglycerols | 0.98792192 | 0.4889 | 0.491 | 0.002051 | 0.1335 | 0.01536 |
| SM C16:1_Sphingolipids | 0.99014196 | 16.27 | 16.29 | −0.01949 | 1.555 | 0.01253 |
| TG(18:3_36:1)_Triacylglycerols | 0.99201564 | 0.9895 | 0.9912 | −0.001749 | 0.1728 | 0.01012 |
| TG(16:0_38:1)_Triacylglycerols | 0.99621549 | 0.3536 | 0.354 | −0.0003897 | 0.08119 | 0.0048 |
| CE(15:1)_Cholesterol Esters | 0.99785969 | 0.6946 | 0.6944 | 0.000208 | 0.0769 | 0.002704 |
| TCA_Bile Acids | 0.99890351 | 0.1121 | 0.1122 | −0.00006463 | 0.04672 | 0.001383 |
| lysoPC a C18:1_ | 0.99928028 | 27.96 | 27.96 | −0.002279 | 2.505 | 0.00091 |
| Glycerophospholipids | | | | | | |
| | | | | | | Candidate |
| Metabolite | df | q value | Ratio | LogFC | -LogP | Biomarker |
| PC aa C24:0_ | 37.12 | 0.24778712 | 0.821 | −0.28455 | 3.306537 | YES |
| Glycerophospholipids | | | | | | |
| 5-AVA_Aminoacids Related | 37.57 | 0.3447046 | 0.535406 | −0.90129 | 2.86214 | YES |
| Cystine_Aminoacids Related | 37.53 | 0.47619131 | 1.811626 | 0.857285 | 2.545715 | YES |
| Cer(d16:1/20:0)_Ceramides | 36.16 | 0.79697636 | 0.687751 | −0.54004 | 2.149197 | YES |
| lysoPC a C26:0_ | 34.35 | 0.79697636 | 0.862109 | −0.21406 | 2.027241 | YES |
| Glycerophospholipids | | | | | | |
| Asp_Aminoacids | 37.31 | 0.79697636 | 0.755938 | −0.40366 | 1.98971 | YES |
| Cys_Aminoacids | 27.49 | 0.79697636 | 1.298348 | 0.376677 | 1.954074 | YES |
| Tyr_Aminoacids | 35.73 | 0.87392388 | 0.830508 | −0.26793 | 1.856054 | YES |
| PC aa C26:0_ | 26.35 | 0.90646623 | 0.740882 | −0.43268 | 1.789023 | YES |
| Glycerophospholipids | | | | | | |
| lysoPC a C28:0_ | 28.76 | >0.99999999 | 0.894034 | 0.1616 | 1.585027 | YES |
| Glycerophospholipids | | | | | | |
| C18:2_Acylcarnitines | 19.34 | >0.99999999 | 1.202679 | 0.266251 | 1.530741 | YES |
| Cer(d18:2/18:1)_Ceramides | 28.41 | >0.99999999 | 0.631966 | −0.66208 | 1.447105 | YES |
| Cer(d16:1/18:0)_Ceramides | 33.39 | >0.99999999 | 0.721292 | −0.47134 | 1.433857 | YES |
| p-Cresol-SO4_Cresols | 30.82 | >0.99999999 | 0.586978 | −0.76862 | 1.432805 | YES |
| PC aa C40:6_ | 30.59 | >0.99999999 | 0.804119 | −0.31452 | 1.388573 | YES |
| Glycerophospholipids | | | | | | |
| C16-OH_Acylcarnitines | 26.66 | >0.99999999 | 0.887287 | −0.17253 | 1.38497 | YES |
| Gly_Aminoacids | 35.99 | >0.99999999 | 0.825252 | −0.27709 | 1.379791 | YES |
| Hypoxanthine_ | 14.88 | >0.99999999 | 1.813365 | 0.85867 | 1.348644 | YES |
| Nucleobases Related | | | | | | |
| EPA_Fatty Acids | 31.9 | >0.99999999 | 0.704287 | −0.50576 | 1.329832 | YES |
| C14:2-OH_Acylcarnitines | 21.24 | >0.99999999 | 0.845894 | −0.24145 | 1.322138 | YES |
| 3-IPA_Indoles Derivatives | 37.96 | >0.99999999 | 0.650287 | −0.62085 | 1.270907 | NO |
| lysoPC a C26:1_ | 22.83 | >0.99999999 | 0.890255 | 0.16771 | 1.24629 | NO |
| Glycerophospholipids | | | | | | |
| C5-M-DC_Acylcarnitines | 35.72 | >0.99999999 | 0.650927 | −0.61943 | 1.137993 | NO |
| CE(18:1)_Cholesterol Esters | 28.88 | >0.99999999 | 0.850075 | −0.23434 | 1.137742 | NO |
| lysoPC a C24:0_ | 23.3 | >0.99999999 | 0.91787 | −0.12364 | 1.135556 | NO |
| Glycerophospholipids | | | | | | |
| CE(14:0)_Cholesterol Esters | 23.23 | >0.99999999 | 0.702614 | −0.5092 | 1.130181 | NO |
| C4_Acylcarnitines | 31.93 | >0.99999999 | 0.861006 | −0.2159 | 1.106642 | NO |
| DHEAS_Hormones | 23.37 | >0.99999999 | 1.595233 | 0.673767 | 1.08108 | NO |
| HexCer(d18:1/24:0)_ | 22.93 | >0.99999999 | 1.173575 | 0.23091 | 1.079792 | NO |
| Glycosylceramides | | | | | | |
| Pro_Aminoacids | 34.35 | >0.99999999 | 0.817411 | −0.29087 | 1.073977 | NO |
| TG(18:1_38:7)_Triacylglycerols | 26.1 | >0.99999999 | 0.779237 | −0.35987 | 1.044172 | NO |
| lysoPC a C28:1_ | 25.06 | >0.99999999 | 0.880321 | −0.1839 | 1.031964 | NO |
| Glycerophospholipids | | | | | | |
| CE(20:5)_Cholesterol Esters | 36.84 | >0.99999999 | 0.724641 | −0.46466 | 1.030567 | NO |
| AABA_Aminoacids Related | 34.54 | >0.99999999 | 0.851388 | −0.23211 | 1.021457 | NO |
| C3-OH_Acylcarnitines | 27.64 | >0.99999999 | 0.920377 | −0.1197 | 1.016329 | NO |
| Sarcosine_Aminoacids Related | 12.24 | >0.99999999 | 2.173242 | 1.119849 | 1.006911 | NO |
| TG(20:5_36:2)_Triacylglycerols | 37.81 | >0.99999999 | 0.681385 | −0.55346 | 1.006574 | NO |
| CE(16:1)_Cholesterol Esters | 28.99 | >0.99999999 | 0.734717 | −0.44474 | 1.001258 | NO |
| PC aa C38:6_ | 27.64 | >0.99999999 | 0.843427 | −0.24567 | 0.996014 | NO |
| Glycerophospholipids | | | | | | |
| C18:1-OH_Acylcarnitines | 26.11 | >0.99999999 | 0.910266 | −0.13564 | 0.967674 | NO |
| BABA_Aminoacids Related | 28.61 | >0.99999999 | 0.808333 | −0.30698 | 0.95962 | NO |
| Cer(d18:1/18:1)_Ceramides | 26.67 | >0.99999999 | 0.783024 | −0.35287 | 0.95578 | NO |
| Cer(d18:1/25:0)_Ceramides | 29.6 | >0.99999999 | 0.868352 | −0.20365 | 0.948919 | NO |
| Choline_Vitamins & Cofactors | 31.91 | >0.99999999 | 0.863784 | −0.21126 | 0.946403 | NO |
| C14:1-OH_Acylcarnitines | 26.09 | >0.99999999 | 0.901438 | −0.1497 | 0.940533 | NO |
| Cortisol_Hormones | 15.23 | >0.99999999 | 1.316598 | 0.396815 | 0.938777 | NO |
| Cer(d16:1/23:0)_Ceramides | 29.76 | >0.99999999 | 0.803396 | −0.31582 | 0.915094 | NO |
| Thr_Aminoacids | 37.39 | >0.99999999 | 0.841356 | −0.24921 | 0.909771 | NO |
| Met_Aminoacids | 37.57 | >0.99999999 | 0.833853 | −0.26214 | 0.900304 | NO |
| PC ae C30:2_ | 24.08 | >0.99999999 | 0.91115 | −0.13424 | 0.884824 | NO |
| Glycerophospholipids | | | | | | |
| Arg_Aminoacids | 29.66 | >0.99999999 | 0.864356 | −0.2103 | 0.882646 | NO |
| C7-DC_Acylcarnitines | 33.41 | >0.99999999 | 0.864564 | −0.20996 | 0.879898 | NO |
| HexCer(d18:2/23:0)_ | 23.34 | >0.99999999 | 1.268081 | 0.342647 | 0.870578 | NO |
| Glycosylceramides | | | | | | |
| Ser_Aminoacids | 35.14 | >0.99999999 | 0.877497 | −0.18853 | 0.868331 | NO |
| GABA_Biogenic Amines | 29.16 | >0.99999999 | 0.898262 | −0.15479 | 0.859863 | NO |
| CE(22:6)_Cholesterol Esters | 21.48 | >0.99999999 | 0.809947 | −0.3041 | 0.830696 | NO |
| C3:1_Acylcarnitines | 26.24 | >0.99999999 | 0.927046 | −0.10929 | 0.820007 | NO |
| Xanthine_Nucleobases Related | 36.8 | >0.99999999 | 1.161011 | 0.215382 | 0.808903 | NO |
| CE(16:0)_Cholesterol Esters | 24.29 | >0.99999999 | 0.867412 | −0.20521 | 0.808211 | NO |
| alpha-AAA_Aminoacids | 32.11 | >0.99999999 | 0.692618 | −0.52987 | 0.799896 | NO |
| Related | | | | | | |
| Ile_Aminoacids | 38 | >0.99999999 | 0.863484 | −0.21176 | 0.788921 | NO |
| HexCer(d18:1/23:0)_ | 30.28 | >0.99999999 | 1.109091 | 0.149378 | 0.786741 | NO |
| Glycosylceramides | | | | | | |
| CE(18:2)_Cholesterol Esters | 25.84 | >0.99999999 | 0.878962 | −0.18613 | 0.773893 | NO |
| PC ae C44:5_ | 30.85 | >0.99999999 | 1.105333 | 0.144482 | 0.755919 | NO |
| Glycerophospholipids | | | | | | |
| PC ae C42:4_ | 19.2 | >0.99999999 | 1.114455 | 0.156338 | 0.753882 | NO |
| Glycerophospholipids | | | | | | |
| HexCer(d18:1/18:0)_ | 23.59 | >0.99999999 | 0.838902 | −0.25343 | 0.74654 | NO |
| Glycosylceramides | | | | | | |
| Phe_Aminoacids | 32.16 | >0.99999999 | 0.910258 | −0.13565 | 0.742945 | NO |
| PC aa C36:5_ | 36.02 | >0.99999999 | 0.822595 | −0.28175 | 0.72107 | NO |
| Glycerophospholipids | | | | | | |
| HexCer(d18:1/22:0)_ | 24.75 | >0.99999999 | 1.106355 | 0.145815 | 0.718226 | NO |
| Glycosylceramides | | | | | | |
| PC ae C44:4_ | 22.13 | >0.99999999 | 1.085744 | 0.118684 | 0.703663 | NO |
| Glycerophospholipids | | | | | | |
| SM C20:2_Sphingolipids | 22.61 | >0.99999999 | 0.86182 | −0.21454 | 0.70067 | NO |
| Cer(d16:1/22:0)_Ceramides | 30.9 | >0.99999999 | 0.84921 | −0.23581 | 0.680775 | NO |
| TG(20:2_32:1)_ | 25.7 | >0.99999999 | 0.751563 | −0.41204 | 0.675899 | NO |
| Triacylglycerols | | | | | | |
| Cer(d18:1/26:0)_Ceramides | 26.34 | >0.99999999 | 0.804801 | −0.3133 | 0.668804 | NO |
| PC ae C38:0_ | 32.28 | >0.99999999 | 0.894143 | −0.16142 | 0.668475 | NO |
| Glycerophospholipids | | | | | | |
| TG(20:5_36:3)_Triacylglycerols | 37.92 | >0.99999999 | 0.76469 | −0.38705 | 0.660828 | NO |
| DHA_Fatty Acids | 35.69 | >0.99999999 | 0.765321 | −0.38586 | 0.649767 | NO |
| beta-Ala_Biogenic Amines | 31.06 | >0.99999999 | 0.543317 | −0.88013 | 0.643886 | NO |
| PC aa C28:1_ | 31.23 | >0.99999999 | 0.886039 | −0.17456 | 0.613371 | NO |
| Glycerophospholipids | | | | | | |
| TrpBetaine_Aminoacids | 17.36 | >0.99999999 | 1.438192 | 0.524256 | 0.606125 | NO |
| Related | | | | | | |
| HexCer(d18:1/24:1)_ | 27.68 | >0.99999999 | 1.062139 | 0.086972 | 0.59963 | NO |
| Glycosylceramides | | | | | | |
| Hex2Cer(d18:1/16:0)_ | 24.41 | >0.99999999 | 1.072805 | 0.101388 | 0.593122 | NO |
| Glycosylceramides | | | | | | |
| SDMA_Aminoacids Related | 29.86 | >0.99999999 | 0.917635 | −0.12401 | 0.58548 | NO |
| Betaine_Aminoacids Related | 31.19 | >0.99999999 | 0.859221 | −0.2189 | 0.585124 | NO |
| 3-Met-His_Aminoacids | 37.65 | >0.99999999 | 0.71511 | −0.48376 | 0.57956 | NO |
| Related | | | | | | |
| H1_Sugars | 36.28 | >0.99999999 | 0.896422 | −0.15775 | 0.576612 | NO |
| TG(22:6_34:1)_ | 37.87 | >0.99999999 | 0.73988 | −0.43464 | 0.576331 | NO |
| Triacylglycerols | | | | | | |
| C5_Acylcarnitines | 25.17 | >0.99999999 | 0.891269 | −0.16607 | 0.571985 | NO |
| PC aa C34:1_ | 34 | >0.99999999 | 0.907275 | −0.14039 | 0.561822 | NO |
| Glycerophospholipids | | | | | | |
| C5-OH (C3-DC-M)_ | 24.71 | >0.99999999 | 0.948768 | −0.07587 | 0.560302 | NO |
| Acylcarnitines | | | | | | |
| TG(18:1_30:2)_Triacylglycerols | 21.42 | >0.99999999 | 1.276912 | 0.352659 | 0.550035 | NO |
| GCA_Bile Acids | 19.89 | >0.99999999 | 1.429348 | 0.515357 | 0.546846 | NO |
| TG(22:6_32:1)_Triacylglycerols | 37.14 | >0.99999999 | 0.765143 | −0.3862 | 0.539162 | NO |
| PC ae C36:1_ | 31.49 | >0.99999999 | 0.916352 | −0.12603 | 0.531892 | NO |
| Glycerophospholipids | | | | | | |
| Cer(d18:1/14:0)_Ceramides | 27.07 | >0.99999999 | 0.889247 | −0.16934 | 0.518654 | NO |
| Glu_Aminoacids | 27.58 | >0.99999999 | 0.822742 | −0.28149 | 0.513877 | NO |
| PC aa C32:1_ | 31.66 | >0.99999999 | 0.835873 | −0.25864 | 0.508239 | NO |
| Glycerophospholipids | | | | | | |
| C16:1-OH_Acylcarnitines | 23.03 | >0.99999999 | 0.938482 | −0.0916 | 0.505286 | NO |
| PC aa C38:5_ | 23.38 | >0.99999999 | 0.903311 | −0.14671 | 0.50303 | NO |
| Glycerophospholipids | | | | | | |
| PC aa C36:2_ | 27.1 | >0.99999999 | 0.93955 | −0.08996 | 0.490641 | NO |
| Glycerophospholipids | | | | | | |
| Hex2Cer(d18:1/24:1)_ | 28.9 | >0.99999999 | 1.086637 | 0.11987 | 0.487253 | NO |
| Glycosylceramides | | | | | | |
| CE(20:4)_Cholesterol Esters | 24.52 | >0.99999999 | 0.887218 | −0.17264 | 0.485777 | NO |
| PC ae C44:6_ | 21.63 | >0.99999999 | 1.079066 | 0.109783 | 0.484703 | NO |
| Glycerophospholipids | | | | | | |
| Ala_Aminoacids | 21.94 | >0.99999999 | 0.89116 | −0.16624 | 0.480069 | NO |
| 1-Met-His_Aminoacids Related | 37.92 | >0.99999999 | 0.910664 | −0.13501 | 0.4785 | NO |
| PC aa C38:0_ | 21.72 | >0.99999999 | 0.914729 | −0.12858 | 0.476849 | NO |
| Glycerophospholipids | | | | | | |
| C10_Acylcarnitines | 12.18 | >0.99999999 | 2.01087 | 1.00782 | 0.475895 | NO |
| C3_Acylcarnitines | 21.77 | >0.99999999 | 0.907326 | −0.14031 | 0.473466 | NO |
| Asn_Aminoacids | 30.57 | >0.99999999 | 0.92016 | −0.12004 | 0.467947 | NO |
| Cer(d18:1/16:0)_Ceramides | 36.25 | >0.99999999 | 1.05694 | 0.079893 | 0.465387 | NO |
| Lys_Aminoacids | 36.03 | >0.99999999 | 0.925571 | −0.11159 | 0.46398 | NO |
| C16:2-OH_Acylcarnitines | 23.36 | >0.99999999 | 0.945455 | −0.08092 | 0.460106 | NO |
| HexCer(d18:1/16:0)_ | 27.22 | >0.99999999 | 1.0552 | 0.077517 | 0.45522 | NO |
| Glycosylceramides | | | | | | |
| C18:1_Acylcarnitines | 20.18 | >0.99999999 | 1.083059 | 0.115112 | 0.448707 | NO |
| lysoPC a C20:3_ | 20.58 | >0.99999999 | 1.093541 | 0.129008 | 0.443533 | NO |
| Glycerophospholipids | | | | | | |
| C2_Acylcarnitines | 28.31 | >0.99999999 | 0.900879 | −0.15059 | 0.442126 | NO |
| TG(18:1_28:1)_Triacylglycerols | 16.6 | >0.99999999 | 1.337325 | 0.41935 | 0.434733 | NO |
| TG(20:3_36:3)_Triacylglycerols | 18.53 | >0.99999999 | 0.870989 | −0.19927 | 0.423471 | NO |
| PC ae C36:3_ | 30.65 | >0.99999999 | 1.066691 | 0.093142 | 0.418885 | NO |
| Glycerophospholipids | | | | | | |
| TG(18:1_38:6)_Triacylglycerols | 30.03 | >0.99999999 | 0.878333 | −0.18716 | 0.413282 | NO |
| TG(18:2_30:1)_Triacylglycerols | 19.76 | >0.99999999 | 1.246944 | 0.318396 | 0.412087 | NO |
| FA(18:2)_Fatty Acids | 30.09 | >0.99999999 | 0.802725 | −0.31702 | 0.410609 | NO |
| CE(20:3)_Cholesterol Esters | 21.77 | >0.99999999 | 0.902116 | −0.14861 | 0.408847 | NO |
| DG(18:1_18:4)_Diacylglycerols | 29.79 | >0.99999999 | 0.834441 | −0.26112 | 0.408672 | NO |
| C8_Acylcarnitines | 12.2 | >0.99999999 | 2.896175 | 1.534149 | 0.406571 | NO |
| Kynurenine_Aminoacids Related | 28.61 | >0.99999999 | 0.915227 | −0.1278 | 0.406429 | NO |
| TG(22:6_34:2)_Triacylglycerols | 37.99 | >0.99999999 | 0.7768 | −0.36439 | 0.403699 | NO |
| DG(18:1_18:3)_Diacylglycerols | 23.75 | >0.99999999 | 0.739932 | −0.43454 | 0.401935 | NO |
| Lac_Carboxylic Acids | 37.29 | >0.99999999 | 1.105026 | 0.14408 | 0.401933 | NO |
| TG(18:2_28:0)_Triacylglycerols | 18.74 | >0.99999999 | 1.294983 | 0.372933 | 0.400649 | NO |
| TG(16:0_40:8)_Triacylglycerols | 38 | >0.99999999 | 0.81586 | −0.29361 | 0.396691 | NO |
| Leu_Aminoacids | 37.9 | >0.99999999 | 0.920278 | −0.11986 | 0.396295 | NO |
| Val_Aminoacids | 35.35 | >0.99999999 | 0.945954 | −0.08016 | 0.394784 | NO |
| C6 (C4:1-DC)_Acylcarnitines | 12.33 | >0.99999999 | 1.294413 | 0.372298 | 0.391348 | NO |
| SM (OH) C14:1_Sphingolipids | 23.52 | >0.99999999 | 0.916964 | −0.12506 | 0.381527 | NO |
| C12_Acylcarnitines | 12.86 | >0.99999999 | 1.322066 | 0.402794 | 0.372692 | NO |
| PC ae C40:6_Glycerophospholipids | 26.7 | >0.99999999 | 0.943925 | −0.08326 | 0.372003 | NO |
| PC ae C40:4_Glycerophospholipids | 24.2 | >0.99999999 | 1.058971 | 0.082663 | 0.370864 | NO |
| 3-IAA_Indoles Derivatives | 13.05 | >0.99999999 | 1.461974 | 0.547917 | 0.370403 | NO |
| PC ae C40:1_Glycerophospholipids | 27.88 | >0.99999999 | 0.939636 | −0.08983 | 0.369373 | NO |
| DCA_Bile Acids | 16.37 | >0.99999999 | 1.372543 | 0.456851 | 0.368524 | NO |
| HexCer(d18:1/18:1)_ | 25.27 | >0.99999999 | 0.915097 | −0.128 | 0.366146 | NO |
| Glycosylceramides | | | | | | |
| HipAcid_Carboxylic Acids | 30.87 | >0.99999999 | 0.747399 | −0.42005 | 0.365263 | NO |
| TG(14:0_35:1)_Triacylglycerols | 19.47 | >0.99999999 | 0.799759 | −0.32236 | 0.364993 | NO |
| HexCer(d16:1/22:0)_ | 23.52 | >0.99999999 | 0.862443 | −0.2135 | 0.363884 | NO |
| Glycosylceramides | | | | | | |
| Cer(d18:1/23:0)_Ceramides | 32.47 | >0.99999999 | 0.929191 | −0.10595 | 0.361378 | NO |
| GCDCA_Bile Acids | 20.52 | >0.99999999 | 1.201696 | 0.265072 | 0.3595 | NO |
| C3-DC (C4-OH)_Acylcarnitines | 16.68 | >0.99999999 | 0.900312 | 0.1515 | 0.358954 | NO |
| Cer(d18:2/24:0)_Ceramides | 27.04 | >0.99999999 | 1.091546 | 0.126373 | 0.356029 | NO |
| CA_Bile Acids | 35.89 | >0.99999999 | 0.546423 | 0.87191 | 0.35512 | NO |
| GLCA_Bile Acids | 32.26 | >0.99999999 | 0.756743 | −0.40212 | 0.352295 | NO |
| TG(22:5_34:1)_Triacylglycerols | 23.55 | >0.99999999 | 0.855556 | 0.22507 | 0.349611 | NO |
| TG(18:1_26:0)_Triacylglycerols | 14.25 | >0.99999999 | 1.437713 | 0.523776 | 0.348848 | NO |
| TG(22:2_32:4)_Triacylglycerols | 22.97 | >0.99999999 | 0.854804 | −0.22633 | 0.344489 | NO |
| Creatinine_Aminoacids Related | 31.18 | >0.99999999 | 1.056666 | 0.07952 | 0.343719 | NO |
| AA_Fatty Acids | 32.41 | >0.99999999 | 0.880465 | −0.18366 | 0.337256 | NO |
| PC aa C36:6_ | 32.65 | >0.99999999 | 0.924641 | −0.11304 | 0.335844 | NO |
| Glycerophospholipids | | | | | | |
| Trp_Aminoacids | 32.9 | >0.99999999 | 1.055794 | 0.078328 | 0.334503 | NO |
| TG(22:6_32:0)_Triacylglycerols | 30.08 | >0.99999999 | 0.797843 | −0.32582 | 0.333947 | NO |
| PC ae C36:4_ | 35.07 | >0.99999999 | 1.06139 | 0.085955 | 0.333394 | NO |
| Glycerophospholipids | | | | | | |
| PC aa C38:4_ | 24.24 | >0.99999999 | 0.925801 | −0.11123 | 0.332912 | NO |
| Glycerophospholipids | | | | | | |
| TG(20:4_34:2)_Triacylglycerols | 15.89 | >0.99999999 | 1.211123 | 0.276346 | 0.332633 | NO |
| TG(14:0_36:4)_Triacylglycerols | 16.78 | >0.99999999 | 1.177544 | 0.23578 | 0.324462 | NO |
| TG(20:4_32:2)_Triacylglycerols | 16.83 | >0.99999999 | 1.233069 | 0.302254 | 0.323739 | NO |
| PC ae C38:2_ | 27.7 | >0.99999999 | 0.951489 | −0.07174 | 0.323339 | NO |
| Glycerophospholipids | | | | | | |
| C14:2_Acylcarnitines | 15 | >0.99999999 | 1.135766 | 0.183666 | 0.319694 | NO |
| TG(17:1_36:4)_Triacylglycerols | 19.46 | >0.99999999 | 1.141443 | 0.190859 | 0.319568 | NO |
| TG(22:4_32:2)_Triacylglycerols | 25.43 | >0.99999999 | 0.837527 | −0.25579 | 0.313192 | NO |
| SM C18:0_Sphingolipids | 24.66 | >0.99999999 | 0.93493 | −0.09707 | 0.310893 | NO |
| Cer(d18:2/18:0)_Ceramides | 24.8 | >0.99999999 | 0.916221 | −0.12623 | 0.307941 | NO |
| TMAO_Amine Oxides | 15.68 | >0.99999999 | 1.38339 | 0.468208 | 0.307288 | NO |
| HexCer(d18:2/24:0)_ | 20.81 | >0.99999999 | 0.923077 | −0.11548 | 0.306724 | NO |
| Glycosylceramides | | | | | | |
| SM (OH) C24:1_Sphingolipids | 25.16 | >0.99999999 | 0.945072 | −0.0815 | 0.305845 | NO |
| TCDCA_Bile Acids | 36.32 | >0.99999999 | 0.810491 | −0.30313 | 0.303826 | NO |
| TG(18:2_32:2)_Triacylglycerols | 15.66 | >0.99999999 | 1.182353 | 0.241661 | 0.301171 | NO |
| t4-OH-Pro_Aminoacids Related | 31.44 | >0.99999999 | 0.784566 | −0.35003 | 0.299105 | NO |
| Cer(d16:1/24:0)_Ceramides | 26.93 | >0.99999999 | 0.909044 | −0.13758 | 0.297642 | NO |
| PC aa C30:0_ | 22.87 | >0.99999999 | 0.932451 | −0.1009 | 0.293427 | NO |
| Glycerophospholipids | | | | | | |
| Cer(d18:2/22:0)_Ceramides | 24.26 | >0.99999999 | 1.071735 | 0.099948 | 0.293131 | NO |
| TG(20:3_34:0)_Triacylglycerols | 23.52 | >0.99999999 | 0.873852 | −0.19454 | 0.29313 | NO |
| ProBetaine_Aminoacids Related | 13.36 | >0.99999999 | 1.960526 | 0.971241 | 0.292921 | NO |
| TG(18:1_33:2)_Triacylglycerols | 24.73 | >0.99999999 | 0.886303 | −0.17413 | 0.290127 | NO |
| TG(18:3_38:5)_Triacylglycerols | 24.26 | >0.99999999 | 0.894137 | −0.16143 | 0.287193 | NO |
| TG(16:1_28:0)_Triacylglycerols | 14.74 | >0.99999999 | 1.627287 | 0.702469 | 0.283793 | NO |
| Cer(d18:2/24:1)_Ceramides | 26.45 | >0.99999999 | 1.052423 | 0.073715 | 0.283445 | NO |
| TG(20:0_32:4)_Triacylglycerols | 17.4 | >0.99999999 | 1.143791 | 0.193823 | 0.280506 | NO |
| HArg_Aminoacids Related | 28.65 | >0.99999999 | 1.097368 | 0.134048 | 0.278422 | NO |
| Cer(d18:1/26:1)_Ceramides | 19.06 | >0.99999999 | 0.881713 | −0.18162 | 0.275756 | NO |
| C5-DC (C6-OH)_Acylcarnitines | 20.24 | >0.99999999 | 0.966206 | −0.0496 | 0.275615 | NO |
| TG(18:2_34:2)_Triacylglycerols | 15.28 | >0.99999999 | 1.151826 | 0.203923 | 0.268773 | NO |
| TG(16:0_40:7)_Triacylglycerols | 30.2 | >0.99999999 | 0.835253 | −0.25972 | 0.268679 | NO |
| PC ae C40:2_ | 27.09 | >0.99999999 | 0.947078 | −0.07844 | 0.267316 | NO |
| Glycerophospholipids | | | | | | |
| TG(16:0_28:1)_Triacylglycerols | 15.11 | >0.99999999 | 1.375952 | 0.46043 | 0.263866 | NO |
| TG(18:2_35:3)_Triacylglycerols | 17.13 | >0.99999999 | 1.129964 | 0.176277 | 0.260735 | NO |
| PC aa C36:1_ | 25.63 | >0.99999999 | 0.94806 | −0.07695 | 0.259542 | NO |
| Glycerophospholipids | | | | | | |
| Taurine_Aminoacids Related | 19.89 | >0.99999999 | 1.070405 | 0.098156 | 0.259265 | NO |
| TG(18:2_38:6)_Triacylglycerols | 26.67 | >0.99999999 | 0.898152 | −0.15497 | 0.257147 | NO |
| DG(18:2_18:2)_Diacylglycerols | 17.01 | >0.99999999 | 1.127203 | 0.172747 | 0.256797 | NO |
| TG(16:1_36:4)_Triacylglycerols | 14.62 | >0.99999999 | 1.154072 | 0.206733 | 0.254485 | NO |
| TG(18:3_34:2)_Triacylglycerols | 16.82 | >0.99999999 | 1.149209 | 0.200641 | 0.254363 | NO |
| CE(18:3)_Cholesterol Esters | 19.83 | >0.99999999 | 0.904645 | −0.14458 | 0.253604 | NO |
| PC ae C32:2_ | 17.2 | >0.99999999 | 1.063098 | 0.088274 | 0.253002 | NO |
| Glycerophospholipids | | | | | | |
| PC ae C42:5_ | 30.52 | >0.99999999 | 1.035351 | 0.05012 | 0.252091 | NO |
| Glycerophospholipids | | | | | | |
| PC ae C32:1_ | 22.67 | >0.99999999 | 1.043423 | 0.061324 | 0.250408 | NO |
| Glycerophospholipids | | | | | | |
| TG(16:0_36:4)_Triacylglycerols | 15.8 | >0.99999999 | 1.14073 | 0.189957 | 0.250176 | NO |
| lysoPC a C16:0_ | 20.43 | >0.99999999 | 1.050154 | 0.070601 | 0.248747 | NO |
| Glycerophospholipids | | | | | | |
| C16:1_Acylcarnitines | 13.56 | >0.99999999 | 1.092107 | 0.127114 | 0.247726 | NO |
| TG(18:2_30:0)_Triacylglycerols | 19.16 | >0.99999999 | 1.189234 | 0.250033 | 0.247435 | NO |
| TG(18:2_34:4)_Triacylglycerols | 16.47 | >0.99999999 | 1.135901 | 0.183837 | 0.246909 | NO |
| Gln_Aminoacids | 23.33 | >0.99999999 | 0.96446 | −0.05221 | 0.246053 | NO |
| TG(18:2_34:3)_Triacylglycerols | 15.67 | >0.99999999 | 1.145601 | 0.196104 | 0.244848 | NO |
| TLCA_Bile Acids | 23.11 | >0.99999999 | 0.826231 | −0.27538 | 0.243951 | NO |
| PC ae C34:2_ | 25.1 | >0.99999999 | 1.04559 | 0.064318 | 0.243881 | NO |
| Glycerophospholipids | | | | | | |
| TG(18:1_32:3)_Triacylglycerols | 17.92 | >0.99999999 | 1.124785 | 0.169649 | 0.23711 | NO |
| TG(20:3_32:0)_Triacylglycerols | 21.48 | >0.99999999 | 0.831673 | −0.26591 | 0.23709 | NO |
| C16:2_Acylcarnitines | 13.35 | >0.99999999 | 1.070055 | 0.097685 | 0.235345 | NO |
| TG(18:1_33:3)_Triacylglycerols | 20.68 | >0.99999999 | 0.882029 | −0.1811 | 0.23515 | NO |
| C18_Acylcarnitines | 19.97 | >0.99999999 | 1.056097 | 0.078742 | 0.235028 | NO |
| TDCA_Bile Acids | 15.47 | >0.99999999 | 1.248626 | 0.320341 | 0.233306 | NO |
| TG(14:0_32:2)_Triacylglycerols | 16.89 | >0.99999999 | 1.215861 | 0.281979 | 0.233057 | NO |
| PC aa C42:5_ | 20.71 | >0.99999999 | 0.956399 | −0.06432 | 0.232876 | NO |
| Glycerophospholipids | | | | | | |
| TG(18:0_38:6)_Triacylglycerols | 21.02 | >0.99999999 | 0.904251 | −0.1452 | 0.232011 | NO |
| lysoPC a C14:0_ | 17.83 | >0.99999999 | 1.075282 | 0.104715 | 0.230162 | NO |
| Glycerophospholipids | | | | | | |
| TG(20:4_32:0)_Triacylglycerols | 19.1 | >0.99999999 | 1.230177 | 0.298866 | 0.228902 | NO |
| TG(20:5_34:1)_Triacylglycerols | 29.54 | >0.99999999 | 0.849917 | −0.23461 | 0.228174 | NO |
| TG(16:0_28:2)_Triacylglycerols | 16.48 | >0.99999999 | 1.238734 | 0.308866 | 0.225663 | NO |
| PC ae C34:3_ | 24.64 | >0.99999999 | 1.05994 | 0.083983 | 0.22438 | NO |
| Glycerophospholipids | | | | | | |
| TG(18:1_33:1)_Triacylglycerols | 27.3 | >0.99999999 | 0.894138 | −0.16143 | 0.223726 | NO |
| TG(17:0_36:3)_Triacylglycerols | 24 | >0.99999999 | 0.920177 | −0.12002 | 0.222974 | NO |
| TG(18:3_32:1)_Triacylglycerols | 18.28 | >0.99999999 | 1.158858 | 0.212704 | 0.221428 | NO |
| Cer(d18:2/20:0)_Ceramides | 30.8 | >0.99999999 | 0.949074 | −0.07541 | 0.219632 | NO |
| SM (OH) C16:1_Sphingolipids | 20.96 | >0.99999999 | 0.948448 | −0.07636 | 0.218459 | NO |
| PC ae C38:4_ | 28.61 | >0.99999999 | 1.041137 | 0.05816 | 0.217125 | NO |
| Glycerophospholipids | | | | | | |
| TG(20:1_34:1)_Triacylglycerols | 21.88 | >0.99999999 | 0.906521 | −0.14159 | 0.216199 | NO |
| C14_Acylcarnitines | 13.62 | >0.99999999 | 1.090443 | 0.124915 | 0.214544 | NO |
| TG(18:3_30:0)_Triacylglycerols | 16.92 | >0.99999999 | 1.204974 | 0.269002 | 0.213679 | NO |
| PC aa C40:5_ | 23 | >0.99999999 | 0.925311 | −0.11199 | 0.213577 | NO |
| Glycerophospholipids | | | | | | |
| PC aa C34:2_ | 24.07 | >0.99999999 | 0.958703 | −0.06084 | 0.213436 | NO |
| Glycerophospholipids | | | | | | |
| TG(17:2_34:3)_Triacylglycerols | 21.46 | >0.99999999 | 0.889622 | −0.16874 | 0.213426 | NO |
| PC aa C34:3_ | 25.89 | >0.99999999 | 0.947974 | −0.07708 | 0.213111 | NO |
| Glycerophospholipids | | | | | | |
| TG(17:0_34:2)_Triacylglycerols | 24.41 | >0.99999999 | 0.899001 | −0.15361 | 0.211448 | NO |
| GDCA_Bile Acids | 16.94 | >0.99999999 | 1.186441 | 0.24664 | 0.210285 | NO |
| PC aa C36:4_ | 24.93 | >0.99999999 | 0.954501 | −0.06718 | 0.210071 | NO |
| Glycerophospholipids | | | | | | |
| TG(17:0_34:3)_Triacylglycerols | 22.19 | >0.99999999 | 0.875435 | −0.19193 | 0.209956 | NO |
| PC aa C34:4_ | 26.58 | >0.99999999 | 1.067496 | 0.094231 | 0.209784 | NO |
| Glycerophospholipids | | | | | | |
| TG(17:2_36:2)_ | 26.18 | >0.99999999 | 0.917717 | −0.12388 | 0.209778 | NO |
| Triacylglycerols | | | | | | |
| PC aa C42:6_ | 22.58 | >0.99999999 | 0.957755 | −0.06227 | 0.20866 | NO |
| Glycerophospholipids | | | | | | |
| TG(18:3_32:0)_ | 17.26 | >0.99999999 | 1.183443 | 0.24299 | 0.208388 | NO |
| Triacylglycerols | | | | | | |
| TG(16:0_34:4)_Triacylglycerols | 17.88 | >0.99999999 | 1.171429 | 0.228269 | 0.208272 | NO |
| SM C26:0_Sphingolipids | 26.32 | >0.99999999 | 0.967039 | −0.04835 | 0.205186 | NO |
| HexCer(d18:1/20:0)_ | 23.5 | >0.99999999 | 0.919049 | −0.12179 | 0.204745 | NO |
| Glycosylceramides | | | | | | |
| TG(18:2_36:3)_Triacylglycerols | 18.91 | >0.99999999 | 1.122461 | 0.166666 | 0.203864 | NO |
| TG(20:1_30:1)_Triacylglycerols | 33.53 | >0.99999999 | 0.914367 | −0.12915 | 0.203262 | NO |
| TG(20:4_32:1)_Triacylglycerols | 18.09 | >0.99999999 | 1.185994 | 0.246097 | 0.202058 | NO |
| TG(18:2_32:0)_Triacylglycerols | 17.39 | >0.99999999 | 1.152332 | 0.204557 | 0.201238 | NO |
| TG(16:0_36:5)_Triacylglycerols | 17.76 | >0.99999999 | 1.120258 | 0.163831 | 0.199968 | NO |
| TG(16:1_38:5)_Triacylglycerols | 18.62 | >0.99999999 | 0.906206 | −0.14209 | 0.199458 | NO |
| Cit_Aminoacids Related | 37.75 | >0.99999999 | 0.95607 | 0.06481 | 0.198837 | NO |
| TG(16:0_30:2)_Triacylglycerols | 20.1 | >0.99999999 | 1.157478 | 0.210985 | 0.197255 | NO |
| SM (OH) C22:1_Sphingolipids | 27.71 | >0.99999999 | 0.960945 | −0.05747 | 0.196497 | NO |
| HCys_Aminoacids Related | 22.21 | >0.99999999 | 1.051492 | 0.072438 | 0.192323 | NO |
| PC ae C38:6_ | 27.03 | >0.99999999 | 0.961972 | −0.05593 | 0.191774 | NO |
| Glycerophospholipids | | | | | | |
| PC ae C42:3_Glycerophospholipids | 21.42 | >0.99999999 | 1.032469 | 0.046099 | 0.191669 | NO |
| TG(18:1_30:1)_Triacylglycerols | 20.3 | >0.99999999 | 1.129429 | 0.175594 | 0.1883 | NO |
| TG(18:0_32:0)_Triacylglycerols | 14.89 | >0.99999999 | 1.434743 | 0.520792 | 0.187563 | NO |
| PC ae C36:5_Glycerophospholipids | 27.67 | >0.99999999 | 1.04549 | 0.064179 | 0.187065 | NO |
| TG(16:0_34:3)_Triacylglycerols | 16.96 | >0.99999999 | 1.150691 | 0.2025 | 0.185933 | NO |
| TG(20:1_32:0)_Triacylglycerols | 20.32 | >0.99999999 | 1.10385 | 0.142544 | 0.185486 | NO |
| TG(17:2_36:4)_Triacylglycerols | 21.62 | >0.99999999 | 0.925553 | −0.11161 | 0.184328 | NO |
| TG(14:0_34:2)_Triacylglycerols | 19.13 | >0.99999999 | 1.142927 | 0.192733 | 0.182728 | NO |
| TG(18:2_36:2)_Triacylglycerols | 17.64 | >0.99999999 | 1.098961 | 0.13614 | 0.181707 | NO |
| SM C24:0_Sphingolipids | 32.25 | >0.99999999 | 1.032883 | 0.046677 | 0.181679 | NO |
| PC aa C32:0_Glycerophospholipids | 21.85 | >0.99999999 | 0.968049 | −0.04685 | 0.181664 | NO |
| PC ae C34:1_Glycerophospholipids | 26.07 | >0.99999999 | 0.967773 | −0.04726 | 0.181266 | NO |
| PC ae C38:1_Glycerophospholipids | 23.93 | >0.99999999 | 0.959027 | −0.06036 | 0.180835 | NO |
| TG(18:1_36:3)_Triacylglycerols | 17.78 | >0.99999999 | 1.101805 | 0.139869 | 0.179411 | NO |
| SM C26:1_Sphingolipids | 23.64 | >0.99999999 | 0.963806 | −0.05319 | 0.178896 | NO |
| His_Aminoacids | 32.24 | >0.99999999 | 0.971186 | −0.04218 | 0.177967 | NO |
| TG(14:0_36:3)_Triacylglycerols | 18.74 | >0.99999999 | 1.095511 | 0.131604 | 0.177708 | NO |
| TG(17:1_34:1)_Triacylglycerols | 23.46 | >0.99999999 | 0.890877 | −0.1667 | 0.177457 | NO |
| TG(18:3_34:3)_Triacylglycerols | 16.94 | >0.99999999 | 1.109444 | 0.149836 | 0.176859 | NO |
| TG(22:4_34:2)_Triacylglycerols | 17.17 | >0.99999999 | 1.102187 | 0.140369 | 0.176813 | NO |
| TG(14:0_34:3)_Triacylglycerols | 17.74 | >0.99999999 | 1.134728 | 0.182347 | 0.17648 | NO |
| Cer(d18:1/18:0)_Ceramides | 26.65 | >0.99999999 | 1.039803 | 0.05631 | 0.174135 | NO |
| TG(16:1_32:2)_Triacylglycerols | 16.27 | >0.99999999 | 1.224967 | 0.292743 | 0.172992 | NO |
| PC aa C36:3_ | 33.87 | >0.99999999 | 0.968033 | −0.04687 | 0.172789 | NO |
| Glycerophospholipids | | | | | | |
| Hex3Cer(d18:1/16:0)_ | 23.04 | >0.99999999 | 1.031583 | 0.044861 | 0.172578 | NO |
| Glycosylceramides | | | | | | |
| Cer(d18:1/24:0)_Ceramides | 26.5 | >0.99999999 | 1.041167 | 0.058202 | 0.171447 | NO |
| TG(18:3_34:0)_Triacylglycerols | 17.27 | >0.99999999 | 1.113411 | 0.154987 | 0.170318 | NO |
| TG(20:4_36:4)_Triacylglycerols | 18.23 | >0.99999999 | 1.070555 | 0.098359 | 0.166429 | NO |
| PC aa C42:1_ | 22.9 | >0.99999999 | 0.975385 | −0.03596 | 0.165721 | NO |
| Glycerophospholipids | | | | | | |
| TG(16:0_32:3)_Triacylglycerols | 18.22 | >0.99999999 | 1.146447 | 0.19717 | 0.165712 | NO |
| TG(18:2_32:1)_Triacylglycerols | 17.3 | >0.99999999 | 1.111441 | 0.152432 | 0.165283 | NO |
| C9_Acylcarnitines | 21.82 | >0.99999999 | 0.971321 | −0.04198 | 0.165126 | NO |
| lysoPC a C18:2_ | 21.23 | >0.99999999 | 1.063095 | 0.08827 | 0.163994 | NO |
| Glycerophospholipids | | | | | | |
| TG(20:1_32:1)_Triacylglycerols | 19.95 | >0.99999999 | 0.902972 | −0.14725 | 0.163377 | NO |
| TG(18:1_35:2)_Triacylglycerols | 24.3 | >0.99999999 | 0.936623 | −0.09446 | 0.162709 | NO |
| TG(16:1_36:1)_Triacylglycerols | 21.58 | >0.99999999 | 0.917738 | −0.12385 | 0.162325 | NO |
| TG(20:4_34:1)_Triacylglycerols | 18.11 | >0.99999999 | 1.102929 | 0.14134 | 0.162169 | NO |
| TG(16:0_32:2)_Triacylglycerols | 19.58 | >0.99999999 | 1.142696 | 0.192442 | 0.162135 | NO |
| PC aa C42:4_ | 24.06 | >0.99999999 | 0.973763 | −0.03836 | 0.161925 | NO |
| Glycerophospholipids | | | | | | |
| TG(18:3_34:1)_Triacylglycerols | 17.93 | >0.99999999 | 1.093597 | 0.129081 | 0.161879 | NO |
| TG(20:5_34:2)_Triacylglycerols | 31.79 | >0.99999999 | 0.886238 | −0.17423 | 0.160802 | NO |
| AconAcid_Carboxylic Acids | 23.97 | >0.99999999 | 0.966457 | −0.04922 | 0.159927 | NO |
| Cer(d18:2/16:0)_Ceramides | 22.02 | >0.99999999 | 1.035509 | 0.05034 | 0.158905 | NO |
| TG(16:0_38:5)_Triacylglycerols | 17.84 | >0.99999999 | 1.103098 | 0.141561 | 0.158901 | NO |
| TG(20:3_34:2)_Triacylglycerols | 18.65 | >0.99999999 | 1.090835 | 0.125432 | 0.157924 | NO |
| SM C18:1_Sphingolipids | 18.74 | >0.99999999 | 0.958424 | −0.06126 | 0.156478 | NO |
| TG(18:1_36:2)_Triacylglycerols | 17.97 | >0.99999999 | 1.097177 | 0.133796 | 0.156125 | NO |
| PC ae C40:5_ | 27.63 | >0.99999999 | 1.025778 | 0.036718 | 0.156059 | NO |
| Glycerophospholipids | | | | | | |
| SM C24:1_Sphingolipids | 24.84 | >0.99999999 | 1.031672 | 0.044984 | 0.155431 | NO |
| TG(18:1_38:5)_Triacylglycerols | 20.04 | >0.99999999 | 0.945808 | −0.08038 | 0.154903 | NO |
| PC ae C42:1_ | 22.52 | >0.99999999 | 0.97295 | −0.03956 | 0.149426 | NO |
| Glycerophospholipids | | | | | | |
| TG(18:1_36:1)_Triacylglycerols | 19.37 | >0.99999999 | 1.07531 | 0.104752 | 0.145535 | NO |
| TG(14:0_34:0)_Triacylglycerols | 14.37 | >0.99999999 | 1.32113 | 0.401772 | 0.144886 | NO |
| TG(18:0_36:1)_Triacylglycerols | 15.63 | >0.99999999 | 1.13643 | 0.184508 | 0.143458 | NO |
| TG(16:1_36:5)_Triacylglycerols | 15.81 | >0.99999999 | 1.088864 | 0.122824 | 0.142993 | NO |
| TG(16:1_34:3)_Triacylglycerols | 15.7 | >0.99999999 | 1.123899 | 0.168512 | 0.14115 | NO |
| TG(18:1_31:0)_Triacylglycerols | 20.99 | >0.99999999 | 0.883008 | −0.1795 | 0.140545 | NO |
| TG(18:1_36:4)_Triacylglycerols | 20.38 | >0.99999999 | 1.077139 | 0.107204 | 0.140448 | NO |
| TG(18:1_36:0)_Triacylglycerols | 16.91 | >0.99999999 | 1.0908 | 0.125386 | 0.138535 | NO |
| TG(16:0_35:2)_Triacylglycerols | 22.56 | >0.99999999 | 0.913629 | −0.13032 | 0.138255 | NO |
| DG(16:0_18:1)_Diacylglycerols | 20.32 | >0.99999999 | 1.083557 | 0.115775 | 0.135537 | NO |
| PC ae C30:0_ | 22.54 | >0.99999999 | 1.029616 | 0.042106 | 0.134737 | NO |
| Glycerophospholipids | | | | | | |
| TG(16:0_32:0)_Triacylglycerols | 18.86 | >0.99999999 | 1.267152 | 0.34159 | 0.134378 | NO |
| lysoPC a C20:4_ | 28.23 | >0.99999999 | 1.034238 | 0.048568 | 0.132026 | NO |
| Glycerophospholipids | | | | | | |
| PC aa C40:2_ | 25.2 | >0.99999999 | 0.978531 | −0.03131 | 0.127906 | NO |
| Glycerophospholipids | | | | | | |
| C14:1_Acylcarnitines | 19.29 | >0.99999999 | 0.970588 | −0.04307 | 0.127514 | NO |
| TG(18:2_36:5)_Triacylglycerols | 37.98 | >0.99999999 | 0.905386 | −0.1434 | 0.126846 | NO |
| Ind-SO4_Indoles Derivatives | 35.71 | >0.99999999 | 0.95615 | −0.06469 | 0.126671 | NO |
| TG(18:0_36:5)_Triacylglycerols | 26.05 | >0.99999999 | 0.945897 | −0.08024 | 0.125428 | NO |
| FA(18:1)_Fatty Acids | 24.42 | >0.99999999 | 1.084848 | 0.117494 | 0.122297 | NO |
| TG(16:0_38:4)_Triacylglycerols | 19 | >0.99999999 | 1.072406 | 0.100851 | 0.121667 | NO |
| PC aa C32:2_ | 20.9 | >0.99999999 | 1.046016 | 0.064905 | 0.120897 | NO |
| Glycerophospholipids | | | | | | |
| TG(17:0_34:1)_Triacylglycerols | 20.91 | >0.99999999 | 0.918031 | −0.12338 | 0.119733 | NO |
| TG(20:4_33:2)_Triacylglycerols | 19.76 | >0.99999999 | 1.062982 | 0.088118 | 0.116902 | NO |
| Putrescine_Biogenic Amines | 27.76 | >0.99999999 | 1.055215 | 0.077537 | 0.116291 | NO |
| TG(16:1_34:2)_Triacylglycerols | 16.86 | >0.99999999 | 1.100116 | 0.137655 | 0.115915 | NO |
| TG(18:2_35:2)_Triacylglycerols | 17.89 | >0.99999999 | 1.056684 | 0.079543 | 0.114695 | NO |
| TG(14:0_34:1)_Triacylglycerols | 18.59 | >0.99999999 | 1.105574 | 0.144796 | 0.114356 | NO |
| Cer(d18:1/22:0)_Ceramides | 27.15 | >0.99999999 | 1.024917 | 0.035507 | 0.114318 | NO |
| TG(20:3_36:4)_Triacylglycerols | 19.06 | >0.99999999 | 0.961242 | −0.05703 | 0.112356 | NO |
| TG(17:0_36:4)_Triacylglycerols | 19.79 | >0.99999999 | 1.051788 | 0.072844 | 0.112194 | NO |
| PC aa C36:0_ | 18.89 | >0.99999999 | 0.961453 | −0.05671 | 0.111355 | NO |
| Glycerophospholipids | | | | | | |
| TG(18:3_36:4)_Triacylglycerols | 37.92 | >0.99999999 | 0.925667 | −0.11143 | 0.109146 | NO |
| TG(18:0_36:2)_Triacylglycerols | 19.34 | >0.99999999 | 1.060278 | 0.084442 | 0.108688 | NO |
| TG(18:1_34:1)_Triacylglycerols | 22.62 | >0.99999999 | 0.95057 | −0.07313 | 0.108271 | NO |
| FA(20:3)_Fatty Acids | 23.72 | >0.99999999 | 1.06023 | 0.084378 | 0.108265 | NO |
| PC ae C40:3_ | 26.08 | >0.99999999 | 1.017652 | 0.025245 | 0.106972 | NO |
| Glycerophospholipids | | | | | | |
| TG(16:1_32:1)_Triacylglycerols | 17.47 | >0.99999999 | 1.146548 | 0.197297 | 0.106258 | NO |
| lysoPC a C17:0_ | 22.95 | >0.99999999 | 1.027586 | 0.039259 | 0.106243 | NO |
| Glycerophospholipids | | | | | | |
| TG(17:0_32:1)_Triacylglycerols | 21.3 | >0.99999999 | 0.883733 | −0.17832 | 0.105902 | NO |
| PC ae C34:0_ | 24.83 | >0.99999999 | 0.973975 | −0.03804 | 0.105748 | NO |
| Glycerophospholipids | | | | | | |
| TG(18:2_36:1)_Triacylglycerols | 18.28 | >0.99999999 | 1.052106 | 0.07328 | 0.10562 | NO |
| TG(20:4_30:0)_Triacylglycerols | 20.48 | >0.99999999 | 1.106609 | 0.146145 | 0.105049 | NO |
| TG(16:0_38:6)_Triacylglycerols | 18.71 | >0.99999999 | 1.073903 | 0.102864 | 0.104777 | NO |
| PC ae C36:2_ | 25.43 | >0.99999999 | 0.979529 | −0.02984 | 0.104386 | NO |
| Glycerophospholipids | | | | | | |
| DG(16:1_18:1)_Diacylglycerols | 17.02 | >0.99999999 | 0.928192 | −0.1075 | 0.103246 | NO |
| TG(18:1_32:2)_Triacylglycerols | 19.22 | >0.99999999 | 1.057721 | 0.080959 | 0.102963 | NO |
| PC aa C40:4_ | 19.65 | >0.99999999 | 1.040367 | 0.057093 | 0.100848 | NO |
| Glycerophospholipids | | | | | | |
| TG(20:0_34:1)_Triacylglycerols | 22.44 | >0.99999999 | 0.942451 | −0.08551 | 0.099738 | NO |
| TG(16:0_38:7)_Triacylglycerols | 20.94 | >0.99999999 | 0.894068 | −0.16154 | 0.099022 | NO |
| TG(17:1_36:5)_Triacylglycerols | 20.45 | >0.99999999 | 1.063492 | 0.088809 | 0.098736 | NO |
| TG(20:3_32:2)_Triacylglycerols | 21.8 | >0.99999999 | 0.931845 | −0.10184 | 0.098471 | NO |
| TG(16:1_36:2)_Triacylglycerols | 18.84 | >0.99999999 | 0.951428 | −0.07183 | 0.098437 | NO |
| TG(18:1_34:4)_Triacylglycerols | 16.74 | >0.99999999 | 1.056196 | 0.078878 | 0.098185 | NO |
| DG(14:0_14:0)_Diacylglycerols | 20.22 | >0.99999999 | 1.017218 | 0.024628 | 0.097786 | NO |
| PC aa C42:2_ | 19.89 | >0.99999999 | 1.017556 | 0.025108 | 0.096019 | NO |
| Glycerophospholipids | | | | | | |
| TG(18:1_30:0)_Triacylglycerols | 18.51 | >0.99999999 | 1.089692 | 0.123921 | 0.095293 | NO |
| TG(18:1_33:0)_Triacylglycerols | 20.42 | >0.99999999 | 0.927782 | −0.10814 | 0.095236 | NO |
| Cer(d18:1/20:0)_Ceramides | 23 | >0.99999999 | 0.97723 | 0.03323 | 0.090173 | NO |
| PC ae C36:0_ | 24.83 | >0.99999999 | 0.982351 | −0.02569 | 0.089579 | NO |
| Glycerophospholipids | | | | | | |
| TG(18:0_36:4)_Triacylglycerols | 17.36 | >0.99999999 | 1.051502 | 0.072452 | 0.089372 | NO |
| TG(16:0_34:0)_Triacylglycerols | 17.53 | >0.99999999 | 1.117673 | 0.160498 | 0.088108 | NO |
| TG(18:0_30:1)_Triacylglycerols | 15.4 | >0.99999999 | 1.12011 | 0.163641 | 0.087752 | NO |
| TG(16:0_36:3)_Triacylglycerols | 17.81 | >0.99999999 | 1.044562 | 0.062898 | 0.084092 | NO |
| TG(18:2_38:5)_Triacylglycerols | 16.56 | >0.99999999 | 1.044294 | 0.062528 | 0.084076 | NO |
| TG(16:0_34:2)_Triacylglycerols | 18.68 | >0.99999999 | 1.065103 | 0.090993 | 0.083252 | NO |
| TG(16:0_40:6)_Triacylglycerols | 19.24 | >0.99999999 | 1.04753 | 0.066992 | 0.080361 | NO |
| TG(20:4_36:3)_Triacylglycerols | 18.29 | >0.99999999 | 0.968885 | −0.0456 | 0.080344 | NO |
| TG(16:0_35:3)_Triacylglycerols | 22.2 | >0.99999999 | 0.949162 | −0.07527 | 0.080194 | NO |
| TG(20:4_36:2)_Triacylglycerols | 18.54 | >0.99999999 | 0.970073 | −0.04383 | 0.079089 | NO |
| PC ae C38:3_ | 33.19 | >0.99999999 | 1.016318 | 0.023352 | 0.079056 | NO |
| Glycerophospholipids | | | | | | |
| TG(18:2_34:1)_Triacylglycerols | 17.61 | >0.99999999 | 1.04232 | 0.059799 | 0.078488 | NO |
| ADMA_Aminoacids Related | 21.69 | >0.99999999 | 0.982573 | −0.02536 | 0.078245 | NO |
| TG(18:2_33:0)_Triacylglycerols | 21.37 | >0.99999999 | 0.949761 | −0.07436 | 0.078168 | NO |
| TG(16:0_38:3)_Triacylglycerols | 21.1 | >0.99999999 | 0.962194 | −0.0556 | 0.078043 | NO |
| TG(18:3_33:2)_Triacylglycerols | 22.24 | >0.99999999 | 0.955459 | −0.06573 | 0.077667 | NO |
| TG(16:0_35:1)_Triacylglycerols | 19.74 | >0.99999999 | 0.935772 | −0.09577 | 0.076971 | NO |
| TG(16:1_36:3)_Triacylglycerols | 16.09 | >0.99999999 | 1.04471 | 0.063103 | 0.076706 | NO |
| TG(18:2_34:0)_Triacylglycerols | 19.22 | >0.99999999 | 1.044607 | 0.062961 | 0.076681 | NO |
| Cer(d18:1/24:1)_Ceramides | 36.05 | >0.99999999 | 1.015032 | 0.021526 | 0.076066 | NO |
| DG(18:1_18:2)_Diacylglycerols | 19.22 | >0.99999999 | 1.036408 | 0.051592 | 0.075232 | NO |
| GUDCA_Bile Acids | 37.47 | >0.99999999 | 0.926929 | −0.10947 | 0.074198 | NO |
| SM (OH) C22:2_Sphingolipids | 17.8 | >0.99999999 | 0.980286 | −0.02873 | 0.072945 | NO |
| PC aa C42:0_ | 22.68 | >0.99999999 | 0.986479 | −0.01964 | 0.072128 | NO |
| Glycerophospholipids | | | | | | |
| TG(16:0_36:2)_Triacylglycerols | 22.04 | >0.99999999 | 0.965605 | −0.0505 | 0.071627 | NO |
| TG(20:0_32:3)_Triacylglycerols | 21.52 | >0.99999999 | 0.966632 | −0.04896 | 0.071384 | NO |
| GLCAS_Bile Acids | 15.56 | >0.99999999 | 0.92702 | −0.10933 | 0.071354 | NO |
| TG(18:2_38:4)_Triacylglycerols | 16.28 | >0.99999999 | 1.037879 | 0.053638 | 0.070238 | NO |
| TG(18:2_35:1)_Triacylglycerols | 23.43 | >0.99999999 | 0.967671 | −0.04741 | 0.068778 | NO |
| TG(18:0_36:3)_Triacylglycerols | 19.48 | >0.99999999 | 1.032086 | 0.045564 | 0.063794 | NO |
| HexCer(d18:2/22:0)_ | 22.96 | >0.99999999 | 0.976211 | −0.03473 | 0.062717 | NO |
| Glycosylceramides | | | | | | |
| PC aa C40:3_ | 25.82 | >0.99999999 | 1.011658 | 0.016722 | 0.062153 | NO |
| Glycerophospholipids | | | | | | |
| TG(18:2_36:4)_Triacylglycerols | 29.17 | >0.99999999 | 1.046858 | 0.066066 | 0.060956 | NO |
| Hex2Cer(d18:1/14:0)_ | 26.38 | >0.99999999 | 0.977555 | −0.03275 | 0.06008 | NO |
| Glycosylceramides | | | | | | |
| TG(20:2_34:3)_Triacylglycerols | 23.17 | >0.99999999 | 0.969917 | −0.04407 | 0.059938 | NO |
| TG(22:5_34:2)_Triacylglycerols | 18.63 | >0.99999999 | 1.036604 | 0.051865 | 0.058956 | NO |
| TG(18:1_35:3)_Triacylglycerols | 19.42 | >0.99999999 | 0.973065 | −0.03939 | 0.057801 | NO |
| Hex3Cer(d18:1/24:1)_ | 18.96 | >0.99999999 | 1.021008 | 0.029995 | 0.057565 | NO |
| Glycosylceramides | | | | | | |
| TG(18:2_31:0)_Triacylglycerols | 22.33 | >0.99999999 | 0.958375 | −0.06134 | 0.056276 | NO |
| TG(14:0_36:1)_Triacylglycerols | 16.6 | >0.99999999 | 1.050923 | 0.071656 | 0.055624 | NO |
| TG(16:0_33:2)_Triacylglycerols | 21.6 | >0.99999999 | 0.954871 | −0.06662 | 0.055283 | NO |
| TG(16:0_33:1)_Triacylglycerols | 20.35 | >0.99999999 | 0.946835 | −0.07881 | 0.053799 | NO |
| TG(16:0_32:1)_Triacylglycerols | 19.86 | >0.99999999 | 1.055773 | 0.0783 | 0.053467 | NO |
| TG(18:1_34:2)_Triacylglycerols | 17.96 | >0.99999999 | 1.027631 | 0.039322 | 0.05189 | NO |
| TG(18:1_32:0)_Triacylglycerols | 19.22 | >0.99999999 | 1.042851 | 0.060533 | 0.051797 | NO |
| PC ae C44:3_ | 25.95 | >0.99999999 | 1.007098 | 0.010204 | 0.050615 | NO |
| Glycerophospholipids | | | | | | |
| PC aa C32:3_ | 18.99 | >0.99999999 | 1.01317 | 0.018876 | 0.049911 | NO |
| Glycerophospholipids | | | | | | |
| TG(16:1_32:0)_Triacylglycerols | 20.49 | >0.99999999 | 1.068019 | 0.094937 | 0.046481 | NO |
| TG(18:1_34:3)_Triacylglycerols | 17.48 | >0.99999999 | 1.025539 | 0.036382 | 0.04546 | NO |
| DG(16:0_18:2)_Diacylglycerols | 21.33 | >0.99999999 | 1.02626 | 0.037397 | 0.04298 | NO |
| lysoPC a C18:0_ | 20.63 | >0.99999999 | 1.011406 | 0.016362 | 0.042095 | NO |
| Glycerophospholipids | | | | | | |
| TG(14:0_38:5)_Triacylglycerols | 18.58 | >0.99999999 | 0.974808 | −0.03681 | 0.04041 | NO |
| TMCA_Bile Acids | 16.81 | >0.99999999 | 1.052189 | 0.073393 | 0.040272 | NO |
| TG(20:3_34:3)_Triacylglycerols | 15.52 | >0.99999999 | 0.976378 | −0.03449 | 0.038478 | NO |
| TG(18:3_35:2)_Triacylglycerols | 19.8 | >0.99999999 | 1.02221 | 0.031691 | 0.036464 | NO |
| lysoPC a C16:1_ | 19.56 | >0.99999999 | 0.988242 | −0.01706 | 0.036076 | NO |
| Glycerophospholipids | | | | | | |
| TG(20:2_34:4)_Triacylglycerols | 27.65 | >0.99999999 | 1.020635 | 0.029468 | 0.035376 | NO |
| CE(15:0)_Cholesterol Esters | 27.7 | >0.99999999 | 1.008618 | 0.01238 | 0.035105 | NO |
| TG(20:2_34:2)_Triacylglycerols | 18.53 | >0.99999999 | 0.981792 | −0.02651 | 0.035093 | NO |
| TG(18:2_36:0)_Triacylglycerols | 18.97 | >0.99999999 | 1.020105 | 0.028718 | 0.034824 | NO |
| TG(18:2_33:1)_Triacylglycerols | 21.16 | >0.99999999 | 0.982205 | −0.0259 | 0.031333 | NO |
| TG(20:4_34:0)_Triacylglycerols | 19.28 | >0.99999999 | 1.022986 | 0.032786 | 0.031137 | NO |
| TG(20:2_34:1)_Triacylglycerols | 20.75 | >0.99999999 | 0.984477 | 0.02257 | 0.03044 | NO |
| TG(18:3_36:2)_Triacylglycerols | 26.43 | >0.99999999 | 0.985936 | −0.02043 | 0.028085 | NO |
| TG(17:1_32:1)_Triacylglycerols | 20.62 | >0.99999999 | 0.974195 | −0.03772 | 0.026044 | NO |
| Orn_Aminoacids Related | 23.81 | >0.99999999 | 1.007612 | 0.01094 | 0.024462 | NO |
| C16_Acylcarnitines | 23.46 | >0.99999999 | 1.005838 | 0.008398 | 0.023902 | NO |
| PC ae C30:1_ | 22.86 | >0.99999999 | 0.994 | −0.00868 | 0.023012 | NO |
| Glycerophospholipids | | | | | | |
| TG(14:0_36:2)_Triacylglycerols | 22.23 | >0.99999999 | 0.986894 | −0.01903 | 0.022683 | NO |
| TG(18:1_32:1)_Triacylglycerols | 20.71 | >0.99999999 | 0.985063 | −0.02171 | 0.021967 | NO |
| TG(22:4_32:0)_Triacylglycerols | 19.92 | >0.99999999 | 1.019051 | 0.027227 | 0.020962 | NO |
| TG(18:2_33:2)_Triacylglycerols | 18.51 | >0.99999999 | 1.011965 | 0.017159 | 0.020628 | NO |
| TG(18:0_32:1)_Triacylglycerols | 17.78 | >0.99999999 | 1.02578 | 0.036722 | 0.020236 | NO |
| TG(16:0_36:6)_Triacylglycerols | 19.31 | >0.99999999 | 0.983494 | −0.02401 | 0.019423 | NO |
| TG(18:0_32:2)_Triacylglycerols | 18.13 | >0.99999999 | 1.018879 | 0.026982 | 0.018631 | NO |
| TG(17:1_36:3)_Triacylglycerols | 19.25 | >0.99999999 | 1.008994 | 0.012917 | 0.017359 | NO |
| TG(17:2_38:5)_Triacylglycerols | 23.74 | >0.99999999 | 1.008541 | 0.012269 | 0.016148 | NO |
| TG(20:4_34:3)_Triacylglycerols | 17.1 | >0.99999999 | 1.010758 | 0.015438 | 0.016027 | NO |
| CO_Acylcarnitines | 27.14 | >0.99999999 | 0.996572 | −0.00495 | 0.015486 | NO |
| TG(18:3_36:3)_Triacylglycerols | 28.47 | >0.99999999 | 1.009251 | 0.013285 | 0.015145 | NO |
| PC ae C38:5_ | 34.22 | >0.99999999 | 1.002688 | 0.003873 | 0.013638 | NO |
| Glycerophospholipids | | | | | | |
| DG(18:1_18:1)_Diacylglycerols | 21.55 | >0.99999999 | 1.006281 | 0.009033 | 0.013303 | NO |
| Cer(d18:2/23:0)_Ceramides | 27.72 | >0.99999999 | 1.003962 | 0.005705 | 0.013286 | NO |
| TG(17:1_34:2)_Triacylglycerols | 20.71 | >0.99999999 | 0.99147 | −0.01236 | 0.012512 | NO |
| TG(16:0_34:1)_Triacylglycerols | 20.57 | >0.99999999 | 1.010705 | 0.015363 | 0.012452 | NO |
| PC ae C42:2_ | 24.84 | >0.99999999 | 1.002633 | 0.003794 | 0.012259 | NO |
| Glycerophospholipids | | | | | | |
| TG(20:3_32:1)_Triacylglycerols | 21.57 | >0.99999999 | 0.990864 | −0.01324 | 0.012119 | NO |
| TG(16:1_34:1)_Triacylglycerols | 19.36 | >0.99999999 | 0.990359 | −0.01398 | 0.010732 | NO |
| TG(16:0_37:3)_Triacylglycerols | 23.04 | >0.99999999 | 0.994232 | −0.00835 | 0.009854 | NO |
| TG(18:1_36:5)_Triacylglycerols | 28.57 | >0.99999999 | 0.994472 | −0.008 | 0.009545 | NO |
| TG(18:0_34:2)_Triacylglycerols | 21.32 | >0.99999999 | 0.992291 | −0.01116 | 0.009416 | NO |
| TG(20:1_34:2)_Triacylglycerols | 27.01 | >0.99999999 | 0.995773 | −0.00611 | 0.009117 | NO |
| Trigonelline_Alkaloids | 13.93 | >0.99999999 | 1.016095 | 0.023035 | 0.00887 | NO |
| PC aa C38:3_ | 23.7 | >0.99999999 | 0.997205 | −0.00404 | 0.007798 | NO |
| Glycerophospholipids | | | | | | |
| TG(16:1_34:0)_Triacylglycerols | 20.5 | >0.99999999 | 0.993532 | −0.00936 | 0.006974 | NO |
| TG(16:0_38:2)_Triacylglycerols | 21.03 | >0.99999999 | 1.003694 | 0.00532 | 0.00693 | NO |
| TG(20:3_34:1)_Triacylglycerols | 21.31 | >0.99999999 | 0.995698 | −0.00622 | 0.006893 | NO |
| SM C16:0_Sphingolipids | 21.09 | >0.99999999 | 0.999086 | −0.00132 | 0.006513 | NO |
| TG(18:0_34:3)_Triacylglycerols | 22.19 | >0.99999999 | 0.995625 | −0.00633 | 0.006138 | NO |
| TG(22:5_32:1)_Triacylglycerols | 17.37 | >0.99999999 | 1.004295 | 0.006184 | 0.005277 | NO |
| SM C16:1_Sphingolipids | 17.42 | >0.99999999 | 1.001229 | 0.001772 | 0.004303 | NO |
| TG(18:3_36:1)_Triacylglycerols | 22.17 | >0.99999999 | 1.001718 | 0.002476 | 0.003481 | NO |
| TG(16:0_38:1)_Triacylglycerols | 21.09 | >0.99999999 | 1.001131 | 0.001631 | 0.001647 | NO |
| CE(15:1)_Cholesterol Esters | 30.61 | >0.99999999 | 0.999712 | −0.00042 | 0.000931 | NO |
| TCA_Bile Acids | 37.65 | >0.99999999 | 1.000892 | 0.001286 | 0.000476 | NO |
| lysoPC a C18:1_ | 28.3 | >0.99999999 | 1 | 0 | 0.000313 | NO |
| Glycerophospholipids | | | | | | |

TABLE S3
123 candidate biomarker proteins significantly differentially abundant (p < 0.05) in DYT1 patients as compared to healthy controls

| Protein | | | | Mean | Mean | SD | SD | HC | DYT1 |
|---|---|---|---|---|---|---|---|---|---|
| Accession | Genes | Protein Descriptions | Protein Names | HC | DYT1 | HC | DYT1 | (N) | (N) |
| Q9NPF8 | ADAP2 | Arf-GAP with dual PH | ADAP2_HUMAN | 11.70 | 11.456 | 0.192 | 0.128 | 27 | 13 |
| | | domain-containing protein 2 | | | | | | | |
| P06454 | PTMA | Prothymosin alpha | PTMA_HUMAN | 9.10 | 8.376 | 0.467 | 0.493 | 27 | 13 |
| P07477 | PRSS1 | Serine protease 1 | TRY1_HUMAN | 11.23 | 10.851 | 0.413 | 0.200 | 27 | 13 |
| Q8WWY7 | WFDC12 | WAP four-disulfide core | WFD12_HUMAN | 5.90 | 4.356 | 1.395 | 1.041 | 27 | 13 |
| | | domain protein 12 | | | | | | | |
| Q9H223 | EHD4 | EH domain-containing | EHD4_HUMAN | 10.31 | 8.194 | 2.126 | 1.318 | 27 | 13 |
| | | protein 4 | | | | | | | |
| P02730 | SLC4A1 | Band 3 anion transport | B3AT_HUMAN | 7.62 | 9.918 | 1.096 | 1.812 | 27 | 13 |
| | | protein | | | | | | | |
| O95866 | MPIG6B | Megakaryocyte and platelet | G6B_HUMAN | 6.14 | 7.965 | 1.617 | 1.326 | 25 | 13 |
| | | inhibitory receptor G6b | | | | | | | |
| Q49A17 | GALNTL6 | Polypeptide N- | GLTL6_HUMAN | 7.57 | 5.463 | 2.819 | 0.571 | 26 | 13 |
| | | acetylgalactosaminyl- | | | | | | | |
| | | transferase-like 6 | | | | | | | |
| P07910 | HNRNPC | Heterogeneous nuclear | HNRPC_HUMAN | 8.47 | 7.723 | 0.772 | 0.530 | 27 | 13 |
| | | ribonucleoproteins C1/C2 | | | | | | | |
| Q13361 | MFAP5 | Microfibrillar-associated | MFAP5_HUMAN | 4.27 | 5.634 | 1.445 | 0.932 | 27 | 12 |
| | | protein 5 | | | | | | | |
| P06239; | LCK; FYN; | Tyrosine-protein kinase | LCK_HUMAN; | 2.13 | 3.537 | 1.083 | 0.955 | 17 | 10 |
| P06241; | YES1; SRC | Lck; Tyrosine-protein kinase | FYN_HUMAN; | | | | | | |
| P07947; | | Fyn; Tyrosine-protein kinase | YES_HUMAN; | | | | | | |
| P12931 | | Yes;Proto-oncogene | SRC_HUMAN | | | | | | |
| | | tyrosine-protein kinase Src | | | | | | | |
| P42356 | PI4KA | Phosphatidylinositol 4-kinase | PI4KA_HUMAN | 11.97 | 11.703 | 0.347 | 0.164 | 27 | 13 |
| | | alpha | | | | | | | |
| P61225 | RAP2B | Ras-related protein Rap-2b | RAP2B_HUMAN | 4.16 | 5.827 | 1.773 | 1.127 | 24 | 11 |
| O75604 | USP2 | Ubiquitin carboxyl-terminal | UBP2_HUMAN | 4.64 | 3.197 | 1.467 | 1.249 | 26 | 13 |
| | | hydrolase 2 | | | | | | | |
| Q10567 | AP1B1 | AP-1 complex subunit beta-1 | AP1B1_HUMAN | 6.19 | 4.856 | 1.309 | 1.217 | 27 | 13 |
| Q9Y5E8 | PCDHB15 | Protocadherin beta-15 | PCDBF_HUMAN | 11.61 | 10.301 | 1.083 | 1.255 | 27 | 13 |
| Q96Q05 | TRAPPC9 | Trafficking protein particle | TPPC9_HUMAN | 6.66 | 5.743 | 1.328 | 0.587 | 27 | 13 |
| | | complex subunit 9 | | | | | | | |
| Q92953 | KCNB2 | Potassium voltage-gated | KCNB2_HUMAN | 5.17 | 7.576 | 1.845 | 2.351 | 26 | 13 |
| | | channel subfamily | | | | | | | |
| | | B member 2 | | | | | | | |
| P14770 | GP9 | Platelet glycoprotein IX | GPIX_HUMAN | 7.19 | 8.645 | 0.915 | 1.464 | 27 | 13 |
| O60613 | SELENOF | Selenoprotein F | SEP15_HUMAN | 7.28 | 6.279 | 0.777 | 0.989 | 27 | 13 |
| Q9H4B7 | TUBB1 | Tubulin beta-1 chain | TBB1_HUMAN | 5.17 | 6.368 | 0.628 | 1.257 | 27 | 13 |
| Q15942 | ZYX | Zyxin | ZYX_HUMAN | 3.49 | 4.425 | 0.988 | 0.890 | 27 | 13 |
| Q9P225 | DNAH2 | Dynein axonemal heavy | DYH2_HUMAN | 8.58 | 7.827 | 0.683 | 0.750 | 27 | 13 |
| | | chain 2 | | | | | | | |
| P98179 | RBM3 | RNA-binding protein 3 | RBM3_HUMAN | 5.04 | 3.694 | 1.496 | 1.288 | 27 | 13 |
| Q13418 | ILK | Integrin-linked protein kinase | ILK_HUMAN | 6.05 | 7.193 | 0.728 | 1.274 | 27 | 13 |
| P16157 | ANK1 | Ankyrin-1 | ANK1_HUMAN | 3.92 | 5.232 | 0.532 | 1.491 | 27 | 13 |
| P08514 | ITGA2B | Integrin alpha-IIb | ITA2B_HUMAN | 8.85 | 10.153 | 1.182 | 1.399 | 27 | 13 |
| P35030 | PRSS3 | Trypsin-3 | TRY3_HUMAN | 9.10 | 8.821 | 0.353 | 0.269 | 27 | 13 |
| P61812 | TGFB2 | Transforming growth factor | TGFB2_HUMAN | 5.40 | 4.406 | 1.263 | 0.958 | 27 | 13 |
| | | beta-2 proprotein | | | | | | | |
| Q9NY56 | OBP2A | Odorant-binding protein 2a | OBP2A_HUMAN | 6.51 | 8.456 | 1.999 | 1.857 | 27 | 11 |
| P27105 | STOM | Stomatin | STOM_HUMAN | 6.89 | 8.193 | 1.019 | 1.505 | 27 | 13 |
| Q9H8M2 | BRD9 | Bromodomain-containing | BRD9_HUMAN | 5.68 | 7.458 | 1.730 | 1.784 | 26 | 11 |
| | | protein 9 | | | | | | | |
| P36957 | DLST | Dihydrolipoyllysine-residue | ODO2_HUMAN | 6.84 | 5.753 | 1.068 | 1.213 | 27 | 13 |
| | | succinyltransferase | | | | | | | |
| | | component | | | | | | | |
| | | of 2-oxoglutarate | | | | | | | |
| | | dehydrogenase complex, | | | | | | | |
| | | mitochondrial | | | | | | | |
| O76009 | KRT33A | Keratin, type I cuticular | KT33A_HUMAN | 5.29 | 4.309 | 1.300 | 0.976 | 27 | 13 |
| | | Ha3-I | | | | | | | |
| Q9HB58 | SP110 | Sp110 nuclear body protein | SP110_HUMAN | 9.75 | 8.490 | 1.609 | 1.286 | 27 | 13 |
| Q9NP78 | ABCB9 | ABC-type oligopeptide | ABCB9_HUMAN | 11.73 | 9.723 | 1.576 | 2.379 | 27 | 13 |
| | | transporter ABCB9 | | | | | | | |
| O75348 | ATP6V1G1 | V-type proton ATPase | VATG1_HUMAN | 3.50 | 2.740 | 1.137 | 0.706 | 27 | 13 |
| | | subunit G 1 | | | | | | | |
| Q9NVJ2 | ARL8B | ADP-ribosylation factor-like | ARL8B_HUMAN | 1.31 | −0.118 | 1.190 | 1.069 | 10 | 9 |
| | | protein 8B | | | | | | | |
| O94979 | SEC31A | Protein transport protein | SC31A_HUMAN | 9.36 | 8.231 | 1.325 | 1.238 | 27 | 13 |
| | | Sec31A | | | | | | | |
| P61599 | NAA20 | N-alpha-acetyltransferase 20 | NAA20_HUMAN | 2.91 | 4.199 | 1.488 | 0.879 | 19 | 7 |
| Q08830 | FGL1 | Fibrinogen-like protein 1 | FGL1_HUMAN | 7.97 | 7.153 | 1.208 | 0.769 | 27 | 13 |
| Q8WVM0 | TFB1M | Dimethyladenosine | TFB1M_HUMAN | 5.26 | 7.004 | 2.319 | 1.479 | 23 | 10 |
| | | transferase | | | | | | | |
| | | 1, mitochondrial | | | | | | | |
| Q96P70 | IPO9 | Importin-9 | IPO9_HUMAN | 8.24 | 7.094 | 1.197 | 1.338 | 27 | 13 |
| P11277 | SPTB | Spectrin beta chain, | SPTB1_HUMAN | 5.61 | 6.706 | 0.642 | 1.382 | 27 | 13 |
| | | erythrocytic | | | | | | | |
| Q9H7D7 | WDR26 | WD repeat-containing | WDR26_HUMAN | 4.70 | 5.387 | 1.062 | 0.604 | 26 | 12 |
| | | protein 26 | | | | | | | |
| P21333 | FLNA | Filamin-A | FLNA_HUMAN | 6.89 | 8.016 | 0.949 | 1.411 | 27 | 13 |
| Q9H4A3 | WNK1 | Serine/threonine-protein | WNK1_HUMAN | 2.63 | 1.769 | 1.467 | 0.677 | 25 | 13 |
| | | kinase WNK1 | | | | | | | |
| Q6ZW13 | C16orf86 | Uncharacterized protein | CP086_HUMAN | 8.57 | 10.022 | 2.248 | 1.419 | 27 | 13 |
| | | C16orf86 | | | | | | | |
| Q8TD19 | NEK9 | Serine/threonine-protein | NEK9_HUMAN | 12.35 | 11.764 | 0.531 | 0.737 | 27 | 13 |
| | | kinase Nek9 | | | | | | | |
| P56192 | MARS1 | Methionine-tRNA ligase, | SYMC_HUMAN | 5.24 | 4.049 | 1.784 | 1.224 | 27 | 13 |
| | | cytoplasmic | | | | | | | |
| P03973 | SLPI | Antileukoproteinase | SLPI_HUMAN | 6.42 | 5.326 | 1.060 | 1.365 | 27 | 13 |
| Q9BYW2 | SETD2 | Histone-lysine N- | SETD2_HUMAN | 10.52 | 8.990 | 2.016 | 1.727 | 27 | 13 |
| | | methyltransferase SETD2 | | | | | | | |
| P25391 | LAMA1 | Laminin subunit alpha-1 | LAMA1_HUMAN | 4.57 | 3.504 | 1.314 | 1.245 | 27 | 13 |
| P39656 | DDOST | Dolichyl- | OST48_HUMAN | 4.66 | 3.994 | 1.027 | 0.675 | 27 | 13 |
| | | diphosphooligosaccharide— | | | | | | | |
| | | protein glycosyltransferase | | | | | | | |
| | | 48 kDa subunit | | | | | | | |
| O94964 | SOGA1 | Protein SOGA1 | SOGA1_HUMAN | 11.58 | 9.962 | 1.105 | 2.121 | 27 | 13 |
| P27361 | MAPK3 | Mitogen-activated protein | MK03_HUMAN | 4.52 | 3.577 | 1.086 | 1.138 | 27 | 13 |
| | | kinase 3 | | | | | | | |
| Q96AQ9 | FAM131C | Protein FAM131C | F131C_HUMAN | 2.56 | 4.777 | 1.271 | 2.288 | 18 | 9 |
| P61586 | RHOA | Transforming protein RhoA | RHOA_HUMAN | 5.98 | 6.529 | 0.528 | 0.693 | 27 | 13 |
| P23490 | LORICRIN | Loricrin | LORI_HUMAN | 7.19 | 6.295 | 1.282 | 0.982 | 27 | 13 |
| PODTE2 | IGHV8-51-1 | Probable non-functional | HV511_HUMAN | 4.13 | 2.889 | 1.387 | 1.273 | 22 | 10 |
| | | immunoglobulin heavy | | | | | | | |
| | | variable 8-51-1 | | | | | | | |
| Q9BX67 | JAM3 | Junctional adhesion | JAM3_HUMAN | 9.50 | 7.895 | 1.319 | 2.121 | 27 | 13 |
| | | molecule C | | | | | | | |
| P11166 | SLC2A1 | Solute carrier family 2, | GTR1_HUMAN | 6.11 | 7.183 | 0.932 | 1.416 | 27 | 13 |
| | | facilitated glucose | | | | | | | |
| | | transporter member 1 | | | | | | | |
| O60256 | PRPSAP2 | Phosphoribosyl | KPRB_HUMAN | 3.70 | 2.595 | 1.437 | 1.247 | 26 | 12 |
| | | pyrophosphate | | | | | | | |
| | | synthase-associated protein 2 | | | | | | | |
| P08567 | PLEK | Pleckstrin | PLEK_HUMAN | 5.76 | 6.963 | 0.973 | 1.607 | 27 | 13 |
| P62195 | PSMC5 | 26S proteasome regulatory | PRS8_HUMAN | 5.11 | 4.508 | 0.950 | 0.634 | 27 | 13 |
| | | subunit 8 | | | | | | | |
| P46013 | MKI67 | Proliferation marker protein | KI67_HUMAN | 9.49 | 7.413 | 2.538 | 2.570 | 27 | 13 |
| | | Ki-67 | | | | | | | |
| P27635 | RPL10 | Large ribosomal subunit | RL10_HUMAN | 6.22 | 5.515 | 1.245 | 0.656 | 27 | 13 |
| | | protein uL16 | | | | | | | |
| P49761 | CLK3 | Dual specificity protein | CLK3_HUMAN | 10.50 | 10.306 | 0.288 | 0.217 | 27 | 13 |
| | | kinase CLK3 | | | | | | | |
| Q96AG4 | LRRC59 | Leucine-rich | LRC59_HUMAN | 2.77 | 1.966 | 1.379 | 0.621 | 23 | 11 |
| | | repeat-containing | | | | | | | |
| | | protein 59 | | | | | | | |
| P02511 | CRYAB | Alpha-crystallin B chain | CRYAB_HUMAN | 4.21 | 3.127 | 1.352 | 1.355 | 26 | 13 |
| P48739 | PITPNB | Phosphatidylinositol transfer | PIPNB_HUMAN | 5.21 | 4.149 | 1.194 | 1.379 | 27 | 13 |
| | | protein beta isoform | | | | | | | |
| O95361 | TRIM16 | Tripartite motif-containing | TRI16_HUMAN | 3.73 | 2.654 | 1.205 | 1.184 | 25 | 10 |
| | | protein 16 | | | | | | | |
| O75608 | LYPLA1 | Acyl-protein thioesterase 1 | LYPA1_HUMAN | 9.55 | 8.175 | 1.001 | 1.936 | 27 | 13 |
| P00451 | F8 | Coagulation factor VIII | FA8_HUMAN | 5.26 | 4.143 | 1.466 | 1.397 | 26 | 13 |
| Q9H4M9; | EHD1; | EH domain-containing | EHD1_HUMAN; | 3.20 | 4.224 | 1.089 | 1.270 | 21 | 12 |
| | | protein 1; | EHD3_HUMAN; | | | | | | |
| Q9NZN3; | EHD3; | EH domain-containing | EHD2_HUMAN | | | | | | |
| | | protein 3; | | | | | | | |
| Q9NZN4 | EHD2 | EH domain-containing | | | | | | | |
| | | protein 2 | | | | | | | |
| Q03252 | LMNB2 | Lamin-B2 | LMNB2_HUMAN | 4.13 | 3.328 | 1.022 | 1.034 | 27 | 13 |
| P04275 | VWF | von Willebrand factor | VWF_HUMAN | 15.47 | 14.625 | 0.979 | 1.124 | 27 | 13 |
| Q13304 | GPR17 | Uracil nucleotide/cysteinyl | GPR17_HUMAN | 6.18 | 4.951 | 2.630 | 0.700 | 27 | 13 |
| | | leukotriene receptor | | | | | | | |
| Q9Y490 | TLN1 | Talin-1 | TLN1_HUMAN | 7.57 | 8.739 | 0.963 | 1.654 | 27 | 13 |
| P22061 | PCMT1 | Protein-L-isoaspartate(D- | PIMT_HUMAN | 4.50 | 3.811 | 1.096 | 0.806 | 27 | 13 |
| | | aspartate) | | | | | | | |
| | | O-methyltransferase | | | | | | | |
| P09622 | DLD | Dihydrolipoyl | DLDH_HUMAN | 4.81 | 3.942 | 1.008 | 1.170 | 27 | 13 |
| | | dehydrogenase, | | | | | | | |
| | | mitochondrial | | | | | | | |
| P10321 | HLA-C | HLA class I | HLAC_HUMAN | 6.84 | 7.954 | 1.483 | 1.432 | 27 | 13 |
| | | histocompatibility | | | | | | | |
| | | antigen, C alpha chain | | | | | | | |
| P13224 | GP1BB | Platelet glycoprotein lb beta | GP1BB_HUMAN | 5.43 | 6.766 | 0.934 | 1.934 | 27 | 13 |
| | | chain | | | | | | | |
| Q02978 | SLC25A11 | Mitochondrial 2- | M2OM_HUMAN | 0.20 | −1.129 | 1.339 | 0.803 | 9 | 6 |
| | | oxoglutarate/malate carrier | | | | | | | |
| | | protein | | | | | | | |
| P40429 | RPL13A | Large ribosomal subunit | RL13A_HUMAN | 5.32 | 4.489 | 1.117 | 1.074 | 27 | 13 |
| | | protein uL13 | | | | | | | |
| Q15223 | NECTIN1 | Nectin-1 | NECT1_HUMAN | 4.67 | 6.641 | 2.064 | 2.433 | 24 | 11 |
| Q9NWF9 | RNF216 | E3 ubiquitin-protein ligase | RN216_HUMAN | 2.76 | 4.837 | 2.507 | 2.306 | 22 | 10 |
| | | RNF216 | | | | | | | |
| Q8TE59 | ADAMTS19 | A disintegrin and | ATS19_HUMAN | 8.43 | 10.077 | 2.597 | 1.703 | 24 | 11 |
| | | metalloproteinase with | | | | | | | |
| | | thrombospondin motifs 19 | | | | | | | |
| Q4KMQ2 | ANO6 | Anoctamin-6 | ANO6_HUMAN | 9.33 | 7.713 | 1.702 | 2.265 | 27 | 13 |
| O00232 | PSMD12 | 26S proteasome non-ATPase | PSD12_HUMAN | 5.33 | 4.269 | 1.359 | 1.415 | 27 | 13 |
| | | regulatory subunit 12 | | | | | | | |
| Q9UBG0 | MRC2 | C-type mannose receptor 2 | MRC2_HUMAN | 4.87 | 5.949 | 1.605 | 1.294 | 27 | 12 |
| Q9Y4G6 | TLN2 | Talin-2 | TLN2_HUMAN | 6.13 | 4.909 | 1.343 | 1.705 | 27 | 13 |
| Q8N128 | FAM177A1 | Protein FAM177A1 | F177A_HUMAN | 4.10 | 5.304 | 1.674 | 1.452 | 23 | 12 |
| P08134 | RHOC | Rho-related GTP-binding | RHOC_HUMAN | 5.12 | 6.044 | 1.036 | 1.289 | 26 | 13 |
| | | protein RhoC | | | | | | | |
| P69905 | HBA1 | Hemoglobin subunit alpha | HBA_HUMAN | 13.50 | 15.348 | 1.728 | 2.673 | 27 | 13 |
| P09110 | ACAA1 | 3-ketoacyl-CoA thiolase, | THIK_HUMAN | 3.91 | 3.044 | 1.127 | 1.103 | 26 | 12 |
| | | peroxisomal | | | | | | | |
| Q8WXE1 | ATRIP | ATR-interacting protein | ATRIP_HUMAN | 10.86 | 10.249 | 0.801 | 0.831 | 27 | 13 |
| Q9Y5Y4 | PTGDR2 | Prostaglandin D2 receptor 2 | PD2R2_HUMAN | 14.27 | 12.868 | 2.287 | 1.695 | 27 | 13 |
| P68402 | PAFAH1B2 | Platelet-activating factor | PA1B2_HUMAN | 3.22 | 4.198 | 1.437 | 1.251 | 25 | 13 |
| | | acetylhydrolase IB subunit | | | | | | | |
| | | alpha2 | | | | | | | |
| Q93009 | USP7 | Ubiquitin carboxyl-terminal | UBP7_HUMAN | 4.22 | 3.505 | 1.083 | 0.916 | 27 | 13 |
| | | hydrolase 7 | | | | | | | |
| P12236 | SLC25A6 | ADP/ATP translocase 3 | ADT3_HUMAN | 4.05 | 4.947 | 1.579 | 0.920 | 26 | 11 |
| P36542 | ATP5F1C | ATP synthase subunit | ATPG_HUMAN | 8.69 | 7.830 | 1.461 | 1.019 | 27 | 13 |
| | | gamma, mitochondrial | | | | | | | |
| Q9BQE3 | TUBA1C | Tubulin alpha-1C chain | TBA1C_HUMAN | 7.71 | 6.242 | 2.092 | 1.957 | 27 | 13 |
| O15254 | ACOX3 | Peroxisomal | ACOX3_HUMAN | 4.92 | 4.229 | 0.919 | 0.955 | 27 | 13 |
| | | acyl-coenzyme A | | | | | | | |
| | | oxidase 3 | | | | | | | |
| Q14997 | PSME4 | Proteasome activator | PSME4_HUMAN | 8.24 | 7.365 | 1.976 | 0.566 | 27 | 13 |
| | | complex subunit 4 | | | | | | | |
| Q16658 | FSCN1 | Fascin | FSCN1_HUMAN | 5.18 | 4.187 | 1.196 | 1.400 | 27 | 13 |
| P02042 | HBD | Hemoglobin subunit delta | HBD_HUMAN | 9.30 | 10.966 | 1.458 | 2.501 | 27 | 13 |
| P16401 | H1-5 | Histone H1.5 | H15_HUMAN | 3.05 | 2.184 | 1.390 | 0.987 | 23 | 12 |
| Q9Y6E2 | BZW2 | elF5-mimic protein 1 | 5MP1_HUMAN | 4.31 | 3.536 | 1.267 | 0.977 | 27 | 13 |
| Q13439 | GOLGA4 | Golgin subfamily A | GOGA4_HUMAN | 10.24 | 11.154 | 1.540 | 1.145 | 27 | 13 |
| | | member 4 | | | | | | | |
| P35573 | AGL | Glycogen debranching | GDE_HUMAN | 6.61 | 6.109 | 0.776 | 0.654 | 27 | 13 |
| | | enzyme | | | | | | | |
| Q8NCA5 | FAM98A | Protein FAM98A | FA98A_HUMAN | 2.94 | 1.241 | 1.549 | 1.694 | 17 | 7 |
| P68363 | TUBA1B | Tubulin alpha-1B chain | TBA1B_HUMAN | 9.36 | 10.079 | 1.044 | 0.982 | 27 | 13 |
| Q13148 | TARDBP | TAR DNA-binding | TADBP_HUMAN | 4.91 | 3.697 | 1.755 | 1.663 | 27 | 13 |
| | | protein 43 | | | | | | | |
| Q9H2S5 | RNF39 | RING finger protein 39 | RNF39_HUMAN | 3.37 | 2.632 | 1.204 | 0.936 | 26 | 13 |
| P62277 | RPS13 | Small ribosomal subunit | RS13_HUMAN | 5.80 | 5.247 | 0.937 | 0.716 | 27 | 13 |
| | | protein uS15 | | | | | | | |
| Q5SW79 | CEP170 | Centrosomal protein of | CE170_HUMAN | 2.36 | 3.627 | 1.966 | 1.247 | 20 | 9 |
| | | 170 kDa | | | | | | | |
| Q9H497 | TOR3A | Torsin-3A | TOR3A_HUMAN | 8.16 | 8.654 | 0.728 | 0.683 | 27 | 13 |
| P22102 | GART | Trifunctional purine | PUR2_HUMAN | 4.51 | 3.587 | 1.208 | 1.332 | 26 | 13 |
| | | biosynthetic | | | | | | | |
| | | protein adenosine-3 | | | | | | | |
| P35579 | MYH9 | Myosin-9 | MYH9_HUMAN | 6.30 | 6.965 | 0.984 | 0.938 | 27 | 13 |
| Q8TB73 | NDNF | Protein NDNF | NDNF_HUMAN | 2.86 | 4.696 | 2.307 | 2.356 | 20 | 11 |
| Q9Y5X9 | LIPG | Endothelial lipase | LIPG_HUMAN | 6.04 | 5.060 | 1.186 | 1.474 | 26 | 13 |
| Q96DR8 | MUCL1 | Mucin-like protein 1 | MUCL1_HUMAN | 7.72 | 6.887 | 1.480 | 1.047 | 27 | 13 |

123 candidate biomarker proteins significantly differentially abundant (p < 0.05) in DYT1 patients as compared to healthy controls

| Protein | | | | | Log2F | | Cohen's | |
|---|---|---|---|---|---|---|---|---|
| Accession | Genes | Protein Descriptions | Protein Names | Pval | C | -LogPval | D | Spooled |
| Q9NPF8 | ADAP2 | Arf-GAP with dual PH | ADAP2_HUMAN | 0.0000310 | −0.2459 | 4.50799 | −1.41017 | 0.17 |
| | | domain-containing protein 2 | | | | | | |
| P06454 | PTMA | Prothymosin alpha | PTMA_HUMAN | 0.0002132 | −0.7203 | 3.67116 | −1.51485 | 0.48 |
| P07477 | PRSS1 | Serine protease 1 | TRY1_HUMAN | 0.0003360 | −0.3818 | 3.47367 | −1.06205 | 0.36 |
| Q8WWY7 | WFDC12 | WAP four-disulfide core | WFD12_HUMAN | 0.0004450 | −1.5491 | 3.35160 | −1.19733 | 1.29 |
| | | domain protein 12 | | | | | | |
| Q9H223 | EHD4 | EH domain-containing | EHD4_HUMAN | 0.0004760 | −2.1126 | 3.32240 | −1.10693 | 1.91 |
| | | protein 4 | | | | | | |
| P02730 | SLC4A1 | Band 3 anion transport | B3AT_HUMAN | 0.0006269 | 2.2980 | 3.20278 | 1.68541 | 1.36 |
| | | protein | | | | | | |
| O95866 | MPIG6B | Megakaryocyte and platelet | G6B_HUMAN | 0.0008356 | 1.8244 | 3.07801 | 1.19553 | 1.53 |
| | | inhibitory receptor G6b | | | | | | |
| Q49A17 | GALNTL6 | Polypeptide N- | GLTL6_HUMAN | 0.0009788 | −2.1098 | 3.00931 | −0.90181 | 2.34 |
| | | acetylgalactosaminyl- | | | | | | |
| | | transferase-like 6 | | | | | | |
| P07910 | HNRNPC | Heterogeneous nuclear | HNRPC_HUMAN | 0.0011664 | −0.7428 | 2.93316 | −1.05382 | 0.70 |
| | | ribonucleoproteins C1/C2 | | | | | | |
| Q13361 | MFAP5 | Microfibrillar-associated | MFAP5_HUMAN | 0.0012911 | 1.3666 | 2.88904 | 1.04018 | 1.31 |
| | | protein 5 | | | | | | |
| P06239; | LCK; FYN; | Tyrosine-protein kinase | LCK_HUMAN; | 0.0020638 | 1.4065 | 2.68533 | 1.35366 | 1.04 |
| P06241; | YES1; SRC | Lck; Tyrosine-protein kinase | FYN_HUMAN; | | | | | |
| P07947; | | Fyn; Tyrosine-protein kinase | YES_HUMAN; | | | | | |
| P12931 | | Yes;Proto-oncogene | SRC_HUMAN | | | | | |
| | | tyrosine-protein kinase Src | | | | | | |
| P42356 | PI4KA | Phosphatidylinositol | PI4KA_HUMAN | 0.0021871 | −0.2654 | 2.66012 | −0.88083 | 0.30 |
| | | 4-kinase alpha | | | | | | |
| P61225 | RAP2B | Ras-related protein Rap-2b | RAP2B_HUMAN | 0.0022055 | 1.6659 | 2.65649 | 1.03822 | 1.60 |
| O75604 | USP2 | Ubiquitin carboxyl-terminal | UBP2_HUMAN | 0.0033893 | −1.4420 | 2.46989 | −1.03018 | 1.40 |
| | | hydrolase 2 | | | | | | |
| Q10567 | AP1B1 | AP-1 complex subunit beta-1 | AP1B1_HUMAN | 0.0038596 | −1.3386 | 2.41346 | −1.04537 | 1.28 |
| Q9Y5E8 | PCDHB15 | Protocadherin beta-15 | PCDBF_HUMAN | 0.0040054 | −1.3119 | 2.39735 | −1.15063 | 1.14 |
| Q96Q05 | TRAPPC9 | Trafficking protein particle | TPPC9_HUMAN | 0.0042581 | −0.9216 | 2.37079 | −0.80345 | 1.15 |
| | | complex subunit 9 | | | | | | |
| Q92953 | KCNB2 | Potassium voltage-gated | KCNB2_HUMAN | 0.0043120 | 2.4057 | 2.36532 | 1.18900 | 2.02 |
| | | channel subfamily | | | | | | |
| | | B member 2 | | | | | | |
| P14770 | GP9 | Platelet glycoprotein IX | GPIX_HUMAN | 0.0044040 | 1.4570 | 2.35616 | 1.30301 | 1.12 |
| O60613 | SELENOF | Selenoprotein F | SEP15_HUMAN | 0.0046314 | −0.9996 | 2.33429 | −1.17650 | 0.85 |
| Q9H4B7 | TUBB1 | Tubulin beta-1 chain | TBB1_HUMAN | 0.0054086 | 1.1985 | 2.26691 | 1.36741 | 0.88 |
| Q15942 | ZYX | Zyxin | ZYX_HUMAN | 0.0059550 | 0.9322 | 2.22512 | 0.97335 | 0.96 |
| Q9P225 | DNAH2 | Dynein axonemal heavy | DYH2_HUMAN | 0.0060304 | −0.7484 | 2.21965 | −1.06141 | 0.71 |
| | | chain 2 | | | | | | |
| P98179 | RBM3 | RNA-binding protein 3 | RBM3_HUMAN | 0.0066236 | −1.3483 | 2.17891 | −0.94058 | 1.43 |
| Q13418 | ILK | Integrin-linked protein kinase | ILK_HUMAN | 0.0082282 | 1.1474 | 2.08469 | 1.22589 | 0.94 |
| P16157 | ANK1 | Ankyrin-1 | ANK1_HUMAN | 0.0083165 | 1.3159 | 2.08006 | 1.39017 | 0.95 |
| P08514 | ITGA2B | Integrin alpha-IIb | ITA2B_HUMAN | 0.0088190 | 1.3017 | 2.05458 | 1.03726 | 1.25 |
| P35030 | PRSS3 | Trypsin-3 | TRY3_HUMAN | 0.0094791 | −0.2795 | 2.02323 | −0.85031 | 0.33 |
| P61812 | TGFB2 | Transforming growth factor | TGFB2_HUMAN | 0.0095869 | −0.9952 | 2.01832 | −0.84664 | 1.18 |
| | | beta-2 proprotein | | | | | | |
| Q9NY56 | OBP2A | Odorant-binding protein 2a | OBP2A_HUMAN | 0.0096276 | 1.9446 | 2.01648 | 0.99207 | 1.96 |
| P27105 | STOM | Stomatin | STOM_HUMAN | 0.0112629 | 1.3063 | 1.94835 | 1.09387 | 1.19 |
| Q9H8M2 | BRD9 | Bromodomain-containing | BRD9_HUMAN | 0.0116939 | 1.7800 | 1.93204 | 1.01960 | 1.75 |
| | | protein 9 | | | | | | |
| P36957 | DLST | Dihydrolipoyllysine-residue | ODO2_HUMAN | 0.0118086 | −1.0854 | 1.92780 | −0.97296 | 1.12 |
| | | succinyltransferase | | | | | | |
| | | component | | | | | | |
| | | of 2-oxoglutarate | | | | | | |
| | | dehydrogenase complex, | | | | | | |
| | | mitochondrial | | | | | | |
| O76009 | KRT33A | Keratin, type I cuticular | KT33A_HUMAN | 0.0118724 | −0.9858 | 1.92546 | −0.81645 | 1.21 |
| | | Ha3-I | | | | | | |
| Q9HB58 | SP110 | Sp110 nuclear body protein | SP110_HUMAN | 0.0125438 | −1.2569 | 1.90157 | 0.82970 | 1.51 |
| Q9NP78 | ABCB9 | ABC-type oligopeptide | ABCB9_HUMAN | 0.0129605 | −2.0118 | 1.88738 | −1.07751 | 1.87 |
| | | transporter ABCB9 | | | | | | |
| O75348 | ATP6V1G1 | V-type proton ATPase | VATG1_HUMAN | 0.0136300 | −0.7626 | 1.86550 | −0.74692 | 1.02 |
| | | subunit G 1 | | | | | | |
| Q9NVJ2 | ARL8B | ADP-ribosylation factor-like | ARL8B_HUMAN | 0.0138009 | −1.4230 | 1.86009 | −1.25395 | 1.13 |
| | | protein 8B | | | | | | |
| O94979 | SEC31A | Protein transport protein | SC31A_HUMAN | 0.0141139 | −1.1276 | 1.85035 | −0.86858 | 1.30 |
| | | Sec31A | | | | | | |
| P61599 | NAA20 | N-alpha-acetyltransferase 20 | NAA20_HUMAN | 0.0142542 | 1.2890 | 1.84606 | 0.94666 | 1.36 |
| Q08830 | FGL1 | Fibrinogen-like protein 1 | FGL1_HUMAN | 0.0142839 | −0.8140 | 1.84515 | −0.74757 | 1.09 |
| Q8WVM0 | TFB1M | Dimethyladenosine | TFB1M_HUMAN | 0.0153477 | 1.7443 | 1.81396 | 0.82682 | 2.11 |
| | | transferase 1, mitochondrial | | | | | | |
| Q96P70 | IPO9 | Importin-9 | IPO9_HUMAN | 0.0159821 | −1.1421 | 1.79637 | −0.91870 | 1.24 |
| P11277 | SPTB | Spectrin beta chain, | SPTB1_HUMAN | 0.0164701 | 1.0913 | 1.78330 | 1.16004 | 0.94 |
| | | erythrocytic | | | | | | |
| Q9H7D7 | WDR26 | WD repeat-containing | WDR26_HUMAN | 0.0169016 | 0.6824 | 1.77207 | 0.72142 | 0.95 |
| | | protein 26 | | | | | | |
| P21333 | FLNA | Filamin-A | FLNA_HUMAN | 0.0180876 | 1.1267 | 1.74262 | 1.00974 | 1.12 |
| Q9H4A3 | WNK1 | Serine/threonine-protein | WNK1_HUMAN | 0.0181770 | −0.8626 | 1.74048 | −0.68455 | 1.26 |
| | | kinase WNK1 | | | | | | |
| Q6ZW13 | C16orf86 | Uncharacterized protein | CP086_HUMAN | 0.0183479 | 1.4469 | 1.73641 | 0.71526 | 2.02 |
| | | C16orf86 | | | | | | |
| Q8TD19 | NEK9 | Serine/threonine-protein | NEK9_HUMAN | 0.0186458 | −0.5905 | 1.72942 | −0.97774 | 0.60 |
| | | kinase Nek9 | | | | | | |
| P56192 | MARS1 | Methionine-tRNA ligase, | SYMC_HUMAN | 0.0193285 | −1.1875 | 1.71380 | −0.72935 | 1.63 |
| | | cytoplasmic | | | | | | |
| P03973 | SLPI | Antileukoproteinase | SLPI_HUMAN | 0.0195699 | −1.0950 | 1.70841 | −0.94014 | 1.16 |
| Q9BYW2 | SETD2 | Histone-lysine N- | SETD2_HUMAN | 0.0197348 | −1.5263 | 1.70477 | −0.79116 | 1.93 |
| | | methyltransferase SETD2 | | | | | | |
| P25391 | LAMA1 | Laminin subunit alpha-1 | LAMA1_HUMAN | 0.0199615 | −1.0641 | 1.69981 | −0.82329 | 1.29 |
| P39656 | DDOST | Dolichyl- | OST48_HUMAN | 0.0206137 | −0.6612 | 1.68584 | −0.71049 | 0.93 |
| | | diphosphooligosaccharide— | | | | | | |
| | | protein glycosyltransferase | | | | | | |
| | | 48 kDa subunit | | | | | | |
| O94964 | SOGA1 | Protein SOGA1 | SOGA1_HUMAN | 0.0206256 | −1.6154 | 1.68559 | −1.07544 | 1.50 |
| P27361 | MAPK3 | Mitogen-activated protein | MK03_HUMAN | 0.0207773 | −0.9403 | 1.68241 | −0.85267 | 1.10 |
| | | kinase 3 | | | | | | |
| Q96AQ9 | FAM131C | Protein FAM131C | F131C_HUMAN | 0.0211069 | 2.2179 | 1.67558 | 1.33180 | 1.67 |
| P61586 | RHOA | Transforming protein RhoA | RHOA_HUMAN | 0.0212053 | 0.5466 | 1.67356 | 0.93332 | 0.59 |
| P23490 | LORICRIN | Loricrin | LORI_HUMAN | 0.0214079 | −0.8919 | 1.66943 | −0.74586 | 1.20 |
| PODTE2 | IGHV8-51-1 | Probable non-functional | HV511_HUMAN | 0.0225152 | −1.2404 | 1.64752 | −0.91616 | 1.35 |
| | | immunoglobulin heavy | | | | | | |
| | | variable 8-51-1 | | | | | | |
| Q9BX67 | JAM3 | Junctional adhesion | JAM3_HUMAN | 0.0228611 | −1.6069 | 1.64090 | −0.99424 | 1.62 |
| | | molecule C | | | | | | |
| P11166 | SLC2A1 | Solute carrier family 2, | GTR1_HUMAN | 0.0231941 | 1.0758 | 1.63462 | 0.97117 | 1.11 |
| | | facilitated glucose | | | | | | |
| | | transporter member 1 | | | | | | |
| O60256 | PRPSAP2 | Phosphoribosyl | KPRB_HUMAN | 0.0234740 | −1.1046 | 1.62941 | −0.79940 | 1.38 |
| | | pyrophosphate | | | | | | |
| | | synthase-associated | | | | | | |
| | | protein 2 | | | | | | |
| P08567 | PLEK | Pleckstrin | PLEK_HUMAN | 0.0236300 | 1.2064 | 1.62654 | 0.99707 | 1.21 |
| P62195 | PSMC5 | 26S proteasome regulatory | PRS8_HUMAN | 0.0242551 | −0.5986 | 1.61520 | −0.69361 | 0.86 |
| | | subunit 8 | | | | | | |
| P46013 | MKI67 | Proliferation marker protein | KI67_HUMAN | 0.0246046 | −2.0752 | 1.60898 | −0.81434 | 2.55 |
| | | Ki-67 | | | | | | |
| P27635 | RPL10 | Large ribosomal subunit | RL10_HUMAN | 0.0247944 | −0.7035 | 1.60565 | −0.64316 | 1.09 |
| | | protein uL16 | | | | | | |
| P49761 | CLK3 | Dual specificity protein | CLK3_HUMAN | 0.0256767 | −0.1915 | 1.59046 | −0.71664 | 0.27 |
| | | kinase CLK3 | | | | | | |
| Q96AG4 | LRRC59 | Leucine-rich repeat- | LRC59_HUMAN | 0.0260475 | −0.8011 | 1.58423 | 0.67021 | 1.20 |
| | | containing protein 59 | | | | | | |
| P02511 | CRYAB | Alpha-crystallin B chain | CRYAB_HUMAN | 0.0265118 | −1.0869 | 1.57656 | −0.80356 | 1.35 |
| P48739 | PITPNB | Phosphatidylinositol transfer | PIPNB_HUMAN | 0.0266324 | −1.0641 | 1.57459 | −0.84745 | 1.26 |
| | | protein beta isoform | | | | | | |
| O95361 | TRIM16 | Tripartite motif-containing | TRI16_HUMAN | 0.0267332 | −1.0807 | 1.57295 | −0.90100 | 1.20 |
| | | protein 16 | | | | | | |
| O75608 | LYPLA1 | Acyl-protein thioesterase 1 | LYPA1_HUMAN | 0.0288812 | −1.3768 | 1.53938 | −1.00708 | 1.37 |
| P00451 | F8 | Coagulation factor VIII | FA8_HUMAN | 0.0288891 | −1.1178 | 1.53927 | 0.77430 | 1.44 |
| Q9H4M9; | EHD1; | EH domain-containing | EHD1_HUMAN; | 0.0291676 | 1.0255 | 1.53510 | 0.88680 | 1.16 |
| Q9NZN3; | EHD3; | protein 1; | EHD3_HUMAN; | | | | | |
| Q9NZN4 | EHD2 | EH domain-containing | EHD2_HUMAN | | | | | |
| | | protein 3; | | | | | | |
| | | EH domain-containing | | | | | | |
| | | protein 2 | | | | | | |
| Q03252 | LMNB2 | Lamin-B2 | LMNB2_HUMAN | 0.0293522 | −0.8070 | 1.53236 | −0.78660 | 1.03 |
| P04275 | VWF | von Willebrand factor | VWF_HUMAN | 0.0301611 | −0.8466 | 1.52055 | −0.82456 | 1.03 |
| Q13304 | GPR17 | Uracil nucleotide/cysteinyl | GPR17_HUMAN | 0.0305433 | −1.2256 | 1.51508 | −0.55443 | 2.21 |
| | | leukotriene receptor | | | | | | |
| Q9Y490 | TLN1 | Talin-1 | TLN1_HUMAN | 0.0309233 | 1.1707 | 1.50971 | 0.95622 | 1.22 |
| P22061 | PCMT1 | Protein-L-isoaspartate(D- | PIMT_HUMAN | 0.0311793 | −0.6930 | 1.50613 | −0.68410 | 1.01 |
| | | aspartate) | | | | | | |
| | | O-methyltransferase | | | | | | |
| P09622 | DLD | Dihydrolipoyl | DLDH_HUMAN | 0.0313943 | −0.8721 | 1.50315 | −0.82123 | 1.06 |
| | | dehydrogenase, | | | | | | |
| | | mitochondrial | | | | | | |
| P10321 | HLA-C | HLA class I | HLAC_HUMAN | 0.0315222 | 1.1156 | 1.50138 | 0.76038 | 1.47 |
| | | histocompatibility | | | | | | |
| | | antigen, C alpha chain | | | | | | |
| P13224 | GP1BB | Platelet glycoprotein lb beta | GP1BB_HUMAN | 0.0320272 | 1.3394 | 1.49448 | 1.00452 | 1.33 |
| | | chain | | | | | | |
| Q02978 | SLC25A11 | Mitochondrial 2- | M2OM_HUMAN | 0.0320353 | −1.3308 | 1.49437 | −1.14444 | 1.16 |
| | | oxoglutarate/malate carrier | | | | | | |
| | | protein | | | | | | |
| P40429 | RPL13A | Large ribosomal subunit | RL13A_HUMAN | 0.0321331 | −0.8343 | 1.49305 | −0.75603 | 1.10 |
| | | protein uL13 | | | | | | |
| Q15223 | NECTIN1 | Nectin-1 | NECT1_HUMAN | 0.0324962 | 1.9705 | 1.48817 | 0.90300 | 2.18 |
| Q9NWF9 | RNF216 | E3 ubiquitin-protein ligase | RN216_HUMAN | 0.0328361 | 2.0819 | 1.48365 | 0.85034 | 2.45 |
| | | RNF216 | | | | | | |
| Q8TE59 | ADAMTS19 | A disintegrin and | ATS19_HUMAN | 0.0339293 | 1.6441 | 1.46943 | 0.69603 | 2.36 |
| | | metalloproteinase with | | | | | | |
| | | thrombospondin motifs 19 | | | | | | |
| Q4KMQ2 | ANO6 | Anoctamin-6 | ANO6_HUMAN | 0.0341306 | −1.6187 | 1.46686 | −0.85299 | 1.90 |
| O00232 | PSMD12 | 26S proteasome non-ATPase | PSD12_HUMAN | 0.0345659 | −1.0595 | 1.46135 | −0.76966 | 1.38 |
| | | regulatory subunit 12 | | | | | | |
| Q9UBG0 | MRC2 | C-type mannose receptor 2 | MRC2_HUMAN | 0.0350169 | 1.0780 | 1.45572 | 0.70974 | 1.52 |
| Q9Y4G6 | TLN2 | Talin-2 | TLN2_HUMAN | 0.0354760 | −1.2179 | 1.45007 | −0.83021 | 1.47 |
| Q8N128 | FAM177A1 | Protein FAM177A1 | F177A_HUMAN | 0.0359640 | 1.2080 | 1.44413 | 0.75331 | 1.60 |
| P08134 | RHOC | Rho-related GTP-binding | RHOC_HUMAN | 0.0360800 | 0.9242 | 1.44273 | 0.82188 | 1.12 |
| | | protein RhoC | | | | | | |
| P69905 | HBA1 | Hemoglobin subunit alpha | HBA_HUMAN | 0.0360832 | 1.8490 | 1.44270 | 0.89187 | 2.07 |
| P09110 | ACAA1 | 3-ketoacyl-CoA thiolase, | THIK_HUMAN | 0.0367973 | −0.8622 | 1.43418 | −0.76986 | 1.12 |
| | | peroxisomal | | | | | | |
| Q8WXE1 | ATRIP | ATR-interacting protein | ATRIP_HUMAN | 0.0368601 | −0.6143 | 1.43344 | −0.75776 | 0.81 |
| Q9Y5Y4 | PTGDR2 | Prostaglandin D2 receptor 2 | PD2R2_HUMAN | 0.0374018 | −1.4000 | 1.42711 | −0.66107 | 2.12 |
| P68402 | PAFAH1B2 | Platelet-activating factor | PA1B2_HUMAN | 0.0382499 | 0.9803 | 1.41737 | 0.71159 | 1.38 |
| | | acetylhydrolase IB subunit | | | | | | |
| | | alpha2 | | | | | | |
| Q93009 | USP7 | Ubiquitin carboxyl-terminal | UBP7_HUMAN | 0.0387727 | −0.7131 | 1.41147 | −0.69010 | 1.03 |
| | | hydrolase 7 | | | | | | |
| P12236 | SLC25A6 | ADP/ATP translocase 3 | ADT3_HUMAN | 0.0388010 | 0.8971 | 1.41116 | 0.63061 | 1.42 |
| P36542 | ATP5F1C | ATP synthase subunit | ATPG_HUMAN | 0.0395570 | −0.8549 | 1.40278 | −0.63923 | 1.34 |
| | | gamma, mitochondrial | | | | | | |
| Q9BQE3 | TUBA1C | Tubulin alpha-1C chain | TBA1C_HUMAN | 0.0396282 | −1.4660 | 1.40200 | −0.71502 | 2.05 |
| O15254 | ACOX3 | Peroxisomal acyl-coenzyme | ACOX3_HUMAN | 0.0398149 | −0.6940 | 1.39995 | −0.74595 | 0.93 |
| | | A oxidase 3 | | | | | | |
| Q14997 | PSME4 | Proteasome activator | PSME4_HUMAN | 0.0400132 | −0.8792 | 1.39780 | −0.52795 | 1.67 |
| | | complex subunit 4 | | | | | | |
| Q16658 | FSCN1 | Fascin | FSCN1_HUMAN | 0.0402241 | −0.9878 | 1.39551 | −0.78162 | 1.26 |
| P02042 | HBD | Hemoglobin subunit delta | HBD_HUMAN | 0.0407735 | 1.6647 | 1.38962 | 0.89882 | 1.85 |
| P16401 | H1-5 | Histone H1.5 | H15_HUMAN | 0.0408222 | −0.8693 | 1.38910 | −0.68442 | 1.27 |
| Q9Y6E2 | BZW2 | elF5-mimic protein 1 | 5MP1_HUMAN | 0.0411924 | −0.7772 | 1.38518 | −0.65704 | 1.18 |
| Q13439 | GOLGA4 | Golgin subfamily A | GOGA4_HUMAN | 0.0429976 | 0.9163 | 1.36656 | 0.64222 | 1.43 |
| | | member 4 | | | | | | |
| P35573 | AGL | Glycogen debranching | GDE_HUMAN | 0.0434439 | −0.4972 | 1.36207 | −0.67228 | 0.74 |
| | | enzyme | | | | | | |
| Q8NCA5 | FAM98A | Protein FAM98A | FA98A_HUMAN | 0.0440644 | −1.7001 | 1.35591 | −1.06958 | 1.59 |
| P68363 | TUBA1B | Tubulin alpha-1B chain | TBA1B_HUMAN | 0.0440820 | 0.7172 | 1.35574 | 0.69976 | 1.02 |
| Q13148 | TARDBP | TAR DNA-binding | TADBP_HUMAN | 0.0444180 | −1.2098 | 1.35244 | −0.70077 | 1.73 |
| | | protein 43 | | | | | | |
| Q9H2S5 | RNF39 | RING finger protein 39 | RNF39_HUMAN | 0.0452747 | −0.7327 | 1.34414 | −0.65205 | 1.12 |
| P62277 | RPS13 | Small ribosomal subunit | RS13_HUMAN | 0.0462425 | −0.5575 | 1.33496 | −0.63829 | 0.87 |
| | | protein uS15 | | | | | | |
| Q5SW79 | CEP170 | Centrosomal protein | CE170_HUMAN | 0.0465918 | 1.2708 | 1.33169 | 0.71258 | 1.78 |
| | | of 170 kDa | | | | | | |
| Q9H497 | TOR3A | Torsin-3A | TOR3A_HUMAN | 0.0470293 | 0.4920 | 1.32763 | 0.68881 | 0.71 |
| P22102 | GART | Trifunctional purine | PUR2_HUMAN | 0.0481546 | −0.9180 | 1.31736 | −0.73470 | 1.25 |
| | | biosynthetic | | | | | | |
| | | protein adenosine-3 | | | | | | |
| P35579 | MYH9 | Myosin-9 | MYH9_HUMAN | 0.0486830 | 0.6672 | 1.31262 | 0.68804 | 0.97 |
| Q8TB73 | NDNF | Protein NDNF | NDNF_HUMAN | 0.0487819 | 1.8398 | 1.31174 | 0.79169 | 2.32 |
| Q9Y5X9 | LIPG | Endothelial lipase | LIPG_HUMAN | 0.0497609 | −0.9820 | 1.30311 | −0.76355 | 1.29 |
| Q96DR8 | MUCL1 | Mucin-like protein 1 | MUCL1_HUMAN | 0.0499774 | −0.8284 | 1.30123 | −0.60988 | 1.36 |

TABLE S4
Common human and murine high priority biomarker candidates having p < 0.05 in human
patient derived EVs by genotype

| | | | | | Ms. MEF Evs | | | Ms. MEF Evs | | |
|---|---|---|---|---|---|---|---|---|---|---|
| | | | | | (DYT1 + Sal/DYT1) | | | (DYT1/DYT1 + Sal) | | |
| | | | | | | | DYT + | | | DYT/ |
| | | Ms. MEF Evs (DYT1/WT) | | | | | Sal/DYT | | | DYT + Sal |
| GN(ms) | GN(Hs) | Log2_FC | -LogP | Cohen's D | Log2_FC | -LogP | Cohen's d | Log2_FC | -LogP | Cohen's d |
| Usp7 | USP7 | −1.83196 | 1.56000 | −4.28537 | 1.36458 | 0.81255 | 1.22883 | −1.36458 | 0.81255 | 1.22883 |
| Sec31a | SEC31A | −0.70512 | 1.35509 | −2.49441 | 0.10512 | 0.25234 | −0.23548 | −0.10512 | 0.25234 | −0.23548 |
| Tardbp | TARDBP | −1.75607 | 2.19418 | −4.49944 | 0.43881 | 0.51506 | 0.45221 | −0.43881 | 0.51506 | 0.45221 |
| Ap1b1 | AP1B1 | −1.26969 | 1.49775 | −3.24890 | 0.58735 | 0.74025 | −0.54786 | −0.58735 | 0.74025 | −0.54786 |
| Zyx | ZYX | 0.26071 | 0.19597 | 0.44667 | −0.21960 | 0.21927 | −0.58141 | 0.21960 | 0.21927 | −0.58141 |
| Rhoa | RHOA | −0.31786 | 0.31879 | −0.66724 | 0.21209 | 0.14088 | −0.65720 | −0.21209 | 0.14088 | −0.65720 |
| Pi4ka | PI4KA | 0.09407 | 0.15239 | 0.34519 | −1.03924 | 0.98155 | 0.76563 | 1.03924 | 0.98155 | 0.76563 |
| Psmc5 | PSMC5 | −0.30564 | 0.59203 | −1.23709 | 0.44581 | 0.48533 | 1.22050 | −0.44581 | 0.48533 | 1.22050 |
| Pcmt1 | PCMT1 | −2.31824 | 1.22855 | −2.45717 | 1.13291 | 1.30031 | −0.27456 | −1.13291 | 1.30031 | −0.27456 |
| Hnrnpc | HNRNPC | 0.13016 | 0.00422 | 0.01111 | −0.28447 | 0.22765 | −0.51106 | 0.28447 | 0.22765 | −0.51106 |
| Gart | GART | −0.83180 | 1.09501 | −2.19455 | 0.05776 | 0.07277 | 0.62606 | −0.05776 | 0.07277 | 0.62606 |
| Mapk3 | MAPK3 | −1.28104 | 0.89687 | −1.66623 | 0.67645 | 0.38235 | −0.31867 | −0.67645 | 0.38235 | −0.31867 |
| Ipo9 | IPO9 | −1.28228 | 1.00760 | −2.19051 | 0.30592 | 0.75428 | 0.16518 | −0.30592 | 0.75428 | 0.16518 |
| Jam3 | JAM3 | 0.96765 | 0.93943 | 1.70926 | −0.61325 | 0.62658 | −0.53874 | 0.61325 | 0.62658 | −0.53874 |
| Cryab | CRYAB | −1.35923 | 1.70906 | −5.18716 | 1.02491 | 1.48280 | 1.10080 | −1.02491 | 1.48280 | 1.10080 |
| Stom | STOM | −0.80186 | 1.29873 | −2.26728 | 0.01864 | 0.02642 | −0.40482 | −0.01864 | 0.02642 | −0.40482 |
| Rps13 | RPS13 | −0.22577 | 0.27913 | −0.56766 | 0.02541 | 0.02518 | −0.14091 | −0.02541 | 0.02518 | −0.14091 |
| DIst | DLST | 0.22173 | 0.30192 | 0.60773 | −0.51724 | 0.93678 | −0.97303 | 0.51724 | 0.93678 | −0.97303 |
| Prpsap2 | PRPSAP2 | 0.20606 | 0.10259 | 0.24014 | −0.08140 | 0.06754 | −1.25719 | 0.08140 | 0.06754 | −1.25719 |
| Wdr26 | WDR26 | −0.88939 | 1.38336 | −2.42537 | −0.03177 | 0.04087 | −0.18952 | 0.03177 | 0.04087 | −0.18952 |
| Psme4 | PSME4 | −1.73147 | 2.10844 | −4.08287 | −0.20994 | 0.30847 | −0.39168 | 0.20994 | 0.30847 | −0.39168 |
| Lipg | LIPG | −1.44483 | 1.59947 | −4.50143 | −0.45066 | 0.30680 | 0.61628 | 0.45066 | 0.30680 | 0.61628 |
| Ndnf | NDNF | −0.53994 | 0.47463 | −1.02277 | −0.57465 | 0.20242 | −0.42270 | 0.57465 | 0.20242 | −0.42270 |
| Rap2b | RAP2B | −0.70500 | 1.11950 | −2.55524 | −0.32881 | 0.48902 | −0.24904 | 0.32881 | 0.48902 | −0.24904 |
| Tln1 | TLN1 | −0.12217 | 0.08691 | −0.21222 | −0.19151 | 1.18568 | 0.56652 | 0.19151 | 1.18568 | 0.56652 |
| Flna | FLNA | −0.29336 | 0.41235 | −0.80625 | −0.07088 | 0.08331 | 0.48283 | 0.07088 | 0.08331 | 0.48283 |
| Mrc2 | MRC2 | 0.45506 | 1.17753 | 2.07349 | 0.01917 | 0.03262 | 0.33011 | −0.01917 | 0.03262 | 0.33011 |
| Slc2a1 | SLC2A1 | 0.01035 | 0.02475 | −0.06082 | 0.10884 | 0.08514 | −0.31722 | −0.10884 | 0.08514 | −0.31722 |
| Rpl10 | RPL10 | −0.39835 | 0.37481 | −0.75924 | −0.05107 | 0.02729 | −0.35966 | 0.05107 | 0.02729 | −0.35966 |
| Ptma | PTMA | 0.51246 | 0.66333 | 1.24099 | 1.23486 | 0.70051 | 0.97291 | −1.23486 | 0.70051 | 0.97291 |
| Bzw2 | BZW2 | −0.67977 | 0.46680 | −0.88912 | −0.03057 | 0.01774 | 0.28149 | 0.03057 | 0.01774 | 0.28149 |
| Lrrc59 | LRRC59 | −0.44884 | 1.01581 | −1.87086 | −0.42262 | 0.96959 | 0.46153 | 0.42262 | 0.96959 | 0.46153 |
| Rpl13a | RPL13A | −0.29771 | 0.31161 | −0.66760 | −0.13842 | 0.06911 | −0.47968 | 0.13842 | 0.06911 | −0.47968 |
| Acaa1a | ACAA1 | −0.01613 | 0.02704 | 0.06606 | −0.80068 | 0.78530 | −0.17381 | 0.80068 | 0.78530 | −0.17381 |
| Hist1h1b | H1-5 | −0.19977 | 0.03883 | −0.09674 | −0.11634 | 0.12784 | 0.47916 | 0.11634 | 0.12784 | 0.47916 |
| Fscn1 | FSCN1 | −0.39839 | 0.63483 | −1.29443 | −0.14138 | 0.31100 | 0.71376 | 0.14138 | 0.31100 | 0.71376 |
| Psmd12 | PSMD12 | −0.95453 | 0.71856 | −1.58047 | −0.20300 | 0.28295 | 1.23395 | 0.20300 | 0.28295 | 1.23395 |
| Mars | MARS1 | −0.31305 | 0.32788 | −0.66819 | −0.16372 | 0.31796 | −0.24558 | 0.16372 | 0.31796 | −0.24558 |
| Tln2 | TLN2 | −1.34102 | 0.73523 | −1.39011 | −0.32362 | 0.17767 | 0.54885 | 0.32362 | 0.17767 | 0.54885 |
| Ar18b | ARL8B | −0.83243 | 0.93080 | −1.64885 | −0.08283 | 0.07471 | −0.57733 | 0.08283 | 0.07471 | −0.57733 |
| Ehd4 | EHD4 | −0.48029 | 0.73070 | −1.30511 | −0.14900 | 0.24806 | −0.47170 | 0.14900 | 0.24806 | −0.47170 |
| Tubb1 | TUBB1 | −0.02556 | 0.08587 | −0.20357 | −0.69486 | 0.58609 | 0.24600 | 0.69486 | 0.58609 | 0.24600 |
| Myh9 | MYH9 | −0.18064 | 0.33123 | −0.66175 | −0.27419 | 0.78198 | −0.28839 | 0.27419 | 0.78198 | −0.28839 |
| Tgfb2 | TGFB2 | −0.50026 | 0.60844 | −1.24575 | 0.30618 | 0.27460 | −0.32927 | −0.30618 | 0.27460 | −0.32927 |
| Pitpnb | PITPNB | 1.19374 | 1.26855 | 2.78119 | 0.09822 | 0.11474 | 0.66440 | −0.09822 | 0.11474 | 0.66440 |
| Rbm3 | RBM3 | −0.00155 | 0.00773 | −0.01922 | 0.83997 | 1.80505 | 0.26069 | −0.83997 | 1.80505 | 0.26069 |
| Ano6 | ANO6 | 0.22219 | 0.44453 | 0.89199 | −0.35353 | 0.59446 | 0.15926 | 0.35353 | 0.59446 | 0.15926 |

TABLE S5
Common human and murine high priority biomarker candidates having p < 0.05 in human patient
derived EVs by genotype

| | | Ms. MEF Evs | | | Ms. MEF Evs | | | Ms. MEF Evs | | |
|---|---|---|---|---|---|---|---|---|---|---|
| | | (WT + ISRIB/WT) | | | (DYT1 + RTV/DYT1) | | | (DYT1/DYT1 + RTV) | | |
| | | | | DYT/ | | | WT + | | | DYT + |
| | | | | DYT + Sal | | | ISRIB/WT | | | RTV/DYT |
| GN(ms) | GN(Hs) | Log2_FC | -LogP | Cohen's d | Log2_FC | -LogP | Cohen's d | Log2_FC | -LogP | Cohen's d |
| Usp7 | USP7 | −1.36458 | 0.81255 | 1.22883 | −0.83467 | 0.94500 | −0.33281 | 1.18870 | 2.78257 | 7.14308 |
| Sec31a | SEC31A | −0.10512 | 0.25234 | −0.23548 | −0.28384 | 0.54453 | −0.37637 | 0.83574 | 2.07149 | 4.40221 |
| Tardbp | TARDBP | −0.43881 | 0.51506 | 0.45221 | −0.06285 | 0.07040 | −0.30873 | 0.22717 | 0.30368 | 0.61164 |
| Ap1b1 | AP1B1 | −0.58735 | 0.74025 | −0.54786 | −0.18130 | 0.43633 | −0.23816 | 1.32802 | 1.51604 | 3.25788 |
| Zyx | ZYX | 0.21960 | 0.21927 | −0.58141 | 0.18010 | 1.17422 | −0.13102 | −0.36661 | 0.35652 | −0.70380 |
| Rhoa | RHOA | −0.21209 | 0.14088 | −0.65720 | −0.00598 | 0.00899 | −1.24978 | 0.19850 | 0.13276 | 0.30393 |
| Pi4ka | PI4KA | 1.03924 | 0.98155 | 0.76563 | 0.38265 | 0.33876 | −0.48932 | −0.35883 | 0.30506 | −0.64528 |
| Psmc5 | PSMC5 | −0.44581 | 0.48533 | 1.22050 | −0.36220 | 0.45096 | 0.42670 | 0.45593 | 1.30065 | 2.88257 |
| Pcmt1 | PCMT1 | −1.13291 | 1.30031 | −0.27456 | −0.06624 | 0.03179 | −0.85534 | 2.54728 | 2.18459 | 5.97504 |
| Hnrnpc | HNRNPC | 0.28447 | 0.22765 | −0.51106 | 1.47817 | 1.15343 | 1.19942 | −0.12734 | 0.08783 | −0.20312 |
| Gart | GART | −0.05776 | 0.07277 | 0.62606 | −0.25456 | 0.28585 | −0.11964 | 0.67696 | 1.11152 | 2.13711 |
| Mapk3 | MAPK3 | −0.67645 | 0.38235 | −0.31867 | −0.16302 | 0.15577 | −0.87389 | 1.98595 | 1.22895 | 2.38401 |
| Ipo9 | IPO9 | −0.30592 | 0.75428 | 0.16518 | −0.53859 | 0.46317 | −0.46607 | 1.12192 | 1.96625 | 4.13545 |
| Jam3 | JAM3 | 0.61325 | 0.62658 | −0.53874 | 0.37305 | 0.54427 | −0.32674 | −1.30149 | 1.28064 | −2.62403 |
| Cryab | CRYAB | −1.02491 | 1.48280 | 1.10080 | 0.11775 | 0.16087 | −0.11649 | 0.42001 | 0.32271 | 0.70885 |
| Stom | STOM | −0.01864 | 0.02642 | −0.40482 | 0.11352 | 0.18187 | −0.72792 | 0.19859 | 0.19272 | 0.41810 |
| Rps13 | RPS13 | −0.02541 | 0.02518 | −0.14091 | 0.28095 | 0.41978 | −0.22693 | 0.02007 | 0.02431 | 0.06202 |
| DIst | DLST | 0.51724 | 0.93678 | −0.97303 | −1.89684 | 1.33271 | 0.68212 | −0.44587 | 0.79988 | −1.43749 |
| Prpsap2 | PRPSAP2 | 0.08140 | 0.06754 | −1.25719 | −0.67247 | 1.13987 | 0.21583 | −0.08407 | 0.05990 | −0.14211 |
| Wdr26 | WDR26 | 0.03177 | 0.04087 | −0.18952 | −0.05756 | 0.07344 | −0.26885 | 1.19994 | 1.94611 | 3.71380 |
| Psme4 | PSME4 | 0.20994 | 0.30847 | −0.39168 | −0.79739 | 1.12059 | −0.81621 | 1.37082 | 1.75533 | 3.22049 |
| Lipg | LIPG | 0.45066 | 0.30680 | 0.61628 | −0.36394 | 0.98353 | 0.73954 | 1.29860 | 1.59228 | 2.83387 |
| Ndnf | NDNF | 0.57465 | 0.20242 | −0.42270 | −0.13752 | 0.12844 | 1.07096 | 0.46965 | 0.16046 | 0.35300 |
| Rap2b | RAP2B | 0.32881 | 0.48902 | −0.24904 | −0.12448 | 0.51405 | 0.37880 | 0.33421 | 0.36102 | 0.71295 |
| Tln1 | TLN1 | 0.19151 | 1.18568 | 0.56652 | −0.05074 | 0.05249 | −0.38999 | 0.14511 | 0.49546 | 1.02612 |
| Flna | FLNA | 0.07088 | 0.08331 | 0.48283 | −0.23408 | 0.30638 | −0.09039 | 0.37214 | 0.76379 | 1.35657 |
| Mrc2 | MRC2 | −0.01917 | 0.03262 | 0.33011 | −0.29423 | 0.58520 | 0.21166 | −0.09834 | 0.18741 | −0.40395 |
| Slc2a1 | SLC2A1 | −0.10884 | 0.08514 | −0.31722 | 0.08012 | 0.09112 | −0.34000 | −0.03153 | 0.01835 | −0.04516 |
| Rpl10 | RPL10 | 0.05107 | 0.02729 | −0.35966 | 0.00077 | 0.00079 | −0.35001 | 0.52142 | 0.37750 | 0.79438 |
| Ptma | PTMA | −1.23486 | 0.70051 | 0.97291 | 2.63837 | 1.48140 | 0.63551 | −0.85319 | 1.34001 | −2.36608 |
| Bzw2 | BZW2 | 0.03057 | 0.01774 | 0.28149 | 0.72645 | 0.61549 | −0.91856 | 0.80819 | 0.89528 | 1.68476 |
| Lrrc59 | LRRC59 | 0.42262 | 0.96959 | 0.46153 | 0.34305 | 0.80715 | −0.68840 | 0.16243 | 0.47061 | 0.88787 |
| Rpl13a | RPL13A | 0.13842 | 0.06911 | −0.47968 | 0.04217 | 0.04759 | 0.31229 | 0.67320 | 0.42863 | 0.92215 |
| Acaa1a | ACAA1 | 0.80068 | 0.78530 | −0.17381 | −0.63046 | 0.51453 | −0.87512 | 0.86753 | 0.77944 | 1.38060 |
| Hist1h1b | H1-5 | 0.11634 | 0.12784 | 0.47916 | −0.08676 | 0.05393 | −0.45565 | 0.98343 | 0.56775 | 1.18239 |
| Fscn1 | FSCN1 | 0.14138 | 0.31100 | 0.71376 | −0.29435 | 0.50422 | −0.97283 | 0.40204 | 1.65736 | 2.99498 |
| Psmd12 | PSMD12 | 0.20300 | 0.28295 | 1.23395 | −0.38045 | 0.24549 | 0.00373 | 1.09018 | 2.74852 | 10.60245 |
| Mars | MARS1 | 0.16372 | 0.31796 | −0.24558 | −0.37169 | 0.36780 | 0.05955 | 0.51481 | 1.16080 | 2.21073 |
| Tln2 | TLN2 | 0.32362 | 0.17767 | 0.54885 | −0.02524 | 0.01246 | −0.74845 | 0.64412 | 0.55718 | 1.03063 |
| Arl8b | ARL8B | 0.08283 | 0.07471 | −0.57733 | −0.29885 | 0.41861 | −0.87781 | 0.40066 | 0.44389 | 0.90322 |
| Ehd4 | EHD4 | 0.14900 | 0.24806 | −0.47170 | −0.09267 | 0.14451 | −1.15914 | 0.07182 | 0.11148 | 0.25575 |
| Tubb1 | TUBB1 | 0.69486 | 0.58609 | 0.24600 | 0.19449 | 0.33345 | 0.30048 | −0.05972 | 0.04740 | −0.11310 |
| Myh9 | MYH9 | 0.27419 | 0.78198 | −0.28839 | 0.05232 | 0.07780 | −0.02365 | −0.00905 | 0.01573 | −0.03876 |
| Tgfb2 | TGFB2 | −0.30618 | 0.27460 | −0.32927 | −0.31899 | 0.47699 | 0.69840 | −0.72175 | 0.74907 | −1.38832 |
| Pitpnb | PITPNB | −0.09822 | 0.11474 | 0.66440 | 1.46149 | 1.31948 | −1.11434 | 0.72960 | 1.55147 | 2.82374 |
| Rbm3 | RBM3 | −0.83997 | 1.80505 | 0.26069 | 0.20282 | 0.68670 | −0.31593 | −0.10483 | 0.20365 | −0.43280 |
| Ano6 | ANO6 | 0.35353 | 0.59446 | 0.15926 | 0.24957 | 0.43649 | 0.83516 | 0.08584 | 0.08539 | 0.20010 |

TABLE S6
Common human and murine high priority biomarker candidates having p < 0.05 in human
patient derived EVs by genotype

| | | Hs. Plasma EVs (DYT1/WT) | | | |
|---|---|---|---|---|---|
| GN(ms) | GN(Hs) | Log2_FC | -LogP | Cohen'sD | Directionality |
| Usp7 | USP7 | -0.713057 | 1.411474 | -0.690103 | DOWN |
| Sec31a | SEC31A | -1.127608 | 1.850351 | -0.868576 | DOWN |
| Tardbp | TARDBP | -1.209829 | 1.352441 | -0.700770 | DOWN |
| Ap1b1 | AP1B1 | -1.338567 | 2.413461 | -1.045373 | DOWN |
| Zyx | ZYX | 0.932239 | 2.225120 | 0.973352 | UP |
| Rhoa | RHOA | 0.546550 | 1.673556 | 0.933321 | UP |
| Pi4ka | PI4KA | -0.265353 | 2.660125 | -0.880825 | DOWN |
| Psmc5 | PSMC5 | -0.598561 | 1.615197 | -0.693610 | DOWN |
| Pcmt1 | PCMT1 | -0.693036 | 1.506134 | -0.684101 | DOWN |
| Hnrnpc | HNRNPC | -0.742818 | 2.933157 | -1.053819 | DOWN |
| Gart | GART | -0.918036 | 1.317362 | -0.734704 | DOWN |
| Mapk3 | MAPK3 | -0.940330 | 1.682411 | -0.852667 | DOWN |
| Ipo9 | IPO9 | -1.142105 | 1.796367 | -0.918702 | DOWN |
| Jam3 | JAM3 | -1.606865 | 1.640903 | -0.994236 | DOWN |
| Cryab | CRYAB | -1.086934 | 1.576561 | -0.803557 | DOWN |
| Stom | STOM | 1.306287 | 1.948349 | 1.093874 | UP |
| Rps13 | RPS13 | -0.557497 | 1.334959 | -0.638292 | DOWN |
| DIst | DLST | -1.085432 | 1.927801 | -0.972956 | DOWN |
| Prpsap2 | PRPSAP2 | -1.104550 | 1.629414 | -0.799400 | DOWN |
| Wdr26 | WDR26 | 0.682418 | 1.772071 | 0.721416 | UP |
| Psme4 | PSME4 | -0.879158 | 1.397797 | -0.527950 | DOWN |
| Lipg | LIPG | -0.982040 | 1.303112 | -0.763546 | DOWN |
| Ndnf | NDNF | 1.839848 | 1.311741 | 0.791691 | UP |
| Rap2b | RAP2B | 1.665914 | 2.656490 | 1.038217 | UP |
| Tln1 | TLN1 | 1.170694 | 1.509714 | 0.956217 | UP |
| Flna | FLNA | 1.126738 | 1.742620 | 1.009737 | UP |
| Mrc2 | MRC2 | 1.078000 | 1.455722 | 0.709737 | UP |
| Slc2a1 | SLC2A1 | 1.075780 | 1.634622 | 0.971169 | UP |
| Rpl10 | RPL10 | -0.703493 | 1.605646 | -0.643162 | DOWN |
| Ptma | PTMA | -0.720314 | 3.671163 | -1.514853 | DOWN |
| Bzw2 | BZW2 | -0.777204 | 1.385183 | -0.657044 | DOWN |
| Lrrc59 | LRRC59 | -0.801068 | 1.584235 | -0.670209 | DOWN |
| Rpl13a | RPL13A | -0.834276 | 1.493048 | -0.756029 | DOWN |
| Acaa1a | ACAA1 | -0.862166 | 1.434184 | -0.769857 | DOWN |
| Hist1h1b | H1-5 | -0.869318 | 1.389104 | -0.684425 | DOWN |
| Fscn1 | FSCN1 | -0.987807 | 1.395513 | -0.781621 | DOWN |
| Psmd12 | PSMD12 | -1.059502 | 1.461352 | -0.769664 | DOWN |
| Mars | MARS1 | -1.187451 | 1.713802 | -0.729345 | DOWN |
| TIn2 | TLN2 | -1.217854 | 1.450066 | -0.830212 | DOWN |
| Ar18b | ARL8B | -1.422978 | 1.860093 | -1.253952 | DOWN |
| Ehd4 | EHD4 | -2.112574 | 3.322399 | -1.106926 | DOWN |
| Tubb1 | TUBB1 | 1.198549 | 2.266911 | 1.367412 | UP |
| Myh9 | MYH9 | 0.667215 | 1.312622 | 0.688036 | UP |
| Tgfb2 | TGFB2 | -0.995168 | 2.018323 | -0.846643 | DOWN |
| Pitpnb | PITPNB | -1.064090 | 1.574590 | -0.847445 | DOWN |
| Rbm3 | RBM3 | -1.348286 | 2.178906 | -0.940579 | DOWN |
| Ano6 | ANO6 | -0.713057 | 1.411474 | -0.690103 | DOWN |

TABLE S7
Common human and murine high priority biomarker candidates having p < 0.05 in human patient derived EVs by genotype

| | | Hs. Plasma | | | | | RTV | Sal | ISRIB and | Hs. Ms | p < 0.05 in |
|---|---|---|---|---|---|---|---|---|---|---|---|
| | | EVs | | DYT1/ | DYT1/ | WT + | Correc- | Correc- | DYT | Direction | Human patient |
| | | (DYT1/ | DYT1/ | DYT1 + | DYT1 + | ISRIB/ | tive | tive | Parallel | Con- | derived Evs |
| GN(ms) | GN(Hs) | WT) | WT | Sal | RTV | WT | Direction | Direction | Direction | served | by Genotype? |
| Usp7 | USP7 | DOWN | DOWN | DOWN | DOWN | DOWN | Yes | Yes | Yes | Yes | YES |
| Sec31a | SEC31A | DOWN | DOWN | DOWN | DOWN | DOWN | Yes | Yes | Yes | Yes | YES |
| Tardbp | TARDBP | DOWN | DOWN | DOWN | DOWN | DOWN | Yes | Yes | Yes | Yes | YES |
| Ap1b1 | AP1B1 | DOWN | DOWN | DOWN | DOWN | DOWN | Yes | Yes | Yes | Yes | YES |
| Zyx | ZYX | UP | UP | UP | UP | UP | Yes | Yes | Yes | Yes | YES |
| Rhoa | RHOA | UP | DOWN | DOWN | DOWN | DOWN | Yes | Yes | Yes | No | YES |
| Pi4ka | PI4KA | DOWN | UP | UP | UP | UP | Yes | Yes | Yes | No | YES |
| Psmc5 | PSMC5 | DOWN | DOWN | DOWN | DOWN | DOWN | Yes | Yes | Yes | Yes | YES |
| Pcmt1 | PCMT1 | DOWN | DOWN | DOWN | DOWN | DOWN | Yes | Yes | Yes | Yes | YES |
| Hnrnpc | HNRNPC | DOWN | UP | UP | UP | UP | Yes | Yes | Yes | No | YES |
| Gart | GART | DOWN | DOWN | DOWN | DOWN | DOWN | Yes | Yes | Yes | Yes | YES |
| Mapk3 | MAPK3 | DOWN | DOWN | DOWN | DOWN | DOWN | Yes | Yes | Yes | Yes | YES |
| Ipo9 | IPO9 | DOWN | DOWN | DOWN | DOWN | DOWN | Yes | Yes | Yes | Yes | YES |
| Jam3 | JAM3 | DOWN | UP | UP | UP | UP | Yes | Yes | Yes | No | YES |
| Cryab | CRYAB | DOWN | DOWN | DOWN | DOWN | UP | Yes | Yes | No | Yes | YES |
| Stom | STOM | UP | DOWN | DOWN | DOWN | UP | Yes | Yes | No | No | YES |
| Rps13 | RPS13 | DOWN | DOWN | DOWN | DOWN | UP | Yes | Yes | No | Yes | YES |
| DIst | DLST | DOWN | UP | UP | UP | DOWN | Yes | Yes | No | No | YES |
| Prpsap2 | PRPSAP2 | DOWN | UP | UP | UP | DOWN | Yes | Yes | No | No | YES |
| Wdr26 | WDR26 | UP | DOWN | UP | DOWN | DOWN | Yes | No | Yes | No | YES |
| Psme4 | PSME4 | DOWN | DOWN | UP | DOWN | DOWN | Yes | No | Yes | Yes | YES |
| Lipg | LIPG | DOWN | DOWN | UP | DOWN | DOWN | Yes | No | Yes | Yes | YES |
| Ndnf | NDNF | UP | DOWN | UP | DOWN | DOWN | Yes | No | Yes | No | YES |
| Rap2b | RAP2B | UP | DOWN | UP | DOWN | DOWN | Yes | No | Yes | No | YES |
| Tln1 | TLN1 | UP | DOWN | UP | DOWN | DOWN | Yes | No | Yes | No | YES |
| Flna | FLNA | UP | DOWN | UP | DOWN | DOWN | Yes | No | Yes | No | YES |
| Mrc2 | MRC2 | UP | UP | DOWN | UP | DOWN | Yes | No | No | Yes | YES |
| Slc2a1 | SLC2A1 | UP | UP | DOWN | UP | UP | Yes | No | Yes | Yes | YES |
| Rpl10 | RPL10 | DOWN | DOWN | UP | DOWN | UP | Yes | No | No | Yes | YES |
| Ptma | PTMA | DOWN | UP | DOWN | UP | UP | Yes | No | Yes | No | YES |
| Bzw2 | BZW2 | DOWN | DOWN | UP | DOWN | UP | Yes | No | No | Yes | YES |
| Lrrc59 | LRRC59 | DOWN | DOWN | UP | DOWN | UP | Yes | No | No | Yes | YES |
| Rpl13a | RPL13A | DOWN | DOWN | UP | DOWN | UP | Yes | No | No | Yes | YES |
| Acaa1a | ACAA1 | DOWN | DOWN | UP | DOWN | DOWN | Yes | No | Yes | Yes | YES |
| Hist1h1b | H1-5 | DOWN | DOWN | UP | DOWN | DOWN | Yes | No | Yes | Yes | YES |
| Fscn1 | FSCN1 | DOWN | DOWN | UP | DOWN | DOWN | Yes | No | Yes | Yes | YES |
| Psmd12 | PSMD12 | DOWN | DOWN | UP | DOWN | DOWN | Yes | No | Yes | Yes | YES |
| Mars | MARS1 | DOWN | DOWN | UP | DOWN | DOWN | Yes | No | Yes | Yes | YES |
| Tln2 | TLN2 | DOWN | DOWN | UP | DOWN | DOWN | Yes | No | Yes | Yes | YES |
| Arl8b | ARL8B | DOWN | DOWN | UP | DOWN | DOWN | Yes | No | Yes | Yes | YES |
| Ehd4 | EHD4 | DOWN | DOWN | UP | DOWN | DOWN | Yes | No | Yes | Yes | YES |
| Tubb1 | TUBB1 | UP | DOWN | UP | UP | UP | No | No | No | No | YES |
| Myh9 | MYH9 | UP | DOWN | UP | UP | UP | No | No | No | No | YES |
| Tgfb2 | TGFB2 | DOWN | DOWN | DOWN | UP | DOWN | No | Yes | Yes | Yes | YES |
| Pitpnb | PITPNB | DOWN | UP | DOWN | DOWN | UP | No | No | Yes | No | YES |
| Rbm3 | RBM3 | DOWN | DOWN | DOWN | UP | UP | No | Yes | No | Yes | YES |
| Ano6 | ANO6 | DOWN | UP | UP | DOWN | UP | No | Yes | Yes | No | YES |

Key:
DYT1/WT Directionality of protein change in MEFs EV dataset between WT and DYT1 derived MEFs
DYT1/DYT1 + Sal Directionality of protein change in MEFs EV dataset in DYT1 derived MEFs treated with salubrinal
DYT1/DYT1 + RTV Directionality of protein change in MEFs EV dataset in DYT1 derived MEFs treated with Ritonavir
WT + ISRIB/WT Directionality of protein change in MEFs EV dataset in WT derived MEFs treated with ISRIB
RTV Corrective Direction Was treatment in MEF's in the direction of WT-like?
Sal Corrective Direction Was treatment in MEF's in the direction of WT-like?
ISRIB and DYT Parallel Direction Was treatment in MEF's in the direction of DYT1-like?
Hs. Ms Direction Conserved Directionality of change in DYT1/WT(HC) the same in Human Plasma derived exosomes the same as in MEF derived Evs?
P < 0.05 in Ms and Hs EV by Genotype Was genetype difference significant in both human and MEF derived Evs? (Non-corrected t.test p < 0.05)

TABLE S8
Protein Abbreviations and Names

| Abbreviation | Protein |
|---|---|
| ABCB9 | ABC-type oligopeptide transporter ABCB9 |
| ACAA1 | Acetyl-CoA acyltransferase 1 |
| ACOX3 | Peroxisomal acyl-coenzyme A oxidase 3 |
| ADAMTS19A | ADAM metallopeptidase with thrombospondin type 1 motif 19 |
| ANK1 | Ankyrin-1 |
| ANO6 | Anoctamin-6 |
| AP1B1 | AP-1 complex subunit beta-1 |
| ARL8B | ADP-ribosylation factor-like protein 8b |
| ATP5F1C | ATP synthase subunit gamma, mitochondrial |
| ATP6V1G1 | V-type proton ATPase subunit G 1 |
| ATRIP | ATR-interacting protein |
| BRD9 | Bromodomain containing 9 |
| BZW2 | Basic leucine zipper and W2 domains 2 |
| C16orf86 | Uncharacterized protein C16orf86 homolog |
| CEP170 | Centrosomal protein of 170 kDa |
| CRYAB | Alpha-crystallin B chain |
| DDOST | Dolichyl-diphosphooligosaccharide-protein glycosyltransferase |
| DLD | Dihydrolipoyl dehydrogenase, mitochondrial |
| DLST | Dihydrolipoyllysine-residue succinyltransferase component of 2-oxoglutarate |
| | dehydrogenase complex |
| DNAH2 | Dynein axonemal heavy chain 2 |
| EHD1 | EH domain-containing protein 1 |
| EHD2 | EH domain-containing protein 2 |
| EHD3 | EH domain-containing protein 3 |
| EHD4 | EH domain-containing protein 4 |
| F8 | Coagulation factor VIII |
| FAM131C | Family with sequence similarity 131 member C |
| FAM177A1 | Family with sequence similarity 177 member A1 |
| FAM98A | Protein FAM98A |
| FGL1 | Fibrinogen-like protein 1 |
| FLNA | Filamin-A |
| FSCN1 | Fascin actin-bundling protein 1 |
| FYN | Tyrosine-protein kinase Fyn |
| GALNTL6 | Polypeptide N-acetylgalactosaminyltransferase-like 6 |
| GART | Trifunctional purine biosynthetic protein adenosine-3 |
| GOLGA4 | Golgin subfamily A member 4 |
| GP1BB | Natriuretic peptides B |
| GP9 | Platelet glycoprotein IX |
| GPR17 | Uracil nucleotide/cysteinyl leukotriene receptor |
| H1-5 | Histone H1.5 |
| HBA1 | Hemoglobin alpha-1 globin chain |
| HBD | Hemoglobin subunit delta |
| HLA-C | HLA-C protein |
| HNRNPC | Heterogeneous nuclear ribonucleoproteins C1/C2 |
| IGHV8-51-1 | Probable non-functional immunoglobulin heavy variable 8-51-1 |
| ILK | Integrin-linked protein kinase |
| IPO9 | Importin-9 |
| ITGA2B | Integrin alpha-Ilb |
| JAM3 | Junctional adhesion molecule C |
| KCNB2 | Potassium voltage-gated channel subfamily B member 2 |
| KRT33A | Keratin, type I cuticular Ha3-1 |
| LAMA1 | Laminin subunit alpha-1 |
| LCK | Tyrosine-protein kinase Lck |
| LIPG | Endothelial lipase |
| LMNB2 | Lamin-B2 |
| LORICRIN | Loricrin |
| LRRC59 | Leucine-rich repeat-containing protein 59 |
| LYPLA1 | Acyl-protein thioesterase 1 |
| MAPK3 | Mitogen-activated protein kinase 3 |
| MARS1 | Methionyl-tRNA synthetase 1 |
| MFAP5 | Microfibril associated protein 5 |
| MKI67 | Proliferation marker protein Ki-67 |
| MPIG6B | Megakaryocyte and platelet inhibitory receptor G6b |
| MRC2 | Mannose receptor C type 2 |
| MUCL1 | Mucin-like protein 1 |
| MYH9 | MYH9 protein |
| NAA20 | N-alpha-acetyltransferase 20 |
| NDNF | Protein NDNF |
| NECTIN1 | Nectin-1 |
| NEK9 | Serine/threonine-protein kinase Nek9 |
| OBP2A | Odorant-binding protein 2a |
| PAFAH1B2 | Platelet-activating factor acetylhydrolase IB subunit alpha2 |
| PCDHB15 | Protocadherin beta 15 |
| PCMT1 | Protein-L-isoaspartate O-methyltransferase |
| PI4KA | Phosphatidylinositol 4-kinase alpha |
| PITPNB | Phosphatidylinositol transfer protein beta isoform |
| PLEK | Pleckstrin |
| PRPSAP2 | Phosphoribosyl pyrophosphate synthase-associated protein 2 |
| PSMC5 | Proteasome 26S subunit, ATPase 5 |
| PSMD12 | 26S proteasome non-ATPase regulatory subunit 12 |
| PSME4 | Proteasome activator complex subunit 4 |
| PTGDR2 | Prostaglandin D2 receptor 2 |
| PTMA | Prothymosin alpha |
| RAP2B | Ras-related protein Rap-2b |
| RBM3 | RNA-binding protein 3 |
| RHOA | Ras homolog family member A |
| RHOC | Rho-related GTP-binding protein RhoC |
| RNF216 | Ring finger protein 216 |
| RNF39 | RING finger protein 39 |
| RPL10 | Ribosomal protein L10 |
| RPL13A | Large ribosomal subunit protein uL 13 |
| RPS13 | Small ribosomal subunit protein Us15 |
| SEC31A | Protein transport protein Sec31A |
| SELENOF | Selenoprotein F |
| SETD2 | Histone-lysine N-methyltransferase SETD2 |
| SLC25A11 | Mitochondrial 2-oxoglutarate/malate carrier protein |
| SLC25A6 | ADP/ATP translocase 3 |
| SLC2A1 | Solute carrier family 2, facilitated glucose transporter member 1 |
| SLC4A1 | Band 3 anion transport protein |
| SLPI | Antileukoproteinase |
| SOGA1 | Suppressor of glucose, autophagy associated 1 |
| SP110 | Sp110 nuclear body protein |
| SPTB | Beta-spectrin |
| SRC | Proto-oncogene tyrosine-protein kinase Src |
| STOM | Stomatin |
| TARDBP | TAR DNA-binding protein |
| TFB1M | Dimethyladenosine transferase 1, mitochondrial |
| TGFB2 | Transforming growth factor beta-2 proprotein |
| TLN1 | Talin-1 |
| TLN2 | Talin-2 |
| TRAPPC9 | Trafficking protein particle complex subunit 9 |
| TRIM16 | Tripartite motif-containing protein 16 |
| TUBA1B | Tubulin alpha-1B chain |
| TUBA1C | Tubulin alpha-1C chain |
| TUBB1 | Tubulin beta-1 chain |
| USP2 | Ubiquitin carboxyl-terminal hydrolase 2 |
| USP7 | Ubiquitin carboxyl-terminal hydrolase 7 |
| VWF | von Willebrand factor |
| WDR26 | WD repeat-containing protein 26 |
| WFDC12 | WAP four-disulfide core domain protein 12 |
| WNK1 | Serine/threonine-protein kinase WNK1 |
| YES1 | Tyrosine-protein kinase Yes |
| ZYX | Zyxin |

1. A method of treating dystonia in a subject, comprising administering to the subject one or more therapeutic agents when one or more dystonia biomarkers in the subject is aberrant compared to a reference level,
wherein the one or more dystonia biomarkers comprises
(i) a protein selected from List-A1, List-A2, List-A3, List-A4, List-B1, List-B2, List-B3, List-C1, List-C2, List-C3, List-D1, List-D2, List-D3, List-E1, List-E2, List-E3, List-F1, List-F2, List-F3, List-G1, List-G2, List-G3, TABLE 4, TABLE 5, TABLE 6, TABLE 7, TABLE 8, TABLE 9, TABLE S1, TABLE S3, TABLE S4, TABLE S5, TABLE S6, TABLE S7, or a combination thereof;
(ii) an miRNA selected from List-MR1, TABLE 2, TABLE 3, or a combination thereof; or
(iii) a metabolite selected from List-ML1, or a combination thereof.

2. A method of treating dystonia in a subject, comprising administering to the subject an amount of one or more therapeutic agents, wherein the amount of the one or more therapeutic agents is increased or reduced from a preliminary dosage amount when one or more dystonia biomarkers in the subject is aberrant compared to a reference level,
wherein the one or more dystonia biomarkers comprises
(i) a protein selected from List-A1, List-A2, List-A3, List-A4, List-B1, List-B2, List-B3, List-C1, List-C2, List-C3, List-D1, List-D2, List-D3, List-E1, List-E2, List-E3, List-F1, List-F2, List-F3, List-G1, List-G2, List-G3, TABLE 4, TABLE 5, TABLE 6, TABLE 7, TABLE 8, TABLE 9, TABLE S1, TABLE S3, TABLE S4, TABLE S5, TABLE S6, TABLE S7, or a combination thereof;
(ii) an miRNA selected from List-MR1, TABLE 2, TABLE 3, or a combination thereof; or
(iii) a metabolite selected from List-ML1, or a combination thereof.

3. The method of claim 2, wherein the preliminary dosage amount is
a) set forth in a Food and Drug Administration (FDA) or European Medical Agency (EMA) approved label for the one or more therapeutic agents; or
b) the starting dosage of the one or more therapeutic agents in a clinical trial.

4. A method of selecting a subject for a clinical trial, the method comprising
a) measuring one or more dystonia biomarkers in the subject from a biological sample obtained from the subject, and
b) enrolling the subject in the clinical trial when the one or more dystonia biomarkers in the subject is aberrant compared to a reference level, or not enrolling the subject in the clinical trial when the one or more dystonia biomarkers in the subject is not aberrant compared to a reference level,
wherein the one or more dystonia biomarkers comprises
(i) a protein selected from List-A1, List-A2, List-A3, List-A4, List-B1, List-B2, List-B3, List-C1, List-C2, List-C3, List-D1, List-D2, List-D3, List-E1, List-E2, List-E3, List-F1, List-F2, List-F3, List-G1, List-G2, List-G3, TABLE 4, TABLE 5, TABLE 6, TABLE 7, TABLE 8, TABLE 9, TABLE S1, TABLE S3, TABLE S4, TABLE S5, TABLE S6, TABLE S7, or a combination thereof;
(ii) an miRNA selected from List-MR1, TABLE 2, TABLE 3, or a combination thereof; or
(iii) a metabolite selected from List-ML1, or a combination thereof.

5. A method of selecting a therapeutic agent for treatment of dystonia in a subject, the method comprising
a) measuring one or more dystonia biomarkers in the subject from a biological sample obtained from the subject, and
b) selecting the therapeutic agent when the one or more dystonia biomarkers in the subject is
i) aberrant compared to a reference level; or
ii) not aberrant compared to a reference level,
wherein the one or more dystonia biomarkers comprises
(i) a protein selected from List-A1, List-A2, List-A3, List-A4, List-B1, List-B2, List-B3, List-C1, List-C2, List-C3, List-D1, List-D2, List-D3, List-E1, List-E2, List-E3, List-F1, List-F2, List-F3, List-G1, List-G2, List-G3, TABLE 4, TABLE 5, TABLE 6, TABLE 7, TABLE 8, TABLE 9, TABLE S1, TABLE S3, TABLE S4, TABLE S5, TABLE S6, TABLE S7, or a combination thereof;
(ii) an miRNA selected from List-MR1, TABLE 2, TABLE 3, or a combination thereof; or
(iii) a metabolite selected from List-ML1, or a combination thereof.

6. A method of predicting responsiveness to a treatment for dystonia, the method comprising
a) obtaining a biosample from a subject having a dystonia;
b) determining the expression level of one or more dystonia biomarkers in the biosample to create a proteomic profile; and
c) comparing the subject's proteomic profile to a proteomic profile of a treatment-responsive subject; and
i) predicting that the subject having a dystonia will be responsive to the treatment when the proteomic profiles are similar; or
ii) predicting that the subject having a dystonia will not be responsive to the treatment when the proteomic profiles are aberrant,
wherein the one or more dystonia biomarkers comprises
(i) a protein selected from List-A1, List-A2, List-A3, List-A4, List-B1, List-B2, List-B3, List-C1, List-C2, List-C3, List-D1, List-D2, List-D3, List-E1, List-E2, List-E3, List-F1, List-F2, List-F3, List-G1, List-G2, List-G3, TABLE 4, TABLE 5, TABLE 6, TABLE 7, TABLE 8, TABLE 9, TABLE S1, TABLE S3, TABLE S4, TABLE S5, TABLE S6, TABLE S7, or a combination thereof;
(ii) an miRNA selected from List-MR1, TABLE 2, TABLE 3, or a combination thereof; or
(iii) a metabolite selected from List-ML1, or a combination thereof.

7. A method of classifying a subject having dystonia, the method comprising
a) measuring one or more dystonia biomarkers in the subject from a biological sample obtained from the subject, and
b) classifying the subject as
i) having the one or more dystonia biomarkers when the one or more dystonia biomarkers in the subject is aberrant compared to a reference level, or
ii) not having the one or more dystonia biomarker when the one or more dystonia biomarkers in the subject is not aberrant compared to a reference level,
wherein the one or more dystonia biomarkers comprises
(a) a protein selected from List-A1, List-A2, List-A3, List-A4, List-B1, List-B2, List-B3, List-C1, List-C2, List-C3, List-D1, List-D2, List-D3, List-E1, List-E2, List-E3, List-F1, List-F2, List-F3, List-G1, List-G2, List-G3, TABLE 4, TABLE 5, TABLE 6, TABLE 7, TABLE 8, TABLE 9, TABLE S1, TABLE S3, TABLE S4, TABLE S5, TABLE S6, TABLE S7, or a combination thereof;
(b) an miRNA selected from List-MR1, TABLE 2, TABLE 3, or a combination thereof; or
(c) a metabolite selected from List-ML1, or a combination thereof.

8. A method of identifying a dystonia biomarker in a subject, the method comprising:
a) obtaining a biosample from a subject having a dystonia;
b) obtaining a biosample from a subject not having a dystonia;
c) determining the reference level of one or more proteins, miRNAs, and/or metabolites in the biosample from a subject having a dystonia; and
d) identifying those proteins, miRNAs, and/or metabolites that are aberrant in the biosample obtained from the subject having a dystonia when compared to the biosample from the subject not having a dystonia; wherein those aberrant proteins, miRNAs, and/or metabolites are biomarkers of dystonia,
optionally wherein the biosample comprises extracellular vesicles,
further optionally wherein the method further comprises treating the subject having a dystonia, wherein treating the subject comprises administering one or more therapeutic agents, further optionally wherein the one or more therapeutic agents modulates the expression level of one or more aberrant dystonia biomarker.

9. The method of claim 8, wherein determining the level of one or more proteins or metabolites in a biosample as a dystonia biomarker comprises using \liquid chromatography with tandem mass spectrometry (LC-MS-MS), parallel reaction monitoring (PRM), or multiple reaction monitoring (MRM); or determining the level of one or more miRNAs in a biosample as a dystonia biomarker can comprise using RNASeq or RT-qPCR,
optionally wherein the biosample comprises extracellular vesicles,
further optionally wherein the method further comprises treating the subject having a dystonia, wherein treating the subject comprises administering one or more therapeutic agents, further optionally wherein the one or more therapeutic agents modulates the expression level of one or more aberrant dystonia biomarker.

10. (canceled)

11. (canceled)

12. The method of claim 1, wherein
a) each of the one or more aberrant dystonia biomarkers is below its reference level;
b) each of the one or more aberrant dystonia biomarkers is above its reference level; or
c) at least one of the one or more aberrant dystonia biomarkers is below its reference level, and at least one of the one or more aberrant dystonia biomarkers is above its reference level.

13. The method of claim 1, wherein the dystonia biomarker is aberrant by at least ±0.5 fold, at least ±1.0 fold, at least ±1.5 fold, at least ±2.0 fold, at least ±3.0 fold, or at least ±4.0 fold relative to a reference level.

14. The method of claim 1, wherein the reference level of the one or more dystonia biomarkers is
a) a control value determined from a subject or group of subjects, each of which does not have dystonia; or
b) a normal or desired value, optionally wherein the normal or desired value is according to a Diagnostic and Statistical Manual (DSM).

15. The method of claim 1, wherein the one or more dystonia biomarkers is an extracellular vesicle (EV) biomarker.

16. The method of claim 1, wherein the one or more dystonia biomarkers is selected from a protein, metabolite, or an miRNA.

17. (canceled)

18. The method of claim 1, wherein the dystonia comprises focal dystonia, blepharospasm, cervical dystonia, oromandibular dystonia, task-specific or occupational dystonia, spasmodic dysphonia, generalized dystonia, segmental dystonia, DYT1-related dystonia, DYT6-related dystonia, DYT28-related dystonia, dopa-responsive dystonia, myoclonic dystonia, X-linked dystonia-Parkinsonism, rapid-onset dystonia-Parkinsonism, paroxysmal dystonia choreoathetosis, paroxysmal kinesigenic dystonia, paroxysmal nonkinesigenic dyskinesia, paroxysmal exertion-induced dyskinesia, primary dystonia, acquired dystonia, tardive dyskinesia, or tardive dystonia.

19. The method of claim 1, wherein the one or more therapeutic agents treats, reduces, ameliorates, prevents, or inhibits a symptom of a neurological disorder.

20. The method of claim 1, wherein an agent that treats, reduces, ameliorates, prevents, or inhibits a symptom of a neurological disorder is an anticholinergic, benzodiazepine, dopaminergic agent, or a dopamine-depleting agent.

21. The method of claim 20, wherein
i) the anticholinergic is selected from trihexyphenidyl (Artane®), benztropine (Cogentin®), or ethopropazine (Parsitan®);
ii) the benzodiazepine is selected from diazepam (Valium®), clonazepam (Klonopin®), lorazepam (Ativan®); or baclofen (Lioresal®);
iii) the dopaminergic agent is selected from levodopa (Sincmet®), bromocriptine (Parlodel®), or tetrabenazine (Xenazine®); or
iv) the dopamine-depleting agent is selected from ritonavir or lopinavir.

22. The method of claim 1, wherein the one or more therapeutic agents is selected from Acamprosate tablets (Campral EC), Adrenaline (epinephrine) (Emerade, EpiPen, Jext), Agomelatine tablets (Valdoxan), Almotriptan (Almogran), Amantadine, Amisulpride (Solian), Amitriptyline (Elavil), Apomorphine (APO-go, Dacepton), Aripiprazole (Abilify), Aripiprazole long-acting injection (Ability Maintena), Asenapine tablets (Sycrest), Atomoxetine (Strattera), Baclofen (Lyflex, Lioresal), Botulinum toxin type A (Botox), Bromocriptine (Parlodel), Buccal midazolam (Buccolam, Epistatus), Buprenorphine (BuTrans, Hapoctasin, Temgesic, Tephine, Transtec), Buspirone, Cabergoline tablets (Dostinex, Cabaser), Carbamazepine (Curatil, Tegretol), Chlordiazepoxide (Librium), Chlorpromazine, Citalopram (Cipramil, Celexa), Clobazam (Frisium, Perizam, Tapclob, Zacco), Clomethiazole, Clomipramine, Clonazepam, Clozapine (Clozaril, Denzapine, Zaponex), Co-beneldopa (Madopar), Co-careldopa (Sinemet), Dantrolene (Dantrium), Dexamfetamine (Amfexa), Diazepam (Diazemuls, Stesolid), Divalproex sodium (Depakote), Donepezil (Aricept), Doxepin capsules, Duloxetine (Cymbalta, Depalta, Duciltia), Eletriptan (Relpax), Entacapone (Comtess), Escitalopram (Cipralex), Eslicarbazepine (Zebinix), Ethosuximide, Fingolimod capsules (Gilenya), Fluoxetine (Olena, Prozac, Prozep), Flupentixol long-acting injection (Depixol, Psytixol), Flupentixol tablets (Depixol, Fluanxol), Fluphenazine long-acting injection (Modecate), Fluvoxamine tablets (Faverin), Frovatriptan for migraine (Migard), Gabapentin (Neurontin), Galantamine (Acumor, Consion, Elmino, Gaalin, Galsya, Galzemic, Gatalin, Gazylan, Lotprosin, Luventa, Reminyl), Haloperidol (Haldol, Serenace), Haloperidol long-acting injection (Haldol Decanoate), Hydromorphone (Palladone), Imipramine tablets and liquid medicine, Lacosamide (Vimpat), Lamotrigine (Lamictal), Levetiracetam for epilepsy (Keppra, Desitrend), Levomepromazine tablets (Nozinan), Lisdexamfetamine (Elvanse), Lithium tablets and liquid medicine (Camcolit, Liskonum, Priadel, Li-Liquid), Lofepramine, Loprazolam, Lorazepam, Lormetazepam tablets, Lurasidone (Latuda), Melatonin tablets (Circadin, Slenyto), Memantine (Ebixa, Nemtadine), Methylphenidate (Concerta, Equasym, Medikinet, Ritalin, Tranquilyn), Mianserin, Midodrine (Bramox), Mirtazapine (Zispin SolTab), Moclobemide (Manerix), Modafinil tablets (Provigil), Morphine (Morphgesic, Oramorph, Zomorph), Naratriptan (Naramig), Neostigmine, Nitrazepam (Mogadon), Nortriptyline tables, Olanzapine (Zalasta, Zyprexa), Olanzapine long-acting injection (Zypadhera), Orlistat capsules (Alii, Beacita, Orlos, Xenical), Orphenadrine, Oxazepam, Oxcarbazepine (Trileptal), Oxycodone (Abtard, Longtec, OxyContin, OxyNorm, Shortec), Paliperidone (Invega), Paliperidone long-acting injection (Xeplion, Trevicta), Paroxetine (Seroxat), Perampanel (Fycompa), Pergolide, Pericyazine, Phenobarbital, Phenytoin (Epanutin), Piracetam (Nootropil), Pizotifen tablets, Pramipexole tablets (Mirapexin, Oprymea, Pipexus, Glepark), Pregabalin (Alzain, Axalid, Lecaent, Lyrica), Primidone, Prochlorperazine (Buccastem, Stemetil), Procyclidine (Kemadrin), Pyridostigmine (Mestinon), Quetiapine (Seroquel), Rasagiline (Azilect), Reboxetine tablets (Edronax), Risperidone (Risperdal), Risperidone long-acting injection (Risperdal Consta), Rivastigmine (Alzest, Exelon, Nimvastid), Rizatriptan for migraine (Maxalt), Ropinirole tablets (Requip, Adartrel), Rotigotine patches (Neupro), Rufmamide for epilepsy (Inovelon), Selegiline (Eldepryl), Sertraline (Lustral, Zoloft), Sodium oxybate (Xyrem), Sodium valproate (Epilim, Episenta, Epival, Convulex), Sulpiride, Sumatriptan (Imigran), Temazepam, Tetrabenazine tablets (Tardiben, Xenazine), Tiagabine (Gabitril), Tizanidine, Tolcapone (Tasmar), Topiramate (Topamax), Topiramate (Topamax), Trazodone (Molipaxin), Trihexyphenidyl, Trimipramine, Valproate semisodium (Belvo, Depakote, Syonell), Venlafaxine (Efexor XL, Effexor XR), Vigabatrin (Sabril, Kigabeq), Vortioxetine (Brintellix), Zolmitriptan (Zomig), Zolpidem tablets (Stilnoct), Zonisamide (Zonegran, Desizon), Zopiclone tablets (Zimovane), or Zuclopenthixol (Clopixol).

23. A pharmaceutical composition for treatment of a dystonia in a subject in need thereof, comprising one or more therapeutic agents as described herein and a pharmaceutically acceptable carrier, wherein the treatment comprises administering the pharmaceutical composition to the subject when one or more dystonia biomarkers in the subject is aberrant compared to a reference level.


SEQUENCE LISTING (14 sequences)

SEQ ID NO: 1 - RNA, 22 nt, Homo sapiens
  source 1..22: mol_type = other RNA
tgaggtagtaggttgtgtggtt

SEQ ID NO: 2 - RNA, 22 nt, Homo sapiens
  source 1..22: mol_type = other RNA
agaggtagtaggttgcatagtt

SEQ ID NO: 3 - RNA, 22 nt, Homo sapiens
  source 1..22: mol_type = other RNA
tgaggtagtagtttgtacagtt

SEQ ID NO: 4 - RNA, 17 nt, Homo sapiens
  source 1..17: mol_type = other RNA
gtgggggagaggctgtc

SEQ ID NO: 5 - RNA, 22 nt, Homo sapiens
  source 1..22: mol_type = other RNA
tggaatgtaaagaagtatgtat

SEQ ID NO: 6 - RNA, 22 nt, Homo sapiens
  source 1..22: mol_type = other RNA
gtgaaatgtttaggaccactag

SEQ ID NO: 7 - RNA, 22 nt, Homo sapiens
  source 1..22: mol_type = other RNA
aatctgagaaggcgcacaaggt

SEQ ID NO: 8 - RNA, 22 nt, Homo sapiens
  source 1..22: mol_type = other RNA
tattgcacattactaagttgca

SEQ ID NO: 9 - RNA, 22 nt, Homo sapiens
  source 1..22: mol_type = other RNA
ttttgtgtctcccattccccag

SEQ ID NO: 10 - RNA, 24 nt, Homo sapiens
  source 1..24: mol_type = other RNA
tgagaaggcacagcttgcacgtga

SEQ ID NO: 11 - RNA, 24 nt, Homo sapiens
  source 1..24: mol_type = other RNA
tggaagactagtgattttgttgtt

SEQ ID NO: 12 - RNA, 23 nt, Homo sapiens
  source 1..23: mol_type = other RNA
tctttggttatctagctgtatga

SEQ ID NO: 13 - RNA, 22 nt, Homo sapiens
  source 1..22: mol_type = other RNA
tgaggtagtaagttgtattgtt

SEQ ID NO: 14 - RNA, 22 nt, Homo sapiens
  source 1..22: mol_type = other RNA
caagctcgcttctatgggtctg